SEQUENCE LISTING (278 sequences)

SEQ ID NO: 1 - DNA, 6492 bp, Homo sapiens
  source 1..6492: mol_type = genomic DNA
ggcggctagggaggtggggcgaggcgaggtttgctggggtgaggcagcggcgcggccgggccgggccgggccacaggcggtggcggcgggaccatggaggcggcggtcgctgctccgcgtccccggctgctcctcctcgtgctggcggcggcggcggcggcggcggcggcgctgctcccgggggcgacggcgttacagtgtttctgccacctctgtacaaaagacaattttacttgtgtgacagatgggctctgctttgtctctgtcacagagaccacagacaaagttatacacaacagcatgtgtatagctgaaattgacttaattcctcgagataggccgtttgtatgtgcaccctcttcaaaaactgggtctgtgactacaacatattgctgcaatcaggaccattgcaataaaatagaacttccaactactgtaaagtcatcacctggccttggtcctgtggaactggcagctgtcattgctggaccagtgtgcttcgtctgcatctcactcatgttgatggtctatatctgccacaaccgcactgtcattcaccatcgagtgccaaatgaagaggacccttcattagatcgcccttttatttcagagggtactacgttgaaagacttaatttatgatatgacaacgtcaggttctggctcaggtttaccattgcttgttcagagaacaattgcgagaactattgtgttacaagaaagcattggcaaaggtcgatttggagaagtttggagaggaaagtggcggggagaagaagttgctgttaagatattctcctctagagaagaacgttcgtggttccgtgaggcagagatttatcaaactgtaatgttacgtcatgaaaacatcctgggatttatagcagcagacaataaagacaatggtacttggactcagctctggttggtgtcagattatcatgagcatggatccctttttgattacttaaacagatacacagttactgtggaaggaatgataaaacttgctctgtccacggcgagcggtcttgcccatcttcacatggagattgttggtacccaaggaaagccagccattgctcatagagatttgaaatcaaagaatatcttggtaaagaagaatggaacttgctgtattgcagacttaggactggcagtaagacatgattcagccacagataccattgatattgctccaaaccacagagtgggaacaaaaaggtacatggcccctgaagttctcgatgattccataaatatgaaacattttgaatccttcaaacgtgctgacatctatgcaatgggcttagtattctgggaaattgctcgacgatgttccattggtggaattcatgaagattaccaactgccttattatgatcttgtaccttctgacccatcagttgaagaaatgagaaaagttgtttgtgaacagaagttaaggccaaatatcccaaacagatggcagagctgtgaagccttgagagtaatggctaaaattatgagagaatgttggtatgccaatggagcagctaggcttacagcattgcggattaagaaaacattatcgcaactcagtcaacaggaaggcatcaaaatgtaattctacagctttgcctgaactctccttttttcttcagatctgctcctgggttttaatttgggaggtcaattgttctacctcactgagagggaacagaaggatattgcttccttttgcagcagtgtaataaagtcaattaaaaacttcccaggatttctttggacccaggaaacagccatgtgggtcctttctgtgcactatgaacgcttctttcccaggacagaaaatgtgtagtctacctttattttttattaacaaaacttgttttttaaaaagatgattgctggtcttaactttaggtaactctgctgtgctggagatcatctttaagggcaaaggagttggattgctgaattacaatgaaacatgtcttattactaaagaaagtgatttactcctggttagtacattctcagaggattctgaaccactagagtttccttgattcagactttgaatgtactgttctatagtttttcaggatcttaaaactaacacttataaaactcttatcttgagtctaaaaatgacctcatatagtagtgaggaacataattcatgcaattgtattttgtatactattattgttctttcacttattcagaacattacatgccttcaaaatgggattgtactataccagtaagtgccacttctgtgtctttctaatggaaatgagtagaattgctgaaagtctctatgttaaaacctatagtgtttgaattcaaaaagcttatttatctgggtaacccaaactttttctgttttgtttttggaagggtttttgtggtatgtcatttggtattctattctgaaaatgcctttctcctaccaaaatgtgcttaagccactaaagaaatgaagtggcattaattagtaaattattagcatggtcatgtttgaatattctcacatcaagcttttgcattttaattgtgttgtctaagtatacttttaaaaaatcaagtggcactctagatgcttatagtactttaatatttgtagcatacagactaatttttctaaaagggaaagtctgtctagctgcttgtgaaaagttatgtggtattctgtaagccatttttttctttatctgttcaaagacttattttttaagacatgaattacatttaaaattagaatatggttaatattaaataataggcctttttctaggaaggcgaaggtagttaataatttgaatagataacagatgtgcaagaaagtcacatttgttatgtatgtaggagtaaacgttcggtggatcctctgtctttgtaactgaggttagagctagtgtggttttgaggtctcactacactttgaggaaggcagcttttaattcagtgtttccttatgtgtgcgtacattgcaactgcttacatgtaatttatgtaatgcattcagtgcacccttgttacttgggagaggtggtagctaaagaacattctgagtataggtttttctccatttacagatgtctttggtcaaatattgaaagcaaacttgtcatggtcttcttacattaagttgaaactagcttataataactggtttttacttccaatgctatgaagtctctgcagggcttttacagttttcgaagtccttttatcactgtgatcttattctgaggggagaaaaaactatcatagctctgaggcaagacttcgactttatagtgctatcagttccccgatacagggtcagagtaacccatacagtattttggtcaggaagagaaagtggccatttacactgaatgagttgcattctgataatgtcttatctcttatacgtagaataaatttgaaagactatttgatcttaaaaccaaagtaattttagaatgagtgacatattacataggaatttagtgtcaatttcatgtgtttaaaaacatcatgggaaaaatgcttagaggttactattttgactacaaagttgagtttttttctgtagttaccataatttcattgaagcaaatgaatgagtttgagaggtttgtttttatagttgtgttgtattacttgtttaataataatctctaattctgtgatcaggtactttttttgtgggggttttttttttgtttttttttttttgttgttgtttttgggccatttctaagcctaccagatctgctttatgaaatccaggggaccaatgcattttatcactaaaactatttttatataattttaagaatataccaaaagttgtctgatttaaagttgtaatacatgatttctcactttcatgtaaggttatccacttttgctgaagatattttttattgaatcaaagattgagttacaattatacttttcttacctaagtggataaaatgtacttttgatgaatcagggaatttttttaaagttggagtttagttctaaattgactttacgtattactgcagttaattccttttttggctagggatggtttgataaaccacaattggctgatattgaaaatgaaagaaacttaaaaggtgggatggatcatgattactgtcgataactgcagataaatttgattagagtaataattttgtcatttaaaaacacagttgtttatactgcccatcctaggatgctcaccttccaagattcaacgtggctaaaacatcttctggtaaattgtgcgtccatattcattttgtcagtagccaggagaaatggggatgggggaaatacgacttagtgaggcatagacatccctggtccatcctttctgtctccagctgtttcttggaacctgctctcctgcttgctggtccctgacgcagagaccgttgcctcccccacagccgtttgactgaaggctgctctggagacctagagtaaaacggctgatggaagttgtgggacccacttccatttccttcagtcattagaggtggaagggaggggtctccaagtttggagattgagcagatgaggcttgggatgcccctgctttgacttcagccatggatgaggagtgggatggcagcaaggtggctcctgtggcagtggagttgtgccagaaacagtggccagttgtatcgcctataagacagggtaaggtctgaagagctgagcctgtaattctgctgtaataatgatagtgctcaagaagtgccttgagttggtgtacagtgccatggccatcaagaatcccagatttcaggttttattacaaaatgtaagtggtcacttggcgattttgtagtacatgcatgagttaccttttttctctatgtctgagaactgtcagattaaaacaagatggcaaagagatcgttagagtgcacaacaaaatcactatcccattagacacatcatcaaaagcttatttttattcttgcactggaagaatcgtaagtcaactgtttcttgaccatggcagtgttctggctccaaatggtagtgattccaaataatggttctgttaacactttggcagaaaatgccagctcagatattttgagatactaaggattatctttggacatgtactgcagcttcttgtctctgttttggattactggaatacccatgggccctctcaagagtgctggacttctaggacattaagatgattgtcagtacattaaacttttcaatcccattatgcaatcttgtttgtaaatgtaaacttctaaaaatatggttaataacattcaacctgtttattacaacttaaaaggaacttcagtgaatttgtttttattttttaacaagatttgtgaactgaatatcatgaaccatgttttgatacccctttttcacgttgtgccaacggaatagggtgtttgatatttcttcatatgttaaggagatgcttcaaaatgtcaattgctttaaacttaaattacctctcaagagaccaaggtacatttacctcattgtgtatataatgtttaatatttgtcagagcattctccaggtttgcagttttatttctataaagtatgggtattatgttgctcagttactcaaatggtactgtattgtttatatttgtaccccaaataacatcgtctgtactttctgttttctgtattgtatttgtgcaggattctttaggctttatcagtgtaatctctgccttttaagatatgtacagaaaatgtccatataaatttccattgaagtcgaatgatactgagaagcctgtaaagaggagaaaaaaacataagctgtgtttccccataagtttttttaaattgtatattgtatttgtagtaatattccaaaagaatgtaaataggaaatagaagagtgatgcttatgttaagtcctaacactacagtagaagaatggaagcagtgcaaataaattacatttttcccaagtgccagtggcatattttaaaataaagtgtatacgttggaatgagtcatgccatatgtagttgctgtagatggcaactagaacctttgagttacaagagtctttagaagttttctaaccctgcctagtgcaagttacaatattatagcgtgttcggggagtgccctcctgtctgcaggtgtgtctctgtgcctgggggcttttctccacatgcttaggggtgtgggtcttccattggggcatgatggacctgtctacaggtgatctctgttgcctttgggtcagcacatttgttagtctcctgggggtgaaaacttggcttacaagagaactggaaaaatgatgagatgtggtccccaaacccttgattgactctggggaggggctttgtgaataggattgctctcacattaaagatagttacttcaatttgaaggctggatttagggatttttttttttccttataacaaagacatcaccaggatatgaagcttttgttgaaagttggaaaaaaagtgaaattaaagacattcccagacaaa

SEQ ID NO: 2 - RNA, 4530 nt, Homo sapiens
  source 1..4530: mol_type = mRNA
actcgcgcgcacggagcgacgacacccccgcgcgtgcacccgctcgggacaggagccggactcctgtgcagcttccctcggccgccgggggcctccccgcgcctcgccggcctccaggccccctcctggctggcgagcgggcgccacatctggcccgcacatctgcgctgccggcccggcgcggggtccggagagggcgcggcgcggaggcgcagccaggggtccgggaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgttgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaatagctcttctggggcaggctgggccatgtccaaagaggctgcccctctcaccaaagaacagaggcagcaggaagctgcccctgaactgatgcttcctggaaaaccaagggggtcactcccctccctgtaagctgtggggataagcagaaacaacagcagcagggagtgggtgacatagagcattctatgcctttgacattgtcataggataagctgtgttagcacttcctcaggaaatgagattgatttttacaatagccaataacatttgcactttattaatgcctgtatataaatatgaatagctatgttttatatatatatatatatatctatatatgtctatagctctatatatatagccataccttgaaaagagacaaggaaaaacatcaaatattcccaggaaattggttttattggagaactccagaaccaagcagagaaggaagggacccatgacagcattagcatttgacaatcacacatgcagtggttctctgactgtaaaacagtgaactttgcatgaggaaagaggctccatgtctcacagccagctatgaccacattgcacttgcttttgcaaaataatcattccctgcctagcacttctcttctggccatggaactaagtacagtggcactgtttgaggaccagtgttcccggggttcctgtgtgcccttatttctcctggacttttcatttaagctccaagccccaaatctggggggctagtttagaaactctccctcaacctagtttagaaactctaccccatctttaataccttgaatgttttgaaccccactttttaccttcatgggttgcagaaaaatcagaacagatgtccccatccatgcgattgccccaccatctactaatgaaaaattgttctttttttcatctttcccctgcacttatgttactattctctgctcccagccttcatccttttctaaaaaggagcaaattctcactctaggctttatcgtgtttactttttcattacacttgacttgattttctagttttctatacaaacaccaatgggttccatctttctgggctcctgattgctcaagcacagtttggcctgatgaagaggatttcaactacacaatactatcattgtcaggactatgacctcaggcactctaaacatatgttttgtttggtcagcacagcgtttcaaaaagtgaagccactttataaatatttggagattttgcaggaaaatctggatccccaggtaaggatagcagatggttttcagttatctccagtccacgttcacaaaatgtgaaggtgtggagacacttacaaagctgcctcacttctcactgtaaacattagctctttccactgcctacctggaccccagtctaggaattaaatctgcacctaaccaaggtcccttgtaagaaatgtccattcaagcagtcattctctgggtatataatatgattttgactaccttatctggtgttaagatttgaagttggccttttattggactaaaggggaactcctttaagggtctcagttagcccaagtttcttttgcttatatgttaatagttttaccctctgcattggagagaggagtgctttactccaagaagctttcctcatggttaccgttctctccatcatgccagccttctcaacctttgcagaaattactagagaggatttgaatgtgggacacaaaggtcccatttgcagttagaaaatttgtgtccacaaggacaagaacaaagtatgagctttaaaactccataggaaacttgttaatcaacaaagaagtgttaatgctgcaagtaatctcttttttaaaactttttgaagctacttattttcagccaaataggaatattagagagggactggtagtgagaatatcagctctgtttggatggtggaaggtctcattttattgagatttttaagatacatgcaaaggtttggaaatagaacctctaggcaccctcctcagtgtgggtgggctgagagttaaagacagtgtggctgcagtagcatagaggcgcctagaaattccacttgcaccgtagggcatgctgataccatcccaatagctgttgcccattgacctctagtggtgagtttctagaatactggtccattcatgagatattcaagattcaagagtattctcacttctgggttatcagcataaactggaatgtagtgtcagaggatactgtggcttgttttgtttatgtttttttttcttattcaagaaaaaagaccaaggaataacattctgtagttcctaaaaatactgacttttttcactactatacataaagggaaagttttattcttttatggaacacttcagctgtactcatgtattaaaataggaatgtgaatgctatatactctttttatatcaaaagtctcaagcacttatttttattctatgcattgtttgtcttttacataaataaaatgtttattagattgaataaagcaaaatactcaggtgagcatcctgcctcctgttcccattcctagtagctaaa

SEQ ID NO: 3 - RNA, 3696 nt, Homo sapiens
  source 1..3696: mol_type = mRNA
ctcttctcccgcgggttggtggacccgctcagtacggagttggggaagctctttcacttcggaggattgctcaacaaccatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagagtgaaaaacaacaaattcagttctgagtatatgcaattagtgtttgaaaagattcttaatagctggctgtaaatactgcttggttttttactgggtacattttatcatttattagcgctgaagagccaacatatttgtagatttttaatatctcatgattctgcctccaaggatgtttaaaatctagttgggaaaacaaacttcatcaagagtaaatgcagtggcatgctaagtacccaaataggagtgtatgcagaggatgaaagattaagattatgctctggcatctaacatatgattctgtagtatgaatgtaatcagtgtatgttagtacaaatgtctatccacaggctaaccccactctatgaatcaatagaagaagctatgaccttttgctgaaatatcagttactgaacaggcaggccactttgcctctaaattacctctgataattctagagattttaccatatttctaaactttgtttataactctgagaagatcatatttatgtaaagtatatgtatttgagtgcagaatttaaataaggctctacctcaaagacctttgcacagtttattggtgtcatattatacaatatttcaattgtgaattcacatagaaaacattaaattataatgtttgactattatatatgtgtatgcattttactggctcaaaactacctacttctttctcaggcatcaaaagcattttgagcaggagagtattactagagctttgccacctctccatttttgccttggtgctcatcttaatggcctaatgcacccccaaacatggaaatatcaccaaaaaatacttaatagtccaccaaaaggcaagactgcccttagaaattctagcctggtttggagatactaactgctctcagagaaagtagctttgtgacatgtcatgaacccatgtttgcaatcaaagatgataaaatagattcttatttttcccccacccccgaaaatgttcaataatgtcccatgtaaaacctgctacaaatggcagcttatacatagcaatggtaaaatcatcatctggatttaggaattgctcttgtcatacccccaagtttctaagatttaagattctccttactactatcctacgtttaaatatctttgaaagtttgtattaaatgtgaattttaagaaataatatttatatttctgtaaatgtaaactgtgaagatagttataaactgaagcagatacctggaaccacctaaagaacttccatttatggaggatttttttgccccttgtgtttggaattataaaatataggtaaaagtacgtaattaaataatgtttttggtatttctggttttctcttttttggtaggggcttgctttttggttttgtcttccttttctctaactgatgctaaatataacttgtctttaatgcttcttggatcccttagaaggtacttcctttttaaccttaacccttttagtagttaaataattatttccataggttgctattgccaagaagacctcttccaaacagcacatgattattcgtcaaacagtttcgtattccagatactggaatgtggataagaaagtatacatttcaaggggtaggttttattattaagaaagccaaatgaggattttgaaatattctttcctgcatattatccattctagctacatgctggccagtgggccacctttcttttctgcaatttaatgctagtaatatattctatttaacccatgagtcccaaagtattagcatttcaacatgtaagcatgtcggtaagatagttgtgctttgcttagggttccctcctgtgttatggtctggaaagtgtctttaggcagaaagtctgagtgatcacagggttcactcattaatttctcttttctgagccatcatagtctgtgctgtctgctctccagttttctatttctagacagaagtagggcaagttaggtactagttattcttcatggccagaagtgcaagttctactttgcaagacaagattaagttagagaacaccctattccactttggtgaactcagagcaagaactttgagttcctttgggaggaagacagtggagaagtctttgtacttggtgatgtggtttttttcctcatggcttcacctagtggccccaagcatgacttctcccatgtcaatgagcacagccacattcccgagttgaggtgaccccacggtccagaatcatcctcattctggtgaacctggttctctttgtggtgggcatactgggtaggagaatcacccaaaggtcacccatgagctgcagaaaaaaaggctatttgcagaaggagctcacagatcacattgaaagcattgcatattcaaacatcttggtcttctttattggcatgcccacagggtcttctgacctctgattagatcagacactttttagatattgaatcatcagtttctgtacaactatctgaataaggtatataatcaatgaaatttagaatttttttctatgcttactcctgattggtaatttgtttgggtttagaattctatacaaggccatttgtaattttcctcagcactttaaaaatattaaaccatgttttcttaa

SEQ ID NO: 4 - RNA, 3470 nt, Homo sapiens
  source 1..3470: mol_type = mRNA
gcatgcgccgcagcgccagcgctctccccggatcgtgcggggcctgagcctctccgccggcgcaggctctgctcgcgccagctcgctcccgcagccatgcccaccaccatcgagcgggagttcgaagagttggatactcagcgtcgctggcagccgctgtacttggaaattcgaaatgagtcccatgactatcctcatagagtggccaagtttccagaaaacagaaatcgaaacagatacagagatgtaagcccatatgatcacagtcgtgttaaactgcaaaatgctgagaatgattatattaatgccagtttagttgacatagaagaggcacaaaggagttacatcttaacacagggtccacttcctaacacatgctgccatttctggcttatggtttggcagcagaagaccaaagcagttgtcatgctgaaccgcattgtggagaaagaatcggttaaatgtgcacagtactggccaacagatgaccaagagatgctgtttaaagaaacaggattcagtgtgaagctcttgtcagaagatgtgaagtcgtattatacagtacatctactacaattagaaaatatcaatagtggtgaaaccagaacaatatctcactttcattatactacctggccagattttggagtccctgaatcaccagcttcatttctcaatttcttgtttaaagtgagagaatctggctccttgaaccctgaccatgggcctgcggtgatccactgtagtgcaggcattgggcgctctggcaccttctctctggtagacacttgtcttgttttgatggaaaaaggagatgatattaacataaaacaagtgttactgaacatgagaaaataccgaatgggtcttattcagaccccagatcaactgagattctcatacatggctataatagaaggagcaaaatgtataaagggagattctagtatacagaaacgatggaaagaactttctaaggaagacttatctcctgcctttgatcattcaccaaacaaaataatgactgaaaaatacaatgggaacagaataggtctagaagaagaaaaactgacaggtgaccgatgtacaggactttcctctaaaatgcaagatacaatggaggagaacagtgagagtgctctacggaaacgtattcgagaggacagaaaggccaccacagctcagaaggtgcagcagatgaaacagaggctaaatgagaatgaacgaaaaagaaaaaggtggttatattggcaacctattctcactaagatggggtttatgtcagtcattttggttggcgcttttgttggctggacactgttttttcagcaaaatgccctataaacaattaattttgcccagcaagcttctgcactagtaactgacagtgctacattaatcataggggtttgtctgcagcaaacgcctcatatcccaaaaacggtgcagtagaatagacatcaaccagataagtgatatttacagtcacaagcccaacatctcaggactcttgactgcaggttcctctgaaccccaaactgtaaatggctgtctaaaataaagacattcatgtttgttaaaaactggtaaattttgcaactgtattcatacatgtcaaacacagtatttcacctgaccaacattgagatatcctttatcacaggatttgtttttggaggctatctggattttaacctgcacttgatataagcaataaatattgtggttttatctacgttattggaaagaaaatgacatttaaataatgtgtgtaatgtataatgtactattgacatgggcatcaacacttttattcttaagcatttcagggtaaatatattttataagtatctatttaatcttttgtagttaactgtactttttaagagctcaatttgaaaaatctgttactaaaaaaataaattgtatgtcgattgaattgtactggatacattttccatttttctaaagagaagtttgatatgagcagttagaagttggaataagcaatttctactatatattgcatttcttttatgttttacagttttccccattttaaaaagaaaagcaaacaaagaaacaaaagtttttcctaaaaatatctttgaaggaaaattctccttactgggatagtcaggtaaacagttggtcaagactttgtaaagaaattggtttctgtaaatcccattattgatatgtttatttttcatgaaaatttcaatgtagttggggtagattatgatttaggaagcaaaagtaagaagcagcattttatgattcataatttcagtttactagactgaagttttgaagtaaacacttttcagtttctttctacttcaataaatagtatgattatatgcaaaccttacattgtcattttaacttaatgaatattttttaaagcaaactgtttaatgaatttaactgctcatttgaatgctagctttcctcagatttcaacattccattcagtgtttaatttgtcttacttaaacttgaaattgttgttacaaatttaattgctaggaggcatggatagcatacattattatggatagcataccttatttcagtggttttcaaactatgctcattggatgtccaggtgggtcaagaggttactttcaaccacagcatctctgccttgtctctttatatgccacataagatttctgcataaggcttaagtattttaaagggggcagttatcatttaaaaacagtttggtcgggcgcggtggctcatgcctgtaatcccagcactttgggaggctgaagtgggcagatcacctgaggtcaggagttcaagaccagcctggccaacgtggtgaaacaccatctctactaaaaatgcaaaaattagctgggcatggtggagggcacctgtaatctcagctactcaggaggctgaggtaggagaattgcttgaacccaggagatggaggttgcagtgagctgagatcacgtcactgcactccagccagggcgacagagcgagactccatctcaaaagaaacaaacaaaaaaaacagtttgggccgggtgtggtggctcacgcttgtaatcccagcacttcggaaggccaaggcgggcggatcacgaggtcaagagatggagactgtcctggccaacatggtgaaatcccttctttactaaaaatacaaaaattatctgggcgtggtggtgcatgcctgtagtcccagctccttgggaggctaaggcaggagaatcacttgaacccgggaggcagaggttgcagtgagccgagattgcaccactgcactccagcctggcaacagagcaagacttcgtctcaaaaaaaaaaaaaaaaaaagtttgaaaaccattggtatagatagatattttgaattgatttgcatagtctccttgaatgtgttaaattatgttgaaagtatgaaagcaggatgtaggtggtactacatattaaataagatttatataaca

SEQ ID NO: 5 - RNA, 4076 nt, Homo sapiens
  source 1..4076: mol_type = mRNA
ctcttcttcttaaacaaaccacaaacggatgtgagggaaggaaggtgtttcttttactcctgagcccagacacctcactctgttccgtctaagcttgttttgctgaacacttttttttaaaaaaggaaaaagaaaaggagttgcttgatgtgagagtgaaatggacgtaagattttatccacctccagcccagcccgccgctgcgcccgacgctccctgtctgggaccttctccctgcctggacccctactattgcaacaagtttgacggtgagaacatgtatatgagcatgacagagccgagccaggactatgtgccagccagccagtcctaccctggtccaagcctggaaagtgaagacttcaacattccaccaattactcctccttccctcccagaccactcgctggtgcacctgaatgaagttgagtctggttaccattctctgtgtcaccccatgaaccataatggcctgctaccatttcatccacaaaacatggacctccctgaaatcacagtctccaatatgctgggccaggatggaacactgctttctaattccatttctgtgatgccagatatacgaaacccagaaggaactcagtacagttcccatcctcagatggcagccatgagaccaaggggccagcctgcagacatcaggcagcagccaggaatgatgccacatggccagctgactaccattaaccagtcacagctaagtgctcaacttggtttgaatatgggaggaagcaatgttccccacaactcaccatctccacctggaagcaagtctgcaactccttcaccatccagttcagtgcatgaagatgaaggcgatgatacctctaagatcaatggtggagagaagcggcctgcctctgatatggggaaaaaaccaaaaactcccaaaaagaagaagaagaaggatcccaatgagccccagaagcctgtgtctgcctatgcgttattctttcgtgatactcaggccgccatcaagggccaaaatccaaacgctacctttggcgaagtctctaaaattgtggcttcaatgtgggacggtttaggagaagagcaaaaacaggtctataaaaagaaaaccgaggctgcgaagaaggagtacctgaagcaactcgcagcatacagagccagccttgtatccaagagctacagtgaacctgttgacgtgaagacatctcaacctcctcagctgatcaattcgaagccgtcggtgttccatgggcccagccaggcccactcggccctgtacctaagttcccactatcaccaacaaccgggaatgaatcctcacctaactgccatgcatcctagtctccccaggaacatagcccccaagccgaataaccaaatgccagtgactgtctctatagcaaacatggctgtgtcccctcctcctcccctccagatcagcccgcctcttcaccagcatctcaacatgcagcagcaccagccgctcaccatgcagcagccccttgggaaccagctccccatgcaggtccagtctgccttacactcacccaccatgcagcaaggatttactcttcaacccgactatcagactattatcaatcctacatctacagctgcacaagttgtcacccaggcaatggagtatgtgcgttcggggtgcagaaatcctcccccacaaccggtggactggaataacgactactgcagtagtgggggcatgcagagggacaaagcactgtaccttacttgagaatctgaacacctcttctttccactgaggaattcagggaagtgttttcaccatggattgctttgtacagtcaaggcagttctccattttattagaaaatacaagttgctaagcacttaggaccatttgagcttgtgggtcacccactctggaagaaatagtcatgcttctttattatttttttaatcctttatggacattgtttttcttcttccctgaaggaaatttggaccattcagattttatgttggttttttgctgtgaagtgctgcgctctagtaactgccttagcaactgtagatgtctcggataaaagtcctggattttccattggttttcataatgggtgtttatatgaaactactaaagactttttaaatggcttgatgtagcagtcatagcaagtttgtaaatagcatctatgttacactctcctagagtataaaatgtgaatgtttttgtagctaaattgtaattgaaactggctcattccagtttattgatttcacaataggggttaaattggcaaacattcatatttttacttcatttttaaaacaactgactgatagttctatattttcaaaatatttgaaaataaaaagtattcccaagtgattttaatttaaaaacaaattggctttgtctcattgatcagacaaaaagaaactagtattaagggaagcgcaaacacatttattttgtactgcagaaaaattgcttttttgtatcactttttgtgtaatggttagtaaatgtcatttaagtccttttatgtataaaactgccaaatgcttacctggtattttattagatgcagaaacagattggaaacagctaaattacaacttttacatatggctctgtcttattgtttcttcatactgtgtctgtatttaatctttttttatggaacctgttgcgcctatttatgaaataataaatataggtgtttgtaagtaaatttgttagtatttgaaagaggtttctttgatgttttaacttttgctggcaaaaaaaaattcacgcttggtgtgaatactttattatttagtttttacagtaacatgaataaagccaaacctgcttttcatttagcagcaaattaaagtaaccagtccttatttctgcatttctttggttgatgcaaacaaaaaactattatatttaagaactttatttcttcatacgacataacagaattgccctccaagtcacacaagctccaagactaaacaaacagacaggtcctctgtcttaaaaaggttacttcttggttctcagctggttctagtcaattctgaaccaccaccccccgccccccgcaaaaaagtaaaagtcaaaccaaacttcctcaagctgcatgcttttcacaaaatccagaaagcatttaagaattgaactaggggctggaagaagtgaaagggaagcatctaaaaatgaaaggtgagtaaccagatagcaaaagaaaagggaaagccatccaaatttgaaagctgttgatagaaattgagattcttgctgtcttttgtgcctctacaagctactactcattccagaattcctgggtcttccaagaggattcttaaggtaccagagatttgctagggaaccaaaagtgcttgagaatctgcctgagggcttgcatagctttcacattaaaaaaagaaaaagctagcagatttactcctttttaggggatcatatcaagaaagttagtctggttggaaaccaagagaatggctgatgtctctttcttggaatatgtgaaataaatttagcagtttaactaaatacaaatatatgcattgtgtaatccactcagaattaaacagacaaaaggtatgcttgctttggaatgattttaggcattgtacaaccttgaatcacttgagcatgtaataactaataaataatgcagatccatgtgattattaaaatgactgtagctgagagctctaattttcctgtcttgaaactgtataagaactcatgtgattaagttcacagtttattgtttgtctgtttagtattttagaaatataccagcactactaattaactaatgtcttttatttattatattatgataaagtaaaaatttcacttgcattaagtctaaactgagaaggtaattactgggaggagaatgagcagctttgactttgacaggcggtttgtgcaggaaagcacagtgccgtgttgtttacagcttttctagagcagctgtgcgaccagggtagagagtgttgaaattcaataccaaatacagtaaaaacaaatgtaaataaaagaaaacacatcatcaataaaactgttattatgcgtgaccgta

SEQ ID NO: 6 - DNA, 22 bp, synthetic construct
  source 1..22: mol_type = other DNA
ttaaatgtaattcatgtcttaa

SEQ ID NO: 7 - DNA, 22 bp, synthetic construct
  source 1..22: mol_type = other DNA
ttcaatgaaattatggtaacta

SEQ ID NO: 8 - DNA, 22 bp, synthetic construct
  source 1..22: mol_type = other DNA
tataattgtaactcaatctttg

SEQ ID NO: 9 - DNA, 22 bp, synthetic construct
  source 1..22: mol_type = other DNA
tttgatttcaaatctctatgag

SEQ ID NO: 10 - DNA, 22 bp, synthetic construct
  source 1..22: mol_type = other DNA
tttaaatgtaattcatgtctta

SEQ ID NO: 11 - DNA, 22 bp, synthetic construct
  source 1..22: mol_type = other DNA
ttaagttgtaataaacaggttg

SEQ ID NO: 12 - DNA, 22 bp, synthetic construct
  source 1..22: mol_type = other DNA
tttaattgactttattacactg

SEQ ID NO: 13 - DNA, 22 bp, synthetic construct
  source 1..22: mol_type = other DNA
ttagtatctcaaaatatctgag

SEQ ID NO: 14 - DNA, 22 bp, synthetic construct
  source 1..22: mol_type = other DNA
ttaattgactttattacactgc

SEQ ID NO: 15 - DNA, 22 bp, synthetic construct
  source 1..22: mol_type = other DNA
ttaaatgacaaaattattactc

SEQ ID NO: 16 - DNA, 22 bp, synthetic construct
  source 1..22: mol_type = other DNA
ttagtaataagacatgtttcat

SEQ ID NO: 17 - DNA, 22 bp, synthetic construct
  source 1..22: mol_type = other DNA
tattcaaattattaactacctt

SEQ ID NO: 18 - DNA, 22 bp, synthetic construct
  source 1..22: mol_type = other DNA
taagtaatcaaaaagggatcca

SEQ ID NO: 19 - DNA, 22 bp, synthetic construct
  source 1..22: mol_type = other DNA
ttgtgtataactttgtctgtgg

SEQ ID NO: 20 - DNA, 22 bp, synthetic construct
  source 1..22: mol_type = other DNA
ttttaaatgtaattcatgtctt

SEQ ID NO: 21 - DNA, 22 bp, synthetic construct
  source 1..22: mol_type = other DNA
ttaacataagcatcactcttct

SEQ ID NO: 22 - DNA, 22 bp, synthetic construct
  source 1..22: mol_type = other DNA
ttaaagtactataagcatctag

SEQ ID NO: 23 - DNA, 22 bp, synthetic construct
  source 1..22: mol_type = other DNA
tatctattcaaattattaacta

SEQ ID NO: 24 - DNA, 22 bp, synthetic construct
  source 1..22: mol_type = other DNA
tcttgtaacacaatagttctcg

SEQ ID NO: 25 - DNA, 22 bp, synthetic construct
  source 1..22: mol_type = other DNA
ttaaacaagtaatacaacacaa

SEQ ID NO: 26 - DNA, 22 bp, synthetic construct
  source 1..22: mol_type = other DNA
taataaaacctgaaatctggga

SEQ ID NO: 27 - DNA, 22 bp, synthetic construct
  source 1..22: mol_type = other DNA
tacattttgtaataaaacctga

SEQ ID NO: 28 - DNA, 22 bp, synthetic construct
  source 1..22: mol_type = other DNA
tagaattacattttgatgcctt

SEQ ID NO: 29 - DNA, 22 bp, synthetic construct
  source 1..22: mol_type = other DNA
tttaagatcaaatagtctttca

SEQ ID NO: 30 - DNA, 22 bp, synthetic construct
  source 1..22: mol_type = other DNA
tacaatttaaaaaaacttatgg

SEQ ID NO: 31 - DNA, 22 bp, synthetic construct
  source 1..22: mol_type = other DNA
taaaattgtcttttgtacagag

SEQ ID NO: 32 - DNA, 22 bp, synthetic construct
  source 1..22: mol_type = other DNA
taagttgtaataaacaggttga

SEQ ID NO: 33 - DNA, 22 bp, synthetic construct
  source 1..22: mol_type = other DNA
taatgtactgacaatcatctta

SEQ ID NO: 34 - DNA, 22 bp, synthetic construct
  source 1..22: mol_type = other DNA
tttcattttcaatatcagccaa

SEQ ID NO: 35 - DNA, 22 bp, synthetic construct
  source 1..22: mol_type = other DNA
ttttaagatcaaatagtctttc

SEQ ID NO: 36 - DNA, 22 bp, synthetic construct
  source 1..22: mol_type = other DNA
tttcttacaagggaccttggtt

SEQ ID NO: 37 - DNA, 22 bp, synthetic construct
  source 1..22: mol_type = other DNA
tataagcaaaagaaacttgggc

SEQ ID NO: 38 - DNA, 22 bp, synthetic construct
  source 1..22: mol_type = other DNA
tttctggttgtcacaggtggaa

SEQ ID NO: 39 - DNA, 22 bp, synthetic construct
  source 1..22: mol_type = other DNA
ttgttgattaacaagtttccta

SEQ ID NO: 40 - DNA, 22 bp, synthetic construct
  source 1..22: mol_type = other DNA
tagagtgagaatttgctccttt

SEQ ID NO: 41 - DNA, 22 bp, synthetic construct
  source 1..22: mol_type = other DNA
ttcaggaatcttctcctccgag

SEQ ID NO: 42 - DNA, 22 bp, synthetic construct
  source 1..22: mol_type = other DNA
tttaatacatgagtacagctga

SEQ ID NO: 43 - DNA, 22 bp, synthetic construct
  source 1..22: mol_type = other DNA
tttctaaactaggttgagggag

SEQ ID NO: 44 - DNA, 22 bp, synthetic construct
  source 1..22: mol_type = other DNA
ttgtcagtgactatcatgtcgt

SEQ ID NO: 45 - DNA, 22 bp, synthetic construct
  source 1..22: mol_type = other DNA
tattttaatacatgagtacagc

SEQ ID NO: 46 - DNA, 22 bp, synthetic construct
  source 1..22: mol_type = other DNA
taagcaaaagaaacttgggcta

SEQ ID NO: 47 - DNA, 22 bp, synthetic construct
  source 1..22: mol_type = other DNA
ttttaatacatgagtacagctg

SEQ ID NO: 48 - DNA, 22 bp, synthetic construct
  source 1..22: mol_type = other DNA
ttaaaaaagagattacttgcag

SEQ ID NO: 49 - DNA, 22 bp, synthetic construct
  source 1..22: mol_type = other DNA
tattcatatttatatacaggca

SEQ ID NO: 50 - DNA, 22 bp, synthetic construct
  source 1..22: mol_type = other DNA
ttttaaaaaagagattacttgc

SEQ ID NO: 51 - DNA, 22 bp, synthetic construct
  source 1..22: mol_type = other DNA
ttatgtaaaagacaaacaatgc

SEQ ID NO: 52 - DNA, 22 bp, synthetic construct
  source 1..22: mol_type = other DNA
ttaaatgaaaagtccaggagaa

SEQ ID NO: 53 - DNA, 22 bp, synthetic construct
  source 1..22: mol_type = other DNA
ttgattaacaagtttcctatgg

SEQ ID NO: 54 - DNA, 22 bp, synthetic construct
  source 1..22: mol_type = other DNA
tttaattcctagactggggtcc

SEQ ID NO: 55 - DNA, 22 bp, synthetic construct
  source 1..22: mol_type = other DNA
tcaaaatcatattatataccca

SEQ ID NO: 56 - DNA, 22 bp, synthetic construct
  source 1..22: mol_type = other DNA
tatatatagagctatagacata

SEQ ID NO: 57 - DNA, 22 bp, synthetic construct
  source 1..22: mol_type = other DNA
tgtaatgaaaaagtaaacacga

SEQ ID NO: 58 - DNA, 22 bp, synthetic construct
  source 1..22: mol_type = other DNA
ttctaaactaggttgagggaga

SEQ ID NO: 59 - DNA, 22 bp, synthetic construct
  source 1..22: mol_type = other DNA
tattctagaaactcaccactag

SEQ ID NO: 60 - DNA, 22 bp, synthetic construct
  source 1..22: mol_type = other DNA
ttgaaatcctcttcatcaggcc

SEQ ID NO: 61 - DNA, 22 bp, synthetic construct
  source 1..22: mol_type = other DNA
tgagaatactcttgaatcttga

SEQ ID NO: 62 - DNA, 22 bp, synthetic construct
  source 1..22: mol_type = other DNA
ttgaatatctcatgaatggacc

SEQ ID NO: 63 - DNA, 22 bp, synthetic construct
  source 1..22: mol_type = other DNA
tttaaaaaagagattacttgca

SEQ ID NO: 64 - DNA, 22 bp, synthetic construct
  source 1..22: mol_type = other DNA
ttcactttttgaaacgctgtgc

SEQ ID NO: 65 - DNA, 22 bp, synthetic construct
  source 1..22: mol_type = other DNA
taatcttttacttctcccactg

SEQ ID NO: 66 - DNA, 22 bp, synthetic construct
  source 1..22: mol_type = other DNA
tatatataaaacatagctattc

SEQ ID NO: 67 - DNA, 22 bp, synthetic construct
  source 1..22: mol_type = other DNA
tatatatataaaacatagctat

SEQ ID NO: 68 - DNA, 22 bp, synthetic construct
  source 1..22: mol_type = other DNA
ttgtggttgatgttgttggcac

SEQ ID NO: 69 - DNA, 22 bp, synthetic construct
  source 1..22: mol_type = other DNA
tagaataaaaataagtgcttga

SEQ ID NO: 70 - DNA, 22 bp, synthetic construct
  source 1..22: mol_type = other DNA
taatgtttacagtgagaagtga

SEQ ID NO: 71 - DNA, 22 bp, synthetic construct
  source 1..22: mol_type = other DNA
tagggaaagatcttgactgcca

SEQ ID NO: 72 - DNA, 22 bp, synthetic construct
  source 1..22: mol_type = other DNA
tagagtttctaaactaggttga

SEQ ID NO: 73 - DNA, 22 bp, synthetic construct
  source 1..22: mol_type = other DNA
ttttagaaaaggatgaaggctg

SEQ ID NO: 74 - DNA, 22 bp, synthetic construct
  source 1..22: mol_type = other DNA
tttagaaaaggatgaaggctgg

SEQ ID NO: 75 - DNA, 22 bp, synthetic construct
  source 1..22: mol_type = other DNA
atagaataaaaataagtgcttg

SEQ ID NO: 76 - DNA, 22 bp, synthetic construct
  source 1..22: mol_type = other DNA
tcttgaatcttgaatatctcat

SEQ ID NO: 77 - DNA, 22 bp, synthetic construct
  source 1..22: mol_type = other DNA
tttcattagtagatggtggggc

SEQ ID NO: 78 - DNA, 22 bp, synthetic construct
  source 1..22: mol_type = other DNA
ttagaaaaggatgaaggctggg

SEQ ID NO: 79 - DNA, 22 bp, synthetic construct
  source 1..22: mol_type = other DNA
atatatatataaaacatagcta

SEQ ID NO: 80 - DNA, 22 bp, synthetic construct
  source 1..22: mol_type = other DNA
ttatattcttctgagaagatga

SEQ ID NO: 81 - DNA, 22 bp, synthetic construct
  source 1..22: mol_type = other DNA
taaaaatcaatctcatttcctg

SEQ ID NO: 82 - DNA, 22 bp, synthetic construct
  source 1..22: mol_type = other DNA
taataaacattttatttatgta

SEQ ID NO: 83 - DNA, 22 bp, synthetic construct
  source 1..22: mol_type = other DNA
tgattaacaagtttcctatgga

SEQ ID NO: 84 - DNA, 22 bp, synthetic construct
  source 1..22: mol_type = other DNA
ttaaaaatctcaataaaatgag

SEQ ID NO: 85 - RNA, 22 nt, synthetic construct
  source 1..22: mol_type = other RNA
tagattttaaacatccttggag

SEQ ID NO: 86 - RNA, 22 nt, synthetic construct
  source 1..22: mol_type = other RNA
tagattttaaacatccttggag

SEQ ID NO: 87 - RNA, 22 nt, synthetic construct
  source 1..22: mol_type = other RNA
ttactcttgatgaagtttgttt

SEQ ID NO: 88 - RNA, 22 nt, synthetic construct
  source 1..22: mol_type = other RNA
ttgaactttctgttctgctgtg

SEQ ID NO: 89 - RNA, 22 nt, synthetic construct
  source 1..22: mol_type = other RNA
ttgtctgtgtactccttccctt

SEQ ID NO: 90 - RNA, 22 nt, synthetic construct
  source 1..22: mol_type = other RNA
tctttgattgcaaacatgggtt

SEQ ID NO: 91 - RNA, 22 nt, synthetic construct
  source 1..22: mol_type = other RNA
ttgatctcatctattttggctt

SEQ ID NO: 92 - RNA, 22 nt, synthetic construct
  source 1..22: mol_type = other RNA
ttaagaatcttttcaaacacta

SEQ ID NO: 93 - RNA, 22 nt, synthetic construct
  source 1..22: mol_type = other RNA
ttctattgattcatagagtggg

SEQ ID NO: 94 - RNA, 22 nt, synthetic construct
  source 1..22: mol_type = other RNA
taatcttaatctttcatcctct

SEQ ID NO: 95 - RNA, 22 nt, synthetic construct
  source 1..22: mol_type = other RNA
ttaacttgacttagtgtcatga

SEQ ID NO: 96 - RNA, 22 nt, synthetic construct
  source 1..22: mol_type = other RNA
ttacataaatatgatcttctca

SEQ ID NO: 97 - RNA, 22 nt, synthetic construct
  source 1..22: mol_type = other RNA
tacataaatatgatcttctcag

SEQ ID NO: 98 - RNA, 22 nt, synthetic construct
  source 1..22: mol_type = other RNA
taaaaatctacaaatatgttgg

SEQ ID NO: 99 - RNA, 22 nt, synthetic construct
  source 1..22: mol_type = other RNA
tttggtttacatctgcacttgg

SEQ ID NO: 100 - RNA, 22 nt, synthetic construct
  source 1..22: mol_type = other RNA
tataatacgacttcacatcttc

SEQ ID NO: 101 - RNA, 22 nt, synthetic construct
  source 1..22: mol_type = other RNA
tagaaagttctttccatcgttt

SEQ ID NO: 102 - RNA, 22 nt, synthetic construct
  source 1..22: mol_type = other RNA
ttctatgtcaactaaactggca

SEQ ID NO: 103 - RNA, 22 nt, synthetic construct
  source 1..22: mol_type = other RNA
ttaaacagcatctcttggtcat

SEQ ID NO: 104 - RNA, 22 nt, synthetic construct
  source 1..22: mol_type = other RNA
tcgaatttccaagtacagcggc

SEQ ID NO: 105 - RNA, 22 nt, synthetic construct
  source 1..22: mol_type = other RNA
ttagaaagttctttccatcgtt

SEQ ID NO: 106 - RNA, 22 nt, synthetic construct
  source 1..22: mol_type = other RNA
tagatgtactgtataatacgac

SEQ ID NO: 107 - RNA, 22 nt, synthetic construct
  source 1..22: mol_type = other RNA
tctgtatactagaatctccctt

SEQ ID NO: 108 - RNA, 22 nt, synthetic construct
  source 1..22: mol_type = other RNA
ttttatgttaatatcatctcct

SEQ ID NO: 109 - RNA, 22 nt, synthetic construct
  source 1..22: mol_type = other RNA
tgagaatctcagttgatctggg

SEQ ID NO: 110 - RNA, 22 nt, synthetic construct
  source 1..22: mol_type = other RNA
tctgacaagagcttcacactga

SEQ ID NO: 111 - RNA, 22 nt, synthetic construct
  source 1..22: mol_type = other RNA
ttctattatagccatgtatgag

SEQ ID NO: 112 - RNA, 22 nt, synthetic construct
  source 1..22: mol_type = other RNA
tgatattttctaattgtagtag

SEQ ID NO: 113 - RNA, 22 nt, synthetic construct
  source 1..22: mol_type = other RNA
taaagtattcacaccaagcgtg

SEQ ID NO: 114 - RNA, 22 nt, synthetic construct
  source 1..22: mol_type = other RNA
tatgactgctacatcaagccat

SEQ ID NO: 115 - RNA, 22 nt, synthetic construct
  source 1..22: mol_type = other RNA
ttaaatgacatttactaaccat

SEQ ID NO: 116 - RNA, 22 nt, synthetic construct
  source 1..22: mol_type = other RNA
ttaaattaaaatcacttgggaa

SEQ ID NO: 117 - RNA, 22 nt, synthetic construct
  source 1..22: mol_type = other RNA
tttgctcttctcctaaaccgtc

SEQ ID NO: 118 - RNA, 22 nt, synthetic construct
  source 1..22: mol_type = other RNA
ttagttaattagtagtgctggt

SEQ ID NO: 119 - RNA, 22 nt, synthetic construct
  source 1..22: mol_type = other RNA
taggtgaggattcattcccggt

SEQ ID NO: 120 - RNA, 22 nt, synthetic construct
  source 1..22: mol_type = other RNA
ttagtcttggagcttgtgtgac

SEQ ID NO: 121 - RNA, 22 nt, synthetic construct
  source 1..22: mol_type = other RNA
tttaaattaaaatcacttggga

SEQ ID NO: 122 - RNA, 22 nt, synthetic construct
  source 1..22: mol_type = other RNA
ttttaaattaaaatcacttggg

SEQ ID NO: 123 - RNA, 22 nt, synthetic construct
  source 1..22: mol_type = other RNA
ttcaattacaatttagctacaa

SEQ ID NO: 124 - RNA, 22 nt, synthetic construct
  source 1..22: mol_type = other RNA
tttattatttcataaataggcg

SEQ ID NO: 125 - RNA, 22 nt, synthetic construct
  source 1..22: mol_type = other RNA
ttacaaacttgctatgactgct

SEQ ID NO: 126 - RNA, 22 nt, synthetic construct
  source 1..22: mol_type = other RNA
tattatttcataaataggcgca

SEQ ID NO: 127 - DNA, 613 bp, synthetic construct
  source 1..613: mol_type = other DNA
gtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgccagtgtgaagctcttgtcagatagtgaagccacagatgtatctgacaagagcttcacactgatgcctactgcctcggacttcaaggggctagaattcgagcaattatcttgtttactaaaactgaataccttgctatctctttgatacatttttacaaagctgaattaaaatggtataaattaaatcactttttcatctgaccagtagtggactagtgtgacgctgctgacccctttctttcccttctacag

SEQ ID NO: 128 - DNA, 354 bp, synthetic construct
  source 1..354: mol_type = other DNA
ccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgac

SEQ ID NO: 129 - DNA, 906 bp, synthetic construct
  source 1..906: mol_type = other DNA
ccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgccagtgtgaagctcttgtcagatagtgaagccacagatgtatctgacaagagcttcacactgatgcctactgcctcggacttcaaggggctagaattcgagcaattatcttgtttactaaaactgaataccttgctatctctttgatacatttttacaaagctgaattaaaatggtataaattaaatcactttgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgac

SEQ ID NO: 130 - RNA, 638 nt, synthetic construct
  source 1..638: mol_type = other RNA
gtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacaga

SEQ ID NO: 131 - RNA, 995 nt, synthetic construct
  source 1..995: mol_type = other RNA
gtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgccagtgtgaagctcttgtcagatagtgaagccacagatgtatctgacaagagcttcacactgatgcctactgcctcggacttcaaggggctagaattcgagcaattatcttgtttactaaaactgaataccttgctatctctttgatacatttttacaaagctgaattaaaatggtataaattaaatcactttgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacag

SEQ ID NO: 132 - RNA, 374 nt, synthetic construct
  source 1..374: mol_type = other RNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctag

SEQ ID NO: 133 - RNA, 1517 nt, synthetic construct
  source 1..1517: mol_type = other RNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgccagtgtgaagctcttgtcagatagtgaagccacagatgtatctgacaagagcttcacactgatgcctactgcctcggacttcaaggggctagaattcgagcaattatcttgtttactaaaactgaataccttgctatctctttgatacatttttacaaagctgaattaaaatggtataaattaaatcactttttcatctgaccagtagtggactagtgtgacgctgctgacccctttctttcccttctacagatccaagctgtgaccggcgcctacacctgcagcccaagcttacctaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttc

SEQ ID NO: 134 - RNA, 1541 nt, synthetic construct
  source 1..1541: mol_type = other RNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttacctaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttc

SEQ ID NO: 135 - RNA, 1578 nt, synthetic construct
  source 1..1578: mol_type = other RNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacacttcaaggggcttgcggccgcaaccatctccatggcgacggtgacttaggagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgacgactgtgacagcagagtatgccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttacctaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttc

SEQ ID NO: 136 - RNA, 1732 nt, synthetic construct
  source 1..1732: mol_type = other RNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggcgacggtgacttaggagtatgtgtttgaatgaggcttcagtactttacagaatcgttgcctgcacatcttggaaacacttgctgggattacttcgacttcttaacccaacagaaggctcgagaaggtatattgctgttgacagtgagcgcaggaaatgagattgatttttttagtgaagccacagatgtataaaaatcaatctcatttcctgtgcctactgcctcggacttcaaggggctagaattcgagcaattatcttgtttactaaaactgaataccttgctatctctttgatacatttttacaaagctgaattaaaatggtataaattaaatcactttgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttacctaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttc

SEQ ID NO: 137 - DNA, 1899 bp, synthetic construct
  source 1..1899: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgccagtgtgaagctcttgtcagatagtgaagccacagatgtatctgacaagagcttcacactgatgcctactgcctcggacttcaaggggctagaattcgagcaattatcttgtttactaaaactgaataccttgctatctctttgatacatttttacaaagctgaattaaaatggtataaattaaatcactttgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttacctaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttc

SEQ ID NO: 138 - DNA, 479 bp, Homo sapiens
  source 1..479: mol_type = other DNA
cgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttc

SEQ ID NO: 139 - RNA, 731 nt, synthetic construct
  source 1..731: mol_type = other RNA
cggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggcgacggtgacttaggagtatgtgtttgaatgaggcttcagtactttacagaatcgttgcctgcacatcttggaaacacttgctgggattacttcgacttcttaacccaacagaaggctcgagaaggtatattgctgttgacagtgagcgcaggaaatgagattgatttttttagtgaagccacagatgtataaaaatcaatctcatttcctgtgcctactgcctcggacttcaaggggctagaattcgagcaattatcttgtttactaaaactgaataccttgctatctctttgatacatttttacaaagctgaattaaaatggtataaattaaatcactttgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgac

SEQ ID NO: 140 - DNA, 508 bp, synthetic construct
  source 1..508: mol_type = other DNA
tgtttgaatgaggcttcagtactttacagaatcgttgcctgcacatcttggaaacacttgctgggattacttcgacttcttaacccaacagaaggctcgagaaggtatattgctgttgacagtgagcgcaggaaatgagattgatttttttagtgaagccacagatgtataaaaatcaatctcatttcctgtgcctactgcctcggacttcaaggggctagaattcgagcaattatcttgtttactaaaactgaataccttgctatctctttgatacatttttacaaagctgaattaaaatggtataaattaaatcactttgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgac

SEQ ID NO: 141 - RNA, 578 nt, synthetic construct
  source 1..578: mol_type = other RNA
ccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacacttcaaggggcttgcggccgcaaccatctccatggcgacggtgacttaggagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgacgactgtgacagcagagtatgccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgac

SEQ ID NO: 142 - DNA, 103 bp, synthetic construct
  source 1..103: mol_type = other DNA
ggggccactagggacaggatgttttagagctagaaatagcaagttaaaataaggctagtccgttatcaacttgaaaaagtggcaccgagtcggtgcttttttt

SEQ ID NO: 143 - DNA, 20 bp, Homo sapiens
  source 1..20: mol_type = genomic DNA
ggggccactagggacaggat

SEQ ID NO: 144 - RNA, 20 nt, synthetic construct
  source 1..20: mol_type = other RNA
gcacctgaataccacgcctg

SEQ ID NO: 145 - RNA, 20 nt, synthetic construct
  source 1..20: mol_type = other RNA
cgcctgcgatgtagtcgatg

SEQ ID NO: 146 - RNA, 20 nt, synthetic construct
  source 1..20: mol_type = other RNA
caggacgggcgagatgtccc

SEQ ID NO: 147 - RNA, 20 nt, synthetic construct
  source 1..20: mol_type = other RNA
ctgaatctttggagtacctg

SEQ ID NO: 148 - RNA, 20 nt, synthetic construct
  source 1..20: mol_type = other RNA
ggccacggagcgagacatct

SEQ ID NO: 149 - RNA, 20 nt, synthetic construct
  source 1..20: mol_type = other RNA
aagtcaacttcaatgtcgga

SEQ ID NO: 150 - RNA, 20 nt, synthetic construct
  source 1..20: mol_type = other RNA
gcttggaggcctgatcagcg

SEQ ID NO: 151 - RNA, 20 nt, synthetic construct
  source 1..20: mol_type = other RNA
cttatctcttcgcagcgagg

SEQ ID NO: 152 - RNA, 20 nt, synthetic construct
  source 1..20: mol_type = other RNA
cacacattactccaacattg

SEQ ID NO: 153 - RNA, 20 nt, synthetic construct
  source 1..20: mol_type = other RNA
ttccgcaaaatagagcccca

SEQ ID NO: 154 - RNA, 20 nt, synthetic construct
  source 1..20: mol_type = other RNA
tgcacagaactatcgtacca

SEQ ID NO: 155 - RNA, 20 nt, synthetic construct
  source 1..20: mol_type = other RNA
gcaataagactctttaaaga

SEQ ID NO: 156 - RNA, 20 nt, synthetic construct
  source 1..20: mol_type = other RNA
caaagagattacgaatgcct

SEQ ID NO: 157 - RNA, 20 nt, synthetic construct
  source 1..20: mol_type = other RNA
caaggcaccccaggtttcca

SEQ ID NO: 158 - RNA, 20 nt, synthetic construct
  source 1..20: mol_type = other RNA
ttacgaatgccttggaaacc

SEQ ID NO: 159 - RNA, 20 nt, synthetic construct
  source 1..20: mol_type = other RNA
cagagacgcatctgaccctc

SEQ ID NO: 160 - RNA, 20 nt, synthetic construct
  source 1..20: mol_type = other RNA
catgcagttctcacacactg

SEQ ID NO: 161 - RNA, 20 nt, synthetic construct
  source 1..20: mol_type = other RNA
gtgtgagaactgcatggaga

SEQ ID NO: 162 - RNA, 20 nt, synthetic construct
  source 1..20: mol_type = other RNA
tctcatttcaggaaaccact

SEQ ID NO: 163 - RNA, 20 nt, synthetic construct
  source 1..20: mol_type = other RNA
agtcatacaccttaaccaag

SEQ ID NO: 164 - RNA, 20 nt, synthetic construct
  source 1..20: mol_type = other RNA
ttcaaggaaaccagttgagg

SEQ ID NO: 165 - RNA, 20 nt, synthetic construct
  source 1..20: mol_type = other RNA
gagccttgcctggaaatctg

SEQ ID NO: 166 - RNA, 20 nt, synthetic construct
  source 1..20: mol_type = other RNA
aagcgtcaaaagtctgccag

SEQ ID NO: 167 - RNA, 20 nt, synthetic construct
  source 1..20: mol_type = other RNA
cgttccaactcgaagtgcca

SEQ ID NO: 168 - RNA, 20 nt, synthetic construct
  source 1..20: mol_type = other RNA
gagcgactgggacacggtga

SEQ ID NO: 169 - RNA, 20 nt, synthetic construct
  source 1..20: mol_type = other RNA
gctgcgcaagaagggcccta

SEQ ID NO: 170 - RNA, 20 nt, synthetic construct
  source 1..20: mol_type = other RNA
ttgttctggccagcagcccc

SEQ ID NO: 171 - RNA, 20 nt, synthetic construct
  source 1..20: mol_type = other RNA
cttccagagccacatcatcg

SEQ ID NO: 172 - RNA, 20 nt, synthetic construct
  source 1..20: mol_type = other RNA
gggactcaccagagagaggt

SEQ ID NO: 173 - RNA, 20 nt, synthetic construct
  source 1..20: mol_type = other RNA
cggtcgaaatagaagcccta

SEQ ID NO: 174 - RNA, 20 nt, synthetic construct
  source 1..20: mol_type = other RNA
aaaaggatattgtgcaactg

SEQ ID NO: 175 - RNA, 20 nt, synthetic construct
  source 1..20: mol_type = other RNA
tgtgcatatttattacatcg

SEQ ID NO: 176 - RNA, 20 nt, synthetic construct
  source 1..20: mol_type = other RNA
tttgtgaagatcttgaccaa

SEQ ID NO: 177 - RNA, 20 nt, synthetic construct
  source 1..20: mol_type = other RNA
tgtcatgctgaaccgcattg

SEQ ID NO: 178 - RNA, 20 nt, synthetic construct
  source 1..20: mol_type = other RNA
ccactctatgaggatagtca

SEQ ID NO: 179 - RNA, 20 nt, synthetic construct
  source 1..20: mol_type = other RNA
ttgacatagaagaggcacaa

SEQ ID NO: 180 - RNA, 20 nt, synthetic construct
  source 1..20: mol_type = other RNA
gagtactacactcagcagca

SEQ ID NO: 181 - RNA, 20 nt, synthetic construct
  source 1..20: mol_type = other RNA
tcacgcacaagaaacgtcca

SEQ ID NO: 182 - RNA, 20 nt, synthetic construct
  source 1..20: mol_type = other RNA
aggtctcggtgaaaccacct

SEQ ID NO: 183 - RNA, 20 nt, synthetic construct
  source 1..20: mol_type = other RNA
agcattatccaaagagtccg

SEQ ID NO: 184 - RNA, 20 nt, synthetic construct
  source 1..20: mol_type = other RNA
atattaattcttaccagtgg

SEQ ID NO: 185 - RNA, 20 nt, synthetic construct
  source 1..20: mol_type = other RNA
agctttaaatcaaggttcat

SEQ ID NO: 186 - RNA, 20 nt, synthetic construct
  source 1..20: mol_type = other RNA
atcccgagccctaaggtgca

SEQ ID NO: 187 - RNA, 20 nt, synthetic construct
  source 1..20: mol_type = other RNA
ggcagcgcggaggacagcgt

SEQ ID NO: 188 - RNA, 20 nt, synthetic construct
  source 1..20: mol_type = other RNA
ctcagggggctactaccacc

SEQ ID NO: 189 - RNA, 20 nt, synthetic construct
  source 1..20: mol_type = other RNA
gtcaccgacgagaccagaag

SEQ ID NO: 190 - RNA, 20 nt, synthetic construct
  source 1..20: mol_type = other RNA
gtcgtggacttcgtactgct

SEQ ID NO: 191 - RNA, 20 nt, synthetic construct
  source 1..20: mol_type = other RNA
taatttttaggcaagtgtcg

SEQ ID NO: 192 - RNA, 20 nt, synthetic construct
  source 1..20: mol_type = other RNA
ttagctgttagacttgaata

SEQ ID NO: 193 - RNA, 20 nt, synthetic construct
  source 1..20: mol_type = other RNA
cgagagccgtcaacttgcgt

SEQ ID NO: 194 - RNA, 20 nt, synthetic construct
  source 1..20: mol_type = other RNA
cggcttcaactgcaaaggtg

SEQ ID NO: 195 - RNA, 20 nt, synthetic construct
  source 1..20: mol_type = other RNA
tatgaaaaagcagagcgact

SEQ ID NO: 196 - RNA, 20 nt, synthetic construct
  source 1..20: mol_type = other RNA
tctggcgggcgagctcacgc

SEQ ID NO: 197 - RNA, 20 nt, synthetic construct
  source 1..20: mol_type = other RNA
ctcacgctggttaccgccta

SEQ ID NO: 198 - RNA, 20 nt, synthetic construct
  source 1..20: mol_type = other RNA
aaagattacgaacttccctg

SEQ ID NO: 199 - RNA, 20 nt, synthetic construct
  source 1..20: mol_type = other RNA
gttaaaaacagacatgccta

SEQ ID NO: 200 - RNA, 20 nt, synthetic construct
  source 1..20: mol_type = other RNA
atgcctaaggaggttgtacc

SEQ ID NO: 201 - RNA, 20 nt, synthetic construct
  source 1..20: mol_type = other RNA
ctccaggtatcccatcgaaa

SEQ ID NO: 202 - RNA, 20 nt, synthetic construct
  source 1..20: mol_type = other RNA
caccaaatacgatagatcag

SEQ ID NO: 203 - RNA, 20 nt, synthetic construct
  source 1..20: mol_type = other RNA
tggcggcgtgaatggcaaga

SEQ ID NO: 204 - RNA, 20 nt, synthetic construct
  source 1..20: mol_type = other RNA
taggatggtagcacacaacc

SEQ ID NO: 205 - RNA, 20 nt, synthetic construct
  source 1..20: mol_type = other RNA
cagcagcagagccccgacgg

SEQ ID NO: 206 - RNA, 20 nt, synthetic construct
  source 1..20: mol_type = other RNA
cggcgtgcgaacggaatgtg

SEQ ID NO: 207 - RNA, 20 nt, synthetic construct
  source 1..20: mol_type = other RNA
tatagacgctgcccgacgtc

SEQ ID NO: 208 - RNA, 20 nt, synthetic construct
  source 1..20: mol_type = other RNA
tccaaagaagggtactgtgg

SEQ ID NO: 209 - RNA, 20 nt, synthetic construct
  source 1..20: mol_type = other RNA
acagtacccttctttggaat

SEQ ID NO: 210 - RNA, 20 nt, synthetic construct
  source 1..20: mol_type = other RNA
gcgacgggcgcatctacgtg

SEQ ID NO: 211 - RNA, 20 nt, synthetic construct
  source 1..20: mol_type = other RNA
cccgacctccataagtcctg

SEQ ID NO: 212 - RNA, 20 nt, synthetic construct
  source 1..20: mol_type = other RNA
ggggtcctcgaagcgcacga

SEQ ID NO: 213 - RNA, 20 nt, synthetic construct
  source 1..20: mol_type = other RNA
tgctctgtttagaagatgac

SEQ ID NO: 214 - RNA, 20 nt, synthetic construct
  source 1..20: mol_type = other RNA
atattcttttctagttaaag

SEQ ID NO: 215 - RNA, 20 nt, synthetic construct
  source 1..20: mol_type = other RNA
cctgtaaagaaacaaaagac

SEQ ID NO: 216 - RNA, 20 nt, synthetic construct
  source 1..20: mol_type = other RNA
tggagaaagacgtaacttcg

SEQ ID NO: 217 - RNA, 20 nt, synthetic construct
  source 1..20: mol_type = other RNA
tctgccctgaggtatgcgat

SEQ ID NO: 218 - RNA, 20 nt, synthetic construct
  source 1..20: mol_type = other RNA
attccgcttggtgaaaacga

SEQ ID NO: 219 - RNA, 20 nt, synthetic construct
  source 1..20: mol_type = other RNA
caggcacaatagaaacaacg

SEQ ID NO: 220 - RNA, 20 nt, synthetic construct
  source 1..20: mol_type = other RNA
ccatttgtaatgctgacttg

SEQ ID NO: 221 - RNA, 20 nt, synthetic construct
  source 1..20: mol_type = other RNA
ctgggtcacttgtgccgtgg

SEQ ID NO: 222 - RNA, 20 nt, synthetic construct
  source 1..20: mol_type = other RNA
gtcagggttctggatatctg

SEQ ID NO: 223 - RNA, 20 nt, synthetic construct
  source 1..20: mol_type = other RNA
tggatttagagtctctcagc

SEQ ID NO: 224 - RNA, 20 nt, synthetic construct
  source 1..20: mol_type = other RNA
ctgcggctgtggtccagctg

SEQ ID NO: 225 - RNA, 20 nt, synthetic construct
  source 1..20: mol_type = other RNA
acaaaactgtgctagacatg

SEQ ID NO: 226 - RNA, 20 nt, synthetic construct
  source 1..20: mol_type = other RNA
ttcttccccagcccaggtaa

SEQ ID NO: 227 - RNA, 20 nt, synthetic construct
  source 1..20: mol_type = other RNA
cgtcatgagcagattaaacc

SEQ ID NO: 228 - RNA, 20 nt, synthetic construct
  source 1..20: mol_type = other RNA
gagagcgcctgcgacccgag

SEQ ID NO: 229 - RNA, 20 nt, synthetic construct
  source 1..20: mol_type = other RNA
ccagcgggtgaagtacacca

SEQ ID NO: 230 - RNA, 20 nt, synthetic construct
  source 1..20: mol_type = other RNA
ggagcgcttttcgccgccag

SEQ ID NO: 231 - RNA, 20 nt, synthetic construct
  source 1..20: mol_type = other RNA
tgaggcctggaccttatgca

SEQ ID NO: 232 - RNA, 20 nt, synthetic construct
  source 1..20: mol_type = other RNA
cctggtggagtgaaccatga

SEQ ID NO: 233 - RNA, 20 nt, synthetic construct
  source 1..20: mol_type = other RNA
caagcacttaggttcccctg

SEQ ID NO: 234 - RNA, 20 nt, synthetic construct
  source 1..20: mol_type = other RNA
ggtctccctacaattcagcg

SEQ ID NO: 235 - RNA, 20 nt, synthetic construct
  source 1..20: mol_type = other RNA
cacagcgcgtgactgcaatg

SEQ ID NO: 236 - RNA, 20 nt, synthetic construct
  source 1..20: mol_type = other RNA
tctggggcaccaattctagg

SEQ ID NO: 237 - RNA, 20 nt, synthetic construct
  source 1..20: mol_type = other RNA
gagccatgcttggcttacga

SEQ ID NO: 238 - RNA, 20 nt, synthetic construct
  source 1..20: mol_type = other RNA
gtacaagtacttatctcatg

SEQ ID NO: 239 - RNA, 20 nt, synthetic construct
  source 1..20: mol_type = other RNA
gagataacaacataacaaca

SEQ ID NO: 240 - RNA, 20 nt, synthetic construct
  source 1..20: mol_type = other RNA
catattccatagtctttggg

SEQ ID NO: 241 - RNA, 20 nt, synthetic construct
  source 1..20: mol_type = other RNA
ctgccccttagcaacttagg

SEQ ID NO: 242 - RNA, 20 nt, synthetic construct
  source 1..20: mol_type = other RNA
tgtttaaaaatatgttgaca

SEQ ID NO: 243 - RNA, 20 nt, synthetic construct
  source 1..20: mol_type = other RNA
ccaggaatggaaactcacgc

SEQ ID NO: 244 - RNA, 20 nt, synthetic construct
  source 1..20: mol_type = other RNA
gaggccgctgaattaacccg

SEQ ID NO: 245 - RNA, 20 nt, synthetic construct
  source 1..20: mol_type = other RNA
atacacgcacacttgcagaa

SEQ ID NO: 246 - RNA, 20 nt, synthetic construct
  source 1..20: mol_type = other RNA
gagcagacagaaacccaggg

SEQ ID NO: 247 - RNA, 20 nt, synthetic construct
  source 1..20: mol_type = other RNA
tgagtctccaaacagaacag

SEQ ID NO: 248 - RNA, 20 nt, synthetic construct
  source 1..20: mol_type = other RNA
taatatcactgacttcacgg

SEQ ID NO: 249 - RNA, 20 nt, synthetic construct
  source 1..20: mol_type = other RNA
tacacacaatgtaagcagca

SEQ ID NO: 250 - RNA, 20 nt, synthetic construct
  source 1..20: mol_type = other RNA
gggagctcaattcgaaacca

SEQ ID NO: 251 - RNA, 20 nt, synthetic construct
  source 1..20: mol_type = other RNA
ttggacaggtgagacagtcg

SEQ ID NO: 252 - RNA, 20 nt, synthetic construct
  source 1..20: mol_type = other RNA
aagctcactcagatagtgtg

SEQ ID NO: 253 - RNA, 20 nt, synthetic construct
  source 1..20: mol_type = other RNA
caggagaaccaccttacacg

SEQ ID NO: 254 - RNA, 20 nt, synthetic construct
  source 1..20: mol_type = other RNA
ggacagaccctgattcacaa

SEQ ID NO: 255 - RNA, 20 nt, synthetic construct
  source 1..20: mol_type = other RNA
acatggcagtctatgaacag

SEQ ID NO: 256 - RNA, 20 nt, synthetic construct
  source 1..20: mol_type = other RNA
cctatagagagtactacttg

SEQ ID NO: 257 - RNA, 20 nt, synthetic construct
  source 1..20: mol_type = other RNA
ccaaccgggtcttcattacg

SEQ ID NO: 258 - RNA, 20 nt, synthetic construct
  source 1..20: mol_type = other RNA
tcaagcgtagagttccgagt

SEQ ID NO: 259 - RNA, 20 nt, synthetic construct
  source 1..20: mol_type = other RNA
tcatgcaattatggacccag

SEQ ID NO: 260 - RNA, 20 nt, synthetic construct
  source 1..20: mol_type = other RNA
cgggaaagtgactggccatg

SEQ ID NO: 261 - RNA, 20 nt, synthetic construct
  source 1..20: mol_type = other RNA
tgagattgaaatcaaatcgg

SEQ ID NO: 262 - RNA, 20 nt, synthetic construct
  source 1..20: mol_type = other RNA
tatgcaatattcatcacgcg

SEQ ID NO: 263 - RNA, 20 nt, synthetic construct
  source 1..20: mol_type = other RNA
aatgtgttaaatcaaatgca

SEQ ID NO: 264 - DNA, 1547 bp, synthetic construct
  source 1..1547: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgcsagtgtgaagctcttgtcagatagtgaagccacagatgtatctgacaagagcttcacactgatgcctactgcctcggacttcaaggggctagaattcgagcaattatcttgtttactaaaactgaataccttgctatctctttgatacatttttacaaagctgaattaaaatggtataaattaaatcactttttcatctgaccagtagtggactagtgtgacgctgctgacccctttctttcccttctacagatccaagctgtgaccggcgcctacacctgcagcccaagcttaccnnnnnnnnnnnnnnnnnnnnnnnnnnnnnntaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttc

SEQ ID NO: 265 - DNA, 1762 bp, synthetic construct
  source 1..1762: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggcgacggtgacttaggagtatgtgtttgaatgaggcttcagtactttacagaatcgttgcctgcacatcttggaaacacttgctgggattacttcgacttcttaacccaacagaaggctcgagaaggtatattgctgttgacagtgagcgcaggaaatgagattgatttttttagtgaagccacagatgtataaaaatcaatctcatttcctgtgcctactgcctcggacttcaaggggctagaattcgagcaattatcttgtttactaaaactgaataccttgctatctctttgatacatttttacaaagctgaattaaaatggtataaattaaatcactttgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttaccnnnnnnnnnnnnnnnnnnnnnnnnnnnnnntaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttc

SEQ ID NO: 266 - DNA, 1608 bp, synthetic construct
  source 1..1608: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacacttcaaggggcttgcggccgcaaccatctccatggcgacggtgacttaggagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgacgactgtgacagcagagtatgccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttaccnnnnnnnnnnnnnnnnnnnnnnnnnnnnnntaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttc

SEQ ID NO: 267 - DNA, 1571 bp, synthetic construct
  source 1..1571: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttaccnnnnnnnnnnnnnnnnnnnnnnnnnnnnnntaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttc

SEQ ID NO: 268 - DNA, 1929 bp, synthetic construct
  source 1..1929: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgccagtgtgaagctcttgtcagatagtgaagccacagatgtatctgacaagagcttcacactgatgcctactgcctcggacttcaaggggctagaattcgagcaattatcttgtttactaaaactgaataccttgctatctctttgatacatttttacaaagctgaattaaaatggtataaattaaatcactttgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttaccnnnnnnnnnnnnnnnnnnnnnnnnnnnnnntaaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttc

SEQ ID NO: 269 - RNA, 1031 nt, synthetic construct
  source 1..1031: mol_type = other RNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgccagtgtgaagctcttgtcagatagtgaagccacagatgtatctgacaagagcttcacactgatgcctactgcctcggacttcaaggggctagaattcgagcaattatcttgtttactaaaactgaataccttgctatctctttgatacatttttacaaagctgaattaaaatggtataaattaaatcactttttcatctgaccagtagtggactagtgtgacgctgctgacccctttctttcccttctacagatccaagctgtgaccggcgcctacacctgcagcccaagcttacc

SEQ ID NO: 270 - RNA, 486 nt, synthetic construct
  source 1..486: mol_type = other RNA
taaaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagaaacggggtttcaccatattggccaggctggtctccaactcctaatctcaggtgatctacccaccttggcctcccaaattgctgggattacaggcgtgaaccactgctcccttccctgtccttc

SEQ ID NO: 271 - RNA, 1055 nt, synthetic construct
  source 1..1055: mol_type = other RNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttacc

SEQ ID NO: 272 - RNA, 486 nt, synthetic construct; identical to SEQ ID NO: 270
  source 1..486: mol_type = other RNA

SEQ ID NO: 273 - RNA, 1092 nt, synthetic construct
  source 1..1092: mol_type = other RNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacacttcaaggggcttgcggccgcaaccatctccatggcgacggtgacttaggagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgacgactgtgacagcagagtatgccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttacc

SEQ ID NO: 274 - RNA, 486 nt, synthetic construct; identical to SEQ ID NO: 270
  source 1..486: mol_type = other RNA

SEQ ID NO: 275 - RNA, 1246 nt, synthetic construct
  source 1..1246: mol_type = other RNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggcgacggtgacttaggagtatgtgtttgaatgaggcttcagtactttacagaatcgttgcctgcacatcttggaaacacttgctgggattacttcgacttcttaacccaacagaaggctcgagaaggtatattgctgttgacagtgagcgcaggaaatgagattgatttttttagtgaagccacagatgtataaaaatcaatctcatttcctgtgcctactgcctcggacttcaaggggctagaattcgagcaattatcttgtttactaaaactgaataccttgctatctctttgatacatttttacaaagctgaattaaaatggtataaattaaatcactttgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttacc

SEQ ID NO: 276 - RNA, 486 nt, synthetic construct; identical to SEQ ID NO: 270
  source 1..486: mol_type = other RNA

SEQ ID NO: 277 - RNA, 1413 nt, synthetic construct
  source 1..1413: mol_type = other RNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtcgactcgttggatccccactacccggatcaacgccctaggtttatgtttggatgaactgacatacgcgtatccgtcttaagaatcttttcaaacactagtagtgaaatatatattaaactagtgtttgaaaagattcttattacggtaacgcggaattcgcaactattttatcaattttttgcgtcgacacttcaaggggcttgcggccgcaaccatctccatggctgtttgaatgaggcttcagtactttacagaatcgttgcctgcacatcttggaaacacttgctgggattacttcgacttcttaacccaacagaaggctcgagaaggtatattgctgttgacagtgagcgccagtgtgaagctcttgtcagatagtgaagccacagatgtatctgacaagagcttcacactgatgcctactgcctcggacttcaaggggctagaattcgagcaattatcttgtttactaaaactgaataccttgctatctctttgatacatttttacaaagctgaattaaaatggtataaattaaatcactttgacggtgacttaggagtatgccggatcaacgccctaggtttatgtttggatgaactgacatacgcgtatccgtctaaaaatcaatctcatttcctggtagtgaaatatatattaaaccaggaaatgagattgatttttttacggtaacgcggaattcgcaactattttatcaattttttgcgtcgacgactgtgacagcagagtatgccggatcaacgccctaggtttatgtttggatgaactgacatacgcgtatccgtcttatgtaaaagacaaacaatgcgtagtgaaatatatattaaacgcattgtttgtcttttacatattacggtaacgcggaattcgcaactattttatcaattttttgcgtcgaccggaactatcttgaagagtagtagtggactagtgtgacgctgctgacccctttctttcccttctacagatccaagctgtgaccggcgcctacacctgcagcccaagcttacc

SEQ ID NO: 278 - RNA, 486 nt, synthetic construct; identical to SEQ ID NO: 270
  source 1..486: mol_type = other RNA

(19) United States
(12) Patent Application Publication
(10) Pub. No.: US 2025/0215438 A1
(43) Pub. Date: Jul. 3, 2025

(54) IMMUNE CELLS HAVING CO-EXPRESSED TGFBR SHRNAS

(71) Applicant: Arsenal Biosciences, Inc., South San Francisco, CA (US)

(72) Inventors: John Gagnon, San Francisco, CA (US); Adam Litterman, San Francisco, CA (US); Amy-Jo Casbon, San Francisco, CA (US); Brenal Singh, San Francisco, CA (US); Jason Hall, South San Francisco, CA (US); Natalie Bezman, Foster City, CA (US); Aaron Cooper, Berkeley, CA (US); Levi Gray-Rupp, Philadelphia, PA (US)

(21) Appl. No.: 19/077,046

(22) Filed: Mar. 11, 2025

Related U.S. Application Data
(63) Continuation of application No. PCT/US2023/074047, filed on Sep. 13, 2023.
(60) Provisional application No. 63/516,484, filed on Jul. 28, 2023.
(60) Provisional application No. 63/495,867, filed on Apr. 13, 2023.
(60) Provisional application No. 63/489,840, filed on Mar. 13, 2023.
(60) Provisional application No. 63/375,519, filed on Sep. 13, 2022.

(51) Int. Cl. C12N 15/113; A61K 35/17; A61P 35/00; C12N 5/0783

(52) U.S. Cl. CPC C12N 15/1138; A61K 35/17; A61P 35/00; C12N 5/0636; C12N 2310/14; C12N 2310/531; C12N 2510/00

(57) ABSTRACT

Provided herein are recombinant nucleic acids that reduce expression of TGFBR1 and/or TGFBR2 and cells comprising such recombinant nucleic acids. Also provided are methods of making and using such cells.

### CROSS REFERENCE TO RELATED APPLICATIONS

This application is a continuation of International Patent Application No. PCT/US2023/074047, filed Sep. 13, 2023, which claims the benefit of and priority to U.S. Provisional Application No. 63/375,519, filed Sep. 13, 2022, U.S. Provisional Application No. 63/489,840, filed Mar. 13, 2023, U.S. Provisional Application No. 63/495,867, filed Apr. 13, 2023, and U.S. Provisional Application No. 63/516,484, filed Jul. 28, 2023, the disclosures of each of which are hereby incorporated by reference in their entireties for all purposes.

### SEQUENCE LISTING

The instant application contains a Sequence Listing which has been submitted electronically and is hereby incorporated by reference in its entirety. Said XML copy, created on Mar. 11, 2025, is named ANB-215USWOC1_sequencelisting.xml, and is 298,845 bytes in size.

### BACKGROUND

Cancer is a disease characterized by uncontrollable growth of cells. Many approaches to treating cancer have been tried, including drugs and radiation therapies. Recent cancer treatments have sought to use the body's own immune cells to attack cancer cells. One promising approach uses T cells that are taken from a patient and genetically engineered to produce chimeric antigen receptors, or CARs, receptor proteins that give the T cells a new ability to target a specific protein. The receptors are chimeric because they combine antigen-binding and T-cell activating functions into a single receptor.

Immunotherapy using CAR-T cells is promising because the modified T cells have the potential to recognize cancer cells in order to more effectively target and destroy them. After the T cells are engineered with the CARs, the resulting CAR-T cells are introduced into patients to attack tumor cells. Once CAR-T cells are infused into a patient, they come in contact with their targeted antigen on a cell. The CAR-T cells bind to the antigen and become activated. Upon antigen engagement, CAR T cells can proliferate exponentially, initiate antitumor cytokine production, and target tumor cell killing.

However, there remain some limitations to CAR T cell-based immunotherapy. In particular, CAR-T cells can lack peripheral survival, can have reduced expansion and effector function, are susceptible to suppression and exhaustion, and may not result in memory T cell persistence. Thus, additional therapies targeting T cell intrinsic pathways are needed to address these roadblocks for CAR-T therapy.

### SUMMARY

In one aspect, provided herein are one or more recombinant nucleic acids comprising a nucleic acid sequence at least 15 nucleotides in length complementary to an mRNA encoding human TGF-β Receptor 2 (TGFBR2) comprising the sequence set forth in SEQ ID NO: 2.

In one aspect, provided herein are one or more recombinant nucleic acids comprising a nucleic acid sequence at least 15 nucleotides in length complementary to an mRNA encoding human TGF-β Receptor 1 (TGFBR1) comprising the sequence set forth in SEQ ID NO: 1.

In one aspect, provided herein are one or more recombinant nucleic acids comprising: a first nucleic acid sequence at least 15 nucleotides in length complementary to an mRNA encoding human TGF-β Receptor 2 (TGFBR2) comprising the sequence set forth in SEQ ID NO: 2, and a second nucleic acid sequence at least 15 nucleotides in length complementary an mRNA encoding human TGF-β Receptor 2 (TGFBR2) comprising the sequence set forth in SEQ ID NO: 2.

In one aspect, provided herein are one or more recombinant nucleic acids comprising: a first nucleic acid sequence at least 15 nucleotides in length complementary an mRNA encoding human TGF-β Receptor 2 (TGFBR2) comprising the sequence set forth in SEQ ID NO: 2, and a second nucleic acid sequence at least 15 nucleotides in length complementary to an mRNA encoding human TGF-β Receptor 1 (TGFBR1) comprising the sequence set forth in SEQ ID NO: 1.

In some embodiments, the nucleic acid sequence is at least 16, 17, 18, 19, 20, 21, or 22 nucleotides in length.

In some embodiments, the nucleic acid is a short hairpin RNA (shRNA), a small interfering RNA (siRNA), a double stranded RNA (dsRNA), or an antisense oligonucleotide.

In some embodiments, the nucleic acid is an shRNA.

In some embodiments, the nucleic acid or first nucleic acid comprises a sequence selected from the group consisting of the sequences set forth in SEQ ID NOs: 6-84.

In some embodiments, the first and second nucleic acids each comprises a sequence selected from the group consisting of the sequences set forth in SEQ ID NOs: 36-84.

In some embodiments, the nucleic acid or first nucleic acid comprises the sequence set forth in SEQ ID NO: 81.

In some embodiments, the nucleic acid or first nucleic acid comprises the sequence set forth in SEQ ID NO: 51 or 53.

In some embodiments, the nucleic acid, first nucleic acid, or second nucleic acid comprises the sequence set forth in SEQ ID NOs: 81 and 51 or 53.

In some embodiments, the nucleic acid comprises the sequence set forth in SEQ ID NO: 128, 129, 130, 139, 140, or 141

In some embodiments, the nucleic acid, first nucleic acid, or second nucleic acid reduces expression of TGFBR2 in a cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the respective nucleic acid.

In some embodiments, the nucleic acid or second nucleic acid comprises a sequence selected from the group consisting of the sequences set forth in SEQ ID NOs: 6-35.

In some embodiments, the nucleic acid or second nucleic acid comprises the sequence set forth in SEQ ID NO: 16 or 28.

In some embodiments, the nucleic acid or second nucleic acid reduces expression of TGFBR 1 in a cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the respective nucleic acid.

In some embodiments, the first nucleic acid comprises the sequence set forth in SEQ ID NO: 81 and the second nucleic acid comprises the sequence set forth in SEQ ID NO: 16 or 28.

In some embodiments, the first nucleic acid comprises the sequence set forth in SEQ ID NO: 81 and the second nucleic acid comprises the sequence set forth in SEQ ID NO: 51.

In some embodiments, the first and second nucleic acid reduces expression of TGFBR1 and TGFBR2 in a cell by at least 10%, 15%, 20%, 25%, 30%, 35%, 40%, 45%, 50%, 55%, 60%, 65%, 75%, 80%, 85%, 90%, 95%, or 99%, as compared to a control cell that does not comprise the respective nucleic acid.

In some embodiments, the first nucleic acid comprises a sequence selected from the group consisting of the sequences set forth in SEQ ID NOs: 36-84 and the second nucleic acid comprises a sequence selected from the group consisting of the sequences set forth in SEQ ID NOs: 6-84.

In some embodiments, the first nucleic acid comprises the sequence set forth in SEQ ID NO: 81 and the second nucleic acid comprises the sequence set forth in SEQ ID NO: 28, 16, 51, or 53.

In some embodiments, the nucleic acid, first nucleic acid, an/or second nucleic acid reduce expression of TFGBR2 in a cell by at least 10%, 15%, 20%, 25%, 30%, 35%, 40%, 45%, 50%, 55%, 60%, 65%, 75%, 80%, 85%, 90%, 95%, or 99% and the second nucleic acid reduces expression of TGFBR1 and/or TGFBR2 in a cell by at least 10%, 15%, 20%, 25%, 30%, 35%, 40%, 45%, 50%, 55%, 60%, 65%, 75%, 80%, 85%, 90%, 95%, or 99%, each as compared to a control cell that does not comprise the respective nucleic acid.

In some embodiments, the one or more nucleic acids further comprises at least a third nucleic acid sequence at least 15 nucleotides in length, wherein the at least a third nucleic acid sequence comprises a nucleic acid sequence complementary to nucleotides 1126 to 1364 of an mRNA encoding human Fas Cell Surface Death Receptor (FAS) comprising the sequence set forth in SEQ ID NO: 3.

In some embodiments, the third nucleic acid comprises a sequence selected from the group consisting of the sequences set forth in SEQ ID NOs: 85-99.

In some embodiments, the nucleic acid comprises the sequence set forth in SEQ ID NO: 92.

In some embodiments, the nucleic acid comprises the sequence set forth in SEQ ID NO: 130, 139, 129, or 141.

In some embodiments, the one or more nucleic acids further comprises at least a third nucleic acid sequence at least 15 nucleotides in length, wherein the third nucleic acid sequence comprises one or more of: (1) a nucleic acid sequence complementary to nucleotides 1126 to 1364 of an mRNA encoding human Fas Cell Surface Death Receptor (FAS) comprising the sequence set forth in SEQ ID NO: 3, (2) a nucleic acid sequence complementary to nucleotides 518 to 559 of an mRNA encoding human Protein Tyrosine Phosphatase Non-Receptor Type 2 (PTPN2) comprising the sequence set forth in SEQ ID NO: 4; or (3) a nucleic acid sequence complementary to nucleotides 1294 to 2141 of an mRNA encoding human Thymocyte Selection Associated High Mobility Group Box (TOX) comprising the sequence set forth in SEQ ID NO: 5.

In some embodiments, the third nucleic acid comprises a nucleic acid sequence complementary to nucleotides 1126 to 1364 of an mRNA encoding human FAS comprising the sequence set forth in SEQ ID NO: 3.

In some embodiments, the third nucleic acid comprises a sequence selected from the group consisting of the sequences set forth in SEQ ID NOs: 85-99.

In some embodiments, the nucleic acid comprises the sequence set forth in SEQ ID NO: 92.

In some embodiments, the first nucleic acid comprises the sequence set forth in SEQ ID NO: 81, the second nucleic acid comprises the sequence set forth in SEQ ID NO: 51, and the third nucleic acid comprises the sequence set forth in SEQ ID NO: 92.

In some embodiments, the one or more recombinant nucleic acids comprises the sequence set forth in SEQ ID NO: 129, 130, 139, or 141.

In some embodiments, the third nucleic acid comprises a nucleic acid sequence complementary to nucleotides 518 to 559 of an mRNA encoding human Protein Tyrosine Phosphatase Non-Receptor Type 2 (PTPN2) comprising the sequence set forth in SEQ ID NO: 4.

In some embodiments, the third nucleic acid comprises a sequence selected from the group consisting of the sequences set forth in SEQ ID NOs: 100-112.

In some embodiments, the nucleic acid comprises the sequence set forth in SEQ ID NO: 110.

In some embodiments, the nucleic acid comprises the sequence set forth in SEQ ID NO: 129.

In some embodiments, the third nucleic acid comprises a nucleic acid sequence complementary to nucleotides 1294 to 2141 of an mRNA encoding human Thymocyte Selection Associated High Mobility Group Box (TOX) comprising the sequence set forth in SEQ ID NO: 5.

In some embodiments, the third nucleic acid comprises a sequence selected from the group consisting of the sequences set forth in SEQ ID NOs: 113-126.

In some embodiments, further comprising at least a third and a fourth nucleic acid sequence at least 15 nucleotides in length, wherein the third nucleic acid sequence comprises a nucleic acid sequence complementary to nucleotides 1126 to 1364 of an mRNA encoding human Fas Cell Surface Death Receptor (FAS) comprising the sequence set forth in SEQ ID NO: 3, and the fourth nucleic acid sequence complementary to nucleotides 518 to 559 of an mRNA encoding human Protein Tyrosine Phosphatase Non-Receptor Type 2 (PTPN2) comprising the sequence set forth in SEQ ID NO: 4.

In some embodiments, the third nucleic acid comprises a sequence selected from the group consisting of the sequences set forth in SEQ ID NOs: 85-99.

In some embodiments, the nucleic acid comprises the sequence set forth in SEQ ID NO: 92.

In some embodiments, the third nucleic acid comprises a sequence selected from the group consisting of the sequences set forth in SEQ ID NOs: 100-112.

In some embodiments, the nucleic acid comprises the sequence set forth in SEQ ID NO: 110.

In some embodiments, the third nucleic acid reduces expression of FAS in a cell by at least 10%, 15%, 20%, 25%, 30%, 35%, 40%, 45%, 50%, 55%, 60%, 65%, 75%, 80%, 85%, 90%, 95%, or 99% and/or reduces expression of PTPN2 in a cell by at least 10%, 15%, 20%, 25%, 30%, 35%, 40%, 45%, 50%, 55%, 60%, 65%, 75%, 80%, 85%, 90%, 95%, or 99%, as compared to a control cell that does not comprise the nucleic acid.

In some embodiments, the recombinant nucleic acid(s) further comprises at least one of a nucleotide sequence encoding a priming receptor comprising a first extracellular antigen-binding domain that specifically binds to a first antigen and a nucleotide sequence encoding a chimeric antigen receptor (CAR) comprising a second extracellular antigen-binding domain that specifically binds to a second antigen, wherein the first antigen and the second antigen are distinct.

In some embodiments, the recombinant nucleic acid comprises, in a 5′ to 3′ direction: the CAR; the one or more recombinant nucleic acids disclosed herein; and the priming receptor.

In some embodiments, the nucleic acid comprises, in a 5′ to 3′ direction: the priming receptor; the one or more recombinant nucleic acids disclosed herein; and the CAR.

In some embodiments, the recombinant nucleic acid further comprises a 5′ homology directed repair arm and/or a 3′ homology directed repair arm complementary to an insertion site in a host cell chromosome.

In some embodiments, the recombinant nucleic acid comprises the 5′ homology directed repair arm and the 3′ homology directed repair arm.

In some embodiments, each of the one or more nucleic acids are encoded on a plurality of different nucleic acid molecules.

In some embodiments, each of the one or more nucleic acids are encoded on the same nucleic acid molecule.

In some embodiments, the one or more recombinant nucleic acids are incorporated into a one or more expression cassettes or expression vectors.

In some embodiments, the expression cassette or the expression vector further comprises a constitutive promoter upstream of the one or more recombinant nucleic acids.

In some embodiments, the expression cassette comprises the sequence set forth in any one of SEQ ID NOs: 133-137.

In some embodiments, the expression vector is a non-viral vector.

In one aspect, provided herein are expression vectors comprising the recombinant nucleic acid(s) disclosed herein.

In some embodiments, the expression vector is a non-viral vector.

In some embodiments, the 5′ and 3′ ends of the recombinant nucleic acid(s) comprise one or more nucleotide sequences that are homologous to genomic sequences flanking an insertion site in a genome of a cell.

In some embodiments, the insertion site is located at a T Cell Receptor Alpha Constant (TRAC) locus or a genomic safe harbor (GSH) locus.

In some embodiments, the GSH locus is the GS94 locus.

In one aspect, provided herein are immune cells comprising one or more recombinant nucleic acids at least 15 nucleotides in length complementary to an mRNA encoding human TGFBR1 comprising the sequence set forth in SEQ ID NO: 1.

In one aspect, provided herein are immune cells comprising one or more recombinant nucleic acids at least 15 nucleotides in length complementary to an mRNA encoding human TGFBR2 comprising the sequence set forth in SEQ ID NO: 2.

In one aspect, provided herein are immune cells comprising one or more recombinant nucleic acids comprising: a first nucleic acid sequence at least 15 nucleotides in length complementary to of an mRNA encoding human TGFBR2 comprising the sequence set forth in SEQ ID NO: 2, and a second nucleic acid sequence at least 15 nucleotides in length, wherein the second nucleic acid sequence is complementary to an mRNA encoding human TGFBR2 comprising the sequence set forth in SEQ ID NO: 2; or complementary to an mRNA encoding human TGFBR1 comprising the sequence set forth in SEQ ID NO: 1.

In some embodiments, the second nucleic acid sequence is complementary to an mRNA encoding human TGFBR2 comprising the sequence set forth in SEQ ID NO: 2.

In some embodiments, the second nucleic acid sequence is complementary to an mRNA encoding human TGFBR1 comprising the sequence set forth in SEQ ID NO: 1.

In one aspect, provided herein are immune cells comprising one or more recombinant nucleic acids comprising: a first nucleic acid sequence at least 15 nucleotides in length complementary to an mRNA encoding human TGFBR2 comprising the sequence set forth in SEQ ID NO: 2, and a second nucleic acid sequence at least 15 nucleotides in length complementary to an mRNA encoding human TGFBR1 comprising the sequence set forth in SEQ ID NO: 1.

In one aspect, provided herein are immune cells comprising one or more recombinant nucleic acids comprising: a first nucleic acid sequence and a second nucleic acid sequence at least 15 nucleotides in length complementary to an mRNA encoding human TGFBR2 comprising the sequence set forth in SEQ ID NO: 2.

In some embodiments, the cell further comprises at least a third nucleic acid sequence at least 15 nucleotides in length, wherein the third nucleic acid sequence is (1) complementary to nucleotides 1126 to 1364 of an mRNA encoding human FAS comprising the sequence set forth in SEQ ID NO: 3; (2) complementary to nucleotides 518 to 559 of an mRNA encoding human PTPN2 comprising the sequence set forth in SEQ ID NO: 4; or (3) complementary to nucleotides 1294 to 2141 of an mRNA encoding human Thymocyte Selection Associated High Mobility Group Box (TOX) comprising the sequence set forth in SEQ ID NO: 5.

In some embodiments, the cell further comprises at least a fourth nucleic acid sequence at least 15 nucleotides in length, wherein the fourth nucleic acid sequence is (1) complementary to nucleotides 1126 to 1364 of an mRNA encoding human FAS comprising the sequence set forth in SEQ ID NO: 3; (2) complementary to nucleotides 518 to 559 of an mRNA encoding human PTPN2 comprising the sequence set forth in SEQ ID NO: 4; or (3) complementary to nucleotides 1294 to 2141 of an mRNA encoding human Thymocyte Selection Associated High Mobility Group Box (TOX) comprising the sequence set forth in SEQ ID NO: 5.

In some embodiments, the first, second, third, and fourth nucleic acids are an shRNA, an siRNA, a dsRNA, or an antisense oligonucleotide.

In some embodiments, the first, second, third, and fourth nucleic acids are shRNA.

In some embodiments, the first nucleic acid comprises a sequence selected from the group consisting of the sequences set forth in SEQ ID NOs: 36-84.

In some embodiments, the first nucleic acid comprises the sequence set forth in SEQ ID NO: 81.

In some embodiments, the second nucleic acid comprises a sequence selected from the group consisting of the sequences set forth in SEQ ID NOs: 6-35.

In some embodiments, the second nucleic acid comprises the sequence set forth in SEQ ID NO: 16 or 28.

In some embodiments, the first nucleic acid comprises a sequence selected from the group consisting of the sequences set forth in SEQ ID NOs: 36-84; and the second nucleic acid sequence comprises a sequence selected from the group consisting of the sequences set forth in SEQ ID NOs: 6-35.

In some embodiments, the first nucleic acid comprises the sequence set forth in SEQ ID NO: 81; and the second nucleic acid comprises the sequence set forth in SEQ ID NO: 16 or 28.

In some embodiments, the second nucleic acid comprises a sequence selected from the group consisting of the sequences set forth in SEQ ID NOs: 36-84.

In some embodiments, the second nucleic acid comprises the sequence set forth in SEQ ID NO: 51 or 53.

In some embodiments, the first nucleic acid and second nucleic acids comprises a sequence selected from the group consisting of the sequences set forth in SEQ ID NOs: 36-84.

In some embodiments, the first nucleic acid comprises the sequence set forth in SEQ ID NO: 81; and the second nucleic acid comprises the sequence set forth in SEQ ID NO: 51 or 53.

In some embodiments, the one or more recombinant nucleic acids comprises the sequence set forth in SEQ ID NO: 128, 129, 130, 139, 140, or 141.

In some embodiments, the first nucleic acid reduces expression of TGFBR2 in a cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the first nucleic acid.

In some embodiments, expression of TGFBR2 in a cell is reduced by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the first nucleic acid.

In some embodiments, the second nucleic acid reduces expression of TGFBR2 in a cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the second nucleic acid.

In some embodiments, expression of TGFBR2 in a cell is reduced by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the second nucleic acid.

In some embodiments, the first nucleic acid reduces expression of TFGBR2 in a cell by at least 10%, 15%, 20%, 25%, 30%, 35%, 40%, 45%, 50%, 55%, 60%, 65%, 75%, 80%, 85%, 90%, 95%, or 99% and the second nucleic acid reduces expression of TGFBR2 in a cell by at least 10%, 15%, 20%, 25%, 30%, 35%, 40%, 45%, 50%, 55%, 60%, 65%, 75%, 80%, 85%, 90%, 95%, or 99%, each as compared to a control cell that does not comprise the respective nucleic acid.

In some embodiments, the second nucleic acid reduces expression of TGFBR1 in a cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the second nucleic acid.

In some embodiments, expression of TGFBR1 in a cell is reduced by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the second nucleic acid.

In some embodiments, the first nucleic acid reduces expression of TFGBR2 in a cell by at least 10%, 15%, 20%, 25%, 30%, 35%, 40%, 45%, 50%, 55%, 60%, 65%, 75%, 80%, 85%, 90%, 95%, or 99% and the second nucleic acid reduces expression of TGFBR1 in a cell by at least 10%, 15%, 20%, 25%, 30%, 35%, 40%, 45%, 50%, 55%, 60%, 65%, 75%, 80%, 85%, 90%, 95%, or 99%, each as compared to a control cell that does not comprise the respective nucleic acid.

In some embodiments, the third nucleic acid sequence is complementary to nucleotides 1126 to 1364 of an mRNA encoding human FAS comprising the sequence set forth in SEQ ID NO: 3.

In some embodiments, the third nucleic acid comprises a sequence selected from the group consisting of the sequences set forth in SEQ ID NOs: 85-99.

In some embodiments, the third nucleic acid comprises the sequence set forth in SEQ ID NO: 92.

In some embodiments, the third nucleic acid reduces expression of FAS in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the third nucleic acid.

In some embodiments, the fourth nucleic acid sequence is complementary to nucleotides 518 to 559 of an mRNA encoding human PTPN2 comprising the sequence set forth in SEQ ID NO: 4.

In some embodiments, the fourth nucleic acid comprises a sequence selected from the group consisting of the sequences set forth in SEQ ID NOs: 100-112.

In some embodiments, the fourth nucleic acid comprises the sequence set forth in SEQ ID NO: 110.

In some embodiments, the one or more recombinant nucleic acids comprises the sequence set forth in SEQ ID NO: 129.

In some embodiments, the fourth nucleic acid reduces expression of PTPN2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the first nucleic acid.

In some embodiments, the fourth nucleic acid sequence is complementary to nucleotides 1294 to 2141 of an mRNA encoding human Thymocyte Selection Associated High Mobility Group Box (TOX) comprising the sequence set forth in SEQ ID NO: 5.

In some embodiments, the fourth nucleic acid comprises a sequence selected from the group consisting of the sequences set forth in SEQ ID NOs: 113-126.

In some embodiments, expression of TGFBR1, TGFBR2, FAS and/or PTPN2 and/or TOX is determined by a nucleic acid assay or a protein assay.

In some embodiments, the nucleic acid assay comprises at least one of polymerase chain reaction (PCR), quantitative PCR (qPCR), RT-qPCR, microarray, gene array, or RNAseq.

In some embodiments, the protein assay comprises at least one of immunoblotting, fluorescence activated cell sorting, flow-cytometry, magnetic-activated cell sorting, or affinity-based cell separation.

In some embodiments, the cell further comprises a priming receptor comprising a first extracellular antigen-binding domain that specifically binds to a first antigen and a chimeric antigen receptor (CAR) comprising a second extracellular antigen-binding domain that specifically binds to a second antigen.

In some embodiments, the immune cell is a primary human immune cell.

In some embodiments, the primary immune cell is a natural killer (NK) cell, a natural killer T (NKT) cell, a T cell, a γδ T cell, a CD8+ T cell, a CD4+ T cell, a primary T cell, a T cell progenitor, or an induced pluripotent stem cell (iPSC).

In some embodiments, the primary immune cell is a primary T cell.

In some embodiments, the primary immune cell is a primary human T cell.

In some embodiments, the immune cell is virus-free.

In some embodiments, the immune cell is an autologous immune cell.

In some embodiments, the immune cell is an allogeneic immune cell.

In one aspect, provided herein are primary immune cells comprising at least one recombinant nucleic acid(s) comprising a first nucleic acid comprising a sequence selected from the group consisting of the sequences set forth in SEQ ID NO: 36-84; and a second nucleic acid comprising a sequence selected from the group consisting of the sequences set forth in SEQ ID NO: 6-84, inserted into a target region of the genome of the primary immune cell, and wherein the primary immune cell does not comprise a viral vector for introducing the recombinant nucleic acid(s) into the primary immune cell.

In one aspect, provided herein are primary immune cells comprising at least one recombinant nucleic acid(s) comprising a first nucleic acid comprising a sequence selected from the group consisting of the sequences set forth in SEQ ID NO: 36-84; and a second nucleic acid comprising a sequence selected from the group consisting of the sequences set forth in SEQ ID NO: 6-84, inserted into a target region of the genome of the primary immune cell, and wherein the primary immune cell does not comprise a viral vector for introducing the recombinant nucleic acid into the primary immune cell.

In one aspect, provided herein are viable, virus-free, primary cells comprising a ribonucleoprotein complex (RNP)-recombinant nucleic acid(s) complex, wherein the RNP comprises a nuclease domain and a guide RNA, wherein recombinant nucleic acid(s) comprises a first nucleic acid comprising a sequence selected from the group consisting of the sequences set forth in SEQ ID NO: 36-84; and a second nucleic acid comprising a sequence selected from the group consisting of the sequences set forth in SEQ ID NO: 6-84, and wherein the 5′ and 3′ ends of the recombinant nucleic acid(s) comprise nucleotide sequences that are homologous to genomic sequences flanking an insertion site in the genome of the primary cell.

In one aspect, provided herein are viable, virus-free, primary cells comprising a ribonucleoprotein complex (RNP)-recombinant nucleic acid(s) complex, wherein the RNP comprises a nuclease domain and a guide RNA, wherein recombinant nucleic acid(s) comprises a first nucleic acid comprising a sequence selected from the group consisting of the sequences set forth in in SEQ ID NO: 36-84; and a second nucleic acid comprising a sequence selected from the group consisting of the sequences set forth in SEQ ID NO: 6-84, and wherein the 5′ and 3′ ends of the recombinant nucleic acid(s) comprise nucleotide sequences that are homologous to genomic sequences flanking an insertion site in the genome of the primary cell.

In some embodiments, the cell further comprises a priming receptor comprising a first extracellular antigen-binding domain that specifically binds to a first antigen and a chimeric antigen receptor (CAR) comprising a second extracellular antigen-binding domain that specifically binds to a second antigen, wherein the first antigen and the second antigen are distinct.

In one aspect, provided herein are populations of cells comprising a plurality of immune cells disclosed herein.

In one aspect, provided herein are pharmaceutical compositions comprising the immune cell disclosed herein or the population of cells disclosed herein, and a pharmaceutically acceptable excipient.

In one aspect, provided herein are pharmaceutical compositions comprising the one or more recombinant nucleic acids disclosed herein or the vector disclosed herein, and a pharmaceutically acceptable excipient.

In one aspect, provided herein are methods of editing an immune cell, comprising: providing a ribonucleoprotein (RNP)-recombinant nucleic acid(s) complex, wherein the RNP comprises a nuclease domain and a guide RNA, wherein the recombinant nucleic acid(s) comprises the recombinant nucleic acid(s) disclosed herein, and wherein the 5′ and 3′ ends of the recombinant nucleic acid(s) comprise nucleotide sequences that are homologous to genomic sequences flanking an insertion site in the genome of the immune cell; non-virally introducing the RNP-recombinant nucleic acid(s) complex into the immune cell, wherein the guide RNA specifically hybridizes to a target region of the genome of the primary immune cell, and wherein the nuclease domain cleaves the target region to create the insertion site in the genome of the immune cell; and editing the immune cell via insertion of the recombinant nucleic acid(s) disclosed herein into the insertion site in the genome of the immune cell.

In some embodiments, non-virally introducing comprises electroporation.

In some embodiments, the nuclease domain comprises a CRISPR-associated endonuclease (Cas), optionally a Cas9 nuclease.

In some embodiments, the target region of the genome of the cell is a T Cell Receptor Alpha Constant (TRAC) locus or a genomic safe harbor (GSH) locus.

In some embodiments, the recombinant nucleic acid(s) is a double-stranded recombinant nucleic acid(s) or a single-stranded recombinant nucleic acid(s).

In some embodiments, the recombinant nucleic acid(s) is a linear recombinant nucleic acid(s) or a circular recombinant nucleic acid(s), optionally wherein the circular recombinant nucleic acid(s) is a plasmid.

In some embodiments, the immune cell is a primary human immune cell.

In some embodiments, the immune cell is an autologous immune cell.

In some embodiments, the immune cell is an allogeneic immune cell.

In some embodiments, the immune cell is a natural killer (NK) cell, a natural killer T (NKT) cell, a T cell, a γδ T cell, a CD8+ T cell, a CD4+ T cell, a primary T cell, a T cell progenitor, or an induced pluripotent stem cell (iPSC).

In some embodiments, the immune cell is a primary T cell.

In some embodiments, the immune cell is a primary human T cell.

In some embodiments, the immune cell is virus-free or does not comprise a viral vector.

In some embodiments, further comprising obtaining the immune cell from a patient and introducing the recombinant nucleic acid(s) in vitro.

In one aspect, provided herein are methods of treating a disease in a subject comprising administering the immune cell(s) disclosed herein or the pharmaceutical composition disclosed herein to the subject.

In some embodiments, the disease is cancer.

In some embodiments, the cancer is a solid cancer or a liquid cancer.

In some embodiments, the cancer is ovarian cancer, fallopian cancer, primary peritoneal cancer, uterine cancer, mesothelioma, cervical cancer, pancreatic, kidney cancer, lung cancer, prostate cancer, bladder cancer, breast cancer, brain cancer, leukemia, or lymphoma.

In some embodiments, the administration of the cell(s) enhances an immune response.

In some embodiments, the enhanced immune response is an adaptive immune response.

In some embodiments, the enhanced immune response is an innate immune response.

In one aspect, provided herein are methods of enhancing an immune response in a subject comprising administering the immune cell(s) disclosed herein or the pharmaceutical composition disclosed herein to the subject.

In some embodiments, the enhanced immune response is an adaptive immune response.

In some embodiments, the enhanced immune response is an innate immune response.

In some embodiments, expression of TGFBR2 in the immune cell is reduced by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the respective nucleic acid.

In some embodiments, expression of TGFBR1 in the immune cell is reduced by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the respective nucleic acid.

In some embodiments, expression of FAS in the immune cell is reduced by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the respective nucleic acid.

In some embodiments, expression of PTPN2 in the immune cell is reduced by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the respective nucleic acid.

In some embodiments, expression of TOX in the immune cell is reduced by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the respective nucleic acid.

In some embodiments, expression of TGFBR1, TGFBR2, FAS, PTPN2, and/or TOX in the immune cell is determined by a nucleic acid assay or a protein assay.

In some embodiments, the nucleic acid assay comprises at least one of polymerase chain reaction (PCR), quantitative PCR (qPCR), RT-qPCR, microarray, gene array, or RNAseq.

In some embodiments, the protein assay comprises at least one of immunoblotting, fluorescence activated cell sorting, flow-cytometry, magnetic-activated cell sorting, or affinity-based cell separation.

In some embodiments, further comprising administering an immunotherapy to the subject concurrently with the immune cell or subsequently to the immune cell.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1A shows an exemplary insertion cassette encoding a logic gate (priming receptor (primeR) and CAR) and an shRNA module. FIG. 1B shows knockdown of TGFBR2 and FAS in T cells incubated with the indicated shRNA module. FIG. 1C shows knockdown of TGFBR2 and FAS in T cells incubated with the indicated shRNA module and 0 ng/ml or 10 ng/ml TGF-β1. FIG. 1D shows knockdown of TGFBR2 and FAS in T cells incubated with the indicated shRNA module and 0 ng/ml or 10 ng/ml TGF-β1 after short RSA (2nd stim). FIG. 1E show the relative TFGBR2 expression after knockdown with the indicated shRNA. FIG. 1F shows the cumulative T cell expansion, tumor cell expansion, and IFNg production in T cells with single TGFBR2 shRNA stimulated in media alone or in the presence of TGF-b. FIG. 1G shows the TFGBR2 expression and pSMAD phosphorylation in T cells after knockdown with the indicated shRNA or FAS-PTPN2-TGFBR2 knockout.

FIG. 2A shows that the indicated shRNAs conferred partial resistance to TGF-β mediated CD103 induction after tumor plus TGF-β1 exposure in short term RSA. FIG. 2B shows that the indicated shRNAs conferred partial resistance to TGF-β mediated CD103 induction after tumor plus TGF-β1 exposure in RPMI-8226 target cells.

FIG. 3 shows TGFBR2 knockdown by the indicated Quad shRNA modules rescued TGF-β-mediated suppression of tumor cell killing by logic gate T cells.

FIG. 4 shows a correlation of CD103 induction on ICTs following short RSA (2-stimulations) after treatment of cells with the indicated shRNAs.

FIG. 5A shows selected TGFBR quads partially impaired CD103 induction during RSA. FIG. 5B shows that selected TGFBR shRNA modules strongly impaired maintenance of PD1 during RSA.

FIG. 6 shows that MULTIseq analysis shows on-target suppression of miR shRNA modules.

FIG. 7 shows that the TGFBR2-TGFBR2 Quad shRNA modules exhibited a reduction in pSMAD2/3 induction in response to TGF-β1.

FIG. 8 shows that TGFBR2 knockdown by selected quad shRNA inhibited TGF-β-mediated suppression of target cell killing by logic gate T cells

FIG. 9A shows that TGFBR2 knock-out in logic gate T cells enhanced antitumor activity in the H1975 xenograft model. FIG. 9B shows that FAS-PTPN2 shRNA enhanced the efficacy of logic gate T cells in the H1975 in vivo model. FIG. 9C shows that TGFBR2 knock-out in logic gate T cells enhanced antitumor activity in the 786-O xenograft model.

FIG. 9D shows expansion of the T cells with TGFBR2 KO on Day 14 in the 786-O xenograft model.

FIG. 10 shows that TGFBR2 knockout T cells expressing the indicated shRNA exhibited enhanced TGI in the 786-O model as compared to WT T cells and T cells expressing only FAS-PTPN2 shRNA.

FIG. 11A shows the average tumor volume of after treatment with the engineered T cells. FIG. 11B shows the individual mouse tumor size after treatment with the logic gate and control shRNA control group. FIG. 11C shows the individual mouse tumor size after treatment with the FAS-PTPN2-TGFBR2_23-TGBR2_16 shRNA logic gate T cell. FIG. 11D shows the individual mouse tumor size after treatment with the FAS-PTPN2 logic gate T cell. FIG. 11E shows the individual mouse tumor size after treatment with the FAS-PTPN2-TGFBR2_23-TGBR2_37 shRNA logic gate T cell. FIG. 11F shows the individual mouse tumor size after treatment with the FAS-PTPN2-TGFBR2_23-TGBR1_13 shRNA logic gate T cell. FIG. 11G shows the individual mouse tumor size after treatment with the FAS-PTPN2-TGFBR2_23-TGBR1_10 shRNA logic gate T cell. FIG. 11H shows the individual mouse tumor size after treatment with the TGFBR2 knockout T cell.

FIG. 12 provides diagram of the various ICT transgene cassettes expressing Logic Gate 1-5 ICs, shRNA, and optional SPAs.

FIG. 13 shows that all ICT cells constitutively expressed the PrimeR construct.

FIG. 14 shows that the ICT cells induced CAR expression when co-cultured with primeR antigen expressing cell lines.

FIG. 15 shows that inclusion of the shRNA module in ICT cells resulted in lower MFI for both FAS and TGFBR2 in ICT cells expressing the priming receptor-CAR logic gate (PrimeR+) normalized to non-edited cells (PrimeR−).

FIG. 16A shows cytotoxicity against parental K562 cells expressing neither CAR antigen or primeR antigen, FIG. 16B shows cytotoxicity against K562 cells expressing only CAR antigen, FIG. 16C cytotoxicity against K562 cells expressing only primeR antigen. FIG. 16D shows cytotoxicity against K562 cells expressing both primeR antigen and CAR antigen.

FIG. 17 shows IFN-γ production from ICTs expressing Logic Gates 1-5 only in supernatants taken from co-cultures where the target cells expressed both primeR antigen and CAR antigen.

FIG. 18A shows that ICTs expressing Logic Gates 1-5 demonstrated in vitro cytotoxicity against the primeR antigen+ cell line expressing endogenous CAR antigen FIG. 18B shows IFNγ, TNFα, GM-CSF, and IL-2 secretion by ICT cells after co-culture with primeR antigen+/CAR antigen+ cells.

FIG. 19 shows that co-culture with HUVEC-primeR antigen+ cells induced expression of the CAR protein on ICT cells and specific killing of CAR antigen+ cells.

FIG. 20A shows the tumor volume post tumor implant in mice treated with ICTs expressing Logic Gates 1-5, RNP or PBS generated from donor 1. FIG. 20B shows the total T cells and expansion of the ICTs on day 12 post inoculation followed by contraction by day 21. FIG. 20C shows total T cells expressing the priming receptor on days 12 and 21. FIG. 20D show the tumor volume post tumor implant in mice treated with ICTs expressing Logic Gates 1-5, RNP or PBS generated from donor 2. FIG. 20E shows the total T cells and expansion of the ICTs on day 12 post inoculation followed by contraction by day 21. FIG. 20F shows total T cells expressing the priming receptor on days 12 and 21.

FIG. 21A shows tumor growth inhibition (TGI) in the single positive CAR antigen-only flank. FIG. 21B shows tumor growth inhibition (TGI) in the dual positive primeR antigen/CAR antigen flank.

FIG. 22 shows that TGFBR knockdown protects ICT cells against TGFβ-mediated inhibition.

FIG. 23 shows that the ICT+shRNA was more potent than a conventional CAR-T benchmark.

FIG. 24 shows in vivo tumor volume in the 786-O xenograft model after injection of the indicated ICTs expressing quad shRNAs.

FIG. 25 shows in vivo tumor volume in the A498 xenograft model after injection of the ICTs expressing quad shRNAs as compared to FAS-PTPN2 shRNA.

FIG. 26 shows a schematic of an exemplary dual shRNA expression cassette for expression of shRNAs against FAS and TGFBR2 in the 3G-E format.

FIG. 27 shows a schematic of an exemplary triple shRNA expression cassette for expression of shRNAs against FAS and TGFBR2 in the 3G-E-3G format.

FIG. 28 shows a schematic of an exemplary triple shRNA expression cassette for expression of shRNAs against TGFBR2 and FAS in the 3G-3G-3G format.

FIG. 29 shows a schematic of an exemplary triple shRNA expression cassette for expression of shRNAs against FAS and TGFBR2 in the 3G-3G-3G format.

FIG. 30 shows a schematic of an exemplary quadruple shRNA expression cassette for expression of shRNAs against FAS, PTPN2, and TGFBR2 in the 3G-E-3G-3G format.

### DETAILED DESCRIPTION

### Definitions

Terms used in the claims and specification are defined as set forth below unless otherwise specified.

As used herein, the term “locus” refers to a specific, fixed physical location on a chromosome where a gene or genetic marker is located.

The term “safe harbor locus” refers to a locus at which genes or genetic elements can be incorporated without disruption to expression or regulation of adjacent genes. These safe harbor loci are also referred to as safe harbor sites (SHS) or genomic safe harbor (GSH) sites. As used herein, a safe harbor locus refers to an “integration site” or “knock-in site” at which a sequence encoding a transgene, as defined herein, can be inserted. In some embodiments the insertion occurs with replacement of a sequence that is located at the integration site. In some embodiments, the insertion occurs without replacement of a sequence at the integration site. Examples of integration sites contemplated are provided in Table D.

As used herein, the term “insert” refers to a nucleotide sequence that is integrated (inserted) at a target locus or safe harbor site. The insert can be used to refer to the genes or genetic elements that are incorporated at the target locus or safe harbor site using, for example, homology-directed repair (HDR) CRISPR/Cas9 genome-editing or other methods for inserting nucleotide sequences into a genomic region known to those of ordinary skill in the art.

The term “inserting” refers to a manipulation of a nucleotide sequence to introduce a non-native sequence. This is done, for example, via the use of restriction enzymes and ligases whereby the DNA sequence of interest, usually encoding the gene of interest, can be incorporated into another nucleic acid molecule by digesting both molecules with appropriate restriction enzymes in order to create compatible overlaps and then using a ligase to join the molecules together. One skilled in the art is very familiar with such manipulations and examples may be found in Sambrook et al. (Sambrook, Fritsch, & Maniatis, “Molecular Cloning: A Laboratory Manual”, 2nd ed., Cold Spring Harbor Laboratory, 1989), which is hereby incorporated by reference in its entirety including any drawings, figures and tables.

The “CRISPR/Cas” system refers to a widespread class of bacterial systems for defense against foreign nucleic acid. CRISPR/Cas systems are found in a wide range of cubacterial and archacal organisms. CRISPR/Cas systems include type I, II, and III sub-types. Wild-type type II CRISPR/Cas systems utilize an RNA-mediated nuclease, Cas9 in complex with guide and activating RNA to recognize and cleave foreign nucleic acid. Guide RNAs having the activity of both a guide RNA and an activating RNA are also known in the art. In some cases, such dual activity guide RNAs are referred to as a small guide RNA (sgRNA).

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10 (5): 726-737; Nat. Rev. Microbiol. 2011 June; 9 (6): 467-477; Hou, et al., Proc Natl Acad Sci USA. 2013 Sep. 24; 110 (39): 15644-9; Sampson et al., Nature. 2013 May 9; 497 (7448): 254-7; and Jinek, et al., Science. 2012 Aug. 17; 337 (6096): 816-21. The Cas9 nuclease domain can be optimized for efficient activity or enhanced stability in the host cell.

As used herein, the term “Cas9” refers to an RNA-mediated nuclease (e.g., of bacterial or archaeal origin, or derived therefrom). Exemplary RNA-mediated nucleases include the foregoing Cas9 proteins and homologs thereof, and include but are not limited to, CPF1 (See, e.g., Zetsche et al., Cell, Volume 163, Issue 3, p 759-771, 22 Oct. 2015). Similarly, as used herein, the term “Cas9 ribonucleoprotein” complex and the like refers to a complex between the Cas9 protein, and a crRNA (e.g., guide RNA or small guide RNA), the Cas9 protein and a trans-activating crRNA (tracrRNA), the Cas9 protein and a small guide RNA, or a combination thereof (e.g., a complex containing the Cas9 protein, a tracrRNA, and a crRNA guide RNA).

As used herein, the phrase “immune cell” is inclusive of all cell types that can give rise to immune cells, including hematopoietic cells such hematopoietic stem cells, pluripotent stem cells, and induced pluripotent stem cells (iPSCs). In some embodiments, the immune cell is a B cell, macrophage, a natural killer (NK) cell, an induced pluripotent stem cell (iPSC), a human pluripotent stem cell (HSPC), a T cell or a T cell progenitor or dendritic cell. In some embodiments, the cell is an innate immune cell.

As used herein, the term “primary” in the context of a primary cell or primary stem cell refers to a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized, e.g., directly without culturing or sub-culturing. In some cases, the primary cells are stimulated, activated, or differentiated. For example, primary T cells can be activated by contact with (e.g., culturing in the presence of) CD3, CD28 agonists, IL-2, IFN-γ, or a combination thereof.

As used herein, the terms “T lymphocyte” and “T cell” are used interchangeably and refer to cells that have completed maturation in the thymus, and identify certain foreign antigens in the body. The terms also refer to the major leukocyte types that have various roles in the immune system, including activation and deactivation of other immune cells. The T cell can be any T cell such as a cultured T cell, e.g., a primary T cell, or a T cell derived from a cultured T cell line, e.g., a Jurkat, SupT1, etc., or a T cell obtained from a mammal. T cells include, but are not limited to, naïve T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. The T cell can be a CD3+ cell. T cells can be CD4⁺, CD8⁺, or CD4⁺ and CD8⁺. The T cell can be any type of T cell, CD4+/CD8+ double positive T cells, CD4+ helper T cells (e.g. Th1 and Th2 cells), CD8+ T cells (e.g. cytotoxic T cells), peripheral Including but not limited to blood mononuclear cells (PBMC), peripheral blood leukocytes (PBL), tumor infiltrating lymphocytes (TIL), memory T cells, naive T cells, regulatory T cells, γδ T cells, etc. It can be any T cell at any stage of development. Additional types of helper T cells include Th3 (Treg) cells, Th17 cells, Th9 cells, or Tfh cells. Additional types of memory T cells include cells such as central memory T cells (Tem cells), effector memory T cells (Tem cells and TEMRA cells). A T cell can also refer to a genetically modified T cell, such as a T cell that has been modified to express a T cell receptor (TCR) or a chimeric antigen receptor (CAR). T cells can also be differentiated from stem cells or progenitor cells.

“CD4+ T cells” refers to a subset of T cells that express CD4 on their surface and are associated with a cellular immune response. CD4+ T cells are characterized by a post-stimulation secretion profile that can include secretion of cytokines such as IFN-γ, TNF-α, IL-2, IL-4 and IL-10. “CD4” is a 55 kD glycoprotein originally defined as a differentiation antigen on T lymphocytes, but was also found on other cells including monocytes/macrophages. The CD4 antigen is a member of the immunoglobulin superfamily and has been implicated as an associative recognition element in MHC (major histocompatibility complex) class II restricted immune responses. On T lymphocytes, the CD4 antigen defines a helper/inducer subset.

“CD8+ T cells” refers to a subset of T cells that express CD8 on their surface, are MHC class I restricted, and function as cytotoxic T cells. The “CD8” molecule is a differentiation antigen present on thymocytes, as well as on cytotoxic and suppressor T lymphocytes. The CD8 antigen is a member of the immunoglobulin superfamily and is an associative recognition element in major histocompatibility complex class I restriction interactions.

As used herein, the phrase “hematopoietic stem cell” refers to a type of stem cell that can give rise to a blood cell. Hematopoietic stem cells can give rise to cells of the myeloid or lymphoid lineages, or a combination thereof. Hematopoietic stem cells are predominantly found in the bone marrow, although they can be isolated from peripheral blood, or a fraction thereof. Various cell surface markers can be used to identify, sort, or purify hematopoietic stem cells. In some cases, hematopoietic stem cells are identified as c-kit⁺ and lin⁻. In some cases, human hematopoietic stem cells are identified as CD34⁺, CD59⁺, Thy1/CD90⁺, CD38^{lo/-}, C-kit/CD117⁺, lin⁻. In some cases, human hematopoietic stem cells are identified as CD34⁻, CD59⁺, Thy1/CD90⁺, CD38^{lo/-}, C-kit/CD117⁺, lin⁻. In some cases, human hematopoietic stem cells are identified as CD133⁺, CD59⁺, Thy1/CD90⁺, CD38^{lo/-}, C-kit/CD117⁺, lin⁻. In some cases, mouse hematopoietic stem cells are identified as CD34^{lo/-}, SCA-1⁺, Thy1^{+/lo}, CD38⁺, C-kit⁺, lin⁻. In some cases, the hematopoietic stem cells are CD150⁺CD48⁻CD244⁻.

As used herein, the phrase “hematopoietic cell” refers to a cell derived from a hematopoictic stem cell. The hematopoietic cell may be obtained or provided by isolation from an organism, system, organ, or tissue (e.g., blood, or a fraction thereof). Alternatively, an hematopoietic stem cell can be isolated and the hematopoietic cell obtained or provided by differentiating the stem cell. Hematopoietic cells include cells with limited potential to differentiate into further cell types. Such hematopoietic cells include, but are not limited to, multipotent progenitor cells, lineage-restricted progenitor cells, common myeloid progenitor cells, granulocyte-macrophage progenitor cells, or megakaryocyte-erythroid progenitor cells. Hematopoietic cells include cells of the lymphoid and myeloid lineages, such as lymphocytes, erythrocytes, granulocytes, monocytes, and thrombocytes.

As used herein, the term “construct” refers to a complex of molecules, including macromolecules or polynucleotides.

As used herein, the term “integration” refers to the process of stably inserting one or more nucleotides of a construct into the cell genome, i.e., covalently linking to a nucleic acid sequence in the chromosomal DNA of the cell. It may also refer to nucleotide deletions at a site of integration. Where there is a deletion at the insertion site, “integration” may further include substitution of the endogenous sequence or nucleotide deleted with one or more inserted nucleotides.

As used herein, the term “exogenous” refers to a molecule or activity that has been introduced into a host cell and is not native to that cell. The molecule can be introduced, for example, by introduction of the encoding nucleic acid into host genetic material, such as by integration into a host chromosome, or as non-chromosomal genetic material, such as a plasmid. Thus, the term, when used in connection with expression of an encoding nucleic acid, refers to the introduction of the encoding nucleic acid into a cell in an expressible form. The term “endogenous” refers to a molecule or activity that is present in a host cell under natural, unedited conditions. Similarly, the term, when used in connection with expression of the encoding nucleic acid, refers to expression of the encoding nucleic acid that is contained within the cell and not introduced exogenously.

The term “heterologous” refers to a nucleic acid or polypeptide sequence or domain which is not native to a flanking sequence, e.g., wherein the heterologous sequence is not found in nature coupled to the nucleic acid or polypeptide sequences occurring at one or both ends.

The term “homologous” refers to a nucleic acid or polypeptide sequence or domain which is native to a flanking sequence, e.g., wherein the homologous sequence is found in nature coupled to the nucleic acid or polypeptide sequences occurring at one or both ends.

As used herein, a “polynucleotide donor construct” refers to a nucleotide sequence (e.g. DNA sequence) that is genetically inserted into a polynucleotide and is exogenous to that polynucleotide. The polynucleotide donor construct is transcribed into RNA and optionally translated into a polypeptide. The polynucleotide donor construct can include prokaryotic sequences, cDNA from eukaryotic mRNA, genomic DNA sequences from eukaryotic (e.g., mammalian) DNA, and synthetic DNA sequences. For example, the polynucleotide donor construct can be a miRNA, shRNA, natural polypeptide (i.e., a naturally occurring polypeptide) or fragment thereof or a variant polypeptide (e.g. a natural polypeptide having less than 100% sequence identity with the natural polypeptide) or fragments thereof.

As used herein, the term “complementary” or “complementarity” refers to specific base pairing between nucleotides or nucleic acids. Complementary nucleotides are, generally, A and T (or A and U), and G and C. The guide RNAs described herein can comprise sequences, for example, DNA targeting sequence that are perfectly complementary or substantially complementary (e.g., having 1-4 mismatches) to a genomic sequence in a cell.

As used herein, the term “transgene” refers to a polynucleotide that has been transferred naturally, or by any of a number of genetic engineering techniques from one organism to another. It is optionally translated into a polypeptide. It is optionally translated into a recombinant protein. A “recombinant protein” is a protein encoded by a gene—recombinant DNA—that has been cloned in a system that supports expression of the gene and translation of messenger RNA (see expression system). The recombinant protein can be a therapeutic agent, e.g. a protein that treats a disease or disorder disclosed herein. As used, transgene can refer to a polynucleotide that encodes a polypeptide.

The terms “protein,” “polypeptide,” and “peptide” are used herein interchangeably.

As used herein, the term “operably linked” or “operatively linked” refers to the binding of a nucleic acid sequence to a single nucleic acid fragment such that one function is affected by the other. For example, if a promoter is capable of affecting the expression of a coding sequence or functional RNA (i.e., the coding sequence or functional RNA is under transcriptional control by the promoter), the promoter is operably linked thereto. Coding sequences can be operably linked to control sequences in both sense and antisense orientation.

As used herein, the term “developmental cell states” refers to, for example, states when the cell is inactive, actively expressing, differentiating, senescent, etc. developmental cell state may also refer to a cell in a precursor state (e.g., a T cell precursor).

As used, the term “encoding” refers to a sequence of nucleic acids which codes for a protein or polypeptide of interest. The nucleic acid sequence may be either a molecule of DNA or RNA. In preferred embodiments, the molecule is a DNA molecule. In other preferred embodiments, the molecule is a RNA molecule. When present as a RNA molecule, it will comprise sequences which direct the ribosomes of the host cell to start translation (e.g., a start codon, ATG) and direct the ribosomes to end translation (e.g., a stop codon). Between the start codon and stop codon is an open reading frame (ORF). Such terms are known to one of ordinary skill in the art.

As used herein, the term “subject” refers to a mammalian subject. Exemplary subjects include humans, monkeys, dogs, cats, mice, rats, cows, horses, camels, goats, rabbits, pigs and sheep. In certain embodiments, the subject is a human. In some embodiments the subject has a disease or condition that can be treated with an engineered cell provided herein or population thereof. In some aspects, the disease or condition is a cancer.

As used herein, the term “promoter” refers to a nucleotide sequence (e.g. DNA sequence) capable of controlling the expression of a coding sequence or functional RNA. The promoter sequence consists of proximal and more distal upstream elements, the latter elements often referred to as enhancers. A promoter can be derived from natural genes in its entirety, can be composed of different elements from different promoters found in nature, and/or may comprise synthetic DNA segments. A promoter, as contemplated herein, can be endogenous to the cell of interest or exogenous to the cell of interest. It is appreciated by those skilled in the art that different promoters can induce gene expression in different tissue or cell types, or at different developmental stages, or in response to different environmental conditions. As is known in the art, a promoter can be selected according to the strength of the promoter and/or the conditions under which the promoter is active, e.g., constitutive promoter, strong promoter, weak promoter, inducible/repressible promoter, tissue specific Or developmentally regulated promoters, cell cycle-dependent promoters, and the like.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter). In some embodiments, the promoter can be a spatially restricted and/or temporally restricted promoter (e.g., a tissue specific promoter, a cell type specific promoter, etc.). See for example US Publication 20180127786, the disclosure of which is herein incorporated by reference in its entirety.

Gene editing, as contemplated herein, may involve a gene (or nucleotide sequence) knock-in or knock-out. As used herein, the term “knock-in” refers to an addition of a DNA sequence, or fragment thereof into a genome. Such DNA sequences to be knocked-in may include an entire gene or genes, may include regulatory sequences associated with a gene or any portion or fragment of the foregoing. For example, a polynucleotide donor construct encoding a recombinant protein may be inserted into the genome of a cell carrying a mutant gene. In some embodiments, a knock-in strategy involves substitution of an existing sequence with the provided sequence, e.g., substitution of a mutant allele with a wild-type copy. On the other hand, the term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant (.e.g., non-coding) sequence.

As used herein, the term “non-homologous end joining” or NHEJ refers to a cellular process in which cut or nicked ends of a DNA strand are directly ligated without the need for a homologous template nucleic acid. NHEJ can lead to the addition, the deletion, substitution, or a combination thereof, of one or more nucleotides at the repair site.

As used herein, the term “homology directed repair” or HDR refers to a cellular process in which cut or nicked ends of a DNA strand are repaired by polymerization from a homologous template nucleic acid. Thus, the original sequence is replaced with the sequence of the template. The homologous template nucleic acid can be provided by homologous sequences elsewhere in the genome (sister chromatids, homologous chromosomes, or repeated regions on the same or different chromosomes). Alternatively, an exogenous template nucleic acid can be introduced to obtain a specific HDR-induced change of the sequence at the target site. In this way, specific mutations can be introduced at the cut site.

As used herein, a single-stranded DNA template or a double-stranded DNA template refers to a DNA oligonucleotide that can be used by a cell as a template for HDR. Generally, the single-stranded DNA template or a double-stranded DNA template has at least one region of homology to a target site. In some cases, the single-stranded DNA template or double-stranded DNA template has two homologous regions flanking a region that contains a heterologous sequence to be inserted at a target cut site.

The terms “vector” and “plasmid” are used interchangeably and as used herein refer to polynucleotide vehicles useful to introduce genetic material into a cell. Vectors can be linear or circular. Vectors can integrate into a target genome of a host cell or replicate independently in a host cell. Vectors can comprise, for example, an origin of replication, a multicloning site, and/or a selectable marker. An expression vector typically comprises an expression cassette. Vectors and plasmids include, but are not limited to, integrating vectors, prokaryotic plasmids, eukaryotic plasmids, plant synthetic chromosomes, episomes, cosmids, and artificial chromosomes.

As used herein, the phrase “introducing” in the context of introducing a nucleic acid or a complex comprising a nucleic acid, for example, an RNP-DNA template complex, refers to the translocation of the nucleic acid sequence or the RNP-DNA template complex from outside a cell to inside the cell. In some cases, introducing refers to translocation of the nucleic acid or the complex from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

As used herein the term “expression cassette” is a polynucleotide construct, generated recombinantly or synthetically, comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in an expression vector.

As used herein, the phrase “subject in need thereof” refers to a subject that exhibits and/or is diagnosed with one or more symptoms or signs of a disease or disorder as described herein.

A “chemotherapeutic agent” refers to a chemical compound useful in the treatment of cancer. Chemotherapeutic agents include “anti-hormonal agents” or “endocrine therapeutics” which act to regulate, reduce, block, or inhibit the effects of hormones that can promote the growth of cancer.

The term “composition” refers to a mixture that contains, e.g., an engineered cell or nucleic acid contemplated herein. In some embodiments, the composition may contain additional components, such as adjuvants, stabilizers, excipients, and the like. The term “composition” or “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The term “in situ” refers to processes that occur in a living cell growing separate from a living organism, e.g., growing in tissue culture.

The term “in vivo” refers to processes that occur in a living organism.

As used herein, the term “ex vivo” generally includes experiments or measurements made in or on living tissue, preferably in an artificial environment outside the organism, preferably with minimal differences from natural conditions.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease.

The term “ameliorating” refers to any therapeutically beneficial result in the treatment of a disease state, e.g., a cancer disease state, lessening in the severity or progression, remission, or cure thereof.

As used herein, the term “effective amount” refers to the amount of a compound (e.g., a compositions described herein, cells described herein) sufficient to effect beneficial or desired results. An effective amount can be administered in one or more administrations, applications or dosages and is not intended to be limited to a particular formulation or administration route.

As used herein, the term “treating” includes any effect, e.g., lessening, reducing, modulating, ameliorating or eliminating, that results in the improvement of the condition, disease, disorder, and the like, or ameliorating a symptom thereof.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

It must be noted that, as used in the specification and the appended claims, the singular forms “a,” “an” and “the” include plural referents unless the context clearly dictates otherwise.

### Recombinant Nucleic Acid Compositions

Transforming Growth Factor Beta Receptor 1 (TGFBR1; HGNC: 11772, NCBI Entrez Gene: 7046, UniProtKB/Swiss-Prot: P36897) is a transmembrane serine/threonine protein kinase and forms a heteromeric complex with TGF-beta receptor type II (TGFRB2) when bound to TGF-beta, transducing the TGF-beta signal from the cell surface to the cytoplasm.

Transforming Growth Factor Beta Receptor 2 (TGFBR2; HGNC: 11773, NCBI Entrez Gene: 7048, UniProtKB/Swiss-Prot: P37173) is a transmembrane serine/threonine protein kinase and forms a heterodimeric complex with TGF-beta receptor type-1 (TGFBR1) when bound to TGF-beta, resulting in transduction of the TGF-beta signal from the cell surface to the cytoplasm.

As used herein, “target gene” refers to a nucleic acid sequence in a cell, wherein the expression of the sequence may be specifically and effectively modulated using the recombinant nucleic acid molecules and methods described herein. In certain embodiments, the target gene may be implicated in the growth (proliferation), maintenance (survival), and/or immune behavior of an individual's immune cells.

In some embodiments, the target gene is Transforming Growth Factor Beta Receptor 1 (TGFBR1). In some embodiments, the target gene is Transforming Growth Factor Beta Receptor 2 (TGFBR2). In some embodiments, two or more recombinant nucleic acid molecules target the TFGBR2 gene.

In some embodiments, more than one target gene is modulated using a recombinant nucleic acid molecule and methods described herein. In some embodiments, at least two target gene are modulated using the recombinant nucleic acid molecules and methods described herein. In some embodiments, the recombinant nucleic acid molecule(s) is an shRNA. In some embodiments, the at least two target genes are at least TGFBR1 and TGFBR2. In some embodiments, the at least two target genes are at least TGFBR1, TGFBR2, FAS and PTPN2. In some embodiments, the at least two target genes are at least TGFBR1, TGFBR2, FAS and TOX. In some embodiments, the at least two target genes are at least TGFBR2, FAS and PTPN2. In some embodiments, the at least two target genes are at least TGFBR2, FAS and TOX.

In one aspect, provided herein are recombinant nucleic acid comprising a nucleic acid sequence at least 15 nucleotides in length complementary to an mRNA encoding human Transforming Growth Factor Beta Receptor 1 (TGFBR1). In one aspect, provided herein are recombinant nucleic acid comprising a nucleic acid sequence at least 15 nucleotides in length complementary to an mRNA encoding human Transforming Growth Factor Beta Receptor 2 (TGFBR2).

In some embodiments, the recombinant nucleic acid comprises a nucleic acid sequence at least 15 nucleotides in length complementary to nucleotides 1589-1610 or 1965-1986 of an mRNA encoding human Transforming Growth Factor Beta Receptor 1 (TGFBR1) comprising the sequence set forth in SEQ ID NO: 1.

In some embodiments, the recombinant nucleic acid comprises a nucleic acid sequence at least 15 nucleotides in length complementary to nucleotides 2215-2236, 4430-4451, or 3761-3782 of an mRNA encoding human Transforming Growth Factor Beta Receptor 2 (TGFBR2) comprising the sequence set forth in SEQ ID NO: 2.

In some embodiments, the nucleic acid or first nucleic acid comprises a sequence selected from the group consisting of the sequences set forth in SEQ ID NOs: 6-84. In some embodiments, the first nucleic acid comprises a sequence selected from the group consisting of the sequences set forth in SEQ ID NOs: 36-84 and the second nucleic acid comprises a sequence selected from the group consisting of the sequences set forth in SEQ ID NOs: 6-84.

In some embodiments, the first and second nucleic acids each comprises a sequence selected from the group consisting of the sequences set forth in SEQ ID NOs: 36-84. In some embodiments, the nucleic acid or first nucleic acid comprises the sequence set forth in SEQ ID NO: 81. In some embodiments, the nucleic acid or first nucleic acid comprises the sequence set forth in SEQ ID NO: 51 or 53. In some embodiments, the nucleic acid, first nucleic acid, or second nucleic acid comprises the sequence set forth in SEQ ID NOs: 81 and 51 or 53.

In some embodiments, the nucleic acid or second nucleic acid comprises a sequence selected from the group consisting of the sequences set forth in SEQ ID NOs: 6-35. In some embodiments, the nucleic acid or second nucleic acid comprises the sequence set forth in SEQ ID NO: 16 or 28. the first nucleic acid comprises the sequence set forth in SEQ ID NO: 81 and the second nucleic acid comprises the sequence set forth in SEQ ID NO: 16 or 28.

In some embodiments, the first nucleic acid comprises the sequence set forth in SEQ ID NO: 81 and the second nucleic acid comprises the sequence set forth in SEQ ID NO: 28, 16, 51, or 53. In some embodiments, the first nucleic acid comprises the sequence set forth in SEQ ID NO: 81 and the second nucleic acid comprises the sequence set forth in SEQ ID NO: 51. In some embodiments, the first nucleic acid comprises the sequence set forth in SEQ ID NO: 81, the second nucleic acid comprises the sequence set forth in SEQ ID NO: 51, and the third nucleic acid comprises the sequence set forth in SEQ ID NO: 92.

In some embodiments, the one or more nucleic acids further comprises at least a third nucleic acid sequence at least 15 nucleotides in length, wherein the at least a third nucleic acid sequence comprises a nucleic acid sequence complementary to nucleotides 1126 to 1364 of an mRNA encoding human Fas Cell Surface Death Receptor (FAS) comprising the sequence set forth in SEQ ID NO: 3. In some embodiments, the third nucleic acid comprises a sequence selected from the group consisting of the sequences set forth in SEQ ID NOs: 85-99. In some embodiments, the nucleic acid comprises the sequence set forth in SEQ ID NO: 92. In some embodiments, the nucleic acid comprises the sequence set forth in SEQ ID NO: 130. In some embodiments, the nucleic acid comprises a sequence selected from the group consisting of the sequences set forth in SEQ ID NOs: 128-131, 140, or 141.

FAS is an apoptosis-inducing TNF receptor superfamily member. PTPN2 is a phosphatase that regulates interferon and many other signaling pathways. TOX is a transcription factor that regulates differentiation of exhausted T cells.

In one aspect, provided herein are recombinant nucleic acid comprising a nucleic acid sequence at least 15 nucleotides in length complementary to an mRNA encoding human FAS, Protein Tyrosine Phosphatase Non-Receptor Type 2 (PTPN2), or thymocyte selection associated high mobility group box (TOX).

In some embodiments, the recombinant nucleic acid comprises a nucleic acid sequence at least 15 nucleotides in length complementary to nucleotides 1126 to 1364 of an mRNA encoding human FAS comprising the sequence set forth in SEQ ID NO: 3.

In some embodiments, the recombinant nucleic acid comprises a nucleic acid sequence at least 15 nucleotides in length complementary to nucleotides 518 to 559 of an mRNA encoding human Protein Tyrosine Phosphatase Non-Receptor Type 2 (PTPN2) comprising the sequence set forth in SEQ ID NO: 4.

In some embodiments, the recombinant nucleic acid comprises a nucleic acid sequence at least 15 nucleotides in length complementary to nucleotides 1294 to 2141 of an mRNA encoding human thymocyte selection associated high mobility group box (TOX) comprising the sequence set forth in SEQ ID NO: 5.

In some embodiments, the nucleic acid comprises a sequence selected from the group consisting of the sequences set forth in SEQ ID NOs: 85-99. In some embodiments, the nucleic acid comprises the sequence set forth in SEQ ID NO: 92. In some embodiments, the nucleic acid reduces expression of FAS in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the nucleic acid comprises a sequence selected from the group consisting of the sequences set forth in SEQ ID NOs: 100-112. In some embodiments, the nucleic acid comprises the sequence set forth in SEQ ID NO: 110. In some embodiments, the nucleic acid reduces expression of PTPN2 in the cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the nucleic acid comprises a sequence selected from the group consisting of the sequences set forth in SEQ ID NOs: 113-126. In some embodiments, the nucleic acid reduces expression of TOX in the cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the nucleic acid.

In some embodiments, the nucleic acid comprises a sequence selected from the group consisting of the sequences set forth in SEQ ID NOs: 127-130.

In some embodiments, the nucleic acid comprises a sequence selected from the group consisting of the sequences set forth in SEQ ID NOs: 127-131 or 133-141. In some embodiments, the nucleic acid comprises a sequence selected from the group consisting of the sequences set forth in SEQ ID NOs: 127-131 and 139-141. In some embodiments, the nucleic acid comprises a sequence selected from the group consisting of the sequences set forth in SEQ ID NOs: 133-137.

In some embodiments, the nucleic acid further comprises at least a third nucleic acid sequence at least 15 nucleotides in length, wherein the at least a third nucleic acid sequence comprises a nucleic acid sequence complementary to nucleotides 1126 to 1364 of an mRNA encoding human Fas Cell Surface Death Receptor (FAS) comprising the sequence set forth in SEQ ID NO: 3.

In some embodiments, the third nucleic acid comprises a sequence selected from the group consisting of the sequences set forth in SEQ ID NOs: 85-99.

In some embodiments, the nucleic acid comprises the sequence set forth in SEQ ID NO: 92.

In some embodiments, the nucleic acid comprises an shRNA module. In some embodiments, the shRNA module comprises a first strand and a second strand of one or more shRNAs. In some embodiments, the shRNA module comprises two shRNAs. In some embodiments, the shRNA module comprises three shRNA sequences. In some embodiments, the shRNA module comprises four shRNA sequences. In some embodiments, the shRNA module further comprises one or more backbones of one or more miRs. In some embodiments, the one or more backbones comprises a 5′ backbone, a loop, and a 3′ backbone. In some embodiments the shRNA module comprises the first and second strands of the one or more shRNAs within the backbone of the one or more miRs. In some embodiments the first strand of an shRNA is located between the 5′ backbone and the loop of a miR and the second strand of the shRNA is located between the loop and the 3′ backbone of the miR.

In some embodiments the shRNA module is a dual shRNA module. In some embodiments the shRNA module comprises, from 5′ to 3′, (1) a 5′ backbone of a first miR (e.g., a miR-3G 5′ backbone), (2) a first strand of a first shRNA, (3) a loop of the first miR (e.g., a miR-3G loop), (4) a second strand of the first shRNA, (5) a 3′ backbone of the first miR (e.g., a miR-3G 3′ backbone), (6) a first spacer, (7) a 5′ backbone of a second miR (e.g., a miR-E 5′ backbone), (8) a first strand of a second shRNA, (9) a loop of the second miR (e.g., a miR-E loop), (10) a second strand of the second shRNA, (11) a 3′ backbone of the second miR (e.g., a miR-E 3′ backbone), (12) a second spacer, and (13) a PPT sequence. In some embodiments, the first and second miRs are identical. In some embodiments, the first miRs is miR-3G and the second miR is miR-E (“3G-E format”). In some embodiments, the first and second miRs are distinct.

In some embodiments the shRNA module is a triple shRNA module. In some embodiments the shRNA module comprises, from 5′ to 3′, (1) a 5′ backbone of a first miR (e.g., a miR-3G 5′ backbone), (2) a first strand of a first shRNA, (3) a loop of the first miR (e.g., a miR-3G loop), (4) a second strand of the first shRNA, (5) a 3′ backbone of the first miR (e.g., a miR-3G 3′ backbone), (6) a first spacer, (7) a 5′ backbone of a second miR (e.g., a miR-E 5′ backbone), (8) a first strand of a second shRNA, (9) a loop of the second miR (e.g., a miR-E loop), (10) a second strand of the second shRNA, (11) a 3′ backbone of the second miR (e.g., a miR-E 3′ backbone), (12) a second spacer, (13) a 5′ backbone of a third miR (e.g., a miR-3G 5′ backbone), (14) a first strand of a third shRNA, (15) a loop of the third miR (e.g., a miR-3G loop), (16) a second strand of the third shRNA, (17) a 3′ backbone of the third miR (e.g., a miR-3G 3′ backbone), and (18) a PPT sequence.). In some embodiments, the first, second, and third miRs are identical. In some embodiments, the first, second, and third miRs are all distinct. In some embodiments, the first and second miRs are identical and the third miR is distinct. In some embodiments, the first and third miRs are identical and the second miR is distinct. In some embodiments, the second and third miRs are identical and the first miR is distinct. In some embodiments, the first and third miRs are miR-3G and the second miR is miR-E (“3G-E-3G format”). In some embodiments, the first, second, and third miRs are all miR-3G (“3G-3G-3G format”).

In some embodiments the shRNA module is a quadruple shRNA module. In some embodiments the shRNA module comprises, from 5′ to 3′,), (1) a 5′ backbone of a first miR (e.g., a miR-3G 5′ backbone), (2) a first strand of a first shRNA, (3) a loop of the first miR (e.g., a miR-3G loop), (4) a second strand of the first shRNA, (5) a 3′ backbone of the first miR (e.g., a miR-3G 3′ backbone), (6) a first spacer, (7) a 5′ backbone of a second miR (e.g., a miR-E 5′ backbone), (8) a first strand of a second shRNA, (9) a loop of the second miR (e.g., a miR-E loop), (10) a second strand of the second shRNA, (11) a 3′ backbone of the second miR (e.g., a miR-E 3′ backbone), (12) a second spacer, (13) a 5′ backbone of a third miR (e.g., a miR-3G 5′ backbone), (14) a first strand of a third shRNA, (15) a loop of the third miR (e.g., a miR-3G loop), (16) a second strand of the third shRNA, (17) a 3′ backbone of the third miR (e.g., a miR-3G 3′ backbone), (18) a third spacer, (19) a 5′ backbone of a fourth miR (e.g., a miR-3G 5′ backbone), (20) a first strand of a fourth shRNA, (21) a loop of the fourth miR (e.g., a miR-3G loop), (22) a second strand of the fourth shRNA, (23) a 3′ backbone of the fourth miR (e.g., a miR-3G 3′ backbone), (24) a PPT sequence. In some embodiments, the first, second, third, and fourth miRs are identical. In some embodiments, the first, second, third, and fourth miRs are all distinct. In some embodiments, a first group of two miRs are identical and a second group of two miRs are identical but distinct from the first group. In some embodiments, a first group of two miRs are identical and the remaining two miRs are each distinct from the first group and each other. In some embodiments, three of the miRs are identical and the last miR is distinct. In some embodiments, the first, third, and fourth miRs are miR-3G and the second miR is miR-E (“3G-E-3G-3G format”).

In some embodiments, the nucleic acid comprises the sequence set forth in SEQ ID NO: 130. In some embodiments, the nucleic acid comprises the sequence set forth in SEQ ID NO: 128. In some embodiments, the nucleic acid comprises the sequence set forth in SEQ ID NO: 140. In some embodiments, the nucleic acid comprises the sequence set forth in SEQ ID NO: 129. In some embodiments, the nucleic acid comprises the sequence set forth in SEQ ID NO: 131. In some embodiments, the nucleic acid comprises the sequence set forth in SEQ ID NO: 139. In some embodiments, the nucleic acid comprises the sequence set forth in SEQ ID NO: 141. In some embodiments, the nucleic acid comprises the sequence set forth in SEQ ID NO: 133. In some embodiments, the nucleic acid comprises the sequence set forth in SEQ ID NO: 134. In some embodiments, the nucleic acid comprises the sequence set forth in SEQ ID NO: 135. In some embodiments, the nucleic acid comprises the sequence set forth in SEQ ID NO: 136. In some embodiments, the nucleic acid comprises the sequence set forth in SEQ ID NO: 137.

In some embodiments, the nucleic acid sequence is at least 16, 17, 18, 19, 20, 21, or 22 nucleotides in length.

In some embodiments, the nucleic acid is a an RNA interference (RNAi) molecule. Exemplary RNAi molecules include short hairpin RNA (shRNA), a small interfering RNA (siRNA), a double stranded RNA (dsRNA), or an antisense oligonucleotide. In some embodiments, the nucleic acid is a short hairpin RNA (shRNA), a small interfering RNA (siRNA), a double stranded RNA (dsRNA), or an antisense oligonucleotide. In some embodiments, the nucleic acid is an shRNA.

Single-stranded hairpin ribonucleic acids (shRNAs) are short duplexes where the sense and antisense strands are linked by a hairpin loop. They consist of a stem-loop structure that can be transcribed in cells from an RNA polymerase II or RNA polymerase III promoter on a plasmid construct. Once expressed, shRNAs are processed into RNAi species.

Expression of shRNA from a plasmid is known to be relatively stable, thereby providing strong advantages over, for example, the use of synthetic siRNAs. shRNA expression units may be incorporated into a variety of plasmids, liposomes, viral vectors, and other vehicles for delivery and integration into a target cell. Expression of shRNA from a plasmid can be stably integrated for constitutive expression. shRNAs are synthesized in the nucleus of cells, further processed and transported to the cytoplasm, and then incorporated into the RNA-induced silencing complex (RISC) for activity. The shRNAs are converted into active siRNA molecules (which are capable of binding to, sequestering, and/or preventing the translation of mRNA transcripts encoded by target genes).

The Argonaute family of proteins is the major component of RISC. Within the Argonaute family of proteins, only Ago2 contains endonuclease activity that is capable of cleaving and releasing the passenger strand from the stem portion of the shRNA molecule. The remaining three members of Argonaute family, Ago1, Ago3 and Ago4, which do not have identifiable endonuclease activity, are also assembled into RISC and are believed to function through a cleavage-independent manner. Thus, RISC can be characterized as having cleavage-dependent and cleavage-independent pathways.

RNAi (e.g., antisense RNA, siRNA, microRNA, shRNA, etc.) are described in International Publication Nos. WO2018232356A1, WO2019084552A1, WO2019226998A1, WO2020014235A1, WO2020123871A1, and WO2020186219A1, each of which is herein incorporated by reference for all purposes.

Antisense oligonucleotide structure and chemical modifications are described in International PCT Publication No. WO20/132521, which is hereby incorporated by reference.

dsRNA and shRNA molecules and methods of use and production are described in U.S. Pat. Nos. 8,829,264; 9,556,431; and 8,252,526, each of which are hereby incorporated by reference

siRNA molecules and methods of use and production are described in U.S. Pat. No. 7,361,752 and US Patent Publication No. US20050048647, both of which are hereby incorporated by reference.

Additional methods and compositions for RNA interference such as shRNA, siRNA, dsRNA, and antisense oligonucleotides are generally known in the art, and are further described in U.S. Pat. Nos. 7,361,752; 8,829,264; 9,556,431; 8,252,526, International PCT Publication No. WO00/44895; International PCT Publication No. WO01/36646; International PCT Publication No. WO99/32619; International PCT Publication No. WO00/01846; International PCT Publication No. WO01/29058; and International PCT Publication No. WO00/44914; International PCT Publication No. WO04/030634; each of which are hereby incorporated by reference.

The nucleic acid sequences (or constructs) that may be used to encode the RNAi molecules, such as an shRNA described herein, may comprise a promoter, which is operably linked (or connected), directly or indirectly, to a sequence encoding the RNAi molecules. Such promoters may be selected based on the host cell and the effect sought. Non-limiting examples of suitable promoters include constitutive and inducible promoters, such as inducible RNA polymerase II (pol II)-based promoters. Non-limiting examples of suitable promoters further include the tetracycline inducible or repressible promoter, EF1a, RNA polymerase I or III-based promoters, the pol II dependent viral promoters, such as the CMV-IE promoter, and the pol III U6 and H1 promoters. The bacteriophage T7 promoter may also be used (in which case it will be appreciated that the T7 polymerase must also be present). The nucleic acid sequences need not be restricted to the use of any single promoter, especially since the nucleic acid sequences may comprise two or more shRNAs (i.e., a combination of effectors), including but not limited to incorporated shRNA molecules. Each incorporated promoter may control one, or any combination of, the shRNA molecule components.

In certain embodiments, the promoter may be preferentially active in the targeted cells, e.g., it may be desirable to preferentially express at least one recombinant nucleic acid in immune cells using an immune cell-specific promoter. Introduction of such constructs into host cells may be effected under conditions whereby the two or more recombinant nucleic acids that are contained within the recombinant nucleic acid precursor transcript initially reside within a single primary transcript, such that the separate RNA molecules (for example, shRNA each comprising its own stem-loop structure) are subsequently excised from such precursor transcript by an endogenous ribonuclease. The resulting mature recombinant nucleic acids (e.g., shRNAs) may then induce degradation, and/or translation repression, of target gene mRNA transcripts produced in the cell. Alternatively, each of the precursor stem-loop structures may be produced as part of a separate transcript, in which case each recombinant nucleic acid sequence will preferably include its own promoter and transcription terminator sequences. Additionally, the multiple recombinant nucleic acid precursor transcripts may reside within a single primary transcript.

The stem-loop structures of the shRNA recombinant nucleic acids described herein may be about 40 to 100 nucleotides long or, preferably, about 50 to 75 nucleotides long. The stem region may be about 15-45 nucleotides in length (or more), or about 20-30 nucleotides in length. In some embodiments, the stem region is 22 nucleotides in length. In some embodiments, the stem region is 15, 16, 17, 18, 19, 20, 21, 22, 23, 24, 25, 26, 27 28 29, 30, 31, 32, 33, 34, 35, 36, 37, 38, 39, 40, 41, 42, 43, 44, or 45 nucleotides in length.

The stem may comprise a perfectly complementary duplex (but for any 3′ tail), however, bulges or interior loops may be present on either arm of the stem. The number of such bulges and asymmetric interior loops are preferably few in number (e.g., 1, 2 or 3) and are about 3 nucleotides or less in size. The terminal loop portion may comprise about 4 or more nucleotides, but preferably not more than about 25. The loop portion will preferably be 6-15 nucleotides in size.

As described herein, the stem regions of the shRNAs comprise passenger strands and guide strands, whereby the guide strands contain sequences complementary to the target mRNA transcript encoded by the target gene(s). Preferably, the G-C content and matching of guide strand and passenger strand is carefully designed for thermodynamically-favorable strand unwind activity with or without endonuclease cleavage. Furthermore, the specificity of the guide strand is preferably confirmed via a BLAST search (www.ncbi.nim.nih.gov/BLAST).

The invention provides that the expression level of multiple target genes may be modulated using the methods and recombinant nucleic acids described herein. For example, the invention provides that a first set of recombinant nucleic acids may be designed to include a sequence (a guide strand) that is designed to reduce the expression level of a first target gene, whereas a second set of recombinant nucleic acids may be designed to include a sequence (a guide strand) that is designed to reduce the expression level of a second target gene. The different sets of recombinant nucleic acids may be expressed and reside within the same, or separate, preliminary transcripts. In certain embodiments, such multiplex approach, i.e., the use of the recombinant nucleic acids described herein to modulate the expression level of two or more target genes, may have an enhanced therapeutic effect on a patient. For example, if a patient is provided with cells expressing the recombinant nucleic acid molecules described herein to treat, prevent, or ameliorate the effects of cancer, it may be desirable to provide the patient with two or more types of recombinant nucleic acid molecules, which are designed to reduce the expression level of multiple genes that are implicated in activation or repression of immune cells.

The one or more recombinant nucleic acid molecule(s) described herein may be capable of reducing target gene expression in a cell by at least more than about 50% as compared to a control cell that does not comprise the recombinant nucleic acid molecule(s). For example, the recombinant nucleic acid molecule(s) (e.g., shRNA) can be capable of reducing expression of a target gene selected from the group consisting of TGFBR1, TGFBR2, FAS, PTPN2, and/or TOX in the cell by at least 10%, 15%, 20%, 25%, 30%, 35%, 40%, 45%, 50%, 55%, 60%, 65%, 75%, 80%, 85%, 90%, 95%, 97%, 98%, 99%, or more as compared to a control cell that does not comprise the respective recombinant nucleic acid molecule(s). The one or more recombinant nucleic acid molecule(s) can be capable of reducing expression of a target gene selected from the group consisting of TGFBR1, TGFBR2, FAS, PTPN2, and/or TOX in the cell by at least between about 10-50%, 10-20%, 10-30%, 10-40%, 20-50%, 30-50%, 40-50%, 10-100%, 50-100%, 50-99%, 50-95%, 50-90%, 50-85%, 50-80%, 50-75%, 50-70%, 50-65%, 50-60%, 50-55%, or as compared to a control cell that does not comprise the respective recombinant nucleic acid molecule(s). In some embodiments, the one or more recombinant nucleic acid molecule(s) reduces expression of TGFBR 1 in the cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the recombinant nucleic acid molecule(s). In some embodiments, the one or more recombinant nucleic acid molecule(s) reduces expression of TGFBR2 in the cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the recombinant nucleic acid molecule(s). In some embodiments, the one or more recombinant nucleic acid molecule(s) reduces expression of FAS in the cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the recombinant nucleic acid molecule(s). In some embodiments, the one or more recombinant nucleic acid molecule(s) reduces expression of PTPN2 in the cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the recombinant nucleic acid molecule(s). In some embodiments, the one or more recombinant nucleic acid molecule(s) reduces expression of TOX in the cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the recombinant nucleic acid molecule(s).

The recombinant nucleic acid molecule(s) may be chemically synthesized, or in vitro transcribed, and may further include one or more modifications to phosphate-sugar backbone or nucleosides residues.

Other methods known in the art for introducing nucleic acids to cells may be used, such as lipid-mediated carrier transport, chemical mediated transport, such as calcium phosphate, and the like. Thus, the recombinant nucleic acid molecule(s) construct may be introduced along with components that perform one or more of the following activities: enhance RNA uptake by the cell, promote annealing of the duplex strands for shRNA, stabilize the annealed shRNA strands, or otherwise increase inhibition of the target gene.

In some embodiments, the one or more recombinant nucleic acid(s) further comprises a 5′ homology directed repair arm and/or a 3′ homology directed repair arm complementary to an insertion site in a host cell chromosome. In some embodiments, the one or more recombinant nucleic acid(s) comprises the 5′ homology directed repair arm and the 3′ homology directed repair arm. In some embodiments, the one or more recombinant nucleic acid(s) is incorporated into an expression cassette or an expression vector. In some embodiments, the expression cassette or the expression vector further comprises a constitutive promoter upstream of the one or more recombinant nucleic acid(s).

In some embodiments, the one or more recombinant nucleic acid(s) comprises at least a first nucleic acid and at least a second nucleic acid. In some embodiments, the one or more recombinant nucleic acid(s) further comprises at least a third nucleic acid and at least a fourth nucleic acid. The first, second, third, and/or fourth nucleic acids can be RNAi molecules, such as shRNA. In some embodiments, the first nucleic acid and the second nucleic acid are incorporated into a single expression cassette or a single expression vector. In some embodiments, the third nucleic acid and the fourth nucleic acid are incorporated into a single expression cassette or a single expression vector. In some embodiments, the first, second, third, and fourth nucleic acid are incorporated into a single expression cassette or a single expression vector. In some embodiments, the expression cassette or the expression vector further comprises a constitutive promoter upstream of the first nucleic acid and/or upstream of the second nucleic acid. In some embodiments, the expression cassette or the expression vector further comprises a constitutive promoter upstream of the third nucleic acid and/or upstream of the fourth nucleic acid. In some embodiments, the expression cassette or the expression vector further comprises a constitutive promoter upstream of the first nucleic acid, upstream of the second nucleic acid, upstream of the third nucleic acid, and/or upstream of the fourth nucleic acid. In some embodiments, the expression vector is a non-viral vector.

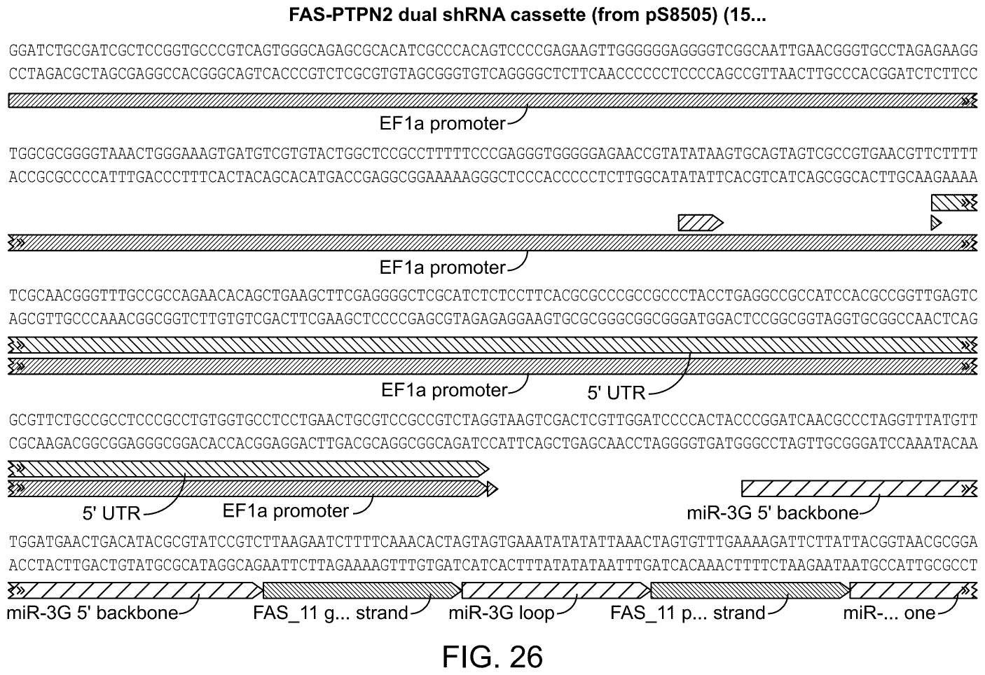

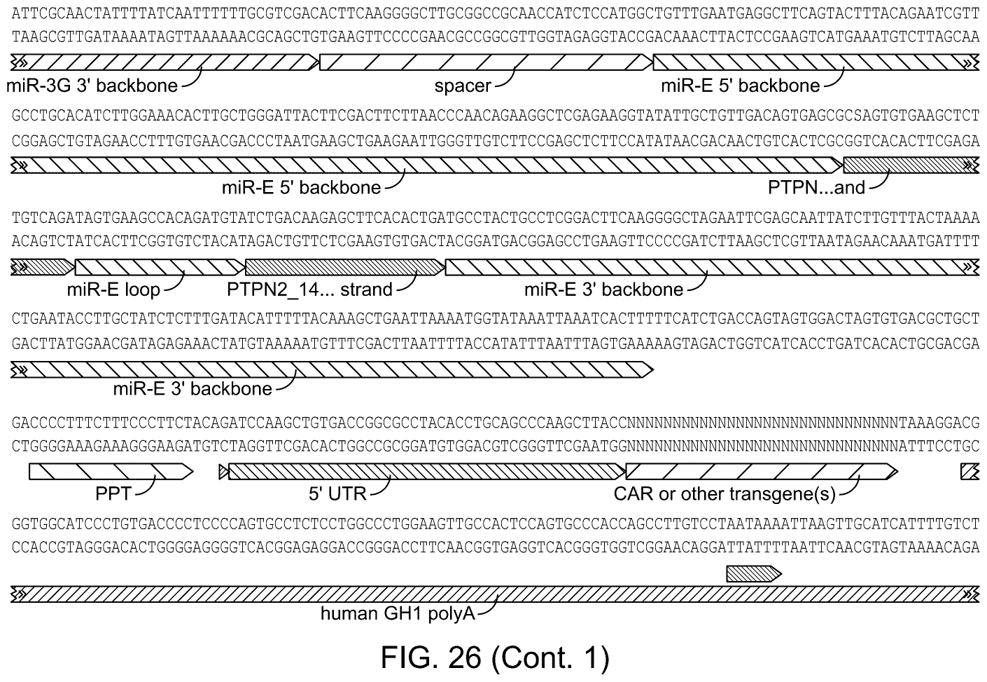

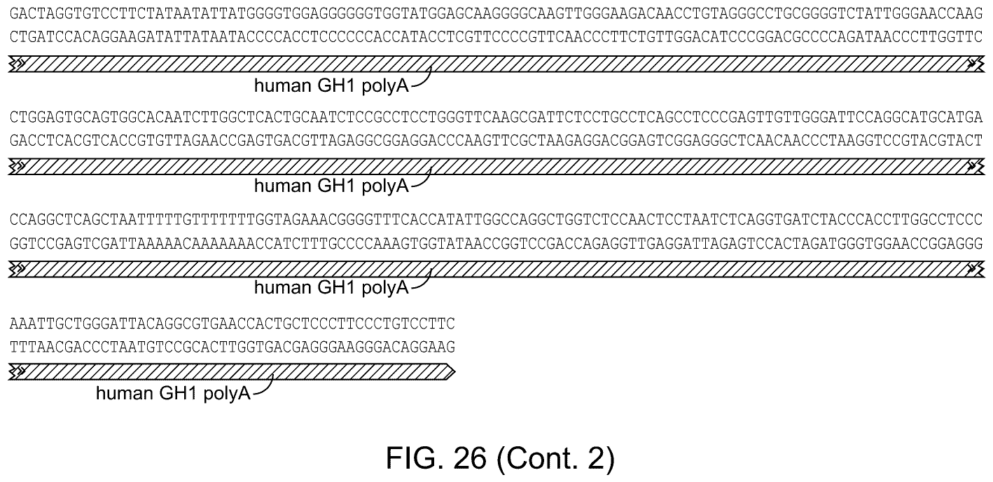

In some embodiments, the expression cassette is a dual shRNA expression cassette. In some embodiments, the dual expression cassette comprises, from 5′ to 3′, (1) a promoter (e.g., an EF1a promoter, e.g., SEQ ID NO: 132), (2) a 5′ backbone of a first miR (e.g., a miR-3G 5′ backbone), (3) a first strand of a first shRNA, (4) a loop of the first miR (e.g., a miR-3G loop), (5) a second strand of the first shRNA, (6) a 3′ backbone of the first miR (e.g., a miR-3G 3′ backbone), (7) a first spacer, (8) a 5′ backbone of a second miR (e.g., a miR-E 5′ backbone), (9) a first strand of a second shRNA, (10) a loop of the second miR (e.g., a miR-E loop), (11) a second strand of the second shRNA, (12) a 3′ backbone of the second miR (e.g., a miR-E 3′ backbone), (13) a second spacer, (14) a PPT sequence, (15) a 5′ untranslated region (UTR), (16) an optional transgene (e.g., encoding a chimeric antigen receptor), and (17) a polyadenylation (polyA) sequence (e.g., a human growth hormone (GH1) polyA sequence, e.g., SEQ ID NO: 138). In some embodiments, the first and second miRs are identical. In some embodiments, the first miRs is miR-3G and the second miR is miR-E (“3G-E format”). In some embodiments, the first and second miRs are distinct. In some embodiments, the dual expression cassette comprises the nucleic acid sequence set forth in SEQ ID NO: 133, or a nucleic acid having at least 80%, at least 85%, at least 90%, at least 91%, at least 92%, at least 93%, at least 94%, at least 95%, at least 96%, at least 97%, at least 98%, at least 99%, at least 99.5%, or at least 99.9% sequence identity thereto. An exemplary dual shRNA expression cassette is shown in FIG. 26.

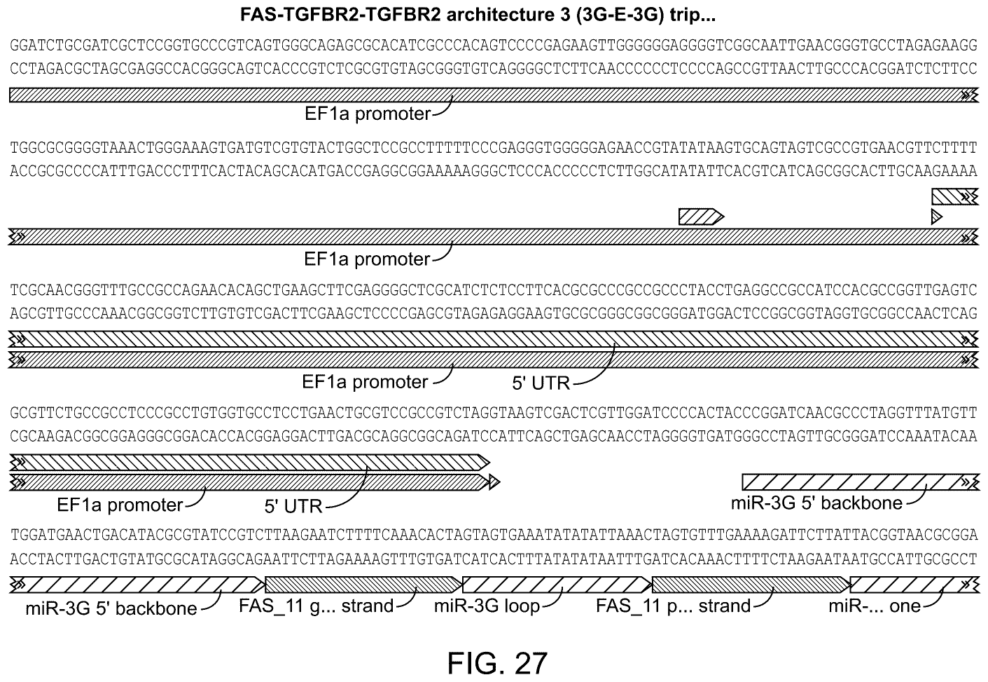

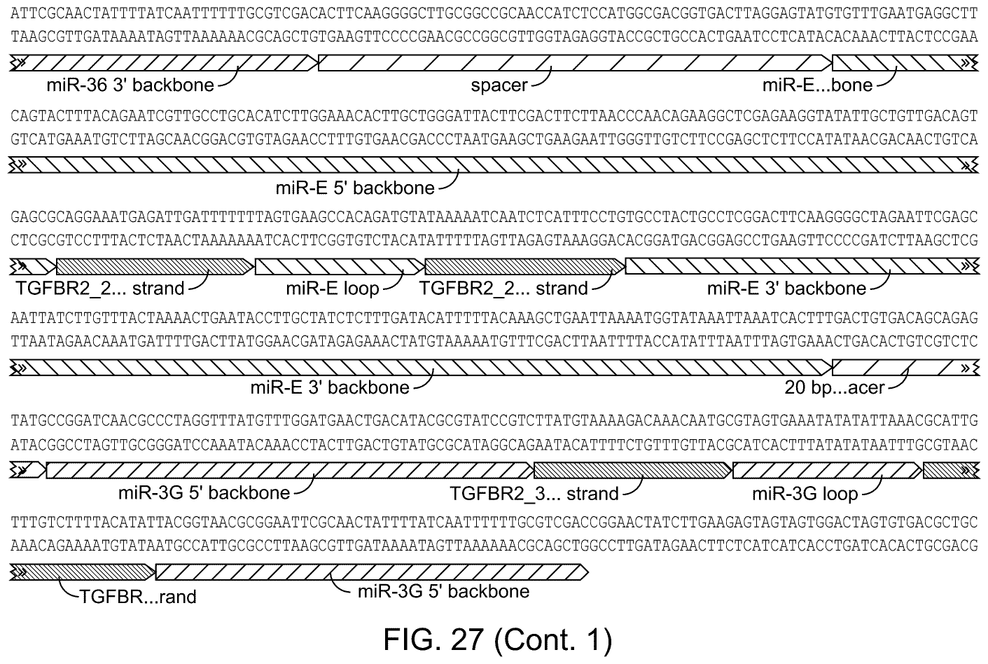

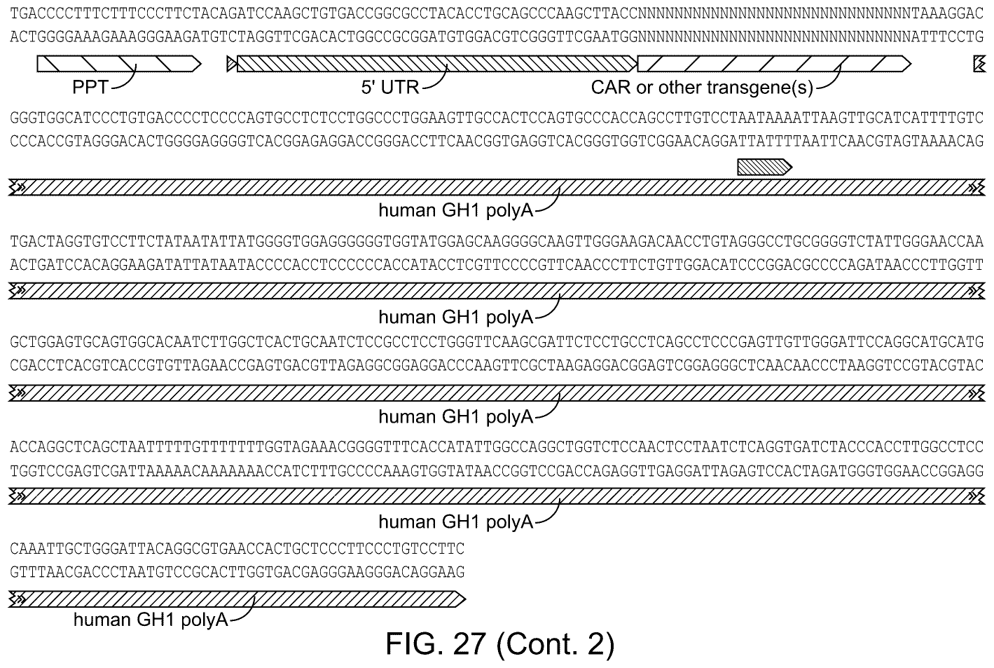

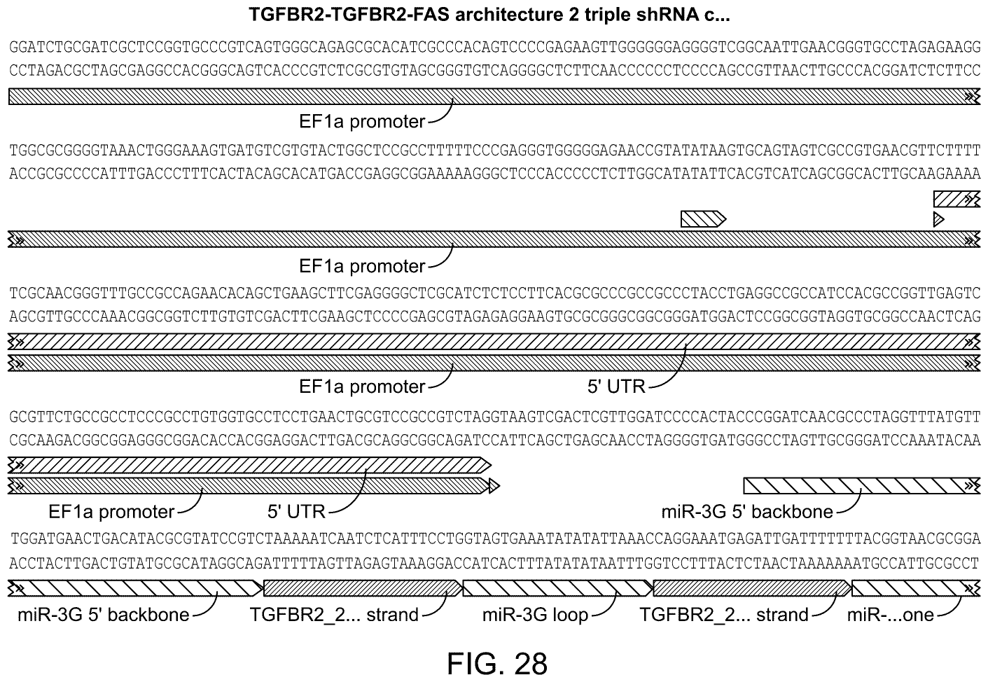

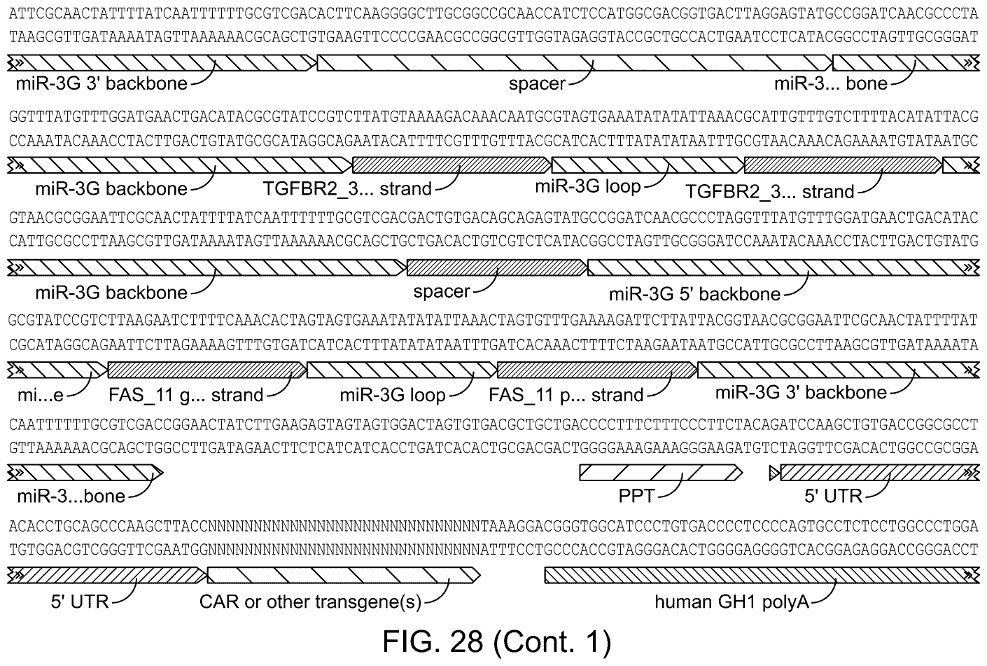

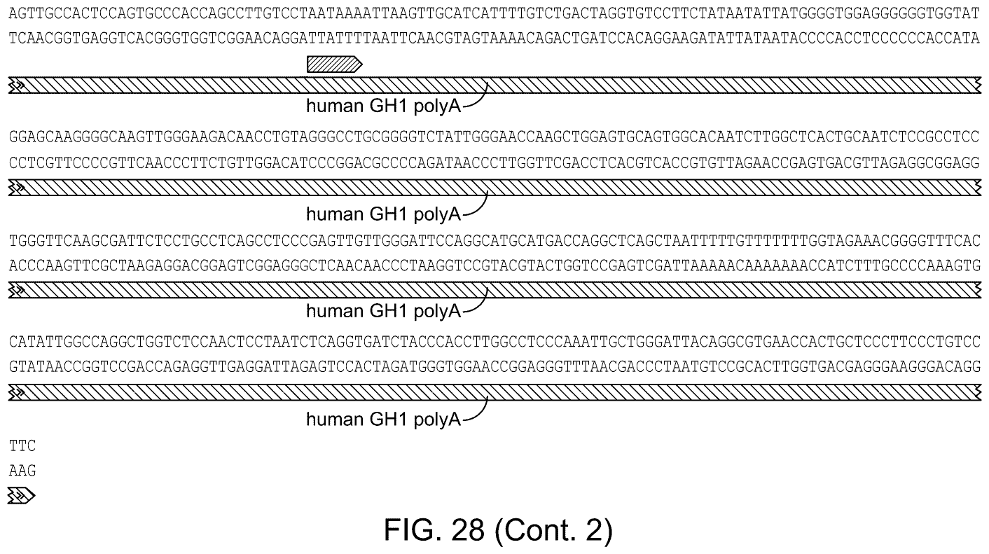

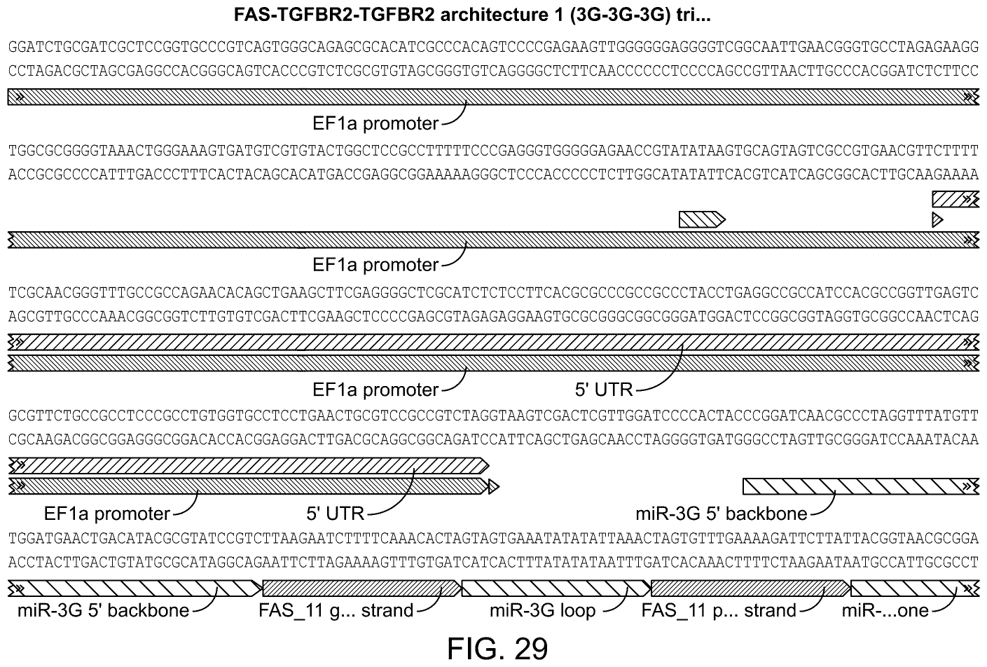

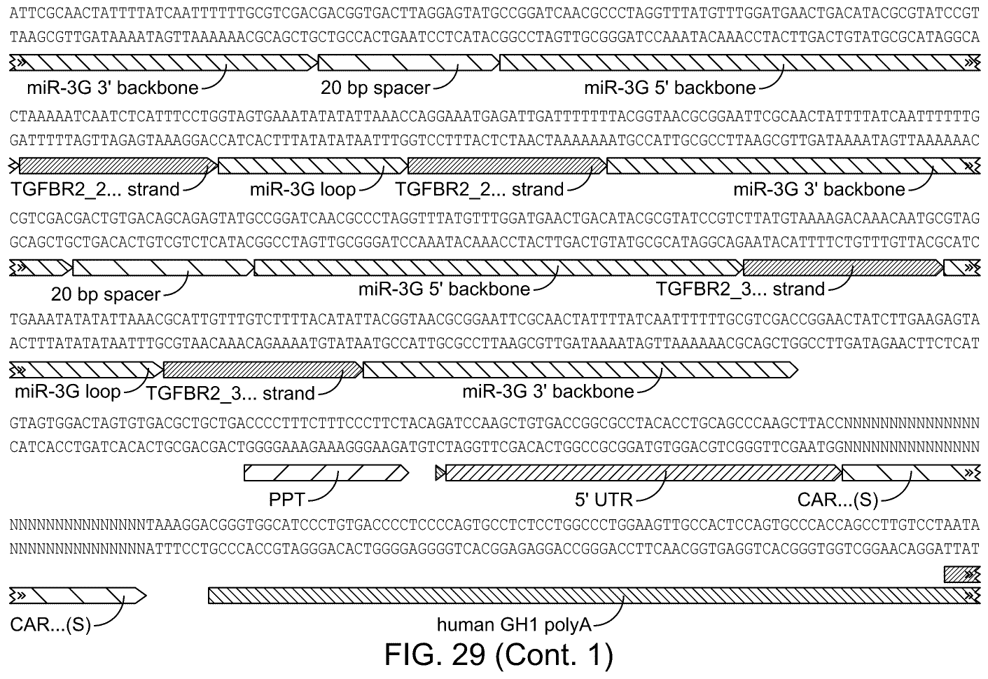

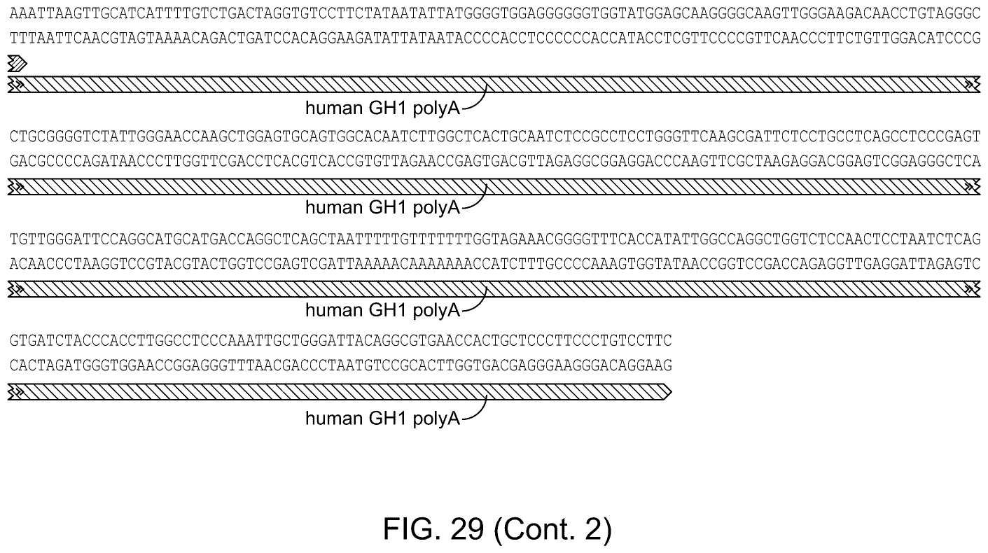

In some embodiments, the expression cassette is a triple shRNA expression cassette. In some embodiments, the triple expression cassette comprises, from 5′ to 3′, (1) a promoter (e.g., an EF1a promoter, e.g., SEQ ID NO: 132), (2) a 5′ backbone of a first miR (e.g., a miR-3G 5′ backbone), (3) a first strand of a first shRNA, (4) a loop of the first miR (e.g., a miR-3G loop), (5) a second strand of the first shRNA, (6) a 3′ backbone of the first miR (e.g., a miR-3G 3′ backbone), (7) a first spacer, (8) a 5′ backbone of a second miR (e.g., a miR-E 5′ backbone), (9) a first strand of a second shRNA, (10) a loop of the second miR (e.g., a miR-E loop), (11) a second strand of the second shRNA, (12) a 3′ backbone of the second miR (e.g., a miR-E 3′ backbone), (13) a second spacer, (14) a 5′ backbone of a third miR (e.g., a miR-3G 5′ backbone), (15) a first strand of a third shRNA, (16) a loop of the third miR (e.g., a miR-3G loop), (17) a second strand of the third shRNA, (18) a 3′ backbone of the third miR (e.g., a miR-3G 3′ backbone), (19) a PPT sequence, (20) a 5′ untranslated region (UTR), (21) an optional transgene (e.g., encoding a chimeric antigen receptor), and (22) a polyadenylation (polyA) sequence (e.g., a human growth hormone (GH1) polyA sequence, e.g., SEQ ID NO: 138). In some embodiments, the first, second, and third miRs are identical. In some embodiments, the first, second, and third miRs are all distinct. In some embodiments, the first and second miRs are identical and the third miR is distinct. In some embodiments, the first and third miRs are identical and the second miR is distinct. In some embodiments, the second and third miRs are identical and the first miR is distinct. In some embodiments, the first and third miRs are miR-3G and the second miR is miR-E (“3G-E-3G format”). In some embodiments, the first, second, and third miRs are all miR-3G (“3G-3G-3G format”). In some embodiments, the triple expression cassette comprises the nucleic acid sequence set forth in any one of SEQ ID NOs: 134-136, or a nucleic acid having at least 80%, at least 85%, at least 90%, at least 91%, at least 92%, at least 93%, at least 94%, at least 95%, at least 96%, at least 97%, at least 98%, at least 99%, at least 99.5%, or at least 99.9% sequence identity thereto. In some embodiments, the triple expression cassette comprises the nucleic acid sequence set forth in SEQ ID NO: 134, or a nucleic acid having at least 80%, at least 85%, at least 90%, at least 91%, at least 92%, at least 93%, at least 94%, at least 95%, at least 96%, at least 97%, at least 98%, at least 99%, at least 99.5%, or at least 99.9% sequence identity thereto. In some embodiments, the triple expression cassette comprises the nucleic acid sequence set forth in SEQ ID NO: 135, or a nucleic acid having at least 80%, at least 85%, at least 90%, at least 91%, at least 92%, at least 93%, at least 94%, at least 95%, at least 96%, at least 97%, at least 98%, at least 99%, at least 99.5%, or at least 99.9% sequence identity thereto. In some embodiments, the triple expression cassette comprises the nucleic acid sequence set forth in SEQ ID NO: 136, or a nucleic acid having at least 80%, at least 85%, at least 90%, at least 91%, at least 92%, at least 93%, at least 94%, at least 95%, at least 96%, at least 97%, at least 98%, at least 99%, at least 99.5%, or at least 99.9% sequence identity thereto. Exemplary triple shRNA expression cassettes are shown in FIGS. 27-29.

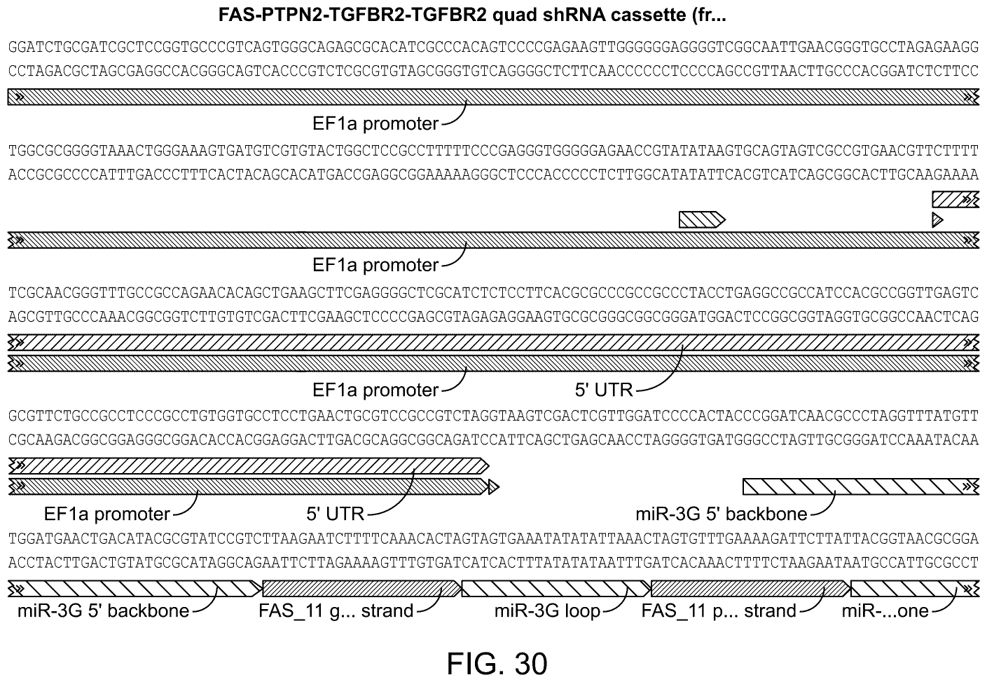

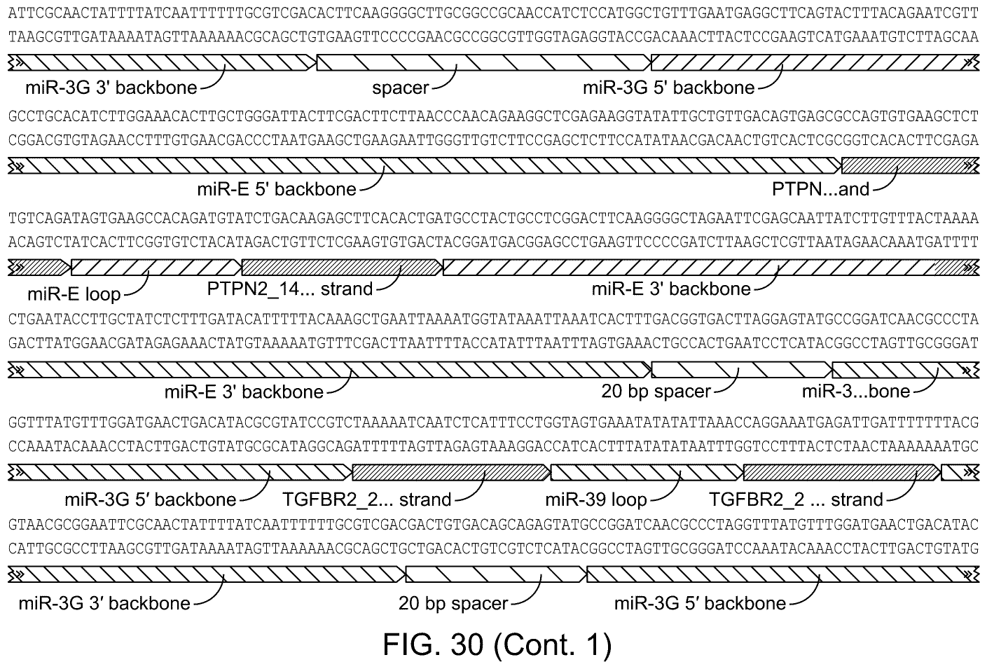

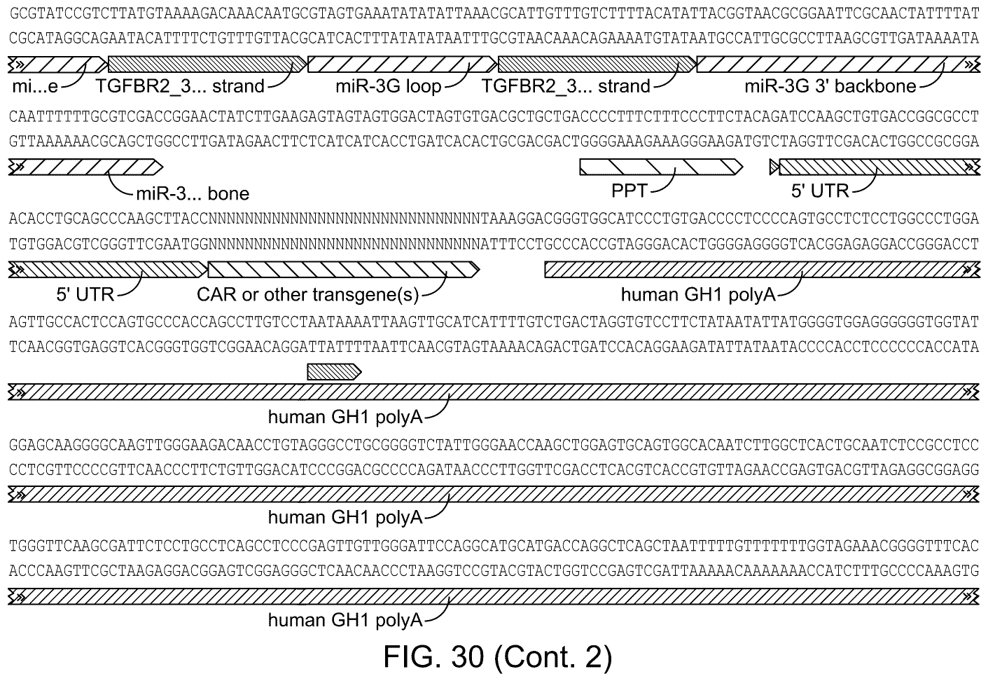

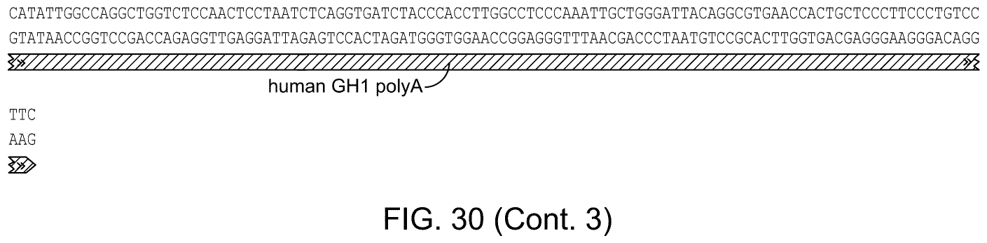

In some embodiments, the expression cassette is a quadruple shRNA expression cassette. In some embodiments, the quadruple expression cassette comprises, from 5′ to 3′, (1) a promoter (e.g., an EF1a promoter, e.g., SEQ ID NO: 132), (2) a 5′ backbone of a first miR (e.g., a miR-3G 5′ backbone), (3) a first strand of a first shRNA, (4) a loop of the first miR (e.g., a miR-3G loop), (5) a second strand of the first shRNA, (6) a 3′ backbone of the first miR (e.g., a miR-3G 3′ backbone), (7) a first spacer, (8) a 5′ backbone of a second miR (e.g., a miR-E 5′ backbone), (9) a first strand of a second shRNA, (10) a loop of the second miR (e.g., a miR-E loop), (11) a second strand of the second shRNA, (12) a 3′ backbone of the second miR (e.g., a miR-E 3′ backbone), (13) a second spacer, (14) a 5′ backbone of a third miR (e.g., a miR-3G 5′ backbone), (15) a first strand of a third shRNA, (16) a loop of the third miR (e.g., a miR-3G loop), (17) a second strand of the third shRNA, (18) a 3′ backbone of the third miR (e.g., a miR-3G 3′ backbone), (19) a third spacer, (20) a 5′ backbone of a fourth miR (e.g., a miR-3G 5′ backbone), (21) a first strand of a fourth shRNA, (22) a loop of the fourth miR (e.g., a miR-3G loop), (23) a second strand of the fourth shRNA, (24) a 3′ backbone of the fourth miR (e.g., a miR-3G 3′ backbone), (25) a PPT sequence, (26) a 5′ untranslated region (UTR), (27) an optional transgene (e.g., a chimeric antigen receptor), and (28) a polyadenylation (polyA) sequence (e.g., a human growth hormone (GH1) polyA sequence, e.g., SEQ ID NO: 138). In some embodiments, the first, second, third, and fourth miRs are identical. In some embodiments, the first, second, third, and fourth miRs are all distinct. In some embodiments, a first group of two miRs are identical and a second group of two miRs are identical but distinct from the first group. In some embodiments, a first group of two miRs are identical and the remaining two miRs are each distinct from the first group and each other. In some embodiments, three of the miRs are identical and the last miR is distinct. In some embodiments, the first, third, and fourth miRs are miR-3G and the second miR is miR-E (“3G-E-3G-3G format”). In some embodiments, the quadruple expression cassette comprises the nucleic acid sequence set forth in SEQ ID NO: 137, or a nucleic acid having at least 80%, at least 85%, at least 90%, at least 91%, at least 92%, at least 93%, at least 94%, at least 95%, at least 96%, at least 97%, at least 98%, at least 99%, at least 99.5%, or at least 99.9% sequence identity thereto. An exemplary quadruple shRNA expression cassette is shown in FIG. 30.

### Recombinant Cells

Also provided herein is a recombinant cell, such as primary sell or an immune cell, comprising at least one recombinant nucleic acid(s) non-virally inserted into a target region of the genome of the cell.

In one aspect, provided herein are immune cells comprising one or more recombinant nucleic acids at least 15 nucleotides in length complementary to an mRNA encoding human TGFBR1 comprising the sequence set forth in SEQ ID NO: 1.

In one aspect, provided herein are immune cells comprising one or more recombinant nucleic acids at least 15 nucleotides in length complementary to an mRNA encoding human TGFBR2 comprising the sequence set forth in SEQ ID NO: 2.

In one aspect, provided herein are immune cells comprising one or more recombinant nucleic acids comprising: a first nucleic acid sequence at least 15 nucleotides in length complementary to an mRNA encoding human TGFBR2 comprising the sequence set forth in SEQ ID NO: 2, and a second nucleic acid sequence at least 15 nucleotides in length, wherein the second nucleic acid sequence is complementary to an mRNA encoding human TGFBR2 comprising the sequence set forth in SEQ ID NO: 2; or complementary to an mRNA encoding human TGFBR1 comprising the sequence set forth in SEQ ID NO: 1.

In one aspect, provided herein are immune cells comprising one or more recombinant nucleic acids comprising: a first nucleic acid sequence at least 15 nucleotides in length complementary to an mRNA encoding human TGFBR2 comprising the sequence set forth in SEQ ID NO: 2, and a second nucleic acid sequence at least 15 nucleotides in length complementary to an mRNA encoding human TGFBR1 comprising the sequence set forth in SEQ ID NO: 1.

In one aspect, provided herein are immune cells comprising one or more recombinant nucleic acids comprising: a first nucleic acid sequence and a second nucleic acid sequence at least 15 nucleotides in length complementary to an mRNA encoding human TGFBR2 comprising the sequence set forth in SEQ ID NO: 2.

In some embodiments, the cell is a primary immune cell. In some embodiments, the cell is a viable, virus-free, primary cell.

In some embodiments, the expression of the gene targeted (e.g., TGFBR1, TGFBR2, FAS, PTPN2, and/or TOX) by the recombinant nucleic acid molecule(s) is reduced or decreased in the target cell. The target gene expression can be reduced by at least about 10%, 15%, 20%, 25%, 30%, 35%, 40%, 45%, 50%, 55%, 60%, 65%, 70%, 75%, 80%, 85%, 90%, 95%, 99%, or more. The target gene expression can be reduced by between about 10-50%, 10-20%, 10-30%, 10-40%, 20-50%, 30-50%, 40-50%, 10-100%, 50-100%, 50-99%, 50-95%, 50-90%, 50-85%, 50-80%, 50-75%, 50-70%, 50-65%, 50-60%, 50-55%, or as compared to a control cell that does not comprise the recombinant nucleic acid molecule(s).

A cell comprising a recombinant nucleic acid molecule(s) insert at a target locus or safe harbor site as described in the present disclosure can be referred to as an engineered cell. In some embodiments, the engineered cell is an immune cell. In some embodiments, the immune cell is any cell that can give rise to a pluripotent immune cell. In some embodiments, the immune cell can be an induced pluripotent stem cell (iPSC) or a human pluripotent stem cell (HSPC). In some embodiments, the immune cell comprises primary hematopoietic cells or primary hematopoictic stem cells. In some embodiments, that engineered cell is a stem cell, a human cell, a primary cell, an hematopoictic cell, an adaptive immune cell, an innate immune cell, a natural killer (NK) cell, a T cell, a CD8+ cell, a CD4+ cell, or a T cell progenitor. In some embodiments, the immune cells are T cells. In some embodiments, the T cells are regulatory T cells, effector T cells, or naïve T cells. In some embodiments, the T cells are CD8⁺ T cells. In some embodiments, the T cells are CD4⁺ T cells. In some embodiments, the T cells are CD4⁺CD8⁺ T cells.

In some embodiments, the engineered cell is a stem cell, a human cell, a primary cell, an hematopoietic cell, an adaptive immune cell, an innate immune cell, a T cell or a T cell progenitor. Non-limiting examples of immune cells that are contemplated in the present disclosure include T cell, B cell, natural killer (NK) cell, NKT/INKT cell, macrophage, myeloid cell, and dendritic cells. Non-limiting examples of stem cells that are contemplated in the present disclosure include pluripotent stem cells (PSCs), embryonic stem cells (ESCs), induced pluripotent stem cells (iPSCs), embryo-derived embryonic stem cells obtained by nuclear transfer (ntES; nuclear transfer ES), male germline stem cells (GS cells), embryonic germ cells (EG cells), hematopoietic stem/progenitor stem cells (HSPCs), somatic stem cells (adult stem cells), hemangioblasts, neural stem cells, mesenchymal stem cells and stem cells of other cells (including osteocyte, chondrocyte, myocyte, cardiac myocyte, neuron, tendon cell, adipocyte, pancreocyte, hepatocyte, nephrocyte and follicle cells and so on). In some embodiments, the engineered cells is a T cell, NK cells, iPSC, and HSPC. In some embodiments, the engineered cells used in the present disclosure are human cell lines grown in vitro (e.g. deliberately immortalized cell lines, cancer cell lines, etc.).

In some embodiments, the immune cell is an autologous immune cell. In some embodiments, the immune cell is an allogeneic immune cell.

Also provided herein are populations of cells comprising a plurality of the engineered cells. In some embodiments, the genome of at least 20%, 30%, 40%, 50%, 60%, 70%, 80%, 90%, 95%, 99% or greater of the cells comprises at least one recombinant nucleic acid molecule(s). In some embodiments, the genome of at least 20%, 30%, 40%, 50%, 60%, 70%, 80%, 90%, 95%, 99% or greater of the cells comprises at least two shRNA molecules. In some embodiments, the genome of at least 20%, 30%, 40%, 50%, 60%, 70%, 80%, 90%, 95%, 99% or greater of the cells comprises at least three, four, five, six, seven, eight, nine, ten or more recombinant nucleic acid molecule(s).

Also provided herein are populations of cells comprising the recombinant nucleic acid(s).

The cell can further comprise chimeric proteins such as chimeric antigen receptors (CAR) or priming receptors. In some embodiments, the cell comprises at least one chimeric antigen receptor. In some embodiments, the cell comprises at least one priming receptor. In some embodiments, the cell comprises at least one chimeric antigen receptor and at least one priming receptor. The at least one recombinant nucleic acid molecule(s) encoding at least one RNAi molecule can encoded on the same DNA template or nucleic acid fragment as the at least one RNAi molecule(s) or on a different DNA template or nucleic acid fragment as the RNAi molecule(s). In the case that the CAR, priming receptor, and RNAi recombinant nucleic acid molecule(s) are encoded on the same DNA template or nucleic acid fragment, the various components can be placed in any order on the DNA template. For example, the DNA template may comprise, in a 5′ to 3′ direction: the CAR, the at least one RNAi recombinant nucleic acid, and the priming receptor. Alternatively, the DNA template may comprise, in a 5′ to 3′ direction: i) the priming receptor, the at least one RNAi recombinant nucleic acid, and the CAR; ii) the at least one RNAi recombinant nucleic acid, the priming receptor, and the CAR; iii) the at least one RNAi recombinant nucleic acid, the CAR, and the priming receptor; iv) the priming receptor, the CAR, and the at least one RNAi recombinant nucleic acid; v) the CAR, the priming receptor, and the at least one RNAi recombinant nucleic acid; vi) the at least one RNAi recombinant nucleic acid, the priming receptor, the CAR; vii) the at least one RNAi recombinant nucleic acid, the CAR, and the priming receptor. In some embodiments, the at least one RNAi recombinant nucleic acid comprises two recombinant nucleic acids. In some embodiments, the recombinant nucleic acid comprises a nucleic acid that is complementary to TGFBR1. In some embodiments, the recombinant nucleic acid comprises a nucleic acid that is complementary to TGFBR2. In some embodiments, the recombinant nucleic acid comprises a nucleic acid that is complementary to FAS. In some embodiments, the recombinant nucleic acid comprises a nucleic acid that is complementary to PTPN2. In some embodiments, the recombinant nucleic acid comprises a nucleic acid that is complementary to TOX.

In some embodiments, the priming receptor comprises a first extracellular antigen-binding domain that specifically binds to a first antigen and the chimeric antigen receptor (CAR) comprises a second extracellular antigen-binding domain that specifically binds to a second antigen.

### Methods of Reducing Gene Expression

Another aspect of the invention provides a method for attenuating expression of a target gene in mammalian cells, comprising introducing into the mammalian cells a recombinant nucleic acid complementary to the target gene mRNA, such as a single-stranded hairpin ribonucleic acid (shRNA), siRNA, dsRNA, or antisense oligonucleotide. In some embodiments, the recombinant nucleic acid complementary to the target gene mRNA is an shRNA. In some embodiments, the shRNA comprises self-complementary sequences of 19 to 100 nucleotides that form a duplex region, which self-complementary sequences hybridize under intracellular conditions to a target gene mRNA transcript. In some embodiments, the shRNA comprises self-complementary sequences of 22 nt. In some embodiments, the shRNA: (i) is a substrate for cleavage by a RNaseIII enzyme to produce a double-stranded RNA product, (ii) does not produce a general sequence-independent killing of the mammalian cells, and (iii) reduces expression of said target gene in a manner dependent on the sequence of said complementary regions.

In some embodiments, the target gene is TGFBR1. In some embodiments, the target gene is TGFBR2. In some embodiments, the target gene is human TGFBR1. In some embodiments, the target gene is human TGFBR2. In some embodiments, the target gene is FAS. In some embodiments, the target gene is human FAS. In some embodiments, the target gene is PTPN2. In some embodiments, the target gene is human PTPN2. In some embodiments, the target gene is TOX. In some embodiments, the target gene is human TOX.

The cell comprising the recombinant nucleic acid can have reduced or decreased expression of a target gene selected from TGFBR1 and/or TGFBR2. In some embodiments, the cell has reduced TGFBR1 and/or TGFBR2 expression of between about 50-100%, 50-99%, 50-95%, 50-90%, 50-85%, 50-80%, 50-75%, 50-70%, 50-65%, 50-60%, 50-55%, as compared to a control cell that does not comprise the respective recombinant nucleic acid molecule(s). In some embodiments, the cell has reduced TGFBR1 expression in the cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the recombinant nucleic acid molecule(s). In some embodiments, the cell has reduced TGFBR2 expression in the cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the recombinant nucleic acid molecule(s). In some embodiments, the cell has reduced TGFBR1 and TGFBR2 expression in the immune cell by at least 10%, 15%, 20%, 25%, 30%, 35%, 40%, 45%, 50%, 55%, 60%, 65%, 75%, 80%, 85%, 90%, 95%, or 99% each, as compared to a control cell that does not comprise the respective recombinant nucleic acid molecule(s).

In some embodiments, expression of TGFBR2 in the cell is reduced by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the first nucleic acid. In some embodiments, the second nucleic acid reduces expression of TGFBR1 in the cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the second nucleic acid. In some embodiments, expression of TGFBR2 and TGFBR1 in the cell are reduced by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the first or second nucleic acid.

The cell comprising the recombinant nucleic acid can also have reduced or decreased expression of a target gene selected from the group consisting of FAS, PTPN2, and TOX. In some embodiments, the cell has reduced FAS, PTPN2, and/or TOX expression of between about 50-100%, 50-99%, 50-95%, 50-90%, 50-85%, 50-80%, 50-75%, 50-70%, 50-65%, 50-60%, 50-55%, as compared to a control cell that does not comprise the recombinant nucleic acid molecule(s). In some embodiments, the cell has reduced FAS expression in the cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the recombinant nucleic acid molecule(s). In some embodiments, the cell has reduced PTPN2 expression in the cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the recombinant nucleic acid molecule(s). In some embodiments, the cell has reduced TOX expression in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the recombinant nucleic acid molecule(s).

In some embodiments, expression of FAS in the immune cell is reduced by at least 50%, 55%, 60%, 65%, 75%, 80%, 85%, 90%, 95%, or 99% as compared to a control cell that does not comprise the first nucleic acid. In some embodiments, the second nucleic acid reduces expression of PTPN2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the second nucleic acid. In some embodiments, expression of PTPN2 in the cell is reduced by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the second nucleic acid.

In some embodiments, the second nucleic acid reduces expression of TOX in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the second nucleic acid. In some embodiments, expression of TOX in the cell is reduced by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the second nucleic acid.

In some embodiments, expression of TGFBR1, TGFBR2, FAS, PTPN2, and/or TOX is determined by a nucleic acid assay or a protein assay. In some embodiments, the nucleic acid assay comprises at least one of polymerase chain reaction (PCR), quantitative PCR (qPCR), RT-qPCR, microarray, gene array, or RNAseq

### Method of Treating Cancer

In another aspect, the invention provides methods of treating an immune-related condition or disease (e.g., cancer) in an individual comprising administering to the individual an effective amount of a composition comprising a cell comprising at least one recombinant nucleic acid that comprises a nucleic acid sequence at least 15 nucleotides in length complementary to a target selected from the group consisting of TGFBR1 and/or TGFBR2.

In some embodiments, the recombinant nucleic acid is an shRNA molecule. In some embodiments, the shRNA is a TGFBR1 shRNA molecule or a TGFBR2 shRNA. In some embodiments, the cell comprises at least a TGFBR2 shRNA molecule. In some embodiments, the cell comprises at least a TGFBR2 shRNA molecule. In some embodiments, the cell comprises at least two TGFBR2 shRNA molecules. In some embodiments, the cell comprises at least a TGFBR2 shRNA molecule and a TGFBR2 shRNA molecule.

In another aspect, the invention provides methods of enhancing an immune response in an individual comprising administering to the individual an effective amount of a composition comprising a cell comprising at least one shRNA molecule, wherein the shRNA molecule is selected from the group consisting of a TGFBR2 shRNA molecule or a TGFBR1 shRNA molecule.

In some embodiments, the methods of treating an individual or of enhancing an immune response in the individual comprise further administering to the individual an effective amount of a composition comprising a cell comprising at least one recombinant nucleic acid that comprises a nucleic acid sequence at least 15 nucleotides in length complementary to a target selected from the group consisting of TGFBR1 and/or TGFRB2.

In some embodiments, the recombinant nucleic acid is an shRNA molecule.

In some embodiments, the shRNA is selected from the group consisting of a FAS shRNA molecule, a PTPN2 shRNA molecule, and a TOX shRNA molecule. In some embodiments, the cell comprises at least a FAS shRNA molecule. In some embodiments, the cell comprises at least a PTPN2 shRNA molecule. In some embodiments, the cell comprises at least a TOX shRNA molecule. In some embodiments, the cell comprises at least a FAS shRNA molecule and a PTPN2 shRNA molecule. In some embodiments, the cell comprises at least a FAS shRNA molecule and a TOX shRNA molecule. In some embodiments, the cell comprises at least a PTPN2 shRNA molecule and a TOX shRNA molecule. In another aspect, the invention provides methods of enhancing an immune response in an individual comprising administering to the individual an effective amount of a composition comprising a cell comprising at least one shRNA molecule, wherein the shRNA molecule is selected from the group consisting of a FAS shRNA molecule, a PTPN2 shRNA molecule, and a TOX shRNA molecule. In some embodiments, the cell comprises at least a TGFBR2 shRNA molecule, a TGFBR2 shRNA molecule, a FAS shRNA molecule, a PTPN2 shRNA molecule, and/or a TOX shRNA molecule.

In some embodiments, the methods provided herein are useful for the treatment of an immune-related condition in an individual. In one embodiment, the individual is a human.

In some embodiments, the methods provided herein (such as methods of enhancing an immune response) are useful for the treatment of cancer and as such an individual receiving the system described herein has cancer. In some embodiments, the cancer is a solid cancer. In some embodiments, the cancer is a liquid cancer. In some embodiments, the cancer is immunoevasive. In some embodiments, the cancer is ovarian cancer, fallopian cancer, primary peritoneal cancer, uterine cancer, mesothelioma, cervical cancer, pancreatic, kidney cancer, lung cancer, prostate cancer, bladder cancer, breast cancer, brain cancer, leukemia, or lymphoma. In some embodiments, the cancer is ovarian, kidney, lung, breast, or prostate cancer.

In some embodiments, the treatment results in a decrease in the cancer volume or size. In some embodiments, the treatment is effective at reducing a cancer volume as compared to the cancer volume prior to administration of the recombinant nucleic acid or recombinant cell. In some embodiments, the treatment results in a decrease in the cancer growth rate. In some embodiments, the treatment is effective at reducing a cancer growth rate as compared to the cancer growth rate prior to administration of the or recombinant cell. In some embodiments, the treatment is effective at eliminating the cancer.

### Method of Immune Modulation

Methods of administration of a cell comprising a recombinant nucleic acid comprising a nucleic acid sequence at least 15 nucleotides in length complementary to TGFBR 1 and/or TGFBR2 can result in modulation of an immune response. Modulation can be an increase or decrease in an immune response. In some embodiments, modulation is an increase in an immune response.

Methods of administration of a cell comprising a recombinant nucleic acid comprising a nucleic acid sequence at least 15 nucleotides in length complementary to FAS, PTPN2, and/or TOX can result in modulation of an immune response. Modulation can be an increase or decrease in an immune response. In some embodiments, modulation is an increase in an immune response.

In one aspect, administration of a cell comprising a system comprising a recombinant nucleic acid comprising a nucleic acid sequence at least 15 nucleotides in length complementary to FAS, PTPN2, and/or TOX as described herein can result in induction of pro-inflammatory molecules, such as cytokines or chemokines. In some embodiments, the cytokine is IFNg. Generally, induced pro-inflammatory molecules are present at levels greater than that achieved with isotype control. Such pro-inflammatory molecules in turn result in activation of anti-tumor immunity, including, but not limited to, T cell activation, T cell proliferation, T cell differentiation, MI-like macrophage activation, and NK cell activation. Thus, the administration of a system comprising a recombinant nucleic acid comprising a nucleic acid sequence at least 15 nucleotides in length complementary to FAS, PTPN2, and/or TOX can induce multiple anti-tumor immune mechanisms that lead to tumor destruction.

In another aspect, provided herein are methods of increasing an immune response in an individual comprising administering to the individual an effective amount of a cell comprising a recombinant nucleic acid comprising a nucleic acid sequence at least 15 nucleotides in length complementary to TGFBR1 and/or TGFBR2. In some embodiments, the method of increasing an immune response in a subject comprises administering to the subject a cell comprising a recombinant nucleic acid comprising a nucleic acid sequence at least 15 nucleotides in length complementary to TGFBR1 and/or TGFBR2.

In another aspect, provided herein are methods of increasing an immune response in an individual comprising administering to the individual an effective amount of a cell comprising a recombinant nucleic acid comprising a nucleic acid sequence at least 15 nucleotides in length complementary to FAS, PTPN2, and/or TOX. In some embodiments, the method of increasing an immune response in a subject comprises administering to the subject a cell comprising a recombinant nucleic acid comprising a nucleic acid sequence at least 15 nucleotides in length complementary to FAS, PTPN2, and/or TOX.

In some embodiments, the cell is present in a pharmaceutical composition further comprising a pharmaceutically acceptable excipient.

In any and all aspects of increasing an immune response as described herein, any increase or decrease or alteration of an aspect of characteristic(s) or function(s) is as compared to a cell not comprising a recombinant nucleic acid comprising a nucleic acid sequence at least 15 nucleotides in length complementary to TGFBR1 and/or TGFBR2.

Increasing an immune response can be both enhancing an immune response or inducing an immune response. For instance, increasing an immune response encompasses both the start or initiation of an immune response, or ramping up or amplifying an on-going or existing immune response. In some embodiments, the treatment induces an immune response. In some embodiments, the induced immune response is an adaptive immune response. In some embodiments, the induced immune response is an innate immune response. In some embodiments, the treatment enhances an immune response. In some embodiments, the enhanced immune response is an adaptive immune response. In some embodiments, the enhanced immune response is an innate immune response. In some embodiments, the treatment increases an immune response. In some embodiments, the increased immune response is an adaptive immune response. In some embodiments, the increased immune response is an innate immune response. In some embodiments, the immune response is started or initiated by administration of a cell a recombinant nucleic acid comprising a nucleic acid sequence at least 15 nucleotides in length complementary to TGFBR1 and/or TGFBR2. In some embodiments, the immune response is enhanced by administration of cell comprising a recombinant nucleic acid comprising a nucleic acid sequence at least 15 nucleotides in length complementary to TGFBR1 and/or TGFBR2.

In another aspect, the present application provides methods of genetically editing a cell with a recombinant nucleic acid comprising a nucleic acid sequence at least 15 nucleotides in length complementary to TGFBR1 and/or TGFBR2, which results in the modulation of the immune function of the cell. The modulation can be increasing an immune response. In some embodiments, the modulation is an increase in immune function. In some embodiments, the modulation of function leads to the activation of a cell comprising the recombinant nucleic acid comprising a nucleic acid sequence at least 15 nucleotides in length complementary to TGFBR1 and/or TGFBR2.

In some embodiments, the cell is a natural killer (NK) cell, a T cell, a CD8+ T cell, a CD4+ T cell, a primary T cell, or a T cell progenitor.

In some embodiments, the modulation of function of the cells comprising the recombinant nucleic acid(s) as described herein leads to an increase in the cells' abilities to stimulate both native and activated T-cells, for example, by increasing cytokine or chemokine secretion by the cells expressing the recombinant nucleic acid(s). In some embodiments, the modulation of function enhances or increases the cells' ability to produce cytokines, chemokines, CARs, or costimulatory or activating receptors. In some embodiments, the modulation increases the T-cell stimulatory function of the cells expressing the recombinant nucleic acid(s), including, for example, the cells' abilities to trigger T-cell receptor (TCR) signaling, T-cell proliferation, or T-cell cytokine production.

In some embodiments, the increased immune response is secretion of cytokines and chemokines. In some embodiments, the recombinant nucleic acid(s) induces increased expression of at least one cytokine or chemokine in a cell as compared to an isotype control cell.

In some embodiments, the enhanced immune response is anti-tumor immune cell recruitment and activation.

In some embodiments, the cell expressing the recombinant nucleic acid(s) induces a memory immune response as compared to an isotype control cell. In general, a memory immune response is a protective immune response upon a subsequent exposure to pathogens or antigens that the immune system encountered previously. Exemplary memory immune responses include the immune response after infection or vaccination with an antigen. In general, memory immune responses are mediated by lymphocytes such as T cells or B cells. In some embodiments, the memory immune response is a protective immune response to cancer, including cancer cell growth, proliferation, or metastasis. In some embodiments, the memory immune response inhibits, prevents, or reduces cancer cell growth, proliferation, or metastasis.

### Methods of Editing Cells

The terms “gene editing” or “genome editing”, as used herein, refer to a type of genetic manipulation in which DNA is inserted, replaced, or removed from the genome using artificially manipulated nucleases or “molecular scissors”. It is a useful tool for elucidating the function and effect of sequence-specific genes or proteins or altering cell behavior (e.g. for therapeutic purposes).

Currently available genome editing tools include zinc finger nucleases (ZFN) and transcription activator-like effector nucleases (TALENs) to incorporate genes at safe harbor loci (.e.g. the adeno-associated virus integration site 1 (AAVS1) safe harbor locus). The DICE (dual integrase cassette exchange) system utilizing phiC31 integrase and Bxb1 integrase is a tool for target integration. Additionally, clustered regularly interspaced short palindromic repeat/Cas9 (CRISPR/Cas9) techniques can be used for targeted gene insertion.

Site specific gene editing approaches can include homology dependent mechanisms or homology independent mechanisms.

All methods known in the art for targeted insertion of gene sequences are contemplated in the methods described herein to insert constructs at gene targets or safe harbor loci.

Provided herein are methods of inserting one or more recombinant RNAi nucleic acids, in the absence of a viral vector. In some embodiments, the one or more recombinant nucleic acids can be inserted into the genome of a primary immune cell, in the absence of a viral vector

Described herein are methods and compositions for achieving integration of a nucleotide sequence encoding one or more recombinant nucleic acids into the genome of a cell. In some methods the efficiency of integration is increased, off-target effects are reduced and/or loss of cell viability is reduced.

A plasmid encoding one or more recombinant nucleic acids is introduced into an immune cell with a nuclease, such as a CRISPR-associated system (Cas). The nuclease can be introduced in a ribonucleoprotein format with a guide RNA (gRNA) that targets a specific site on the genome of the immune cell. The nuclease cuts the genomic DNA at this specific site. The specific site may be a portion of the genome that encodes an endogenous immune cell receptor. Thus, cutting the genome at this site will cause the immune cell to no longer express an endogenous immune cell receptor.

The plasmid may include 5′ and 3′ homology-directed repair arms complementary to sequences at a specific site on the genome of the immune cell. The complementary sequences are on either side of the site cut by the nuclease, which allows the plasmid to be incorporated at a specified insertion site on the immune cell's genome. Once the plasmid is incorporated, the cell will express the shRNA.

Initially, an immune cell, such as a T cell, is activated. The immune cell may be obtained from a patient. Thus, the present disclosure provides methods in which immune cells, such as T cells, are harvested from a patient. Then, the plasmid that encodes the one or more recombinant nucleic acids is introduced into a T cell. Advantageously, the plasmids of the present disclosure can be introduced using electroporation. When introducing the plasmid via electroporation, the nuclease may also be introduced. By using electroporation, methods of the present disclosure avoid the use of viral vectors for introducing transgenes, which is a known bottleneck in immune cell engineering. The immune cells are then expanded and co-cultured to create a sufficient quantity of engineered immune cells to be used as a therapeutic treatment.

Methods for editing the genome of a cell can include a) providing a Cas9 ribonucleoprotein complex (RNP)-DNA template complex comprising: (i) the RNP, wherein the RNP comprises a Cas9 nuclease domain and a guide RNA, wherein the guide RNA specifically hybridizes to a target region of the genome of the cell, and wherein the Cas9 nuclease domain cleaves the target region to create an insertion site in the genome of the cell; and (ii) a double-stranded or single-stranded DNA template, wherein the 5′ and 3′ ends of the DNA template comprise nucleotide sequences that are homologous to genomic sequences flanking the insertion site, and wherein the molar ratio of RNP to DNA template in the complex is from about 3:1 to about 100:1; and b) introducing the RNP-DNA template complex into the cell.

In some embodiments, the methods described herein provide an efficiency of delivery of the RNP-DNA template complex of at least about 20%, 25%, 30%, 35%, 40%, 45%, 50%, 55%, 60%, 65%, 70%, 75%, 80%, 85%, 90%, 95%, 97.5%, 99%, 99.5%, 99%, or higher. In some cases, the efficiency is determined with respect to cells that are viable after introducing the RNP-DNA template into the cell. In some cases, the efficiency is determined with respect to the total number of cells (viable or non-viable) in which the RNP-DNA template is introduced into the cell.

As another example, the efficiency of delivery can be determined by quantifying the number of genome edited cells in a population of cells (as compared to total cells or total viable cells obtained after the introducing step). Various methods for quantifying genome editing can be utilized. These methods include, but are not limited to, the use of a mismatch-specific nuclease, such as T7 endonuclease I; sequencing of one or more target loci (e.g., by sanger sequencing of cloned target locus amplification fragments); and high-throughput deep sequencing.

In some embodiments, loss of cell viability is reduced as compared to loss of cell viability after introduction of naked DNA into a cell or introduction of DNA into a cell using a viral vector. The reduction can be a reduction of at least 10%, 20%, 30%, 40%, 50%, 60%, 70%, 80%, 90%, 100% or any percentage in between these percentages. In some embodiments, off-target effects of integration are reduced as compared to off-target integration after introduction of naked DNA into a cell or introduction of DNA into a cell using a viral vector. The reduction can be a reduction of at least 10%, 20%, 30%, 40%, 50%, 60%, 70%, 80%, 90%, 100% or any percentage in between these percentages.

In some cases, the methods described herein provide for high cell viability of cells to which the RNP-DNA template has been introduced. In some cases, the viability of the cells to which the RNP-DNA template has been introduced is at least about 20%, 25%, 30%, 35%, 40%, 45%, 50%, 55%, 60%, 65%, 70%, 75%, 80%, 85%, 90%, 95%, 97.5%, 99%, 99.5%, 99%, or higher. In some cases, the viability of the cells to which the RNP-DNA template has been introduced is from about 20% to about 99%, from about 30% to about 90%, from about 35% to about 85% or 90% or higher, from about 40% to about 85% or 90% or higher, from about 50% to about 85% or 90% or higher, from about 50% to about 85% or 90% or higher, from about 60% to about 85% or 90% or higher, or from about 70% to about 85% or 90% or higher.

In the methods provided herein, the molar ratio of RNP to DNA template can be from about 3:1 to about 100:1. For example, the molar ratio can be from about 5:1 to 10:1, from about 5:1 to about 15:1, 5:1 to about 20:1; 5:1 to about 25:1; from about 8:1 to about 12:1; from about 8:1 to about 15:1, from about 8:1 to about 20:1, or from about 8:1 to about 25:1.

In some embodiments, the DNA template is at a concentration of about 2.5 pM to about 25 pM. For example, the concentration of DNA template can be about 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25 pM or any concentration in between these concentrations.

In some embodiments, the amount of DNA template is about 1 μg to about 10 μg. For example, the amount of DNA template can be about 1 μg to about 2 μg, about 1 μg to about 3 μg, about 1 μg to about 4 μg, about 1 μg to about 5 μg, about 1 μg to about 6 μg, about 1 μg to about 7 μg, about 1 μg to about 8 μg, about 1 μg to about 9 μg, about 1 μg to about 10 μg. In some embodiments the amount of DNA template is about 2 μg to about 3 μg, about 2 μg to about 4 μg, about 2 μg to about 5 μg, about 2 μg to about 6 μg, about 2 μg to about 7 μg, about 2 μg to about 8 μg, about 2 μg to about 9 μg, or 2 μg to about 10 μg. In some embodiments the amount of DNA template is about 3 μg to about 4 μg, about 3 μg to about 5 μg, about 3 μg to about 6 μg, about 3 μg to about 7 μg, about 3 μg to about 8 μg, about 3 μg to about 9 μg, or about 3 μg to about 10 μg. In some embodiments, the amount of DNA template is about 4 μg to about 5 μg, about 4 μg to about 6 μg, about 4 μg to about 7 μg, about 4 μg to about 8 μg, about 4 μg to about 9 μg, or about 4 μg to about 10 μg. In some embodiments, the amount of DNA template is about 5 μg to about 6 μg, about 5 μg to about 7 μg, about 5 μg to about 8 μg, about 5 μg to about 9 μg, or about 5 μg to about 10 μg. In some embodiments, the amount of DNA template is about 6 μg to about 7 μg, about 6 μg to about 8 μg, about 6 μg to about 9 μg, or about 6 μg to about 10 μg. In some embodiments, the amount of DNA template is about 7 μg to about 8 μg, about 7 μg to about 9 μg, or about 7 μg to about 10 μg. In some embodiments, the amount of DNA template is about 8 μg to about 9 μg, or about 8 μg to about 10 μg. In some embodiments, the amount of DNA template is about 9 μg to about 10 μg.

In some embodiments, the DNA template encodes an shRNA molecule or a fragment thereof. In some embodiments, the DNA template encodes at least one shRNA molecule. In some embodiments, the DNA template encodes at least two shRNA molecules. In some embodiments, the DNA template encodes one, two, three, four, five, six, seven, eight, nine, ten, or more shRNA molecules.

In some embodiments, the DNA template includes regulatory sequences, for example, a promoter sequence and/or an enhancer sequence to regulate expression of the heterologous protein or fragment thereof after insertion into the genome of a cell.

In some cases, the DNA template is a linear DNA template. In some cases, the DNA template is a single-stranded DNA template. In some cases, the single-stranded DNA template is a pure single-stranded DNA template. As used herein, by “pure single-stranded DNA” is meant single-stranded DNA that substantially lacks the other or opposite strand of DNA. By “substantially lacks” is meant that the pure single-stranded DNA lacks at least 100-fold more of one strand than another strand of DNA.

In some cases, the RNP-DNA template complex is formed by incubating the RNP with the DNA template for less than about one minute to about thirty minutes, at a temperature of about 20° C. to about 25° C. For example, the RNP can be incubated with the DNA template for about 5 seconds, 10 seconds, 15 seconds, 20 seconds, 25 seconds, 30 seconds, 35 seconds, 40 seconds, 45 seconds, 50 seconds, 55 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes or 30 minutes or any amount of time in between these times, at a temperature of about 20° C., 21° C., 22° C., 23° C., 24° C., or 25° C. In another example, the RNP can be incubated with the DNA template for less than about one minute to about one minute, for less than about one minute to about 5 minutes, for less than about 1 minute to about 10 minutes, for about 5 minutes to 10 minutes, for about 5 minutes to 15 minutes, for about 10 to about 15 minutes, for about 10 minutes to about 20 minutes, or for about 10 minutes to about 30 minutes, at a temperature of about 20° C. to about 25° C. In some embodiments, the RNP-DNA template complex and the cell are mixed prior to introducing the RNP-DNA template complex into the cell.

In some embodiments introducing the RNP-DNA template complex comprises electroporation. Methods, compositions, and devices for electroporating cells to introduce a RNP-DNA template complex can include those described in the examples herein. Additional or alternative methods, compositions, and devices for electroporating cells to introduce a RNP-DNA template complex can include those described in WO/2006/001614 or Kim, J. A. et al. Biosens. Bioelectron. 23, 1353-1360 (2008). Additional or alternative methods, compositions, and devices for electroporating cells to introduce a RNP-DNA template complex can include those described in U.S. Patent Appl. Pub. Nos. 2006/0094095; 2005/0064596; or 2006/0087522. Additional or alternative methods, compositions, and devices for electroporating cells to introduce a RNP-DNA template complex can include those described in Li, L. H. et al. Cancer Res. Treat. 1, 341-350 (2002); U.S. Pat. Nos. 6,773,669; 7,186,559; 7,771,984; 7,991,559; 6,485,961; 7,029,916; and U.S. Patent Appl. Pub. Nos: 2014/0017213; and 2012/0088842, all of which are hereby incorporated by reference. Additional or alternative methods, compositions, and devices for electroporating cells to introduce a RNP-DNA template complex can include those described in Geng, T. et al. J. Control Release 144, 91-100 (2010); and Wang, J., et al. Lab. Chip 10, 2057-2061 (2010), all of which are hereby incorporated by reference.

In some embodiments, the Cas9 protein can be in an active endonuclease form, such that when bound to target nucleic acid as part of a complex with a guide RNA or part of a complex with a DNA template, a double strand break is introduced into the target nucleic acid. The double strand break can be repaired by NHEJ to introduce random mutations, or HDR to introduce specific mutations. Various Cas9 nucleases can be utilized in the methods described herein. For example, a Cas9 nuclease that requires an NGG protospacer adjacent motif (PAM) immediately 3′ of the region targeted by the guide RNA can be utilized. Such Cas9 nucleases can be targeted to any region of a genome that contains an NGG sequence. As another example, Cas9 proteins with orthogonal PAM motif requirements can be utilized to target sequences that do not have an adjacent NGG PAM sequence. Exemplary Cas9 proteins with orthogonal PAM sequence specificities include, but are not limited to, CFP1, those described in Nature Methods 10, 1116-1121 (2013), and those described in Zetsche et al., Cell, Volume 163, Issue 3, p 759-771, 22 Oct. 2015, both of which are hereby incorporated by reference.

In some cases, the Cas9 protein is a nickase, such that when bound to target nucleic acid as part of a complex with a guide RNA, a single strand break or nick is introduced into the target nucleic acid. A pair of Cas9 nickases, each bound to a structurally different guide RNA, can be targeted to two proximal sites of a target genomic region and thus introduce a pair of proximal single stranded breaks into the target genomic region. Nickase pairs can provide enhanced specificity because off-target effects are likely to result in single nicks, which are generally repaired without lesion by base-excision repair mechanisms. Exemplary Cas9 nickases include Cas9 nucleases having a D10A or H840A mutation.

In some embodiments, the RNP comprises a Cas9 nuclease. In some embodiments, the RNP comprises a Cas9 nickase. In some embodiments, the RNP-DNA template complex comprises at least two structurally different RNP complexes. In some embodiments, the at least two structurally different RNP complexes contain structurally different Cas9 nuclease domains In some embodiments, the at least two structurally different RNP complexes contain structurally different guide RNAs. In some embodiments, wherein the at least two structurally different RNP complexes contain structurally different guide RNAs, each of the structurally different RNP complexes comprises a Cas9 nickase, and the structurally different guide RNAs hybridize to opposite strands of the target region.

In some cases, a plurality of RNP-DNA templates comprising structurally different ribonucleoprotein complexes is introduced into the cell. For example a Cas9 protein can be complexed with a plurality (e.g., 2, 3, 4, 5, or more, e.g., 2-10, 5-100, 20-100) of structurally different guide RNAs to target insertion of a DNA template at a plurality of structurally different target genomic regions.

In the methods and compositions provided herein, cells include, but are not limited to, eukaryotic cells, prokaryotic cells, animal cells, plant cells, fungal cells and the like. Optionally, the cell is a mammalian cell, for example, a human cell. The cell can be in vitro, ex vivo or in vivo. The cell can also be a primary cell, a germ cell, a stem cell or a precursor cell. The precursor cell can be, for example, a pluripotent stem cell, or a hematopoietic stem cell. In some embodiments, the cell is a primary hematopoietic cell or a primary hematopoietic stem cell. In some embodiments, the primary hematopoietic cell is an immune cell. In some embodiments, the immune cell is a T cell. In some embodiments, the T cell is a regulatory T cell, an effector T cell, or a naïve T cell. In some embodiments, the T cell is a CD4+ T cell. In some embodiments, the T cell is a CD8+ T cell. In some embodiments, the T cell is a CD4+CD8+ T cell. In some embodiments, the T cell is a CD4-CD8-T cell. Populations of any of the cells modified by any of the methods described herein are also provided. In some embodiments, the methods further comprise expanding the population of modified cells.

In some cases, the cells are removed from a subject, modified using any of the methods described herein and administered to the patient. In other cases, any of the constructs described herein is delivered to the patient in vivo. See, for example, U.S. Pat. No. 9,737,604 and Zhang et al. “Lipid nanoparticle-mediated efficient delivery of CRISPR/Cas9 for tumor therapy,” NPG Asia Materials Volume 9, page e441 (2017), both of which are hereby incorporated by reference.

In some embodiments, the RNP-DNA template complex is introduced into about 1×10⁵ to about 2×10⁶ cells. For example, the RNP-DNA template complex can be introduced into about 1×10⁵ to about 5×10⁵ cells, about 1×10⁵ to about 1×10⁶, 1×10⁵ to about 1.5×10⁶, 1×10⁵ to about 2×10⁶, about 1×10⁶ to about 1.5×10⁶ cells or about 1×10⁶ to about 2×10⁶.

In some cases, the methods and compositions described herein can be used for generation, modification, use, or control of recombinant immune cells, such as chimeric antigen receptor T cells (CAR T cells). Such CAR T cells can be used to treat or prevent cancer, an infectious disease, or autoimmune disease in a subject. For example, in some embodiments, one or more gene products are inserted or knocked-in to a T cell to express a heterologous protein (e.g., a chimeric antigen receptor (CAR) or a priming receptor).

### Insertion Sites

Methods for editing the genome of an immune cell include a method of editing the genome of a human T cell comprise inserting a nucleic acid sequence or construct into a target region in exon 1 of the TCR-α subunit (TRAC) gene in the human immune cell. In some embodiments, the target region is in exon 1 of the constant domain of TRAC gene. In other embodiments, the target region is in exon 1, exon 2 or exon 3, prior to the start of the sequence encoding the TCR-α transmembrane domain.

Methods for editing the genome of an immune cell also include a method of editing the genome of a human immune T cell comprise inserting a nucleic acid sequence or construct into a target region in exon 1 of a TCR-β subunit (TRBC) gene in the human T cell. In some embodiments, the target region is in exon 1 of the TRBC1 or TRBC2 gene.

Methods for editing the genome of an immune cell, specifically, include a method of editing the genome of a human immune cell comprise inserting a nucleic acid sequence or construct into a target region of a genomic safe harbor (GSH).

Methods for editing the genome of a T cell also include a method of editing the genome of a human T cell comprise inserting a nucleic acid sequence or construct into a GS94 target region (locus chr11: 128340000-128350000).

In some embodiments, the target region is the GS94 locus.

Gene editing therapies include, for example, vector integration and site specific integration. Site-specific integration is a promising alternative to random integration of viral vectors, as it mitigates the risks of insertional mutagenesis or insertional oncogenesis (Kolb et al. Trends Biotechnol. 2005 23:399-406; Porteus et al. Nat Biotechnol. 2005 23:967-973; Paques et al. Curr Gen Ther. 2007 7:49-66). However, site specific integration continues to face challenges such as poor knock-in efficiency, risk of insertional oncogenesis, unstable and/or anomalous expression of adjacent genes or the transgene, low accessibility (e.g. within 20 kB of adjacent genes), etc. These challenges can be addressed, in part, through the identification and use of safe harbor loci or safe harbor sites (SHS), which are sites in which genes or genetic elements can be incorporated without disruption to expression or regulation of adjacent genes.

The most widely used of the putative human safe harbor sites is the AAVS1 site on chromosome 19q, which was initially identified as a site for recurrent adeno-associated virus insertion. Other potential SHS have been identified on the basis of homology, with sites first identified in other species (e.g., the human homolog of the permissive murine Rosa26 locus) or among the growing number of human genes that appear non-essential under some circumstances. One putative SHS of this type is the CCR5 chemokine receptor gene, which, when disrupted, confers resistance to human immunodeficiency virus infection. Additional potential genomic SHS have been identified in human and other cell types on the basis of viral integration site mapping or gene-trap analyses, as was the original murine Rosa26 locus. The three top SHS, AAVS1, CCR5, and Rosa26, are in close proximity to many protein coding genes and regulatory elements. (See Sadelain, M., et al. (2012). Safe harbours for the integration of new DNA in the human genome. Nature reviews Cancer, 12 (1), 51-58, the relevant disclosures of which are herein incorporated by reference in their entirety).

The AAVS1 (also known as the PPP1R12C locus) on human chromosome 19 is a known SHS for hosting transgenes (e.g. DNA transgenes) with expected function. It is at position 19q13.42. It has an open chromatin structure and is transcription-competent. The canonical SHS locus for AAVS1 is chr19: 55,625,241-55,629,351. See Pellenz et al. “New Human Chromosomal Sites with “Safe Harbor” Potential for Targeted Transgene Insertion.” Human gene therapy vol. 30,7 (2019): 814-828, the relevant disclosures of which are herein incorporated by reference. An exemplary AAVS1 target gRNA and target sequence are provided below:

AAVS1-gRNA sequence:
ggggccactagggacaggatGTTTTAGAGCTAGAA
ATAGCAAGTTAAAATAAGGCTAGTCCGTTATCAAC
TTGAAAAAGTGGCACCGAGTCGGTGCTTTTTTT
AAVS1 target sequence:
ggggccactagggacaggat

CCR5, which is located on chromosome 3 at position 3p21.31, encodes the major co-receptor for HIV-1. Disruption at this site in the CCR5 gene has been beneficial in HIV/AIDS therapy and prompted the development of zinc-finger nucleases that target its third exon. The canonical SHS locus for CCR5 is chr3: 46,414,443-46,414,942. See Pellenz et al. “New Human Chromosomal Sites with “Safe Harbor” Potential for Targeted Transgene Insertion.” Human gene therapy vol. 30,7 (2019): 814-828, the relevant disclosures of which are herein incorporated by reference.

The mouse Rosa26 locus is particularly useful for genetic modification as it can be targeted with high efficiency and is expressed in most cell types tested. Irion et al. 2007 (“Identification and targeting of the ROSA26 locus in human embryonic stem cells.” Nature biotechnology 25.12 (2007): 1477-1482, the relevant disclosure of which are herein incorporated by reference) identified the human homolog, human ROSA26, in chromosome 3 (position 3p25.3). The canonical SHS locus for human Rosa26 (hRosa26) is chr3: 9,415,082-9,414,043. See Pellenz et al. “New Human Chromosomal Sites with “Safe Harbor” Potential for Targeted Transgene Insertion.” Human gene therapy vol. 30,7 (2019): 814-828, the relevant disclosures of which are herein incorporated by reference.

Additional examples of safe harbor sites are provided in Pellenz et al. “New Human Chromosomal Sites with “Safe Harbor” Potential for Targeted Transgene Insertion.” Human gene therapy vol. 30,7 (2019): 814-828, the relevant disclosures of which are herein incorporated by reference. Examples of additional integration sites are provided in Table 1.

In some embodiments, the safe harbor sites allow for high transgene expression (sufficient to allow for transgene functionality or treatment of a disease of interest) and stable expression of the transgene over several days, weeks or months. In some embodiments, knockout of the gene at the safe harbor locus confers benefit to the function of the cell, or the gene at the safe harbor locus has no known function within the cell. In some embodiments the safe harbor locus results in stable transgene expression in vitro with or without CD3/CD28 stimulation, negligible off-target cleavage as detected by iGuide-Seq or CRISPR-Seq, less off-target cleavage relative to other loci as detected by iGuide-Seq or CRISPR-Seq, negligible transgene-independent cytotoxicity, negligible transgene-independent cytokine expression, negligible transgene-independent chimeric antigen receptor expression, negligible deregulation or silencing of nearby genes, and positioned outside of a cancer-related gene.

As used, a “nearby gene” can refer to a gene that is within about 100 KB, about 125 KB, about 150 KB, about 175 kB, about 200 kB, about 225 kB, about 250 KB, about 275 kB, about 300 KB, about 325 kB, about 350 kB, about 375 kB, about 400 kB, about 425 kB, about 450 kB, about 475 kB, about 500 KB, about 525 kB, about 550 KB away from the safe harbor locus (integration site).

In some embodiments, the present disclosure contemplates nucleic acid inserts that comprise one or more recombinant RNAi nucleic acids, such as at least one shRNA molecule. The integration of the one or more recombinant RNAi nucleic acids can result in, for example, enhanced therapeutic properties. These enhanced therapeutic properties, as used herein, refer to an enhanced therapeutic property of a cell when compared to a typical immune cell of the same normal cell type. For example, an NK cell having “enhanced therapeutic properties” has an enhanced, improved, and/or increased treatment outcome when compared to a typical, unmodified and/or naturally occurring NK cell. The therapeutic properties of immune cells can include, but are not limited to, cell transplantation, transport, homing, viability, self-renewal, persistence, immune response control and regulation, survival, and cytotoxicity. The therapeutic properties of immune cells are also manifested by: antigen-targeted receptor expression; HLA presentation or lack thereof; tolerance to the intratumoral microenvironment; induction of bystander immune cells and immune regulation; improved target specificity with reduction; resistance to treatments such as chemotherapy.

As used herein, the term “insert size” refers to the length of the nucleotide sequence being integrated (inserted) at the target locus or safe harbor site.

The inserts of the present disclosure refer to nucleic acid molecules or polynucleotide inserted at a target locus or safe harbor site. In some embodiments, the nucleotide sequence is a DNA molecule, e.g., genomic DNA, or comprises deoxy-ribonucleotides. In some embodiments, the insert comprises a smaller fragment of DNA, such as a plastid DNA, mitochondrial DNA, or DNA isolated in the form of a plasmid, a fosmid, a cosmid, a bacterial artificial chromosome (BAC), a yeast artificial chromosome (YAC), and/or any other sub-genome segment of DNA. The nucleotides in the insert are contemplated as naturally occurring nucleotides, non-naturally occurring, and modified nucleotides. Nucleotides may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications. The polynucleotides can be in any topological conformation, including single-stranded, double-stranded, partially duplexed, triplexed, hairpinned, circular conformations, and other three-dimension conformations contemplated in the art.

The inserts can have coding and/or non-coding regions. The insert can comprises a non-coding sequence (e.g., control elements, e.g., a promoter sequence). In some embodiments, the insert encodes one or more recombinant RNAi nucleic acids.

In some embodiments, the nucleic acid sequence is inserted into the genome of the immune cell via non-viral delivery. In non-viral delivery methods, the nucleic acid can be naked DNA, or in a non-viral plasmid or vector. Non-viral delivery techniques can be site-specific integration techniques, as described herein or known to those of ordinary skill in the art. Examples of site-specific techniques for integration into the safe harbor loci include, without limitation, homology-dependent engineering using nucleases and homology independent targeted insertion using Cas9 or other CRISPR endonucleases.

In some embodiments, the insert is integrated at a safe harbor site by introducing into the engineered cell, (a) a targeted nuclease that cleaves a target region in the safe harbor site to create the insertion site; and (b) the nucleic acid sequence (insert), wherein the insert is incorporated at the insertion site by, e.g., HDR. Examples of non-viral delivery techniques that can be used in the methods of the present disclosure are provided in U.S. application Ser. Nos. 16/568,116 and 16/622,843, the relevant disclosures of which are herein incorporated by reference in their entirety.

Examples of integration sites contemplated are provided in Table D.

TABLE D
sgRNA sequences

| | | | | | Median (% |
|---|---|---|---|---|---|
| | | | | | Modified), |
| | | sgRNA | | | summarized |
| | | start | sgRNA | | from 2 |
| sgRNA | sgRNA | coor | Target | Integration | donors, 2 |
| ID | Sequence | GRCH38 | Loci | Site | primersets |
| sgRNA_1 | GCACC | chr16: | APRT | APRT | 79.28 |
| | TGAAT | 88811818 | | | |
| | ACCAC | | | | |
| | GCCTG | | | | |
| sgRNA_2 | CGCCT | chr16: | APRT | APRT | 78.60 |
| | GCGAT | 88811551 | | | |
| | GTAGT | | | | |
| | CGATG | | | | |
| sgRNA_3 | CAGGA | chr16: | APRT | APRT | 85.25 |
| | CGGGC | 88811640 | | | |
| | GAGAT | | | | |
| | GTCCC | | | | |
| sgRNA_4 | CTGAA | chr15: | B2M | B2M | 78.51 |
| | TCTTT | 44715425 | | | |
| | GGAGT | | | | |
| | ACCTG | | | | |
| sgRNA_5 | GGCCA | chr15: | B2M | B2M | 94.75 |
| | CGGAG | 44711550 | | | |
| | CGAGA | | | | |
| | CATCT | | | | |
| sgRNA_6 | AAGTC | chr15: | B2M | B2M | 70.97 |
| | AACTT | 44715515 | | | |
| | CAATG | | | | |
| | TCGGA | | | | |
| sgRNA_7 | GCTTG | chr19: | CAPNS1 | CAPNS1 | 89.34 |
| | GAGGC | 36141111 | | | |
| | CTGAT | | | | |
| | CAGCG | | | | |
| sgRNA_8 | CTTAT | chr19: | CAPNS1 | CAPNS1 | 91.09 |
| | CTCTT | 36142301 | | | |
| | CGCAG | | | | |
| | CGAGG | | | | |
| sgRNA_9 | CACAC | chr19: | CAPNS1 | CAPNS1 | 71.98 |
| | ATTAC | 36142676 | | | |
| | TCCAA | | | | |
| | CATTG | | | | |
| sgRNA_10 | TTCCG | chr3: | CBLB | CBLB | 91.55 |
| | CAAAA | 105746019 | | | |
| | TAGAG | | | | |
| | CCCCA | | | | |
| sgRNA_11 | TGCAC | chr3: | CBLB | CBLB | 91.43 |
| | AGAAC | 105751622 | | | |
| | TATCG | | | | |
| | TACCA | | | | |
| sgRNA_12 | GCAAT | chr3: | CBLB | CBLB | 76.18 |
| | AAGAC | 105853470 | | | |
| | TCTTT | | | | |
| | AAAGA | | | | |
| sgRNA_13 | CAAAG | chr1: | CD2 | CD2 | 89.80 |
| | AGATT | 116754658 | | | |
| | ACGAA | | | | |
| | TGCCT | | | | |
| sgRNA_14 | CAAGG | chr1: | CD2 | CD2 | 92.70 |
| | CACCC | 116754663 | | | |
| | CAGGT | | | | |
| | TTCCA | | | | |
| sgRNA_15 | TTACG | chr1: | CD2 | CD2 | 92.82 |
| | AATGC | 116754666 | | | |
| | CTTGG | | | | |
| | AAACC | | | | |
| sgRNA_16 | CAGAG | chr11: | CD3E | CD3E | 90.96 |
| | ACGCA | 118315540 | | | |
| | TCTGA | | | | |
| | CCCTC | | | | |
| sgRNA_17 | CATGC | chr11: | CD3E | CD3E | 87.47 |
| | AGTTC | 118313715 | | | |
| | TCACA | | | | |
| | CACTG | | | | |
| sgRNA_18 | GTGTG | chr11: | CD3E | CD3E | 86.65 |
| | AGAAC | 118313715 | | | |
| | TGCAT | | | | |
| | GGAGA | | | | |
| sgRNA_19 | TCTCA | chr11: | CD3G | CD3G | 87.24 |
| | TTTCA | 118349748 | | | |
| | GGAAA | | | | |
| | CCACT | | | | |
| sgRNA_20 | AGTCA | chr11: | CD3G | CD3G | 87.99 |
| | TACAC | 118349754 | | | |
| | CTTAA | | | | |
| | CCAAG | | | | |
| sgRNA_21 | TTCAA | chr11: | CD3G | CD3G | 86.55 |
| | GGAAA | 118352458 | | | |
| | CCAGT | | | | |
| | TGAGG | | | | |
| sgRNA_22 | GAGCC | chr11: | CD5 | CD5 | 84.03 |
| | TTGCC | 61118177 | | | |
| | TGGAA | | | | |
| | ATCTG | | | | |
| sgRNA_23 | AAGCG | chr11: | CD5 | CD5 | 89.19 |
| | TCAAA | 61118324 | | | |
| | AGTCT | | | | |
| | GCCAG | | | | |
| sgRNA_24 | CGTTC | chr11: | CD5 | CD5 | 83.11 |
| | CAACT | 61118121 | | | |
| | CGAAG | | | | |
| | TGCCA | | | | |
| sgRNA_25 | GAGCG | chr9: | EDF1 | EDF1 | 88.84 |
| | ACTGG | 136866246 | | | |
| | GACAC | | | | |
| | GGTGA | | | | |
| sgRNA_26 | GCTGC | chr9: | EDF1 | EDF1 | 91.04 |
| | GCAAG | 136866211 | | | |
| | AAGGG | | | | |
| | CCCTA | | | | |
| sgRNA_27 | TTGTT | chr9: | EDF1 | EDF1 | 85.98 |
| | CTGGC | 136863433 | | | |
| | CAGCA | | | | |
| | GCCCC | | | | |
| sgRNA_28 | CTTCC | chr19: | FTL | FTL | 93.10 |
| | AGAGC | 48965791 | | | |
| | CACAT | | | | |
| | CATCG | | | | |
| sgRNA_29 | GGGAC | chr19: | FTL | FTL | 88.86 |
| | TCACC | 48965601 | | | |
| | AGAGA | | | | |
| | GAGGT | | | | |
| sgRNA_30 | CGGTC | chr19: | FTL | FTL | 93.14 |
| | GAAAT | 48965770 | | | |
| | AGAAG | | | | |
| | CCCTA | | | | |
| sgRNA_31 | AAAAG | chr10: | PTEN | PTEN | 92.37 |
| | GATAT | 87933015 | | | |
| | TGTGC | | | | |
| | AACTG | | | | |
| sgRNA_32 | TGTGC | chr10: | PTEN | PTEN | 90.64 |
| | ATATT | 87933183 | | | |
| | TATTA | | | | |
| | CATCG | | | | |
| sgRNA_33 | TTTGT | chr10: | PTEN | PTEN | 85.36 |
| | GAAGA | 87933087 | | | |
| | TCTTG | | | | |
| | ACCAA | | | | |
| sgRNA_34 | TGTCA | chr18: | PTPN2 | PTPN2 | 87.94 |
| | TGCTG | 12830972 | | | |
| | AACCG | | | | |
| | CATTG | | | | |
| sgRNA_35 | CCACT | chr18: | PTPN2 | PTPN2 | 92.45 |
| | CTATG | 12859219 | | | |
| | AGGAT | | | | |
| | AGTCA | | | | |
| sgRNA_36 | TTGAC | chr18: | PTPN2 | PTPN2 | 93.96 |
| | ATAGA | 12836828 | | | |
| | AGAGG | | | | |
| | CACAA | | | | |
| sgRNA_37 | GAGTA | chr12: | PTPN6 | PTPN6 | 89.61 |
| | CTACA | 6952098 | | | |
| | CTCAG | | | | |
| | CAGCA | | | | |
| sgRNA_38 | TCACG | chr12: | PTPN6 | PTPN6 | 82.74 |
| | CACAA | 6954872 | | | |
| | GAAAC | | | | |
| | GTCCA | | | | |
| sgRNA_39 | AGGTC | chr12: | PTPN6 | PTPN6 | 91.27 |
| | TCGGT | 6951610 | | | |
| | GAAAC | | | | |
| | CACCT | | | | |
| sgRNA_40 | AGCAT | chr1: | PTPRC | PTPRC | 88.88 |
| | TATCC | 198696873 | | | |
| | AAAGA | | | | |
| | GTCCG | | | | |
| sgRNA_41 | ATATT | chr1: | PTPRC | PTPRC | 88.95 |
| | AATTC | 198692370 | | | |
| | TTACC | | | | |
| | AGTGG | | | | |
| sgRNA_42 | AGCTT | chr1: | PTPRC | PTPRC | 96.89 |
| | TAAAT | 198756176 | | | |
| | CAAGG | | | | |
| | TTCAT | | | | |
| sgRNA_43 | ATCCC | chr11: | PTPRC | PTPRC | 84.08 |
| | GAGCC | 67436325 | AP | AP | |
| | CTAAG | | | | |
| | GTGCA | | | | |
| sgRNA_44 | GGCAG | chr11: | PTPRC | PTPRC | 97.74 |
| | CGCGG | 67436285 | AP | AP | |
| | AGGAC | | | | |
| | AGCGT | | | | |
| sgRNA_45 | CTCAG | chr11: | PTPRC | PTPRC | 91.50 |
| | GGGGC | 67436170 | AP | AP | |
| | TACTA | | | | |
| | CCACC | | | | |
| sgRNA_46 | GTCAC | chr5: | RPS23 | RPS23 | 79.40 |
| | CGACG | 82277810 | | | |
| | AGACC | | | | |
| | AGAAG | | | | |
| sgRNA_47 | GTCGT | chr5: | RPS23 | RPS23 | 83.07 |
| | GGACT | 82277843 | | | |
| | TCGTA | | | | |
| | CTGCT | | | | |
| sgRNA_48 | TAATT | chr5: | RPS23 | RPS23 | 61.94 |
| | TTTAG | 82277860 | | | |
| | GCAAG | | | | |
| | TGTCG | | | | |
| sgRNA_49 | TTAGC | chr14: | RTRAF | RTRAF | 85.50 |
| | TGTTA | 51993810 | | | |
| | GACTT | | | | |
| | GAATA | | | | |
| sgRNA_50 | CGAGA | chr14: | RTRAF | RTRAF | 85.64 |
| | GCCGT | 51989652 | | | |
| | CAACT | | | | |
| | TGCGT | | | | |
| sgRNA_51 | CGGCT | chr14: | RTRAF | RTRAF | 88.77 |
| | TCAAC | 51989700 | | | |
| | TGCAA | | | | |
| | AGGTG | | | | |
| sgRNA_52 | TATGA | chr15: | SERF2 | SERF2 | 89.61 |
| | AAAAG | 43793025 | | | |
| | CAGAG | | | | |
| | CGACT | | | | |
| sgRNA_53 | TCTGG | chr15: | SERF2 | SERF2 | 86.73 |
| | CGGGC | 43792989 | | | |
| | GAGCT | | | | |
| | CACGC | | | | |
| sgRNA_54 | CTCAC | chr15: | SERF2 | SERF2 | 80.57 |
| | GCTGG | 43792977 | | | |
| | TTACC | | | | |
| | GCCTA | | | | |
| sgRNA_55 | AAAGA | chr12: | SLC38A1 | SLC38A1 | 92.24 |
| | TTACG | 46207559 | | | |
| | AACTT | | | | |
| | CCCTG | | | | |
| sgRNA_56 | GTTAA | chr12: | SLC38A1 | SLC38A1 | 91.51 |
| | AAACA | 46229232 | | | |
| | GACAT | | | | |
| | GCCTA | | | | |
| sgRNA_57 | ATGCC | chr12: | SLC38A1 | SLC38A1 | 79.48 |
| | TAAGG | 46229246 | | | |
| | AGGTT | | | | |
| | GTACC | | | | |
| sgRNA_58 | CTCCA | chr18: | SMAD2 | SMAD2 | 79.53 |
| | GGTAT | 47869418 | | | |
| | CCCAT | | | | |
| | CGAAA | | | | |
| sgRNA_59 | CACCA | chr18: | SMAD2 | SMAD2 | 86.61 |
| | AATAC | 47870532 | | | |
| | GATAG | | | | |
| | ATCAG | | | | |
| sgRNA_60 | TGGCG | chr18: | SMAD2 | SMAD2 | 82.91 |
| | GCGTG | 47896729 | | | |
| | AATGG | | | | |
| | CAAGA | | | | |
| sgRNA_61 | TAGGA | chr16: | SOCS1 | SOCS1 | 92.25 |
| | TGGTA | 11255478 | | | |
| | GCACA | | | | |
| | CAACC | | | | |
| sgRNA_62 | CAGCA | chr16: | SOCS1 | SOCS1 | 83.79 |
| | GCAGA | 11255432 | | | |
| | GCCCC | | | | |
| | GACGG | | | | |
| sgRNA_63 | CGGCG | chr16: | SOCS1 | SOCS1 | 84.24 |
| | TGCGA | 11255296 | | | |
| | ACGGA | | | | |
| | ATGTG | | | | |
| sgRNA_64 | TATAG | chr15: | SRP14 | SRP14 | 95.12 |
| | ACGCT | 40038895 | | | |
| | GCCCG | | | | |
| | ACGTC | | | | |
| sgRNA_65 | TCCAA | chr15: | SRP14 | SRP14 | 92.14 |
| | AGAAG | 40038368 | | | |
| | GGTAC | | | | |
| | TGTGG | | | | |
| sgRNA_66 | ACAGT | chr15: | SRP14 | SRP14 | 65.82 |
| | ACCCT | 40038358 | | | |
| | TCTTT | | | | |
| | GGAAT | | | | |
| sgRNA_67 | GCGAC | chr12: | SRSF9 | SRSF9 | 83.68 |
| | GGGCG | 120469572 | | | |
| | CATCT | | | | |
| | ACGTG | | | | |
| sgRNA_68 | CCCGA | chr12: | SRSF9 | SRSF9 | 92.56 |
| | CCTCC | 120465700 | | | |
| | ATAAG | | | | |
| | TCCTG | | | | |
| sgRNA_69 | GGGGT | chr12: | SRSF9 | SRSF9 | 89.94 |
| | CCTCG | 120469426 | | | |
| | AAGCG | | | | |
| | CACGA | | | | |
| sgRNA_70 | TGCTC | chr5: | SUB1 | SUB1 | 79.36 |
| | TGTTT | 32591641 | | | |
| | AGAAG | | | | |
| | ATGAC | | | | |
| sgRNA_71 | ATATT | chr5: | SUB1 | SUB1 | 70.93 |
| | CTTTT | 32591566 | | | |
| | CTAGT | | | | |
| | TAAAG | | | | |
| sgRNA_72 | CCTGT | chr5: | SUB1 | SUB1 | 93.66 |
| | AAAGA | 32591614 | | | |
| | AACAA | | | | |
| | AAGAC | | | | |
| sgRNA_73 | TGGAG | chr4: | TET2 | TET2 | 83.53 |
| | AAAGA | 105234315 | | | |
| | CGTAA | | | | |
| | CTTCG | | | | |
| sgRNA_74 | TCTGC | chr4: | TET2 | TET2 | 90.97 |
| | CCTGA | 105234747 | | | |
| | GGTAT | | | | |
| | GCGAT | | | | |
| sgRNA_75 | ATTCC | chr4: | TET2 | TET2 | 89.62 |
| | GCTTG | 105235656 | | | |
| | GTGAA | | | | |
| | AACGA | | | | |
| sgRNA_76 | CAGGC | chr3: | TIGIT | TIGIT | 92.65 |
| | ACAAT | 114295571 | | | |
| | AGAAA | | | | |
| | CAACG | | | | |
| sgRNA_77 | CCATT | chr3: | TIGIT | TIGIT | 60.75 |
| | TGTAA | 114295700 | | | |
| | TGCTG | | | | |
| | ACTTG | | | | |
| sgRNA_78 | CTGGG | chr3: | TIGIT | TIGIT | 87.99 |
| | TCACT | 114295634 | | | |
| | TGTGC | | | | |
| | CGTGG | | | | |
| sgRNA_79 | GTCAG | chr14: | TRAC | TRAC | 98.20 |
| | GGTTC | 22547508 | | | |
| | TGGAT | | | | |
| | ATCTG | | | | |
| sgRNA_80 | TGGAT | chr14: | TRAC | TRAC | 88.15 |
| | TTAGA | 22547541 | | | |
| | GTCTC | | | | |
| | TCAGC | | | | |
| sgRNA_81 | CTGCG | chr14: | TRAC | TRAC | 94.77 |
| | GCTGT | 22550661 | | | |
| | GGTCC | | | | |
| | AGCTG | | | | |
| sgRNA_82 | ACAAA | chr14: | TRAC | TRAC | 87.86 |
| | ACTGT | 22547658 | | | |
| | GCTAG | | | | |
| | ACATG | | | | |
| sgRNA_83 | TTCTT | chr14: | TRAC | TRAC | 89.85 |
| | CCCCA | 22547778 | | | |
| | GCCCA | | | | |
| | GGTAA | | | | |
| sgRNA_84 | CGTCA | chr14: | TRAC | TRAC | 95.81 |
| | TGAGC | 22550625 | | | |
| | AGATT | | | | |
| | AAACC | | | | |
| sgRNA_85 | GAGAG | chr19: | TRIM28 | TRIM28 | 89.44 |
| | CGCCT | 58544980 | | | |
| | GCGAC | | | | |
| | CCGAG | | | | |
| sgRNA_86 | CCAGC | chr19: | TRIM28 | TRIM28 | 94.79 |
| | GGGTG | 58544869 | | | |
| | AAGTA | | | | |
| | CACCA | | | | |
| sgRNA_87 | GGAGC | chr19: | TRIM28 | TRIM28 | 91.81 |
| | GCTTT | 58544839 | | | |
| | TCGCC | | | | |
| | GCCAG | | | | |
| sgRNA_88 | TGAGG | chr10: | chr10: | desert_1 | 69.44 |
| | CCTGG | 33134193 | 33130000- | (GS88) | |
| | ACCTT | | 33140000 | | |
| | ATGCA | | | | |
| sgRNA_89 | CCTGG | chr10: | chr10: | desert_1 | 95.25 |
| | TGGAG | 33132917 | 33130000- | (GS89) | |
| | TGAAC | | 33140000 | | |
| | CATGA | | | | |
| sgRNA_90 | CAAGC | chr10: | chr10: | desert_1 | 91.13 |
| | ACTTA | 33134633 | 33130000- | (GS90) | |
| | GGTTC | | 33140000 | | |
| | CCCTG | | | | |
| sgRNA_91 | GGTCT | chr10: | chr10: | desert_2 | 92.02 |
| | CCCTA | 72294568 | 72290000- | (GS91) | |
| | CAATT | | 72300000 | | |
| | CAGCG | | | | |
| sgRNA_92 | CACAG | chr10: | chr10: | desert_2 | 90.22 |
| | CGCGT | 72298268 | 72290000- | (GS92) | |
| | GACTG | | 72300000 | | |
| | CAATG | | | | |
| sgRNA_93 | TCTGG | chr10: | chr10: | desert_2 | 86.35 |
| | GGCAC | 72292786 | 72290000- | (GS93) | |
| | CAATT | | 72300000 | | |
| | CTAGG | | | | |
| sgRNA_94 | GAGCC | chr11: | chr11: | desert_3 | 91.24 |
| | ATGCT | 128342576 | 128340000- | (GS94) | |
| | TGGCT | | 128350000 | | |
| | TACGA | | | | |
| sgRNA_95 | GTACA | chr11: | chr11: | desert_3 | 89.02 |
| | AGTAC | 128343592 | 128340000- | (GS95) | |
| | TTATC | | 128350000 | | |
| | TCATG | | | | |
| sgRNA_96 | GAGAT | chr11: | chr11: | desert_3 | 96.47 |
| | AACAA | 128347170 | 128340000- | (GS96) | |
| | CATAA | | 128350000 | | |
| | CAACA | | | | |
| sgRNA_97 | CATAT | chr11: | chr11: | desert_4 | 88.54 |
| | TCCAT | 65425000 | 65425000- | (GS97) | |
| | AGTCT | | 65427000 | | |
| | TTGGG | | (NEAT1) | | |
| sgRNA_98 | CTGCC | chr11: | chr11: | desert_4 | 92.76 |
| | CCTTA | 65425507 | 65425000- | (GS98) | |
| | GCAAC | | 65427000 | | |
| | TTAGG | | (NEAT1) | | |
| sgRNA_99 | TGTTT | chr11: | chr11: | desert_4 | 90.76 |
| | AAAAA | 65426264 | 65425000- | (GS99) | |
| | TATGT | | 65427000 | | |
| | TGACA | | (NEAT1) | | |
| sgRNA_100 | CCAGG | chr15: | chr15: | desert_5 | 87.84 |
| | AATGG | 92830315 | 92830000- | (GS100) | |
| | AAACT | | 92840000 | | |
| | CACGC | | | | |
| sgRNA_101 | GAGGC | chr15: | chr15: | desert_5 | 85.32 |
| | CGCTG | 92831850 | 92830000- | (GS101) | |
| | AATTA | | 92840000 | | |
| | ACCCG | | | | |
| sgRNA_102 | ATACA | chr15: | chr15: | desert_5 | 99.92 |
| | CGCAC | 92831131 | 92830000- | (GS102) | |
| | ACTTG | | 92840000 | | |
| | CAGAA | | | | |
| sgRNA_103 | GAGCA | chr16: | chr16: | desert_6 | 87.92 |
| | GACAG | 11225670 | 11220000- | (GS103) | |
| | AAACC | | 11230000 | | |
| | CAGGG | | | | |
| sgRNA_104 | TGAGT | chr16: | chr16: | desert_6 | 88.53 |
| | CTCCA | 11226284 | 11220000- | (GS104) | |
| | AACAG | | 11230000 | | |
| | AACAG | | | | |
| sgRNA_105 | TAATA | chr16: | chr16: | desert_6 | 87.65 |
| | TCACT | 11225029 | 11220000- | (GS105) | |
| | GACTT | | 11230000 | | |
| | CACGG | | | | |
| sgRNA_106 | TACAC | chr2: | chr2: | desert_7 | 71.79 |
| | ACAAT | 87467461 | 87460000- | (GS106) | |
| | GTAAG | | 87470000 | | |
| | CAGCA | | | | |
| sgRNA_107 | GGGAG | chr2: | chr2: | desert_7 | 65.89 |
| | CTCAA | 87468809 | 87460000- | (GS107) | |
| | TTCGA | | 87470000 | | |
| | AACCA | | | | |
| sgRNA_108 | TTGGA | chr2: | chr2: | desert_7 | 72.64 |
| | CAGGT | 87467001 | 87460000- | (GS108) | |
| | GAGAC | | 87470000 | | |
| | AGTCG | | | | |
| sgRNA_109 | AAGCT | chr3: | chr3: | desert_8 | 76.89 |
| | CACTC | 186511316 | 186510000- | (GS109) | |
| | AGATA | | 186520000 | | |
| | GTGTG | | | | |
| sgRNA_110 | CAGGA | chr3: | chr3: | desert_8 | 86.31 |
| | GAACC | 186515260 | 186510000- | (GS110) | |
| | ACCTT | | 186520000 | | |
| | ACACG | | | | |
| sgRNA_111 | GGACA | chr3: | chr3: | desert_8 | 85.47 |
| | GACCC | 186519655 | 186510000- | (GS111) | |
| | TGATT | | 186520000 | | |
| | CACAA | | | | |
| sgRNA_112 | ACATG | chr3: | chr3: | desert_9 | 87.77 |
| | GCAGT | 59451154 | 59450000- | (GS112) | |
| | CTATG | | 59460000 | | |
| | AACAG | | | | |
| sgRNA_113 | CCTAT | chr3: | chr3: | desert_9 | 79.33 |
| | AGAGA | 59456416 | 59450000- | (GS113) | |
| | GTACT | | 59460000 | | |
| | ACTTG | | | | |
| sgRNA_114 | CCAAC | chr3: | chr3: | desert_9 | 92.21 |
| | CGGGT | 59457029 | 59450000- | (GS114) | |
| | CTTCA | | 59460000 | | |
| | TTACG | | | | |
| sgRNA_115 | TCAAG | chr8: | chr8: | desert_10 | 93.07 |
| | CGTAG | 127993006 | 127980000- | (GS115) | |
| | AGTTC | | 128000000 | | |
| | CGAGT | | | | |
| sgRNA_116 | TCATG | chr8: | chr8: | desert_10 | 89.40 |
| | CAATT | 127994663 | 127980000- | (GS116) | |
| | ATGGA | | 128000000 | | |
| | CCCAG | | | | |
| sgRNA_117 | CGGGA | chr8: | chr8: | desert_10 | 87.45 |
| | AAGTG | 127996766 | 127980000- | (GS117) | |
| | ACTGG | | 128000000 | | |
| | CCATG | | | | |
| sgRNA_118 | TGAGA | chr9: | chr9: | desert_11 | 84.84 |
| | TTGAA | 7974159 | 7970000- | (GS118) | |
| | ATCAA | | 7980000 | | |
| | ATCGG | | | | |
| sgRNA_119 | TATGC | chr9: | chr9: | desert_11 | 85.44 |
| | AATAT | 7977914 | 7970000- | (GS119) | |
| | TCATC | | 7980000 | | |
| | ACGCG | | | | |
| sgRNA_120 | AATGT | chr9: | chr9: | desert_11 | 83.48 |
| | GTTAA | 7976895 | 7970000- | (GS120) | |
| | ATCAA | | 7980000 | | |
| | ATGCA | | | | |

### CRISPR-Cas Editing

One effective example of gene editing is the CRISPR-Cas approach (e.g. CRISPR-Cas9). This approach incorporates the use of a guide polynucleotide (e.g. guide ribonucleic acid or gRNA) and a Cas endonuclease (e.g. Cas9 endonuclease).

As used herein, a polypeptide referred to as a “Cas endonuclease” or having “Cas endonuclease activity” refers to a CRISPR-related (Cas) polypeptide encoded by a Cas gene, wherein a Cas polypeptide is a target DNA sequence that can be cleaved when operably linked to one or more guide polynucleotides (see, e.g., U.S. Pat. No. 8,697,359). Also included in this definition are variants of Cas endonuclease that retain guide polynucleotide-dependent endonuclease activity. The Cas endonuclease used in the donor DNA insertion method detailed herein is an endonuclease that introduces double-strand breaks into DNA at the target site (e.g., within the target locus or at the safe harbor site).

As used herein, the term “guide polynucleotide” relates to a polynucleotide sequence capable of complexing with a Cas endonuclease and allowing the Cas endonuclease to recognize and cleave a DNA target site. The guide polynucleotide can be a single molecule or a double molecule. The guide polynucleotide sequence can be an RNA sequence, a DNA sequence, or a combination thereof (RNA-DNA combination sequence). A guide polynucleotide comprising only ribonucleic acid is also referred to as “guide RNA”. In some embodiments, a polynucleotide donor construct is inserted at a safe harbor locus using a guide RNA (gRNA) in combination with a Cas endonuclease (e.g. Cas9 endonuclease).

The guide polynucleotide includes a first nucleotide sequence domain (also referred to as a variable targeting domain or VT domain) that is complementary to a nucleotide sequence in the target DNA, and a second nucleotide that interacts with a Cas endonuclease polypeptide. It can be a double molecule (also referred to as a double-stranded guide polynucleotide) comprising a sequence domain (referred to as a Cas endonuclease recognition domain or CER domain). The CER domain of this double molecule guide polynucleotide comprises two separate molecules that hybridize along the complementary region. The two separate molecules can be RNA sequences, DNA sequences and/or RNA-DNA combination sequences.

Genome editing using CRISPR-Cas approaches relies on the repair of site-specific DNA double-strand breaks (DSBs) induced by the RNA-guided Cas endonuclease (e.g. Cas 9 endonuclease). Homology-directed repair (HDR) of these DSBs enables precise editing of the genome by introducing defined genomic changes, including base substitutions, sequence insertions, and deletions. Conventional HDR-based CRISPR/Cas9 genome-editing involves transfecting cells with Cas9, gRNA and donor DNA containing homologous arms matching the genomic locus of interest.

HITI (homology independent targeted insertion) uses a non-homologous end joining (NHEJ)-based homology-independent strategy and the method can be more efficient than HDR. Guide RNAs (gRNAs) target the insertion site. For HITI, donor plasmids lack homology arms and DSB repair does not occur through the HDR pathway. The donor polynucleotide construct can be engineered to include Cas9 cleavage site(s) flanking the gene or sequence to be inserted. This results in Cas9 cleavage at both the donor plasmid and the genomic target sequence. Both target and donor have blunt ends and the linearized donor DNA plasmid is used by the NHEJ pathway resulting integration into the genomic DSB site. (See, for example, Suzuki, K., et al. (2016). In vivo genome editing via CRISPR/Cas9 mediated homology-independent targeted integration. Nature, 540 (7631), 144-149, the relevant disclosures of which are herein incorporated in their entirety).

Methods for conducing gene editing using CRISPR-Cas approaches are known to those of ordinary skill in the art. (See, for example, U.S. application Ser. Nos. 16/312,676, 15/303,722, and 15/628,533, the disclosures of which are herein incorporated by reference in their entirety). Additionally, uses of endonucleases for inserting transgenes into safe harbor loci are described, for example, in U.S. application Ser. No. 13/036,343, the disclosures of which are herein incorporated by reference in their entirety.

The guide RNAs and/or mRNA (or DNA) encoding an endonuclease can be chemically linked to one or more moieties or conjugates that enhance the activity, cellular distribution, or cellular uptake of the oligonucleotide. Non-limiting examples of such moieties include lipid moieties such as a cholesterol moiety, cholic acid, a thioether, a thiocholesterol, an aliphatic chain (e.g., dodecandiol or undecyl residues), a phospholipid, e.g., di-hexadecyl-rac-glycerol or triethylammonium 1,2-di-O-hexadecyl-rac-glycero-3-H-phosphonate, a polyamine or a polyethylene glycol chain, adamantane acetic acid, a palmityl moiety and an octadecylamine or hexylamino-carbonyl-t oxycholesterol moiety. See for example US Patent Publication No. 20180127786, the disclosure of which is herein incorporated by reference in its entirety.

### Therapeutic Applications

For therapeutic applications, the engineered cells, populations thereof, or compositions thereof are administered to a subject, generally a mammal, generally a human, in an effective amount.

The engineered cells may be administered to a subject by infusion (e.g., continuous infusion over a period of time) or other modes of administration known to those of ordinary skill in the art.

The engineered cells provided herein not only find use in gene therapy but also in non-pharmaceutical uses such as, e.g., production of animal models and production of recombinant cell lines expressing a recombinant nucleic acid of interest.

The engineered cells of the present disclosure can be any cell, generally a mammalian cell, generally a human cell that has been modified by integrating a transgene at a safe harbor locus described herein. Exemplary cells are provided in the Recombinant Cells section.

The engineered cells, compositions and methods of the present disclosure are useful for therapeutic applications such as immune or T cell therapy. In some embodiments, the insertion of a sequence encoding an shRNA molecule within a safe harbor locus maintains the TCR expression relative to instances when there is no insertion and enables transgene expression while maintaining TCR function.

In some embodiments, the present disclosure provides methods of treating a subject in need of treatment by administering to the subject a composition comprising any of the engineered cells described herein. In some embodiments, administration of the engineered cell composition results in a desired pharmacological and/or physiological effect. That effect can be partial or complete cure of the disease and/or adverse effects resulting from the disease. In some embodiments, treatment encompasses any treatment of a disease in a subject (e.g., mammal, e.g., human). Further, treatment may stabilize or reduce undesirable clinical symptoms in subjects (e.g., patients). The cells provided herein populations thereof, or compositions thereof may be administered during or after the occurrence of the disease.

In certain embodiments, the subject has a disease, condition, and/or injury that can be treated and/or ameliorated by cell therapy. In some embodiments, the subject in need of cell therapy is a subject having an injury, disease, or condition, thereby causing cell therapy (e.g., therapy in which cellular material is administered to the subject). However, it is contemplated that it is possible to treat, ameliorate and/or reduce the severity of at least one symptom associated with the injury, disease or condition.

### Method of Administration

An effective amount of the immune cell comprising the system may be administered for the treatment of cancer. The appropriate dosage of the immune cell comprising the system may be determined based on the type of cancer to be treated, the type of the immune cell comprising the system, the severity and course of the cancer, the clinical condition of the individual, the individual's clinical history and response to the treatment, and the discretion of the attending physician.

### Pharmaceutical Compositions

The engineered recombinant cells or recombinant nucleic acids provided herein can be administered as part of a pharmaceutical compositions. These compositions can comprise, in addition to one or more of the recombinant cells, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes. The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

Various modes of administering the additional therapeutic agents are contemplated herein. In some embodiments, the additional therapeutic agent is administered by any suitable mode of administration.

A composition can be administered alone or in combination with other treatments, either simultaneously or sequentially dependent upon the condition to be treated.

### Kits and Articles of Manufacture

The present application provides kits comprising any one or more of the recombinant nucleic acids or cell compositions described herein along with instructions for use. The instructions for use can be present in the kits as a package insert, in the labeling of the container of the kit or components thereof, or can be in digital form (e.g. on a CD-ROM, via a link on the internet). A kit can include one or more of a genome-targeting nucleic acid, a polynucleotide encoding a genome-targeting nucleic acid, a site-directed polypeptide, and/or a polynucleotide encoding a site-directed polypeptide. Additional components within the kits are also contemplated, for example, buffer (such as reconstituting buffer, stabilizing buffer, diluting buffer), and/or one or more control vectors.

In some embodiments, the kits further contain a component selected from any of secondary antibodies, reagents for immunohistochemistry analysis, pharmaceutically acceptable excipient and instruction manual and any combination thereof. In one specific embodiment, the kit comprises a pharmaceutical composition comprising any one or more of the recombinant nucleic acids or cell compositions described herein, with one or more pharmaceutically acceptable excipients.

The present application also provides articles of manufacture comprising any one of the recombinant nucleic acids or cell compositions or kits described herein. Examples of an article of manufacture include vials (including sealed vials).

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. Sec, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Characterization of TGFRB1 and TGFRB2 shRNA In Vitro

### Materials

### T Cell Generation:

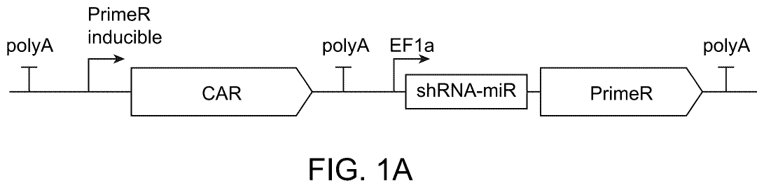

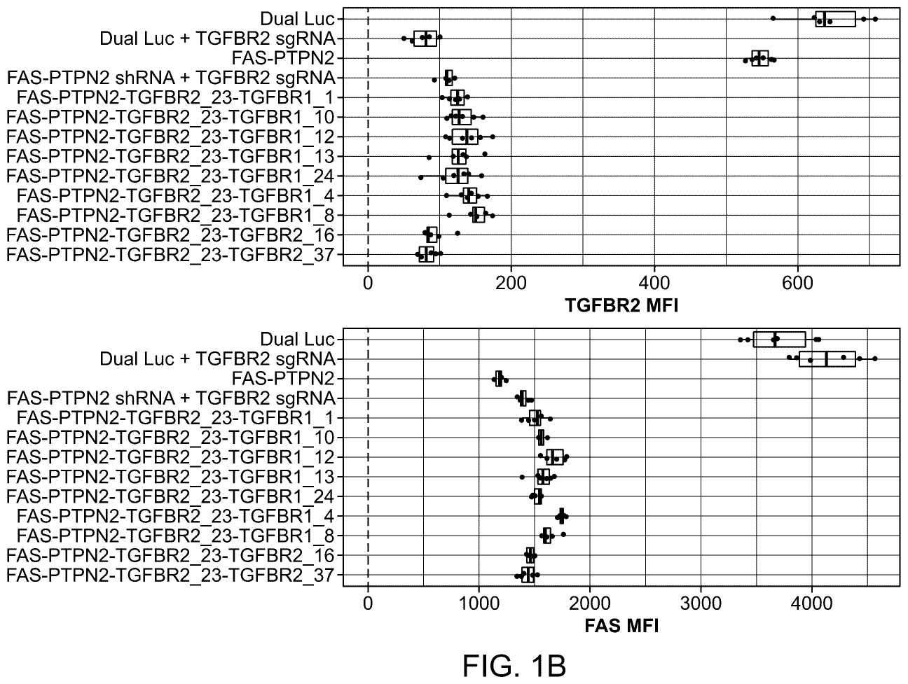

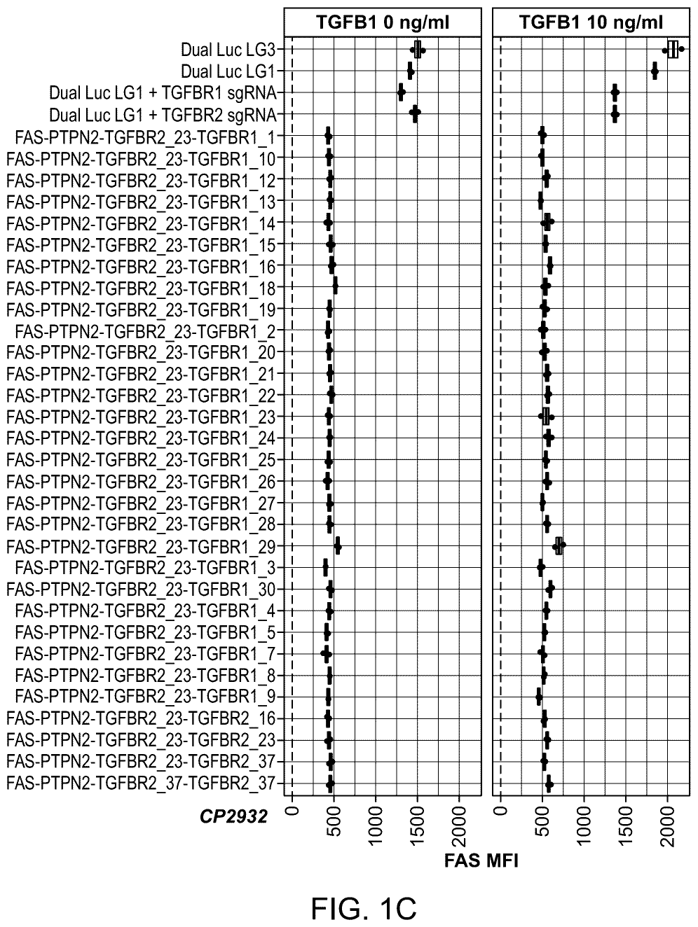

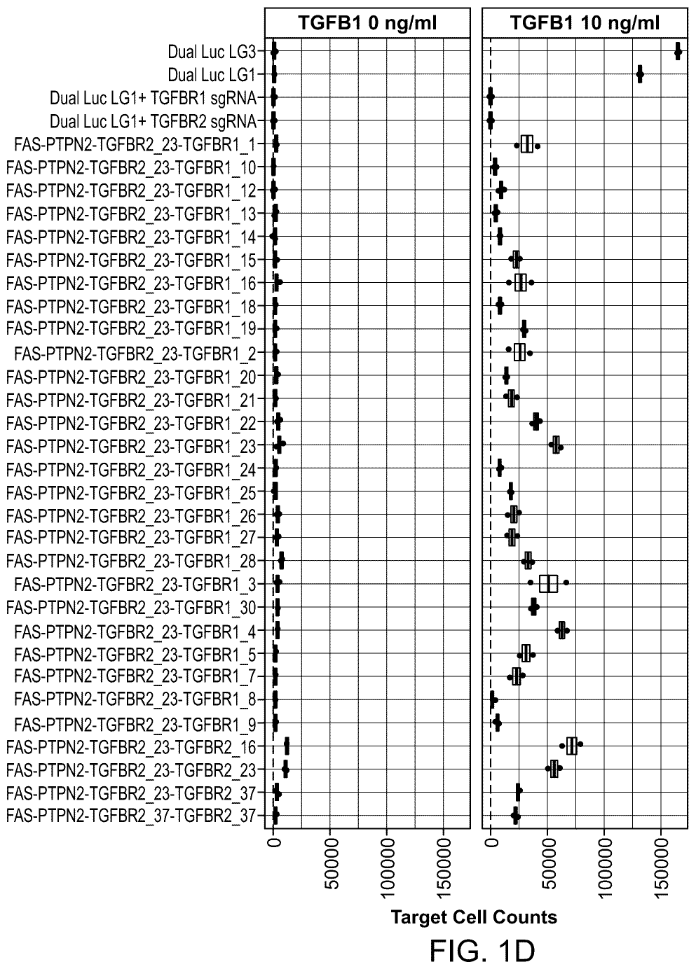

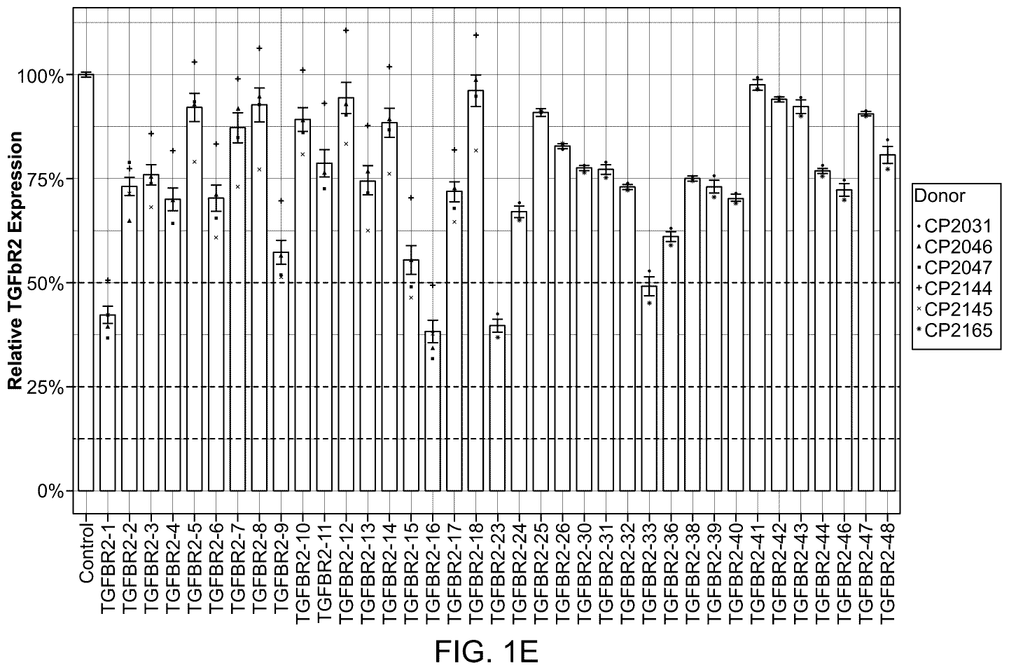

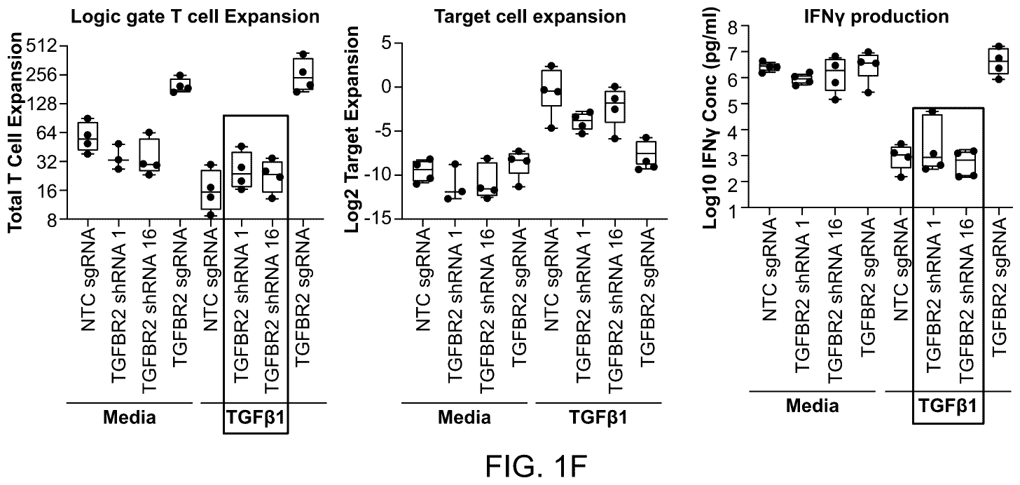

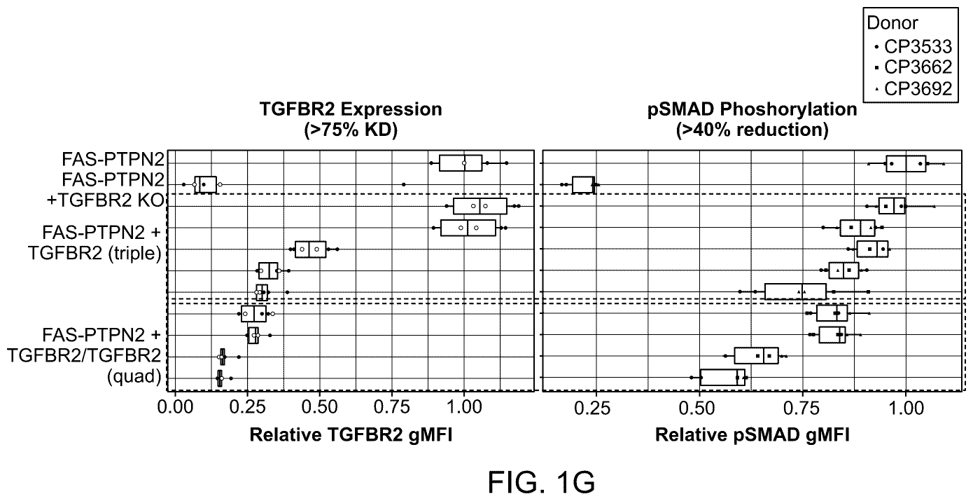

Engineered T cells were generated using CITE non-viral gene delivery. Briefly, pan-T cells were isolated from a healthy human donor using the Miltenyi StraightFrom® Leukopak® CD4/CD8 MicroBead Kit. Isolated T cells were stimulated with anti-CD3/anti-CD28 beads. Two day after stimulation, cells were resuspended in a solution containing S. pyogenes Cas9 complexed with GS94 guide RNA and donor DNA template encoding transgene of interest. Cells were subsequently electroporated using the Lonza 4-D Nucleofector and recovered in fresh media supplemented with IL-7 and IL-15. Cells were counted and fresh media added every 2-3 days following electroporation. All constructs tested encoded a logic gate expressing a PrimeR receptor to ALPG and a CAR targeting MSLN. An exemplary construct format is shown in FIG. 1A. The shRNA cassette encoded in the cassette was variable between constructs. T cells expressing the ALPG/MSLN logic gate and an shRNA cassette are referred to as Integrated Circuit T cells (ICTs). shRNAs used in the cassette targeted TGFBR1, TGFBR2, FAS, and/or PTPN2. 37 single candidate shRNAs targeting TGFBR2 were screened for their ability to reduce TGFBR2 surface expression. Dual shRNA cassettes contained an shRNA targeting TGFBR1 and an shRNA targeting TGFBR2 or two shRNA targeting TGFBR2. “Quad” shRNA cassettes were made by combining a TGFBR1/TGFBR2 or TGFBR2/TGFBR2 shRNA in combination with shRNA targeting FAS and PTPN2. “Triple” shRNA cassettes were made by combining a TGFBR2 shRNA in combination with shRNAs targeting FAS and PTPN2.

Repetitive Stimulation Assay with K562 Tumor Cells & Flow Cytometry:

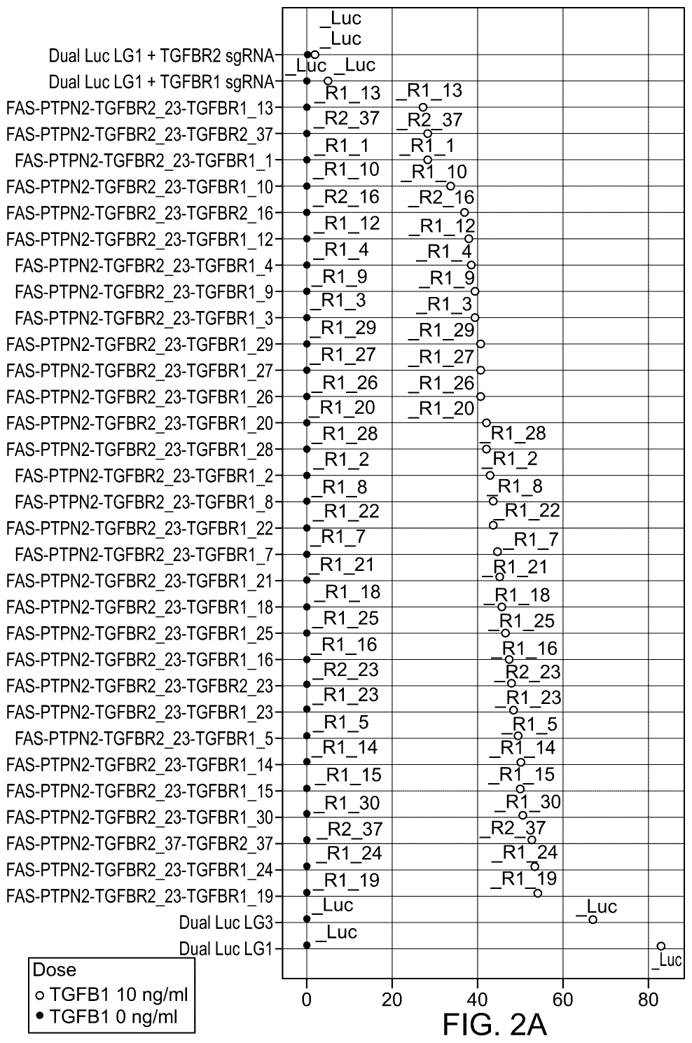

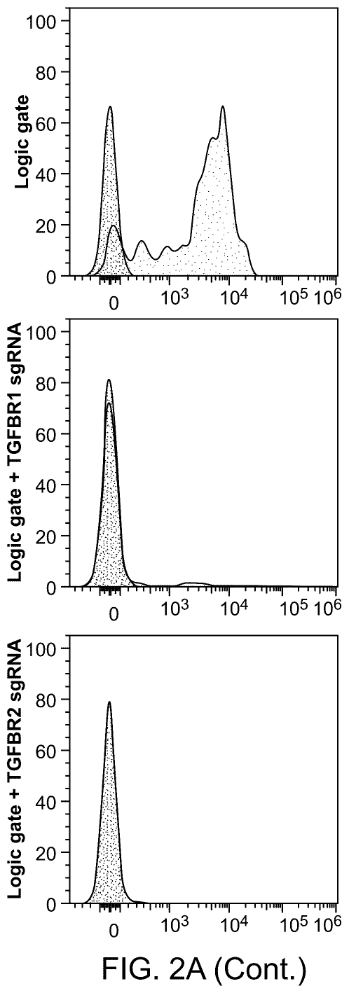

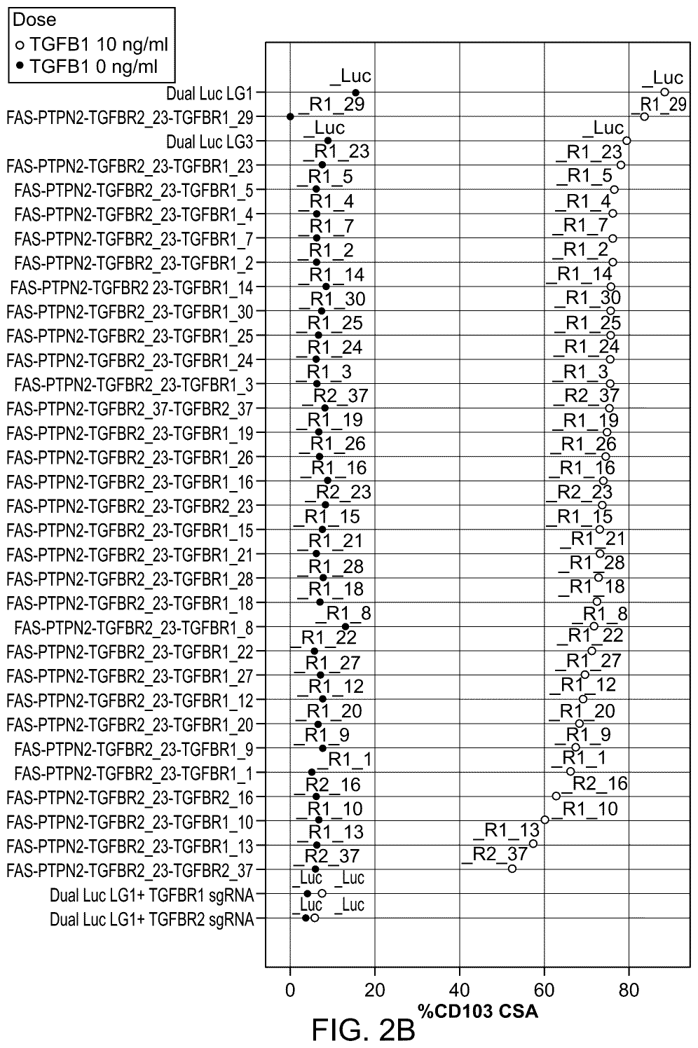

On day 5 post-electroporation, edited cells were enriched via bead-based positive selection for Myc+ cells (a Myc tag was expressed on the priming receptor). T cells were co-cultured with K562 tumor cells engineered to express ALPG and MSLN at a 2:1 effector: target ratio in either plain media or media containing 10 ng/mL TGFβ1 for 6 days. Three days after the first stimulation the T cells and tumors were quantified via flow cytometry, T cells were normalized to a defined concentration, and restimulated at a 2:1 E:T ratio with or without added TGFβ1. On day 6 cells were harvested and flow cytometry performed to assess expression of FAS, CD103, and the number of viable tumor cells (FIGS. 1C, 2A, 2B).

Longitudinal Repetitive Stimulation Assay with K562 Tumor Cells & Flow Cytometry

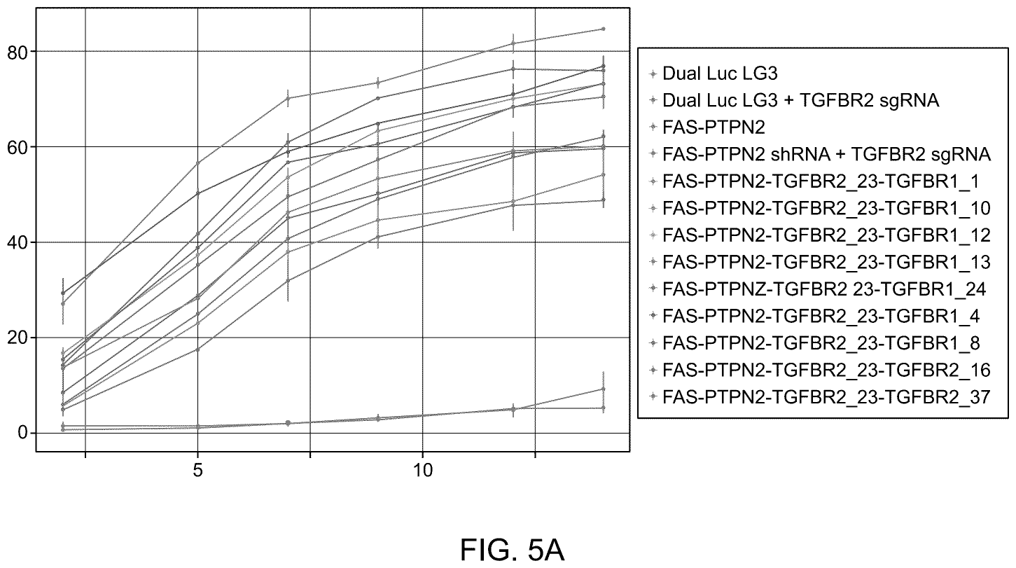

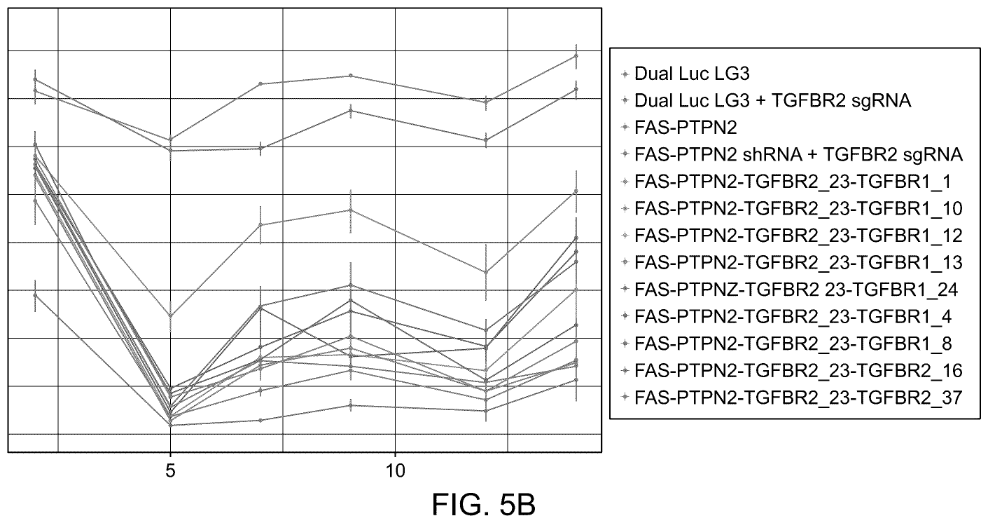

On day 4 post-electroporation, edited cells were enriched via bead-based positive selection for Myc+ cells. T cells were co-cultured with K562 tumor cells engineered to express ALPG and MSLN at a 2:1 effector: target ratio in either plain media or media containing 10 ng/mL TGFβ1. T cells and tumors were quantified via flow cytometry every 2-3 days. At each timepoint T cells were normalized to a defined concentration and restimulated at a 2:1 E:T ratio with or without added TGFβ1. Six total stimulations were conducted over a 14 day period. The frequency of CD103+ T cells (FIG. 5A) and PD-1+ T cells (FIG. 5B) in the plus TGFβ1 condition was quantified longitudinally by flow cytometry.

Co-Culture with RPMI-8226 Tumor Cells & Flow Cytometry:

Cells isolated as above were co-cultured with RPMI-8226 tumor cells at 1:2.5 E:T for 6 days in the presence or absence of 10 ng/ml exogenous TGFb1. On day 6, cells were harvested and flow cytometry performed to assess expression of CD103.

Edited logic gate T cells were co-cultured with K562 target cells in the absence or presence of TGF-β1 for 14 days. Surface expression for CD103 and PD1 were measured by flow cytometry. AUC/AAUC (10 ng/ml TGF-1 vs 0) calculated for CD103 and PD1 on 2nd-6th stimulations.

Validation of shRNA Target Knock-Down by MULTI-Seq:

At the conclusion of the 14 day repetitive stimulation assay (RSA) conducted in the presence or absence of TGFβ, cells were harvested and processed for MULTISEQ.

Cultured cells were counted on the Attune and normalized to the lowest T cell count of approximately 85,000 cells per well. 100 μL of normalized cells in media was transferred to a 96 well round bottom plate.

The cells in 96 well plates were washed twice, and resuspended in 180 μL of 1×PBS into cell suspensions. For anchor labeling, 22 μL of a 2 μM anchor-barcode mixture (equimolar amounts of anchor LMO and sample barcode oligonucleotide in 1×PBS) was mixed with each cell sample, and incubated on ice for 5 minutes. 22 μL of co-anchor solution (2 μM of co-anchor LMO in 1×PBS) was added to the anchor-barcoded sample and incubated on ice for an additional five minutes. After co-anchor labeling, cells were spun down in 4° C. at 400×g for 10 min. Supernatant was removed followed by two consecutive washes in cold 1×PBS buffer with 1% BSA. Cells were then resuspended in cold 1×PBS with 1% BSA, pooled into a single tube, filtered and sorted for live T-cells. Sorted cells were counted on a hemocytometer and loaded onto a 10× microfluidic chip K, targeting 40,000 cells per lane. Library preparation was performed as described in Chromium Next GEM Single Cell 5′ Reagent Kits v2 protocol with the addition of a MULTI-Seq primer (˜25 nM final concentration) to the cDNA amplification reaction.

Per the 10×cDNA amplification clean-up protocol, 0.6×SPRIselect reagent was added to the sample. Sample was mixed, incubated and placed on the 10× magnet until the solution cleared. 80 μL of the supernatant was transferred into a new 1.5 mL tube. This contains the MULTI-seq barcode sequence fraction. 260 μL of SPRIselect and 180 μL of 100% isopropanol was added to the MULTI-seq barcode fraction. Samples were mixed well, incubated and placed on the 10× magnet. Supernatant was discarded and the beads were washed twice with 80% ethanol. Beads were air dried and resuspended in Qiagen elution buffer. Sample was quantified using the Qubit fluorometer.

Samples were normalized to 3.5 ng in a total sample volume of 18.75 μL for index PCR. Universal i5 primer was paired with unique i7 indexes for multiplexing of samples for sequencing. After index PCR, samples were subjected to a 1.6×SPRIselect clean-up. Final samples were then quantified by Qubit and Tapestation. Molar concentration from Tapestation was calculated from 100 to 1000 bp. MULTI-seq barcode samples were pooled to target 2,000 reads per cell. Samples were sequenced on the Illumina Novaseq Platform as indicated in the Chromium Next GEM Single Cell 5′ Reagent Kits v2 protocol.

Log 2(CPKM+1) were plotted for relevant target genes (FAS, PTPN2, TGFBR1, TGFBR2)

### Phospho-Smad2/3 Assessment by Flow Cytometry:

2×10⁵ myc-enriched cells per donor were resuspended in serum-free-cytokine-free media and rested overnight. The following day cells were treated with 10 ng/ml TGF-β1 for 2 hours and stained for surface Myc expression and live/dead stain. Cells were fixed and permeabilized per manufacturer protocol using BE Perm Buffer III. Cells were stained for pSMAD2/3 using clone 072-670 (BD). Samples were washed and acquired on an Attune Nxt cytometer. pSMAD2/3 MFI was quantified relative to control cells lacking TGFBR shRNA.

### Results

To ensure that the shRNA miR Quads (fas-ptpn2-tgfbr2-tfgrb2 or fas-ptpn2-tgfbr2-tgfrb1 shRNA) worked across all of the targets, both TGFBR2 and FAS knockdown was assessed after cell transfection with the Quad shRNA constructs. Quad shRNAs conferred enhanced knockdown of TGFBR2 and maintained knockdown of T cell intrinsic target FAS (FIGS. 1B and 1C). The TGFBR2 knockdown was enhanced by incorporating multiple TGFBR2 shRNAs. In addition, the FAS knockdown was preserved with “Quad” shRNA-miR module and was comparable between all TGFBR shRNA constructs. Targeting TGFBR2 with 2 shRNAs can improve the KD at the protein level.

TGFBR2 knockdown also partially inhibited TGF-β-mediated suppression of target cell killing in a short repetitive stimulation assay (FIG. 1D).

Four lead TGFBR2 shRNA candidates achieved greater than 50% knockdown of TGFBR2 surface expression in T cells (FIG. 1E). The single TGFBR2 knockdown T cells were also assessed in the repetitive stimulation assay with soluble TGF-b. FIG. 1F shows the cumulative T cell expansion, tumor cell expansion, and IFNg production in T cells stimulated in media alone or in the presence of TGF-b. Knockdown of TGFBR2 via a single shRNA did not improve ICT function in the presence of TGF-b. However, ICTs with TGFBR2 knockout (TGFBR2 sgRNA) demonstrated functional recovery in all metrics (increased T cell expansion, reduced target cell expansion, increased IFNg production). Thus, the dual TGFBR2 shRNA constructs showed improved T cell function as compared to knockdown via a single TGFBR2 shRNA.

The “triplet” shRNA cassette also reduced TGFBR2 expression and pSMAD phosphorylation as compared to FAS-PTPN2 shRNAs alone (FIG. 1G).

To assess the resistance of the TGFRB-targeting shRNA Quads (to the downstream effects of TGF-β signaling, edited logic gate T cells were co-cultured with Prime-Cytolytic+K562s in the absence or presence of TGF-β. As shown in FIG. 2A, the Quad shRNAs conferred partial resistance to TGF-mediated CD103 induction after tumor plus TGF-β1 exposure in short term RSA. Thus, TGFBR1 and TGFBR2 knockout protected logic gate T cells from TGF-β-mediated CD103 induction. The TGFBR knockdown resulted in partial reduction of CD103 induction by TGF-β.

The assay was repeated with RPMI-8226 target cells. As shown in FIG. 2B, the Quad shRNAs conferred partial resistance to TGF-β mediated CD103 induction after tumor plus TGF-β1 exposure in RPMI-8226 target cells.

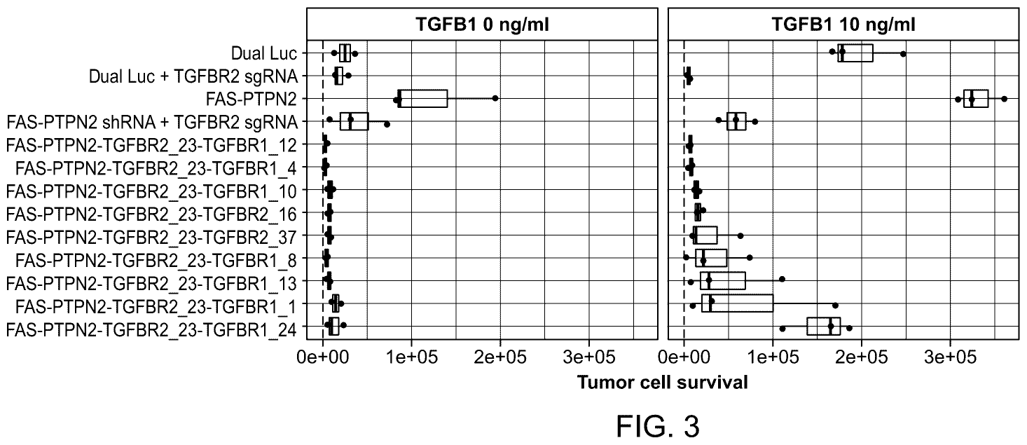

TGFBR2 knockdown rescued TGF-β-mediated suppression of tumor cell killing by logic gate T cells (FIG. 3). Thus, TGFBR shRNAs rescued antitumor activity of logic gate T cells in presence of TGF-β1.

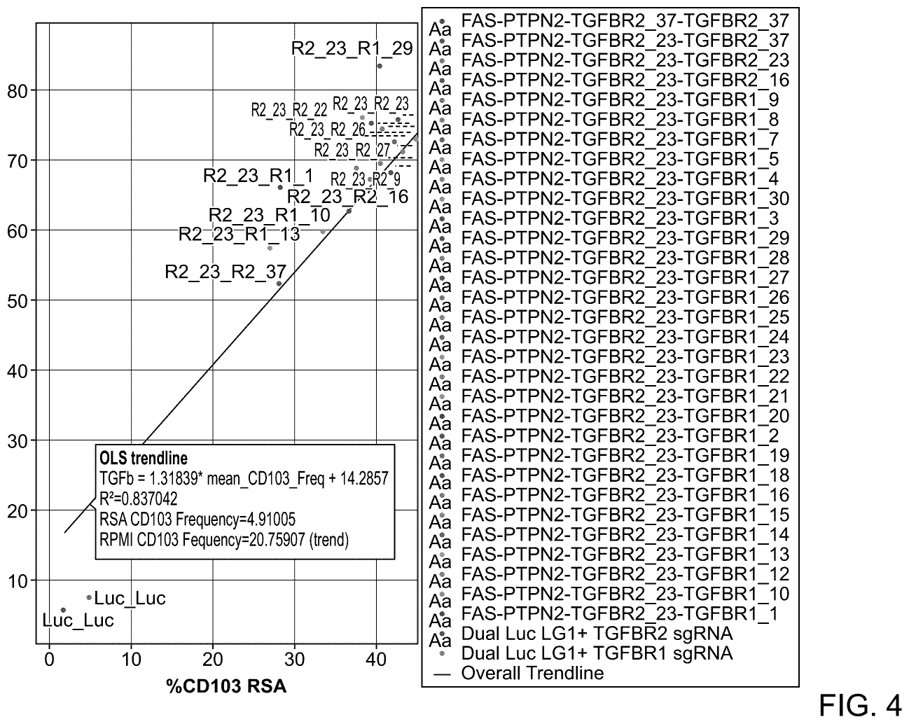

PD1 surface expression was also used to assess TGFBR shRNA Quad activity. A bivariate plot of PD1 vs CD103 expression shows abrogation of TGFB signaling which resulted in attenuated PD-1 expression (data not shown). A correlation between PD1 and CD103 induction was also observed after knockdown with the Quad shRNA modules (FIGS. 2A, 2B, and 4). A strong correlation of CD103 induction on Integrated Circuit T cell (ICT) following short RSA (2-stimulations) and CSA was also observed (FIG. 4). Thus, resistance to CD103 and PD1 induction are correlated. Without wishing to be bound by theory, PD1 induction can be a useful metric to rank TGFBR shRNA modules.

Select quad shRNA modules were chosen for further characterization. As shown in FIG. 5A, selected TGFBR quads partially impaired CD103 induction during RSA. TGFBR2 knockout longitudinally protected the logic gate T cells from TGF-β-mediated CD103 induction. TGFBR knockdown resulted in partial reduction of CD103 induction by TGF-β1. The selected quad TGFBR shRNA modules strongly impaired maintenance of PD1 during RSA (FIG. 5B)

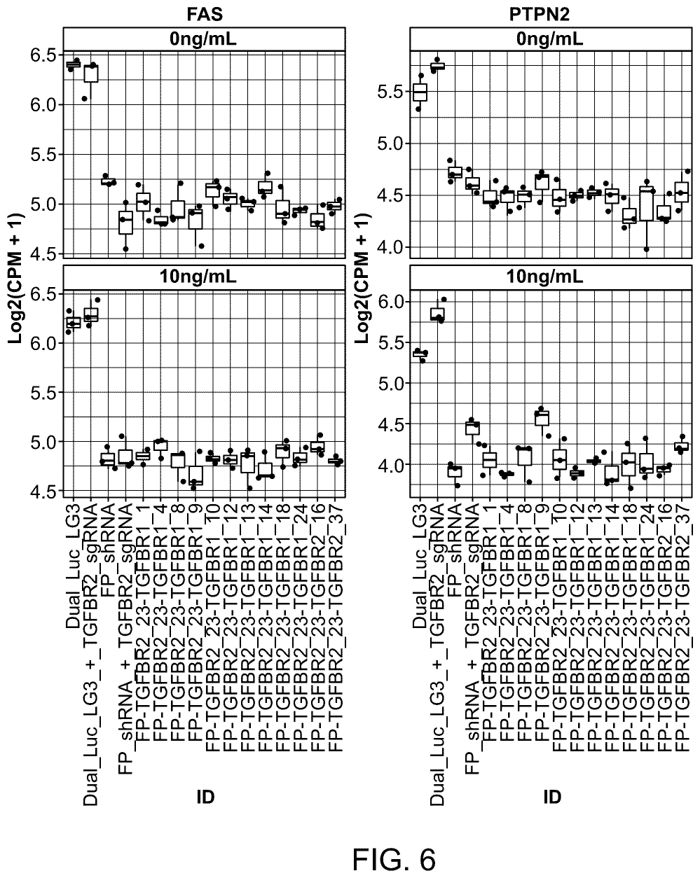

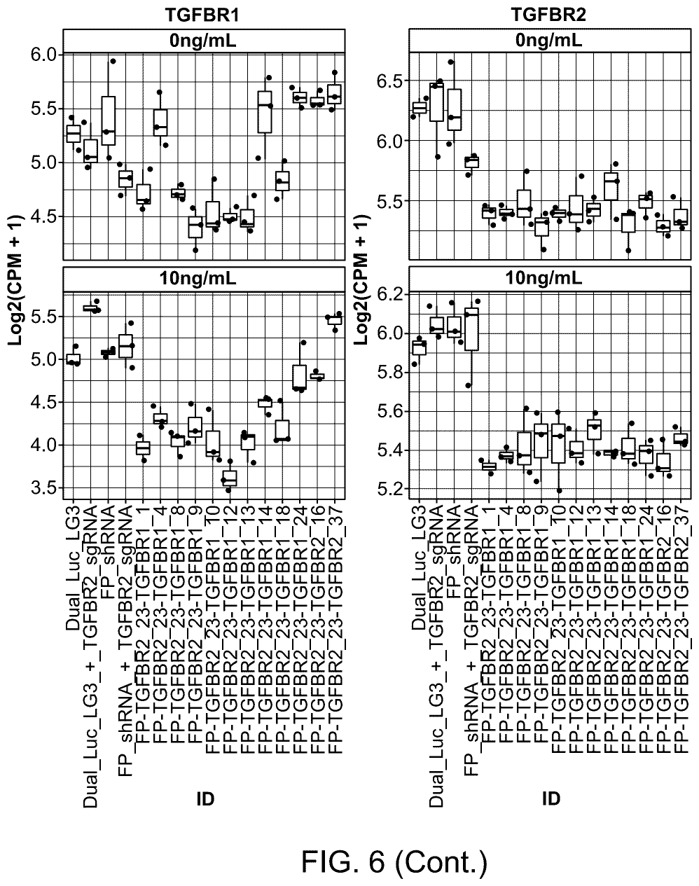

MULTIseq revealed on-target suppression of Quad miR shRNA modules (FIG. 6). KD of FAS and PTPN2 was retained in the Quad constructs. KD of TGFBR2 was also consistent across the Quads.

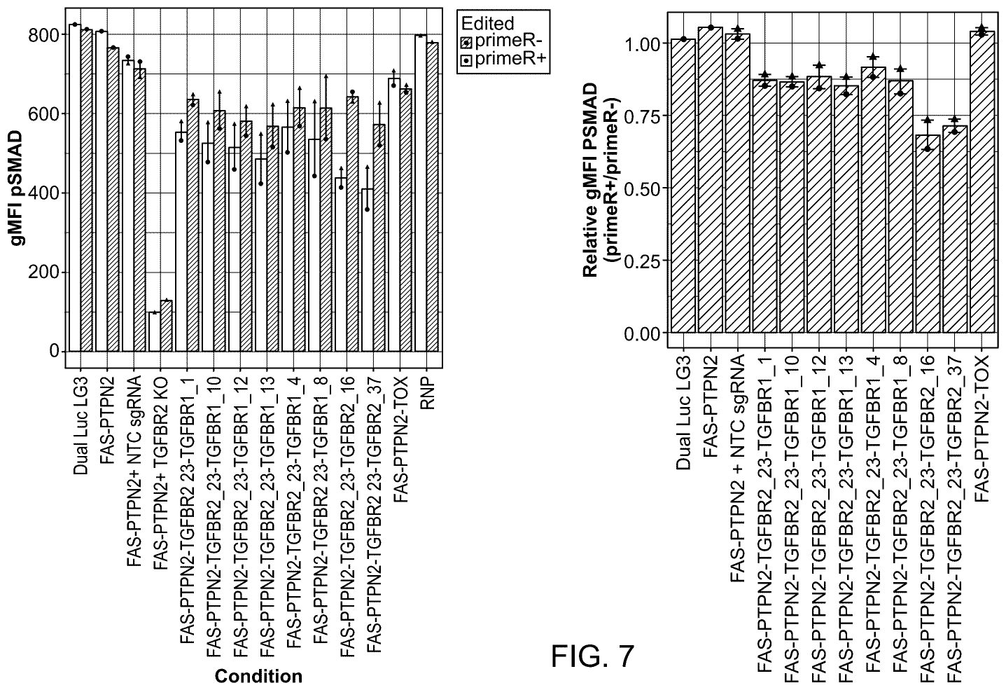

The TGFBR2-TGFBR2 Quads exhibited stronger temporal inhibition against TGF-β signaling based on pSMAD2/3 phosphorylation. As shown in FIG. 7, the TGFBR2-TGFBR2 Quad shRNA modules (left panel) exhibited a reduction in pSMAD2/3 induction in response to TGF-β1. Thus, R2-R2 shRNA Quads (left panel) exhibited a greater inhibition to TGF-β1 induced signaling than the R2-R1 Quads (right panel). This demonstrated that inclusion of multiple shRNAs targeting TGFBR enhanced target knockdown and provided superior resistance to suppressive TGFB.

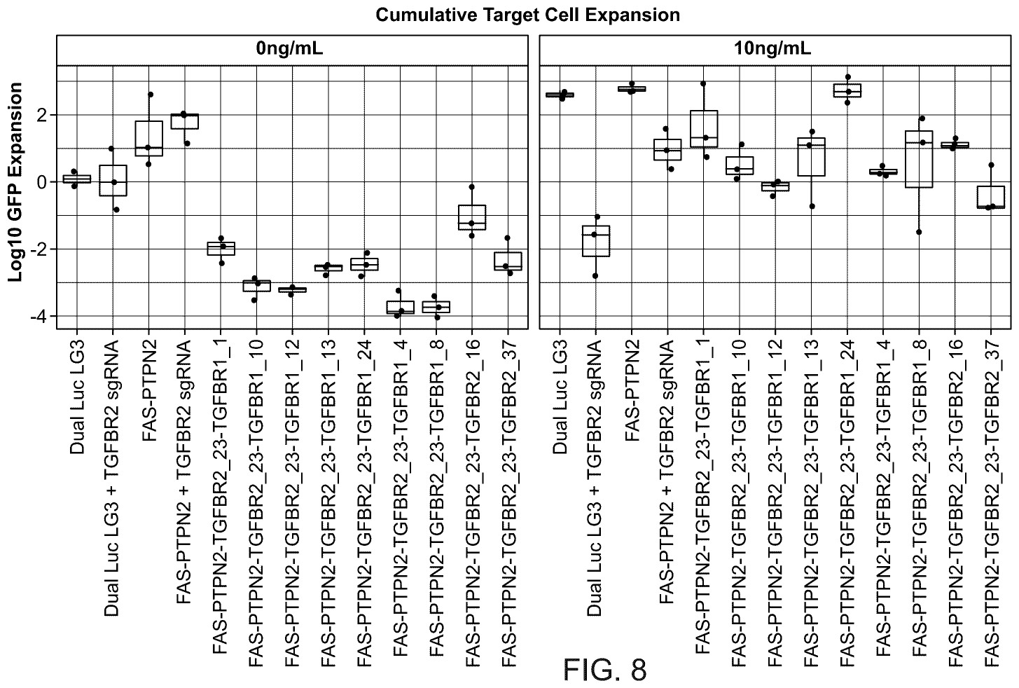

TGFBR2 knockdown by selected quad shRNA inhibited TGF-β-mediated suppression of target cell killing by logic gate T cells (FIG. 8). TGFBR knockdown resulted in significant rescue from TGF-β-mediated suppression of target cell killing by logic gate T cells.

### Example 2: Characterization of TGFRB1 and TGFRB2 Knockout In Vivo

### Materials

### H1975 Mouse Model

H1975 cells were engineered to co-express ALPG and MSLN for targeting by the logic gate CAR T cell. Briefly, ICT cells with a TGFBR2 knockout (KO) were generated by stimulating pan-T cells with aCD3/aCD28 and electroporating with Cas9 RNP containing the GS94 guide RNA, donor DNA template, and sgRNA targeting TGFBR2 where applicable. Cells were expanded for 7 days in IL-7 and IL-15 and cryopreserved prior to use. Mice were inoculated subcutaneously with the H1975^{ALPG/MSLN} cells. T cells with a TGFBR2 knock-out and expressing an ALPG/MSLN logic gate (ALPG priming receptor and MSLN CAR) were injected with 5×10⁵, 2×10⁶ or 4×10⁶ T cells when xenografts reached 160 mm³, following randomization. WT T cells with no TGFBR2 knockout or ALPG/MSLN logic gate were used as a control. N=7 mice/group, 1 donor.

### 786-O Mouse Model

A TGF-B secreting model of human renal cell carcinoma (RCC) was developed in NSG MHC I/II double knockout mice. 2e6 768-O cells engineered to express ALPG and MSLN proteins were injected into the flank, and mice were staged when tumors reached 300 mm³. ICT cells with a TGFBR2 knock-out via CRISPR and expressing an exemplary ALPG/MSLN logic gate (ALPG priming receptor and MSLN CAR) were administered by tail vein injection at a “stress dose” level of 3e5 cells/mouse. Control T cells used were RNP only cells and edited T cells expressing the logic gate and a non-coding control (NTC) sgRNA. Peripheral blood was drawn at day 14 for PK analysis of edited T cells. Tumor volume was measured for 40 days post ICT injection, 68 days total post tumor cell injection. N=7 mice/group.

### Results

### H1975 Mouse Model

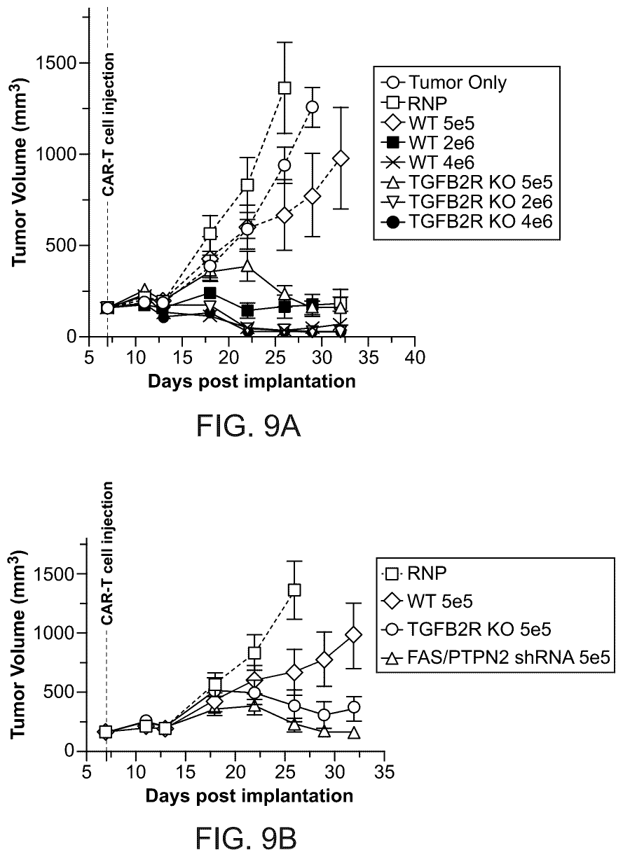

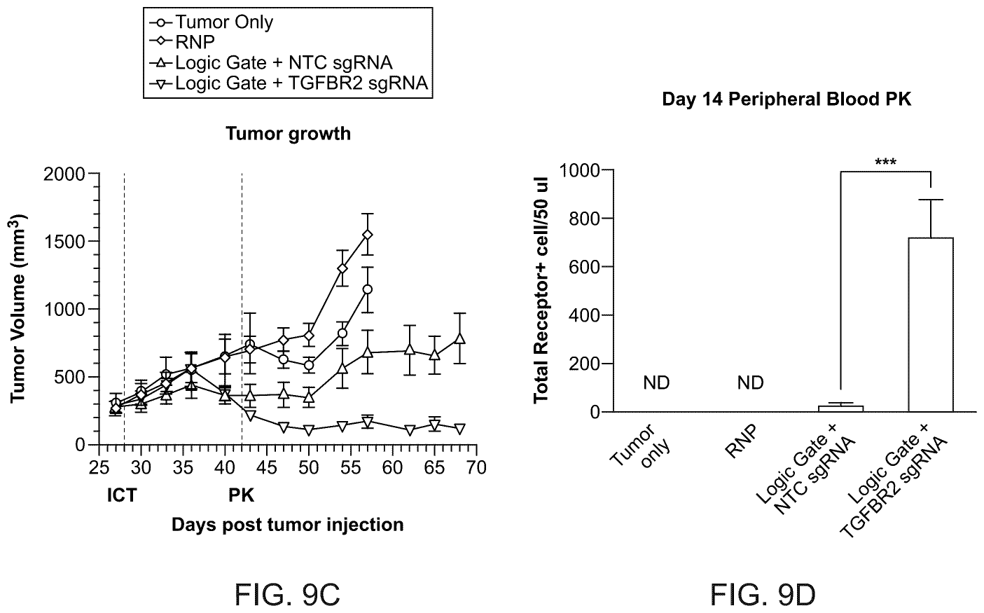

TGFBR2 knock-out in logic gate T cells resulted in antitumor activity in the H1975 xenograft model (FIG. 9A). TGFBR2 knock out (KO) logic gate T cells induced tumor growth inhibition (TGI). The dose of 5×10⁵ engineered cells yielded TGI separation between the TGFBR2 KO and WT logic gate cells at the same cell dose, indicating that the KO of TGFBR2 increased T cell efficacy against target cells.

TGFBR2 KO enhanced the efficacy of logic gate T cells (ICTs) in the H1975 model comparable to shRNA knockdown of FAS-PTPN2 (FIG. 9B).

### 786-O Mouse Model

The non-edited cells (RNP only) demonstrated no anti-tumor effect, while the logic gate only T cells exhibited some tumor control. In contrast, the T cells with the logic gate and TFGBR2 knock out (TGFBR2 sgRNA) exhibited significantly enhanced tumor control (FIG. 9C). Edited ICT cells in the peripheral blood were measured by flow cytometry on check bleed samples isolated on day 14 post ICT injection and compared across experimental arms. ICT cells with TGFBR2 knockout demonstrated significantly higher levels of ICT expansion in the blood (FIG. 9D). Student's t-test; p<0.05. Thus, TGFBR2 knockout improved ICT efficacy in an RCC model with secreted TGF-β.

### Example 3: Characterization of TGFRB1 and TGFRB2 shRNA in Combination with FAS and PTPN2 shRNA In Vivo

### Materials and Methods

### 786-O Mouse Model

Briefly, ICT cells were generated by stimulating pan-T cells with aCD3/aCD28 and electroporating with Cas9 RNP containing the GS94 guide RNA and donor DNA template encoding the ALPG/MSLN logic gate and indicated shRNA cassettes. TGFBR2 KO samples were also treated with sgRNA targeting TGFBR2. Cells were expanded for 7 days in IL-7 and IL-15 and cryopreserved prior to use.

Mice were inoculated subcutaneously with 786-O^{ALPG/MSLN}. ICTs were injected when xenografts reached 300 mm³, following randomization. N=7 mice/group, 1 donor. Tumor burden was measured longitudinally via caliper.

### H1975 Mouse Model

ICT cells were produced as described above.

Mice were inoculated subcutaneously with H1975^{ALPG/MSLN}. Engineered T cells were injected when xenografts reached 145 mm³, following randomization. T cells were engineered to express the ALPG/MSLN logic gate alone with a control shRNA, or in combination with FAS-PTPN2 shRNA, or FAS-PTPN2-TGFBR2_23-TGBR1_10 shRNA, FAS-PTPN2-TGFBR2_23-TGBR1_13 shRNA, FAS-PTPN2-TGFBR2_23-TGBR2_16 shRNA, or FAS-PTPN2-TGFBR2_23-TGBR2_37 shRNA. A TGFBR2 knockout T cell was used as a positive control. N=7 mice/group, 1 donor, 2 doses.

### Results

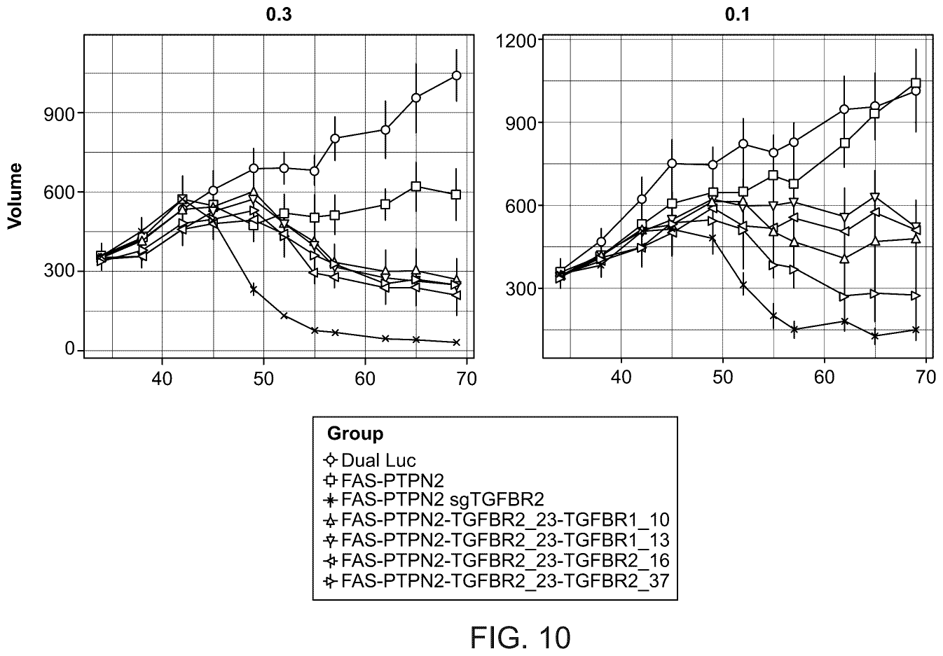

TGFBR2 knockout T cells expressing FAS-PTPN2-TGFBR2_23-TGBR1_10 shRNA, FAS-PTPN2-TGFBR2_23-TGBR1_13 shRNA, FAS-PTPN2-TGFBR2_23-TGBR2_16 shRNA, or FAS-PTPN2-TGFBR2_23-TGBR2_37 shRNA exhibited enhanced TGI in the 786-O model as compared to WT T cells and T cells expressing only FAS-PTPN2 shRNA (FIG. 10).

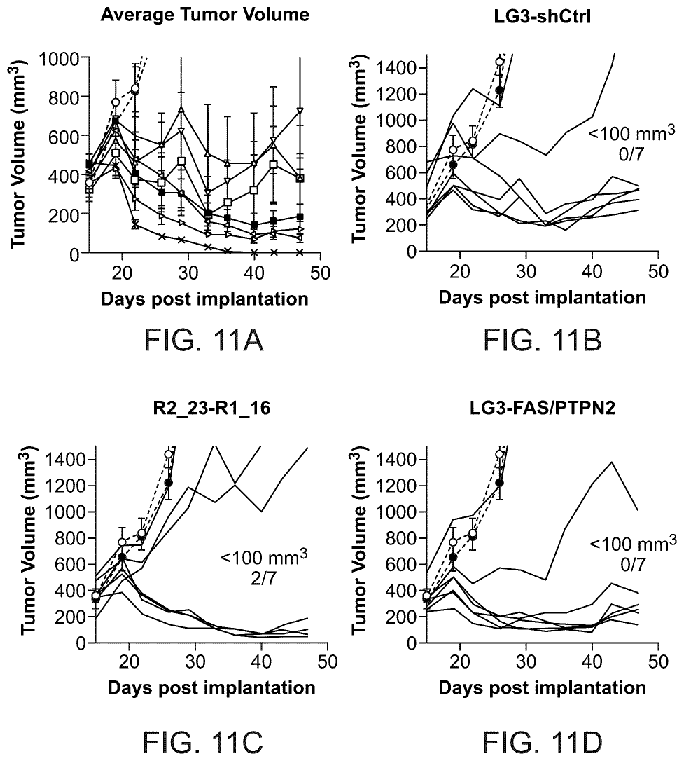

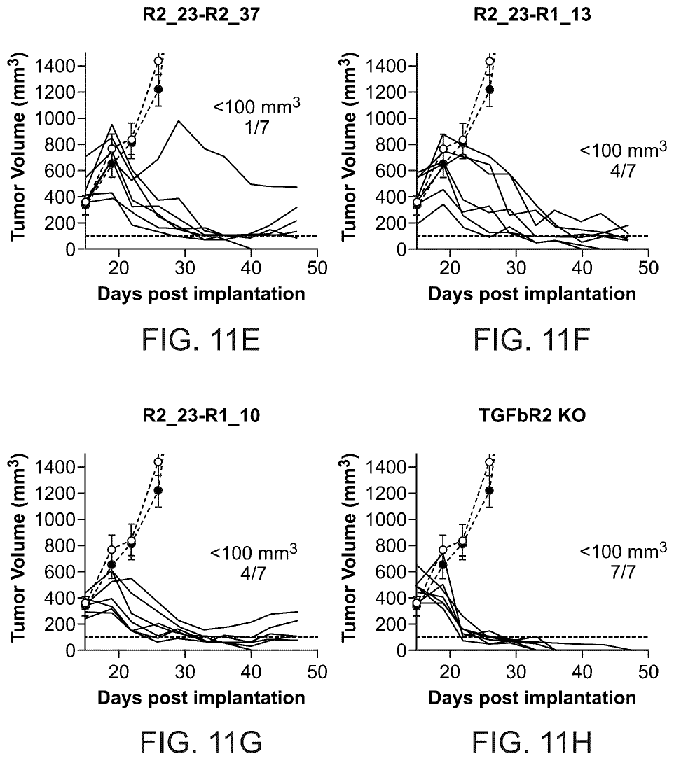

TGFBR2 knockout T cells expressing FAS-PTPN2-TGFBR2_23-TGBR1_10 shRNA, FAS-PTPN2-TGFBR2_23-TGBR1_13 shRNA, FAS-PTPN2-TGFBR2_23-TGBR2_16 shRNA, or FAS-PTPN2-TGFBR2_23-TGBR2_37 shRNA exhibited enhanced TGI in the H1975 in vivo model as compared to WT T cells and T cells expressing only FAS-PTPN2 shRNA. FIG. 11A shows the average tumor volume of after treatment with the engineered T cells. FIG. 11B shows the individual mouse tumor size after treatment with the logic gate and control shRNA control group. FIG. 11C shows the individual mouse tumor size after treatment with the FAS-PTPN2-TGFBR2_23-TGBR2_16 shRNA logic gate T cell. FIG. 11D shows the individual mouse tumor size after treatment with the FAS-PTPN2 logic gate T cell. FIG. 11E shows the individual mouse tumor size after treatment with the FAS-PTPN2-TGFBR2_23-TGBR2_37 shRNA logic gate T cell. FIG. 11F shows the individual mouse tumor size after treatment with the FAS-PTPN2-TGFBR2_23-TGBR1_13 shRNA logic gate T cell. FIG. 11G shows the individual mouse tumor size after treatment with the FAS-PTPN2-TGFBR2_23-TGBR1_10 shRNA logic gate T cell. FIG. 11H shows the individual mouse tumor size after treatment with the TGFBR2 knockout T cell.

### Example 4: In Vitro Characterization of a Second Logic Gate in Combination with FAS and TGFBR2 shRNA

### Materials and Methods

### PrimeR/CAR ICT Construct Expression in T Cells

Integrated circuit T (ICT) cells targeting a second exemplary primeR antigen (priming receptor) and second exemplary CAR antigen were generated through site directed CRISPR mediated knock in (KI). T cells were activated for two days using CD3-CD28 beads. At day 2, beads were removed followed by the delivery of the ICT transgene to the GS94 site in the genome of the T cells. Transgene integration was performed using a CRISPR-based process and electroporation step that combined activated T cells, CRISPR/Cas9 RNP targeting the GS94 non-coding autosomal integration site, and plasmid DNA constituting a repair template to effect insertion of the transgene cassette via cellular DNA repair machinery.

The GS94 CRISPR/Cas9 RNP used was generated by complexing single guide RNA (sgRNA) with recombinant Streptococcus pyogenes Cas9 (SpCas9). The sgRNA contained a protospacer sequence directing the CRISPR/Cas9 RNP to the GS94-transgene integration site. The plasmid DNA repair template contained the ICT transgene cassette, flanked by 450 base pair (bp) sequences homologous to the regions flanking the integration site to effect repair-mediated insertion.

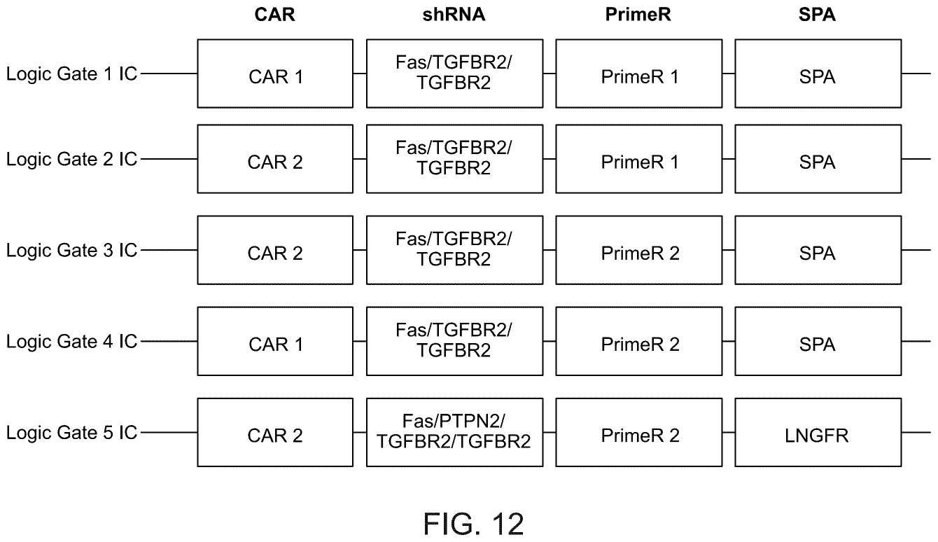

A diagram of the various ICT transgene cassettes generated is provided in FIG. 12. The ICT constructs 1, 2, 3, and 4 comprised a constitutively expressed priming receptor, an inducible CAR (forming a Logic Gate or “LG”), constitutively expressed shRNAs targeting FAS (1 shRNA) and TGFBR2 (2 shRNA), and a synthetic pathway activator (SPA). One ICT also included an shRNA targeting PTPN2 in addition to FAS and TGFBR2 and LNGFR in place of the SPA (LG 5 IC). The sequence of the FAS and dual TGFBR2 shRNA cassette (triple shRNA) is provided in SEQ ID NO: 130, while the sequence of the FAS/PTPN2/dual TGFBR2 shRNA cassette (quad shNRA) is provided in SEQ ID NO: 131. The full transgene cassettes comprising a logic gate with the shRNA and optional SPA are termed Logic Gate 1 integrated circuit (“IC” or LG 1 IC), Logic Gate 2 IC (LG 2 IC), Logic Gate 3 IC (LG 3 IC), Logic Gate 4 IC (LG 4 IC), or Logic Gate 5 IC (LG 5 IC).

Following electroporation, cells were recovered and expanded in T cell media for 7 days. When indicated, negative control T cells were generated using a mock electroporation process that edited T cells with ribonucleoprotein (RNP) in the absence of donor plasmid and are referred to as “RNP control”.

ICT cells were assessed for transgene KI and the expression of the PrimeR and CAR using flow based staining. Constructs contained tags myc and flag on the distal extracellular portion of the PrimeR and CAR respectively following the signal peptide. ICT cells at day 7 post activation were stained with myc, flag and CD3 antibodies for 30 min at 4c. Following activation, cells were washed in FACs buffer and run by flow cytometry. ICTs were analyzed for PrimeR and CAR expression following gating each sample for live CD3+ cells.

### ICT Induction of CARS

ICTs were generated as described above from the T cells of 2 donors. On day 11 post activation, ICTs were measured for CAR and PrimeR expression by Flag and Myc staining. % KI was quantify by summing the % of T cells in a sample that were PrimeR+ or CAR+. Before co-culture setup, ICTs were normalized to the same % KI using the addition of donor matched RNP only cell. 1×107 ICTs were co-cultured with 1×107 target cells or media for 72 hours and stained to calculate the % of CAR+ cells using flag staining. Basal CAR expression was measured during assay set up.

shRNA Knockdown

ICT cells contain a constitutive shRNA module targeting knockdown of FAS and TGFBR2, whereas cells without a transgene KI (PrimeR negative cells) have normal expression of FAS and TGFBR2 and can be used as an internal control. Multicolor flow cytometry was performed on four productions of ICT cells to characterize transgene expression and assess shRNA-miR knockdown of FAS and TGFBR2. Antibodies against CD4, CD8, CD95 (FAS) and TGFBR2 were used in the flow cytometry. The panel also included rh-primeR antigen for PrimeR detection and rhCAR antigen for CAR detection as well as Zombi NIR for live vs dead cell staining.

Surface protein knockdown of FAS and TGFBR2 in ICT cells was determined using flow cytometry. Cells were stained with anti-FAS and anti-TGFBR2 antibodies, and geometric mean fluorescence intensity (gMFI) was measured for both PrimeR-positive and PrimeR-negative subsets of ICT cells. Data are representative of 4 donors. The formula used to calculate % KD (percent knockdown)=100% (1−(MFI PrimeR+)/(MFI PrimeR−)).

### Synthetic Pathway Activators

Synthetic Pathway Activators (SPAs) constitutively drive STAT signaling without the need for external cytokine input. SPAs can be designed to engage activity of multiple STAT family transcription factors at variable levels through rational design. Exemplary Class I SPAs primarily increase pSTAT3 activity and exemplary Class II SPAs primarily increase pSTAT5 activity.

To demonstrate the ability of the SPA module to drive constitutive STAT3 phosphorylation, ICTs expressing the SPA module under non-stimulated conditions were fixed, permeabilized, and stained for pSTAT3 and the myc epitope tag to distinguish between edited and non-edited cells (data not shown).

### Cytotoxicity, Engineered K562 Cells

ICT cells expressing the integrated circuits comprising Logic Gate 1 IC, Logic Gate 2 IC, Logic Gate 3 IC, Logic Gate 4 IC, or Logic Gate 5 IC with shRNA and optionally a SPA were co-cultured with K562_EFG, K562_EFG_CAR antigen, K562_EFG_primeR antigen, or K562_EFG_CAR antigen_primeR antigen at varying E:T ratios for 72 hours at 37° C. Following incubation, cytotoxicity was measured using a luciferase reporter assay. Data are presented as the mean±standard deviation of 4 donors.

### Cytokine Secretion

To further assess the specificity and function of ICT cells expressing Logic Gate 1-5 ICs, supernatants were collected from K562 target cytotoxicity co-cultures (Effector: Target ratio of 1:1, 72 hour co-culture). Following incubation, supernatants were collected at endpoint and cytokine release levels were measured using a Luminex assay. Data from 4 donors are shown.

### Cytotoxicity in Endogenous/Engineered Cells

ICT cells expressing LG 1-5 ICs were co-cultured with CAR antigen_primeR antigen cells (cells endogenously expresses CAR antigen and engineered to express primeR antigen) at varying E:T ratios for 72 hours at 37° C. Following incubation, cytotoxicity was measured using a luciferase reporter assay. Data are presented as the mean±standard deviation of 4 donors. Prior to luciferase readout described above, supernatants were collected at endpoint and cytokines (B) IFN-g, (C) TNFα, (D) GM-CSF and (E) IL-2 were measured using a Luminex assay. Data from 4 donors are shown.

### Mixed Co-Culture Cytotoxicity

ICT cells expressing Logic Gates 1-5 were co-cultured with primeR antigen+/CAR antigen-HUVEC cells and luciferase expressing primeR antigen-/CAR antigen+ cells (K562-EFG-CAR antigen) at varying E:T ratios for 72 hours at 37° C. Following incubation, cytotoxicity was measured using a luciferase reporter assay. Data are from one normal donor. ICT-mediated CAR antigen+ target cell killing was evaluated relative to an RNP-electroporated negative control using a luciferase reporter assay.

### Results

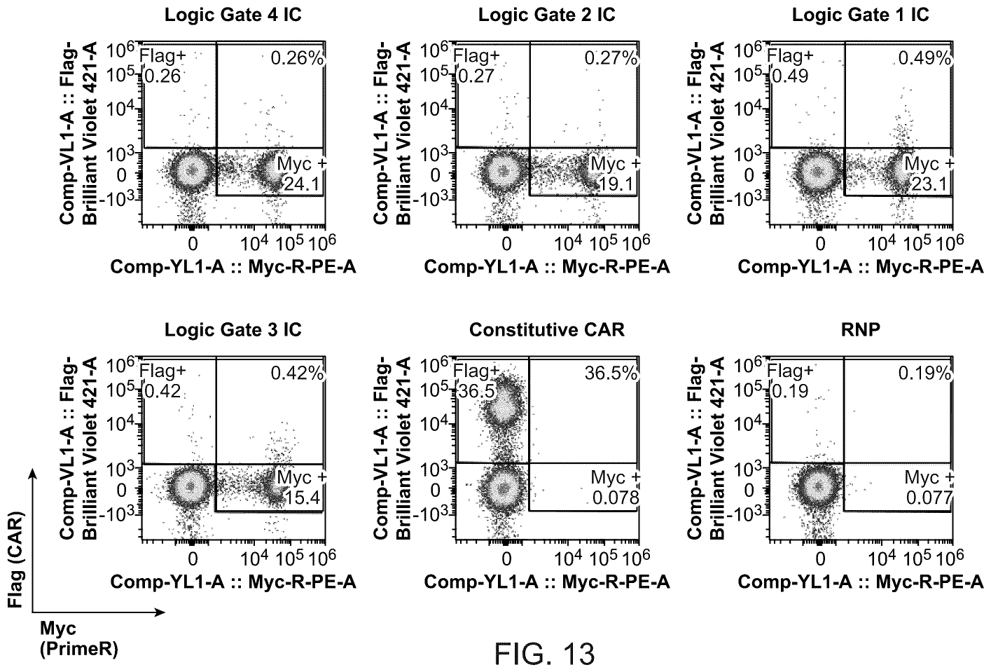

All ICT cells constitutively expressed the PrimeR construct as shown by myc expression (FIG. 13). The inducible CAR was not expressed at basal state in the ICT cells, as indicated by the lack of FLAG expression (FIG. 13), indicating that the priming receptor had not induced expression of the CAR.

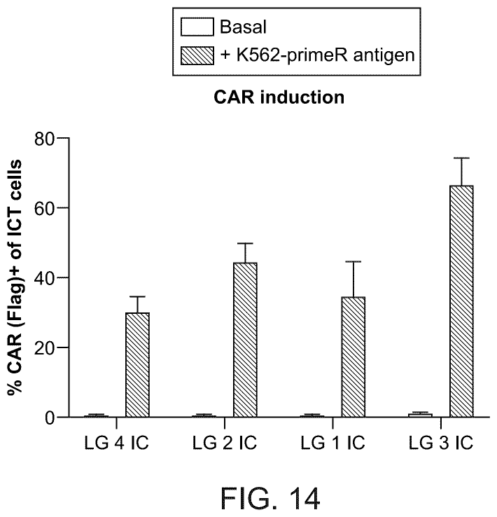

As shown in FIG. 14, the ICT cells induced CAR expression when co-cultured with primeR antigen expressing cell lines. Numbers shown in FIG. 14 are the (% CAR)/(% KI normalized to at the start of the assay)*100. Thus, the logic gate circuit functioned correctly by not expressing the CAR in the absence of binding of the primeR to its cognate antigen (FIG. 13) and induction of expression of the CAR upon binding of the primeR to its cognate ligand on a target cell (FIG. 14).

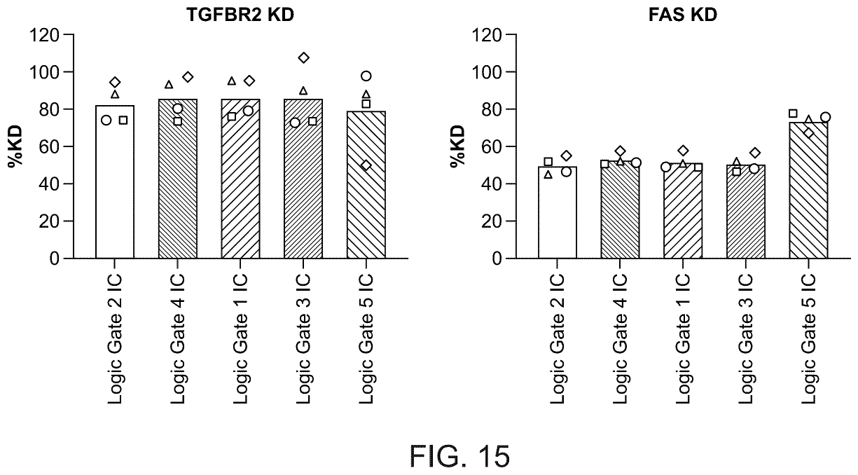

Inclusion of the shRNA module in ICT cells showed lower MFI for both FAS and TGFBR2 in ICT cells expressing the priming receptor-CAR logic gate (PrimeR+) normalized to non-edited cells (PrimeR−), indicating knockdown of both FAS and TGFBR2 in ICT PrimeR+ cells (FIG. 15).

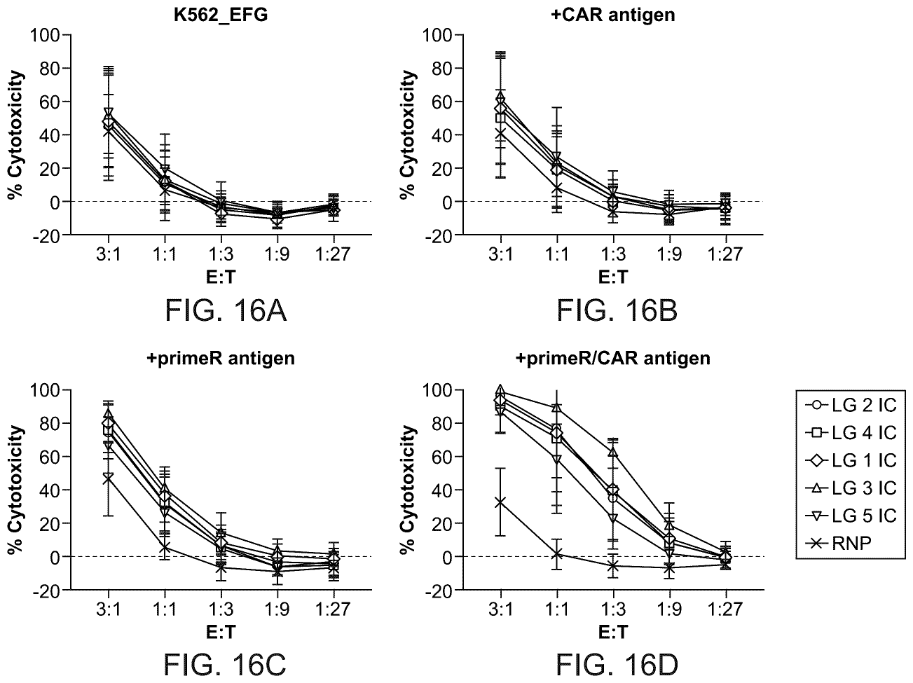

ICTs expressing LG 1-5 ICs demonstrated cytotoxicity against only dual CAR antigen and primeR antigen expressing cells as compared to unedited control cells (RNP). FIG. 16A shows cytotoxicity against parental K562 cells expressing neither CAR antigen or primeR antigen, FIG. 16B shows cytotoxicity against K562 cells expressing only CAR antigen, FIG. 16C shows cytotoxicity against K562 cells expressing only primeR antigen, and FIG. 16D shows cytotoxicity against K562 cells expressing both primeR antigen and CAR antigen. As shown in FIG. 16D, the ICTs exhibited cytotoxicity against only cells expressing both primeR antigen and CAR antigen as compared to unedited cells (RNP).

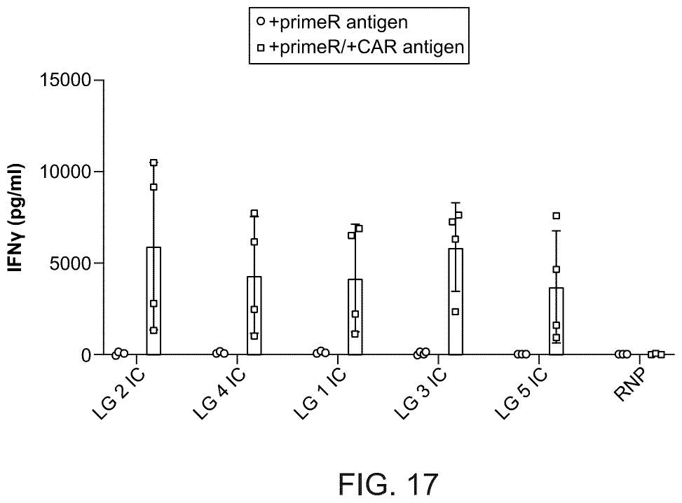

IFN-γ production from ICTs expressing LG 1-5 ICs was observed only in supernatants taken from co-cultures where the target cells expressed both primeR antigen and CAR antigen (FIG. 17). Results from the cytokine analysis were consistent with the cytotoxicity data. Together, these data further demonstrate that ICT activity is driven by co-expression of primeR antigen and CAR antigen.

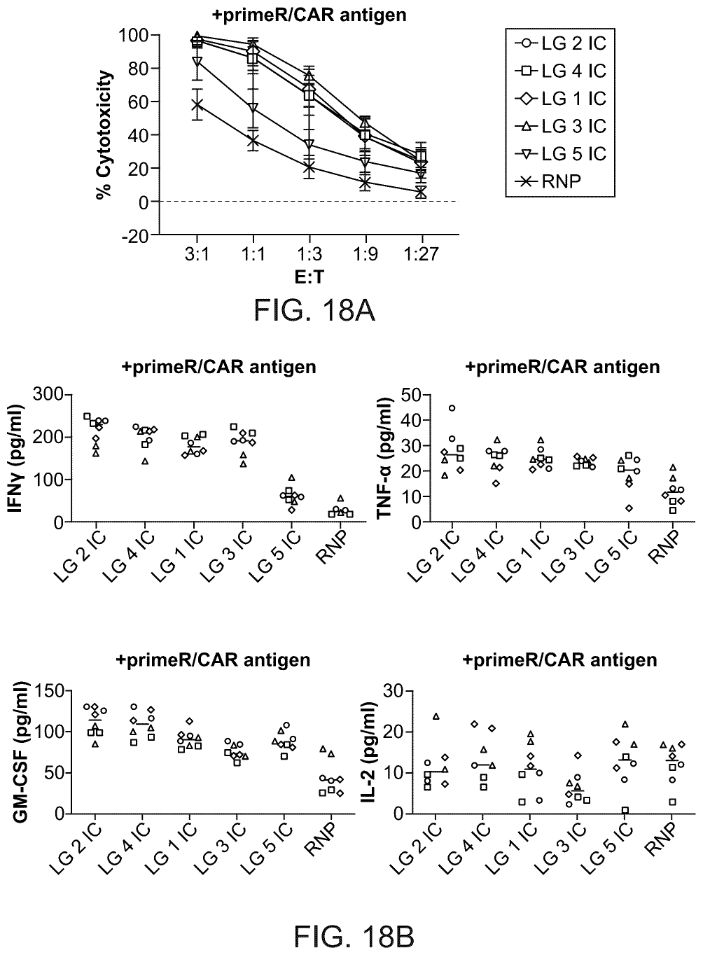

ICTs expressing LG 1-5 ICs demonstrated in vitro cytotoxicity against the primeR antigen-med cell line expressing endogenous CAR antigen (FIG. 18A). ICTs also secrete cytokines after co-culture. FIG. 18B shows IFNγ, TNFα, GM-CSF, and IL-2 secretion by ICT cells after co-culture with primeR antigen+/CAR antigen+ cells. Thus, ICTs expressing LG 1-5 ICs secreted cytokines and killed ccRCC cell lines that express endogenous CAR antigen in the presence of primeR antigen.

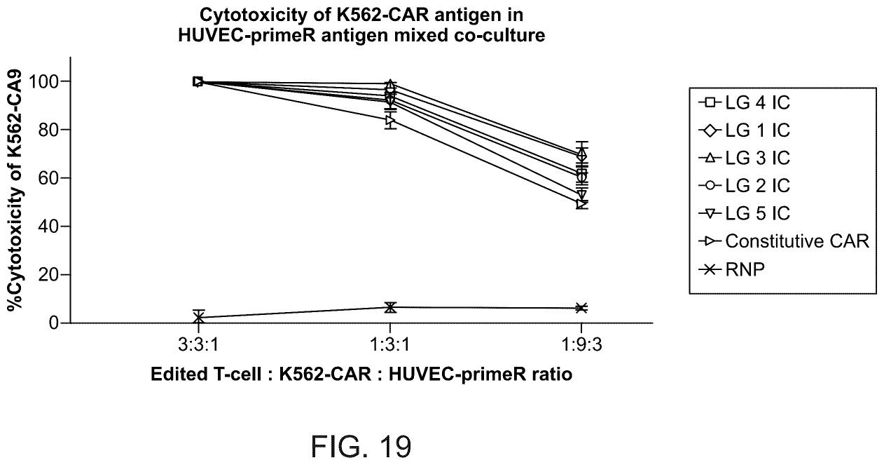

Co-culture with HUVEC-primeR antigen induced expression of the CAR protein on ICT cells and specific killing of CAR antigen+ cells was confirmed (FIG. 19). Thus, ICTs expressing LG 1-5 ICs were capable of inducing CAR expression through interaction with primeR antigen+ endothelial cells and subsequently specifically engaging and killing CAR antigen+ tumor cells. Therefore, without wishing to be bound by theory, ICTs can be primed by binding to endothelial cells expressing primeR antigen in order to express the CAR and then kill CAR antigen+ target tumor cells.

Thus, logic gated ICT cells that utilize the presence of two antigens to trigger tumor cell killing to improve the therapeutic index of CAR T cells were developed, thereby enhancing tumor specificity. Induction of the CAR was gated on the expression of primeR antigen found on the tumor neovasculature of ccRCC. In this example, the PrimeR antigen and CAR antigen were not known to be co-expressed in normal tissues. When the priming receptor (PrimeR) binds its cognate antigen, PrimeR engagement triggers proteolytic release of a transcription factor that induces expression of a CAR. The feasibility of vascular priming was confirmed using a transwell assay where ICTs were primed by a primeR antigen expressing endothelial cell line and then migrated across the transwell membrane to kill CAR antigen expressing RCC cells.

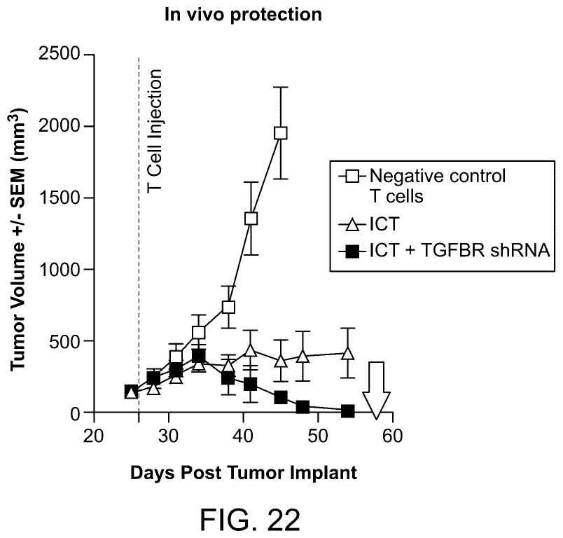

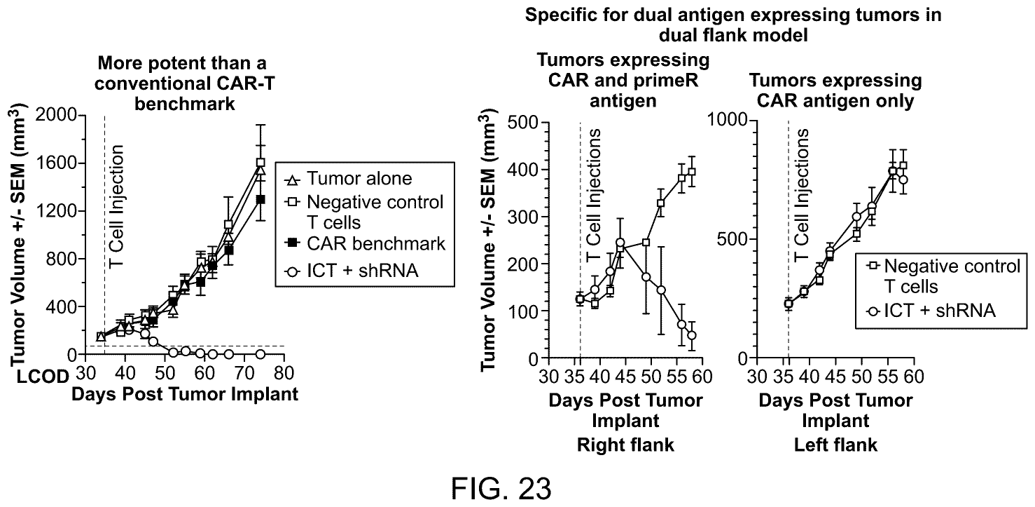

To further increase the potency and persistence of the ICT cells, an shRNA cassette targeting both FAS and TGFBR2, a receptor used for TGFB signaling in T cells, was inserted into the ICTs. Addition of FAS/TGFBR2 shRNAs enhanced antitumor activity of primeR antigen×CAR antigen logic gate expressing T cells during in vitro chronic stimulation assays conducted in the presence of exogenous TGFb (data not shown). Furthermore, FAS/TGFBR shRNA containing ICTs demonstrated enhanced antitumor activity in multiple xenograft RCC models (Example 5, FIG. 22). Collectively, these results demonstrate that primeR antigen-CAR antigen ICT cells can (i) selectively target antigens that cannot generally be safely targeted by conventional CARs; and (ii) overcome multiple suppressive mechanisms in the tumor microenvironment.

### Example 5: In Vivo Efficacy of primeR and CAR Logic Gate T Cells Expressing FAS and TGFBR2 shRNA

### Materials and Methods

### A498 RCC Efficacy Model

Human ccRCC cells express endogenous levels of the CAR antigen and were engineered to express physiological levels of primeR antigen. 2×10⁶ primeR antigen+/CAR antigen+ cells were inoculated into the right dorsal flank of five-six weeks old, female NSG MHC I/II DKO mice. Day 35 post tumor inoculation, mean tumor volume of 150 mm³ was reached and tumor-bearing animals were randomized into various treatment groups such that mean tumor volume per group was within 10% of the overall mean. Seven mice/group were injected intravenously with a single dose of 0.15×10⁶ of PrimeR+ ICT cells expressing one of the five LG ICTs described in Example 4 (LG 1 IC, LG 2 IC, LG 3 IC, LG 4 IC, or LG 5 IC), RNP or PBS. The study was repeated with ICTs generated from two different normal donors. Tumor volumes and body weight were recorded bi-weekly. Tumor volume was calculated as per formula ½*L*W², where L is tumor length and W is tumor width.

Blood pharmacokinetics demonstrated the expansion of ICTs on day 14 followed by complete contraction by day 42 post T cell injection. PrimeR+ ICTs in mouse blood were quantified to track expansion of ICTs using flow cytometry with count bright beads for T cell quantification/volume. Man and SEM plotted.

### Dual Flank Model

Human ccRCC 786-O cells were engineered to express either CAR and primeR antigen or CAR only. 2×10⁶ 786-O-CAR+ and 786-O-CAR+-primeR antigen+ cells were inoculated into the left and right dorsal flank respectively of five-six weeks old, female NSG MHC I/II DKO mice. Day 35 post tumor inoculation, mean tumor volume of 150-200 mm³ was reached on each flank and tumor-bearing animals were randomized into various treatment groups such that mean tumor volume per group on the right flank was within 10% of the overall mean. Seven mice/group were injected intravenously with a single dose of 0.25×10⁶ or 1×10⁶ of PrimeR+ ICT cells, constitutive CAR T cells, RNP or PBS control. Tumor volumes and body weight were recorded bi-weekly. Tumor volume was calculated as per formula ½*L*W², where L is tumor length and W is tumor width. (B) tumor volumes on the 786-O CAR antigen only flank (left), and (C) tumor volumes on the 786-O-CAR+/primeR antigen+ flank (right). Data represents a single donor study with 7 mice per group, mean and SEM plotted.

### Results

### A498 RCC Efficacy Model

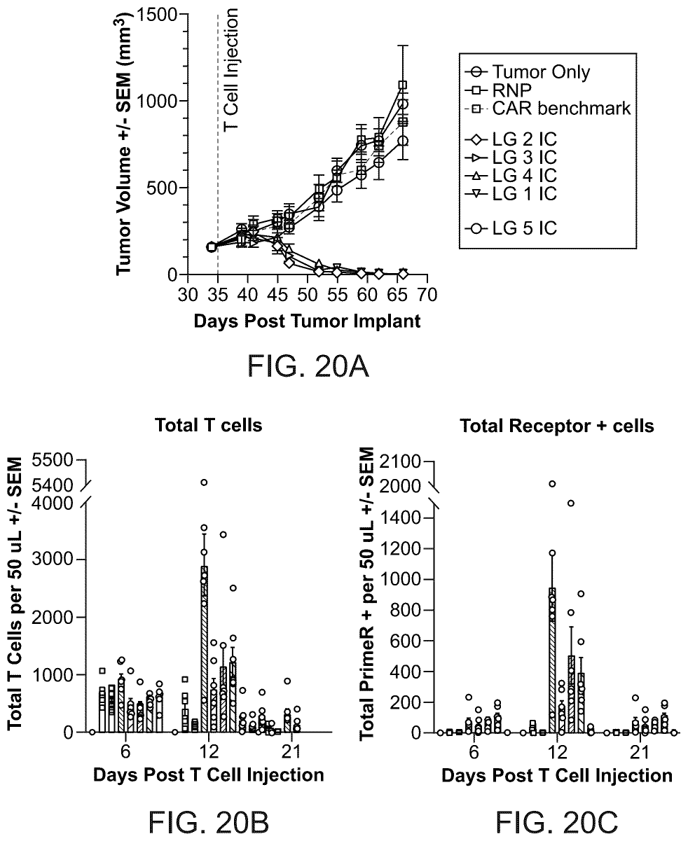

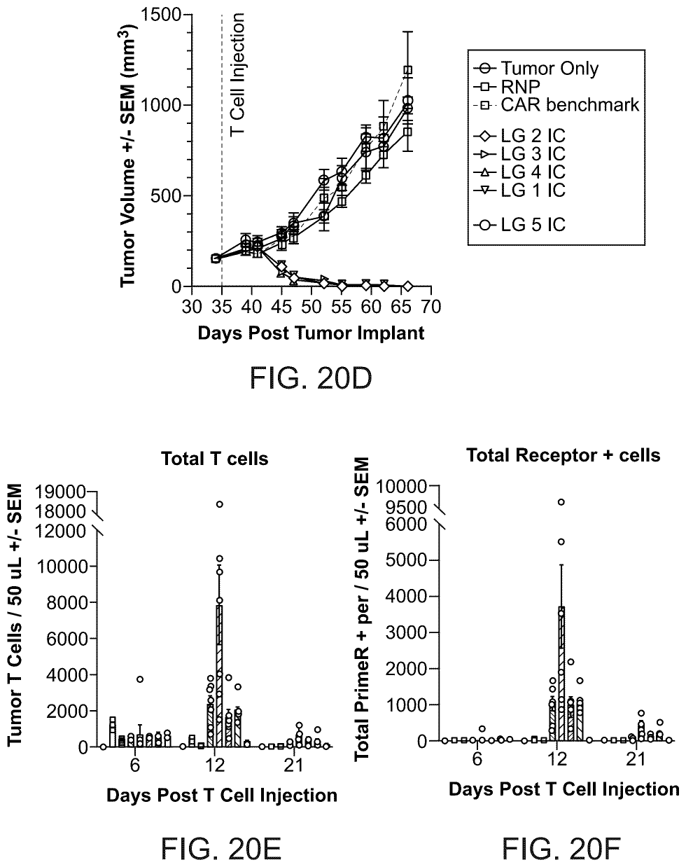

ICTs expressing LG 1-5 ICs showed tumor elimination in a ccRCC model. FIGS. 20A and 20D show the tumor volume post tumor implant in mice treated with ICTs expressing Logic Gates 1-5, RNP or PBS generated from T cells from either donor 1 (FIGS. 20A-C) or donor 2 (FIGS. 20D-F). FIGS. 20B and 20E show the total T cells and expansion of the ICTs on day 12 post inoculation followed by contraction by day 21. FIGS. 20C and 20F show total T cells expressing the priming receptor on days 12 and 21. In both replicates, the ICT cells demonstrated significant tumor-growth inhibition in mice (P<0.05).

### Dual Flank Model

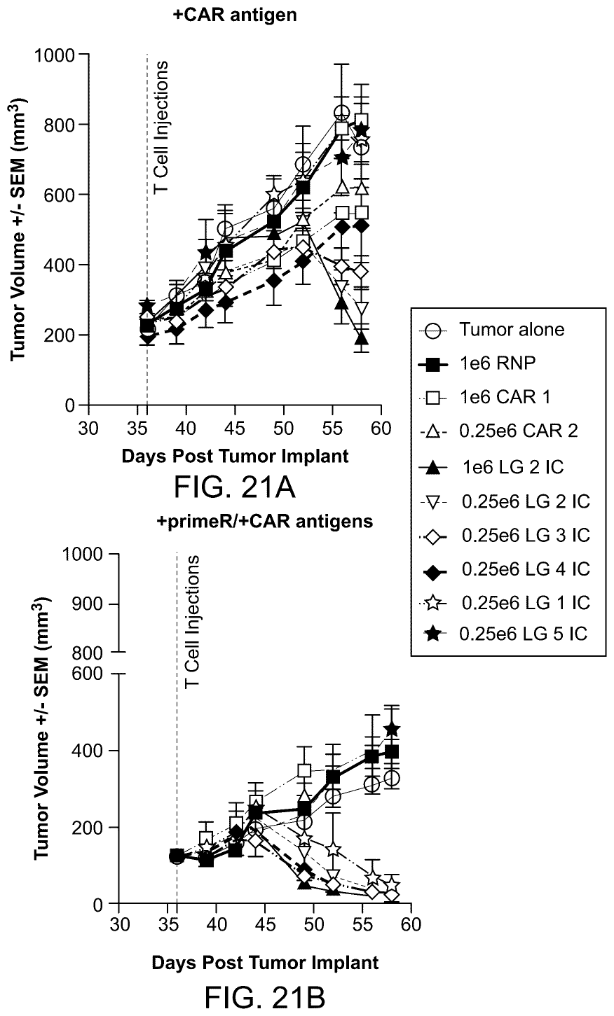

The ICTs expressing LG 1-5 ICs showed specificity in a dual flank model (FIG. 21A-B). Greater tumor growth inhibition (TGI) was observed in the dual positive primeR antigen+/CAR antigen+ flank (FIG. 21B) than the single positive CAR-only flank (FIG. 21A). Thus, the dual flank xenograft model shows that logic gated circuits (ICTs) more selectively killed tumors that express both CAR antigen and primeR antigen, and not tumors that express CAR antigen alone.

### Example 6: Synthesis and Characterization of primeR and CAR Logic Gate T Cells with TGFBR Knockdown or a Synthetic Pathway Activator

T cells expressing the primeR and CAR logic gate (also called integrated circuit T cells (ICTs)) as well as a synthetic pathway activator and FAS and TGFBR shRNA or FAS, TGFBR2, and PTPN2 shRNA were constructed and characterized. The results are provided in FIGS. 22 and 23. FIG. 22 shows that TGFBR knockdown protected ICT cells against TGFβ-mediated inhibition in vitro. FIG. 23 shows that ICT cells are a potent and specific cell therapy in a Renal Cell Carcinoma model in vivo. ICT cells expressing a CAR and primeR logic gate demonstrated significant and specific tumor reduction against tumor cells expressing both CAR antigen and primeR antigen in a dual flank model as described in Example 5. The ICT cells were also more potent than a conventional CAR T cell used as a benchmark.

### Example 7: In Vivo Efficacy of primeR and CAR Logic Gate T Cells Expressing FAS and TGFBR2 shRNA in RCC Models

### Materials and methods

786-O ccRCC Model

2e6 768-O cells engineered to express ALPG and MSLN proteins were injected into the mouse flank, and mice were staged when tumors reached 300 mm³. ICTs expressing an exemplary, corresponding primeR+CAR logic gate and one of four selected quad shRNAs (FAS/PTPN2/TGFBR2/TGFBR2 shRNA, see Example 3, and FIG. 10) were administered by tail vein injection at a stress dose of 3e5 cells/mouse 28 days after the tumor inoculation. Blood was collected on day 42 after the tumor inoculation for PK analysis (data not shown). Tumor volume was measured for 62 days post T cell injection on day 28 for a total of 90 days. Control T cells used were unedited (RNP) or ICTs expressing the logic gate with luciferase (dual luc), a FAS/PTPN2 shRNA module, or had FAS/PTPN2/TFGBR2 knockout via CRISPR. N=7

A498 ccRCC Model

An additional in vivo model of RCC was developed to assess the efficacy of the ICTs with TGFBR2 shRNA. A498 ccRCC cells express endogenous levels of a second exemplary CAR antigen and were engineered to express physiological levels of a second exemplary primeR antigen. The engineered A498 cells were injected into the mouse flank and mice were staged when tumors reached 150 mm³. ICTs expressing a primeR+CAR logic gate and a quad shRNA (FAS/PTPN2/TGFBR2/TGFBR2 shRNA, SEQ ID NO: 131) as well as the exemplary SPA described in Example 4 were administered by tail vein injection at a stress dose of 3e5 cells/mouse 25 days after the tumor inoculation. Blood was collected on day 39 after the tumor inoculation for PK analysis (data not shown). Tumor volume was measured for 28 days post T cell injection for a total of 54 days. Control T cells used were unedited T cells (RNP) or ICTs expressing the logic gate with FAS/PTPN2 shRNA module and the SPA. N=7.

### Results

786-0 ccRCC Model

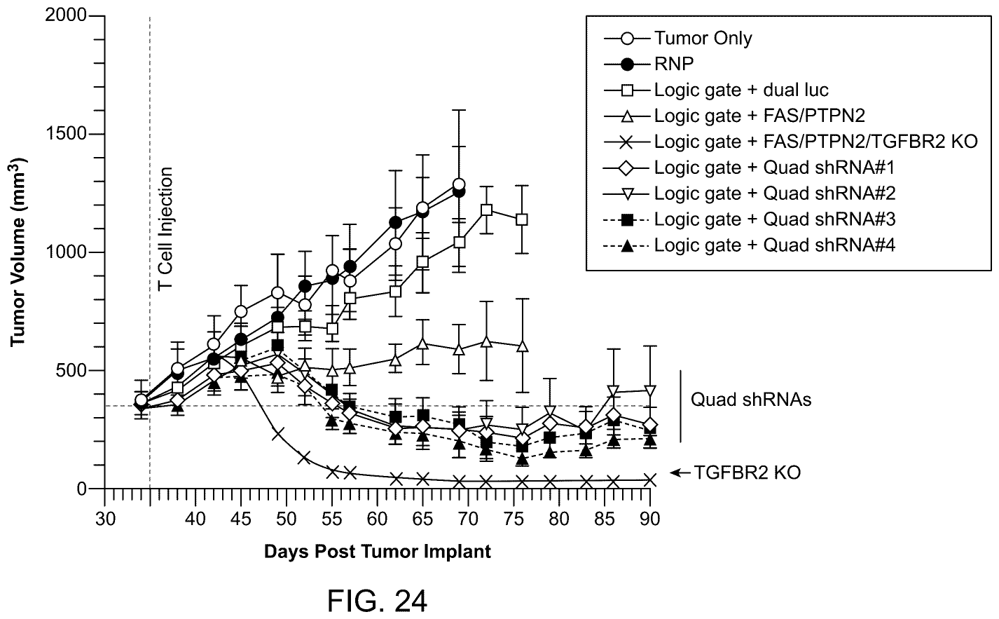

The ICTs with any of the quad shRNA cassettes demonstrated significantly improved tumor clearance as compared to the control T cells in the 786-0 ccRCC model (FIG. 24). The ICTs expressing only the FAS/PTPN2 shRNA demonstrated intermediate levels of tumor clearance as compared to the control cells and the ICTs expressing the quad shRNAs. Thus, inclusion of TGFBR2 shRNA in the ICTs resulted in increased tumor clearance as compared to ICTs comprising the FAS/PTPN2 shRNA alone. ICTs expressing only the logic gate did not demonstrate an antitumor effect in the 786-0 RCC model at the 3e5 stress dose used in the study. Non-edited T cells did not demonstrate an antitumor effect in the 786-O RCC model.

A498 ccRCC Model

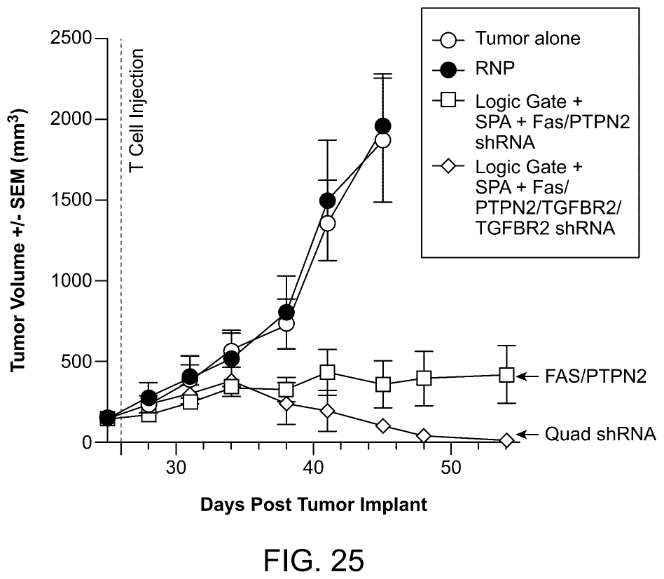

The ICTs with any of the quad shRNA cassettes demonstrated significantly improved tumor clearance and potent anti-tumor response as compared to the control T cells in the A498 ccRCC model (FIG. 25). The ICTs expressing only the FAS/PTPN2 shRNA demonstrated improved tumor clearance as compared to the control cells, but not as significant tumor reduction as the cells expressing the quad shRNA. Thus, the inclusion of TGFBR2 shRNA in the ICTs resulted in increased tumor clearance as compared to ICTs comprising the FAS/PTPN2 shRNA alone. Non-edited T cells did not demonstrate an antitumor effect in the 786-O RCC model.

Thus, this data shows enhancement of anti-tumor efficacy by shRNA knock down of TFGBR2 in two different RCC xenograft models that secrete TGF-β.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

INFORMAL SEQUENCE LISTING

| SEQ | | |
|---|---|---|
| ID | | |
| NO | Name | Sequence |
| 1 | TGF-β | ggcggctagggaggtggggcgaggcgaggtttgctggggtgaggc |
| | Receptor 1 | agcggcgcggccgggccgggccgggccacaggcggtggcggcggg |
| | (TGFBR1) | accatggaggcggcggTCGCTGCTCCGCGTCCCCGGCTGCTCCTC |
| | cDNA | CTCGTGCTggcggcggcggcggcggcggcggcggcgctgctcccg |
| | | ggggcgacggCGTTACAGTGTTTCTGCCACCTCTGTACAAAAGAC |
| | | AATTTTACTTGTGTGACAGATGGGCTCTGCTTTGTCTCTGTCACA |
| | | GAGACCACAGACAAAGTTATACACAACAGCATGTGTATAGCTGAA |
| | | ATTGACTTAATTCCTCGAGATAGGCCGTTTGTATGTGCACCCTCT |
| | | TCAAAAACTGGGTCTGTGACTACAACATATTGCTGCAATCAGGAC |
| | | CATTGCAATAAAATAGAACTTCCAACTACTGTAAAGTCATCACCT |
| | | GGCCTTGGTCCTGTGGAACTGGCAGCTGTCATTGCTGGACCAGTG |
| | | TGCTTCGTCTGCATCTCACTCATGTTGATGGTCTATATCTGCCAC |
| | | AACCGCACTGTCATTCACCATCGAGTGCCAAATGAAGAGGACCCT |
| | | TCATTAGATCGCCCTTTTATTTCAGAGGGTACTACGTTGAAAGAC |
| | | TTAATTTATGATATGACAACGTCAGGTTCTGGCTCAGGTTTACCA |
| | | TTGCTTGTTCAGAGAACAATTGCGAGAACTATTGTGTTACAAGAA |
| | | AGCATTGGCAAAGGTCGATTTGGAGAAGTTTGGAGAGGAAAGTGG |
| | | CGGGGAGAAGAAGTTGCTGTTAAGATATTCTCCTCTAGAGAAGAA |
| | | CGTTCGTGGTTCCGTGAGGCAGAGATTTATCAAACTGTAATGTTA |
| | | CGTCATGAAAACATCCTGGGATTTATAGCAGCAGACAATAAAGAC |
| | | AATGGTACTTGGACTCAGCTCTGGTTGGTGTCAGATTATCATGAG |
| | | CATGGATCCCTTTTTGATTACTTAAACAGATACACAGTTACTGTG |
| | | GAAGGAATGATAAAACTTGCTCTGTCCACGGCGAGCGGTCTTGCC |
| | | CATCTTCACATGGAGATTGTTGGTACCCAAGGAAAGCCAGCCATT |
| | | GCTCATAGAGATTTGAAATCAAAGAATATCTTGGTAAAGAAGAAT |
| | | GGAACTTGCTGTATTGCAGACTTAGGACTGGCAGTAAGACATGAT |
| | | TCAGCCACAGATACCATTGATATTGCTCCAAACCACAGAGTGGGA |
| | | ACAAAAAGGTACATGGCCCCTGAAGTTCTCGATGATTCCATAAAT |
| | | ATGAAACATTTTGAATCCTTCAAACGTGCTGACATCTATGCAATG |
| | | GGCTTAGTATTCTGGGAAATTGCTCGACGATGTTCCATTGGTGGA |
| | | ATTCATGAAGATTACCAACTGCCTTATTATGATCTTGTACCTTCT |
| | | GACCCATCAGTTGAAGAAATGAGAAAAGTTGTTTGTGAACAGAAG |
| | | TTAAGGCCAAATATCCCAAACAGATGGCAGAGCTGTGAAGCCTTG |
| | | AGAGTAATGGCTAAAATTATGAGAGAATGTTGGTATGCCAATGGA |
| | | GCAGCTAGGCTTACAGCATTGCGGATTAAGAAAACATTATCGCAA |
| | | CTCAGTCAACAGGAAGGCATCAAAATGTAATTCTACAGCTTTGCC |
| | | TGAACTCTCCTTTTTTCTTCAGATCTGCTCCTGGGTTTTAATTTG |
| | | GGAGGTCAATTGTTCTACCTCACTGAGAGGGAACAGAAGGATATT |
| | | GCTTCCTTTTGCAGCAGTGTAATAAAGTCAATTAAAAACTTCCCA |
| | | GGATTTCTTTGGACCCAGGAAACAGCCATGTGGGTCCTTTCTGTG |
| | | CACTATGAACGCTTCTTTCCCAGGACAGAAAATGTGTAGTCTACC |
| | | TTTATTTTTTATTAACAAAACTTGTTTTTTAAAAAGATGATTGCT |
| | | GGTCTTAACTTTAGGTAACTCTGCTGTGCTGGAGATCATCTTTAA |
| | | GGGCAAAGGAGTTGGATTGCTGAATTACAATGAAACATGTCTTAT |
| | | TACTAAAGAAAGTGATTTACTCCTGGTTAGTACATTCTCAGAGGA |
| | | TTCTGAACCACTAGAGTTTCCTTGATTCAGACTTTGAATGTACTG |
| | | TTCTATAGTTTTTCAGGATCTTAAAACTAACACTTATAAAACTCT |
| | | TATCTTGAGTCTAAAAATGACCTCATATAGTAGTGAGGAACATAA |
| | | TTCATGCAATTGTATTTTGTATACTATTATTGTTCTTTCACTTAT |
| | | TCAGAACATTACATGCCTTCAAAATGGGATTGTACTATACCAGTA |
| | | AGTGCCACTTCTGTGTCTTTCTAATGGAAATGAGTAGAATTGCTG |
| | | AAAGTCTCTATGTTAAAACCTATAGTGTTTGAATTCAAAAAGCTT |
| | | ATTTATCTGGGTAACCCAAACTTTTTCTGTTTTGTTTTTGGAAGG |
| | | GTTTTTGTGGTATGTCATTTGGTATTCTATTCTGAAAATGCCTTT |
| | | CTCCTACCAAAATGTGCTTAAGCCACTAAAGAAATGAAGTGGCAT |
| | | TAATTAGTAAATTATTAGCATGGTCATGTTTGAATATTCTCACAT |
| | | CAAGCTTTTGCATTTTAATTGTGTTGTCTAAGTATACTTTTAAAA |
| | | AATCAAGTGGCACTCTAGATGCTTATAGTACTTTAATATTTGTAG |
| | | CATACAGACTAATTTTTCTAAAAGGGAAAGTCTGTCTAGCTGCTT |
| | | GTGAAAAGTTATGTGGTATTCTGTAAGCCATTTTTTTCTTTATCT |
| | | GTTCAAAGACTTATTTTTTAAGACATGAATTACATTTAAAATTAG |
| | | AATATGGTTAATATTAAATAATAGGCCTTTTTCTAGGAAGGCGAA |
| | | GGTAGTTAATAATTTGAATAGATAACAGATGTGCAAGAAAGTCAC |
| | | ATTTGTTATGTATGTAGGAGTAAACGTTCGGTGGATCCTCTGTCT |
| | | TTGTAACTGAGGTTAGAGCTAGTGTGGTTTTGAGGTCTCACTACA |
| | | CTTTGAGGAAGGCAGCTTTTAATTCAGTGTTTCCTTATGTGTGCG |
| | | TACATTGCAACTGCTTACATGTAATTTATGTAATGCATTCAGTGC |
| | | ACCCTTGTTACTTGGGAGAGGTGGTAGCTAAAGAACATTCTGAGT |
| | | ATAGGTTTTTCTCCATTTACAGATGTCTTTGGTCAAATATTGAAA |
| | | GCAAACTTGTCATGGTCTTCTTACATTAAGTTGAAACTAGCTTAT |
| | | AATAACTGGTTTTTACTTCCAATGCTATGAAGTCTCTGCAGGGCT |
| | | TTTACAGTTTTCGAAGTCCTTTTATCACTGTGATCTTATTCTGAG |
| | | GGGAGAAAAAACTATCATAGCTCTGAGGCAAGACTTCGACTTTAT |
| | | AGTGCTATCAGTTCCCCGATACAGGGTCAGAGTAACCCATACAGT |
| | | ATTTTGGTCAGGAAGAGAAAGTGGCCATTTACACTGAATGAGTTG |
| | | CATTCTGATAATGTCTTATCTCTTATACGTAGAATAAATTTGAAA |
| | | GACTATTTGATCTTAAAACCAAAGTAATTTTAGAATGAGTGACAT |
| | | ATTACATAGGAATTTAGTGTCAATTTCATGTGTTTAAAAACATCA |
| | | TGGGAAAAATGCTTAGAGGTTACTATTTTGACTACAAAGTTGAGT |
| | | TTTTTTCTGTAGTTACCATAATTTCATTGAAGCAAATGAATGAGT |
| | | TTGAGAGGTTTGTTTTTATAGTTGTGTTGTATTACTTGTTTAATA |
| | | ATAATCTCTAATTCTGTGATCAGGTACtttttttgtgggggtttt |
| | | ttttttgtttttttttttttgttgttgtttttGGGCCATTTCTAA |
| | | GCCTACCAGATCTGCTTTATGAAATCCAGGGGACCAATGCATTTT |
| | | ATCACTAAAACTATTTTTATATAATTTTAAGAATATACCAAAAGT |
| | | TGTCTGATTTAAAGTTGTAATACATGATTTCTCACTTTCATGTAA |
| | | GGTTATCCACTTTTGCTGAAGATATTTTTTATTGAATCAAAGATT |
| | | GAGTTACAATTATACTTTTCTTACCTAAGTGGATAAAATGTACTT |
| | | TTGATGAATCAGGGAATTTTTTTAAAGTTGGAGTTTAGTTCTAAA |
| | | TTGACTTTACGTATTACTGCAGTTAATTCCTTTTTTGGCTAGGGA |
| | | TGGTTTGATAAACCACAATTGGCTGATATTGAAAATGAAAGAAAC |
| | | TTAAAAGGTGGGATGGATCATGATTACTGTCGATAACTGCAGATA |
| | | AATTTGATTAGAGTAATAATTTTGTCATTTAAAAACACAGTTGTT |
| | | TATACTGCCCATCCTAGGATGCTCACCTTCCAAGATTCAACGTGG |
| | | CTAAAACATCTTCTGGTAAATTGTGCGTCCATATTCATTTTGTCA |
| | | GTAGCCAGGAGAAATGGGGATGGGGGAAATACGACTTAGTGAGGC |
| | | ATAGACATCCCTGGTCCATCCTTTCTGTCTCCAGCTGTTTCTTGG |
| | | AACCTGCTCTCCTGCTTGCTGGTCCCTGACGCAGAGACCGTTGCC |
| | | TCCCCCACAGCCGTTTGACTGAAGGCTGCTCTGGAGACCTAGAGT |
| | | AAAACGGCTGATGGAAGTTGTGGGACCCACTTCCATTTCCTTCAG |
| | | TCATTAGAGGTGGAAGGGAGGGGTCTCCAAGTTTGGAGATTGAGC |
| | | AGATGAGGCTTGGGATGCCCCTGCTTTGACTTCAGCCATGGATGA |
| | | GGAGTGGGATGGCAGCAAGGTGGCTCCTGTGGCAGTGGAGTTGTG |
| | | CCAGAAACAGTGGCCAGTTGTATCGCCTATAAGACAGGGTAAGGT |
| | | CTGAAGAGCTGAGCCTGTAATTCTGCTGTAATAATGATAGTGCTC |
| | | AAGAAGTGCCTTGAGTTGGTGTACAGTGCCATGGCCATCAAGAAT |
| | | CCCAGATTTCAGGTTTTATTACAAAATGTAAGTGGTCACTTGGCG |
| | | ATTTTGTAGTACATGCATGAGTTACCTTTTTTCTCTATGTCTGAG |
| | | AACTGTCAGATTAAAACAAGATGGCAAAGAGATCGTTAGAGTGCA |
| | | CAACAAAATCACTATCCCATTAGACACATCATCAAAAGCTTATTT |
| | | TTATTCTTGCACTGGAAGAATCGTAAGTCAACTGTTTCTTGACCA |
| | | TGGCAGTGTTCTGGCTCCAAATGGTAGTGATTCCAAATAATGGTT |
| | | CTGTTAACACTTTGGCAGAAAATGCCAGCTCAGATATTTTGAGAT |
| | | ACTAAGGATTATCTTTGGACATGTACTGCAGCTTCTTGTCTCTGT |
| | | TTTGGATTACTGGAATACCCATGGGCCCTCTCAAGAGTGCTGGAC |
| | | TTCTAGGACATTAAGATGATTGTCAGTACATTAAACTTTTCAATC |
| | | CCATTATGCAATCTTGTTTGTAAATGTAAACTTCTAAAAATATGG |
| | | TTAATAACATTCAACCTGTTTATTACAACTTAAAAGGAACTTCAG |
| | | TGAATTTGTTTTTATTTTTTAACAAGATTTGTGAACTGAATATCA |
| | | TGAACCATGTTTTGATACCCCTTTTTCACGTTGTGCCAACGGAAT |
| | | AGGGTGTTTGATATTTCTTCATATGTTAAGGAGATGCTTCAAAAT |
| | | GTCAATTGCTTTAAACTTAAATTACCTCTCAAGAGACCAAGGTAC |
| | | ATTTACCTCATTGTGTATATAATGTTTAATATTTGTCAGAGCATT |
| | | CTCCAGGTTTGCAGTTTTATTTCTATAAAGTATGGGTATTATGTT |
| | | GCTCAGTTACTCAAATGGTACTGTATTGTTTATATTTGTACCCCA |
| | | AATAACATCGTCTGTACTTTCTGTTTTCTGTATTGTATTTGTGCA |
| | | GGATTCTTTAGGCTTTATCAGTGTAATCTCTGCCTTTTAAGATAT |
| | | GTACAGAAAATGTCCATATAAATTTCCATTGAAGTCGAATGATAC |
| | | TGAGAAGCCTGTAAAGAGGAGAAAAAAACATAAGCTGTGTTTCCC |
| | | CATAAGTTTTTTTAAATTGTATATTGTATTTGTAGTAATATTCCA |
| | | AAAGAATGTAAATAGGAAATAGAAGAGTGATGCTTATGTTAAGTC |
| | | CTAACACTACAGTAGAAGAATGGAAGCAGTGCAAATAAATTACAT |
| | | TTTTCCCAAGTGCCAGTGGCATATTTTAAAATAAAGTGTATACGT |
| | | TGGAATGAGTCATGCCATATGTAGTTGCTGTAGATGGCAACTAGA |
| | | ACCTTTGAGTTACAAGAGTCTTTAGAAGTTTTCTAACCCTGCCTA |
| | | GTGCAAGTTACAATATTATAGCGTGTTCGGGGAGTGCCCTCCTGT |
| | | CTGCAGGTGTGTCTCTGTGCCTGGGGGCTTTTCTCCACATGCTTA |
| | | GGGGTGTGGGTCTTCCATTGGGGCATGATGGACCTGTCTACAGGT |
| | | GATCTCTGTTGCCTTTGGGTCAGCACATTTGTTAGTCTCCTGGGG |
| | | GTGAAAACTTGGCTTACAAGAGAACTGGAAAAATGATGAGATGTG |
| | | GTCCCCAAACCCTTGATTGACTCTGGGGAGGGGCTTTGTGAATAG |
| | | GATTGCTCTCACATTAAAGATAGTTACTTCAATTTGAAGGCTGGA |
| | | TTTAGGGAtttttttttttCCTTATAACAAAGACATCACCAGGAT |
| | | ATGAAGCTTTTGTTGAAAGTTGGAAAAaaagtgaaattaaagaca |
| | | ttcccagacaaa |
| 2 | TGF-β | ACTCGCGCGCACGGAGCGACGACACCCCCGCGCGTGCACCCGCTC |
| | Receptor 2 | GGGACAGGAGCCGGACTCCTGTGCAGCTTCCCTCGGCCGCCGGGG |
| | (TGFBR2) | GCCTCCCCGCGCCTCGCCGGCCTCCAGGCCCCCTCCTGGCTGGCG |
| | cDNA | AGCGGGCGCCACATCTGGCCCGCACATCTGCGCTGCCGGCCCGGC |
| | | GCGGGGTCCGGAGAGGGCGCGGCGCGGAGGCGCAGCCAGGGGTCC |
| | | GGGAAGGCGCCGTCCGCTGCGCTGGGGGCTCGGTCTATGACGAGC |
| | | AGCGGGGTCTGCCATGGGTCGGGGGCTGCTCAGGGGCCTGTGGCC |
| | | GCTGCACATCGTCCTGTGGACGCGTATCGCCAGCACGATCCCACC |
| | | GCACGTTCAGAAGTCGGTTAATAACGACATGATAGTCACTGACAA |
| | | CAACGGTGCAGTCAAGTTTCCACAACTGTGTAAATTTTGTGATGT |
| | | GAGATTTTCCACCTGTGACAACCAGAAATCCTGCATGAGCAACTG |
| | | CAGCATCACCTCCATCTGTGAGAAGCCACAGGAAGTCTGTGTGGC |
| | | TGTATGGAGAAAGAATGACGAGAACATAACACTAGAGACAGTTTG |
| | | CCATGACCCCAAGCTCCCCTACCATGACTTTATTCTGGAAGATGC |
| | | TGCTTCTCCAAAGTGCATTATGAAGGaaaaaaaaaaGCCTGGTGA |
| | | GACTTTCTTCATGTGTTCCTGTAGCTCTGATGAGTGCAATGACAA |
| | | CATCATCTTCTCAGAAGAATATAACACCAGCAATCCTGACTTGTT |
| | | GCTAGTCATATTTCAAGTGACAGGCATCAGCCTCCTGCCACCACT |
| | | GGGAGTTGCCATATCTGTCATCATCATCTTCTACTGCTACCGCGT |
| | | TAACCGGCAGCAGAAGCTGAGTTCAACCTGGGAAACCGGCAAGAC |
| | | GCGGAAGCTCATGGAGTTCAGCGAGCACTGTGCCATCATCCTGGA |
| | | AGATGACCGCTCTGACATCAGCTCCACGTGTGCCAACAACATCAA |
| | | CCACAACACAGAGCTGCTGCCCATTGAGCTGGACACCCTGGTGGG |
| | | GAAAGGTCGCTTTGCTGAGGTCTATAAGGCCAAGCTGAAGCAGAA |
| | | CACTTCAGAGCAGTTTGAGACAGTGGCAGTCAAGATCTTTCCCTA |
| | | TGAGGAGTATGCCTCTTGGAAGACAGAGAAGGACATCTTCTCAGA |
| | | CATCAATCTGAAGCATGAGAACATACTCCAGTTCCTGACGGCTGA |
| | | GGAGCGGAAGACGGAGTTGGGGAAACAATACTGGCTGATCACCGC |
| | | CTTCCACGCCAAGGGCAACCTACAGGAGTACCTGACGCGGCATGT |
| | | CATCAGCTGGGAGGACCTGCGCAAGCTGGGCAGCTCCCTCGCCCG |
| | | GGGGATTGCTCACCTCCACAGTGATCACACTCCATGTGGGAGGCC |
| | | CAAGATGCCCATCGTGCACAGGGACCTCAAGAGCTCCAATATCCT |
| | | CGTGAAGAACGACCTAACCTGCTGCCTGTGTGACTTTGGGCTTTC |
| | | CCTGCGTCTGGACCCTACTCTGTCTGTGGATGACCTGGCTAACAG |
| | | TGGGCAGGTGGGAACTGCAAGATACATGGCTCCAGAAGTCCTAGA |
| | | ATCCAGGATGAATTTGGAGAATGTTGAGTCCTTCAAGCAGACCGA |
| | | TGTCTACTCCATGGCTCTGGTGCTCTGGGAAATGACATCTCGCTG |
| | | TAATGCAGTGGGAGAAGTAAAAGATTATGAGCCTCCATTTGGTTC |
| | | CAAGGTGCGGGAGCACCCCTGTGTCGAAAGCATGAAGGACAACGT |
| | | GTTGAGAGATCGAGGGCGACCAGAAATTCCCAGCTTCTGGCTCAA |
| | | CCACCAGGGCATCCAGATGGTGTGTGAGACGTTGACTGAGTGCTG |
| | | GGACCACGACCCAGAGGCCCGTCTCACAGCCCAGTGTGTGGCAGA |
| | | ACGCTTCAGTGAGCTGGAGCATCTGGACAGGCTCTCGGGGAGGAG |
| | | CTGCTCGGAGGAGAAGATTCCTGAAGACGGCTCCCTAAACACTAC |
| | | CAAATAGCTCTTCTGGGGCAGGCTGGGCCATGTCCAAAGAGGCTG |
| | | CCCCTCTCACCAAAGAACAGAGGCAGCAGGAAGCTGCCCCTGAAC |
| | | TGATGCTTCCTGGAAAACCAAGGGGGTCACTCCCCTCCCTGTAAG |
| | | CTGTGGGGATAAGCAGAAACAACAGCAGCAGGGAGTGGGTGACAT |
| | | AGAGCATTCTATGCCTTTGACATTGTCATAGGATAAGCTGTGTTA |
| | | GCACTTCCTCAGGAAATGAGATTGATTTTTACAATAGCCAATAAC |
| | | ATTTGCACTTTATTAATGCCTGtatataaatatgaatagctatgt |
| | | tttatatatatatatatatatctatatatgtctatagctctatat |
| | | atataGCCATACCTTGAAAAGAGACAAGGAAAAACATCAAATATT |
| | | CCCAGGAAATTGGTTTTATTGGAGAACTCCAGAACCAAGCAGAGA |
| | | AGGAAGGGACCCATGACAGCATTAGCATTTGACAATCACACATGC |
| | | AGTGGTTCTCTGACTGTAAAACAGTGAACTTTGCATGAGGAAAGA |
| | | GGCTCCATGTCTCACAGCCAGCTATGACCACATTGCACTTGCTTT |
| | | TGCAAAATAATCATTCCCTGCCTAGCACTTCTCTTCTGGCCATGG |
| | | AACTAAGTACAGTGGCACTGTTTGAGGACCAGTGTTCCCGGGGTT |
| | | CCTGTGTGCCCTTATTTCTCCTGGACTTTTCATTTAAGCTCCAAG |
| | | CCCCAAATCTGGGGGGCTAGTTTAGAAACTCTCCCTCAACCTAGT |
| | | TTAGAAACTCTACCCCATCTTTAATACCTTGAATGTTTTGAACCC |
| | | CACTTTTTACCTTCATGGGTTGCAGAAAAATCAGAACAGATGTCC |
| | | CCATCCATGCGATTGCCCCACCATCTACTAATGAAAAATTGTTCT |
| | | TTTTTTCATCTTTCCCCTGCACTTATGTTACTATTCTCTGCTCCC |
| | | AGCCTTCATCCTTTTCTAAAAAGGAGCAAATTCTCACTCTAGGCT |
| | | TTATCGTGTTTACTTTTTCATTACACTTGACTTGATTTTCTAGTT |
| | | TTCTATACAAACACCAATGGGTTCCATCTTTCTGGGCTCCTGATT |
| | | GCTCAAGCACAGTTTGGCCTGATGAAGAGGATTTCAACTACACAA |
| | | TACTATCATTGTCAGGACTATGACCTCAGGCACTCTAAACATAtg |
| | | ttttgtttggtcagcacagcgtttcaaaaagtgaagccactttat |
| | | aaatatttggagattttgcaggaaaatctggatccccagGTAAGG |
| | | ATAGCAGATGGTTTTCAGTTATCTCCAGTCCACGTTCACAAAATG |
| | | TGAAGGTGTGGAGACACTTACAAAGCTGCCTCACTTCTCACTGTA |
| | | AACATTAGCTCTTTCCACTGCCTACCTGGACCCCAGTCTAGGAAT |
| | | TAAATCTGCACCTAACCAAGGTCCCTTGTAAGAAATGTCCATTCA |
| | | AGCAGTCATTCTCTGGGTATATAATATGATTTTGACTACCTTATC |
| | | TGGTGTTAAGATTTGAAGTTGGCCTTTTATTGGACTAAAGGGGAA |
| | | CTCCTTTAAGGGTCTCAGTTAGCCCAAGTTTCTTTTGCTTATATG |
| | | TTAATAGTTTTACCCTCTGCATTGGAGAGAGGAGTGCTTTACTCC |
| | | AAGAAGCTTTCCTCATGGTTACCGTTCTCTCCATCATGCCAGCCT |
| | | TCTCAACCTTTGCAGAAATTACTAGAGAGGATTTGAATGTGGGAC |
| | | ACAAAGGTCCCATTTGCAGTTAGAAAATTTGTGTCCACAAGGACA |
| | | AGAACAAAGTATGAGCTTTAAAACTCCATAGGAAACTTGTTAATC |
| | | AACAAAGAAGTGTTAATGCTGCAAGTAATCTCTTTTTTAAAACTT |
| | | TTTGAAGCTACTTATTTTCAGCCAAATAGGAATATTAGAGAGGGA |
| | | CTGGTAGTGAGAATATCAGCTCTGTTTGGATGGTGGAAGGTCTCA |
| | | TTTTATTGAGATTTTTAAGATACATGCAAAGGTTTGGAAATAGAA |
| | | CCTCTAGGCACCCTCCTCAGTGTGGGTGGGCTGAGAGTTAAAGAC |
| | | AGTGTGGCTGCAGTAGCATAGAGGCGCCTAGAAATTCCACTTGCA |
| | | CCGTAGGGCATGCTGATACCATCCCAATAGCTGTTGCCCATTGAC |
| | | CTCTAGTGGTGAGTTTCTAGAATACTGGTCCATTCATGAGATATT |
| | | CAAGATTCAAGAGTATTCTCACTTCTGGGTTATCAGCATAAACTG |
| | | GAATGTAGTGTCAGAGGATACTGTGGCTTGTTTTGTTTATGtttt |
| | | tttttCTTATTCAAGAAAAAAGACCAAGGAATAACATTCTGTAGT |
| | | TCCTAAAAATACTGACTTTTTTCACTACTATACATAAAGGGAAAG |
| | | TTTTATTCTTTTATGGAACACTTCAGCTGTACTCATGTATTAAAA |
| | | TAGGAATGTGAATGCTATATACTCTTTTTATATCAAAAGTCTCAA |
| | | GCACTTATTTTTATTCTATGCATTGTTTGTCTTTTACATAAATAA |
| | | AATGTTTATTAGATTGAATAAAGCAAAATACTCAGGTGAGCATCC |
| | | TGCCTCCTGTTCCCATTCCTAGTAGCTAAA |
| 3 | FAS mRNA | CTCTTCTCCCGCGGGTTGGTGGACCCGCTCAGTACGGAGTTGGGG |
| | NCBI | AAGCTCTTTCACTTCGGAGGATTGCTCAACAACCATGCTGGGCAT |
| | NM_000043.6 | CTGGACCCTCCTACCTCTGGTTCTTACGTCTGTTGCTAGATTATC |
| | | GTCCAAAAGTGTTAATGCCCAAGTGACTGACATCAACTCCAAGGG |
| | | ATTGGAATTGAGGAAGACTGTTACTACAGTTGAGACTCAGAACTT |
| | | GGAAGGCCTGCATCATGATGGCCAATTCTGCCATAAGCCCTGTCC |
| | | TCCAGGTGAAAGGAAAGCTAGGGACTGCACAGTCAATGGGGATGA |
| | | ACCAGACTGCGTGCCCTGCCAAGAAGGGAAGGAGTACACAGACAA |
| | | AGCCCATTTTTCTTCCAAATGCAGAAGATGTAGATTGTGTGATGA |
| | | AGGACATGGCTTAGAAGTGGAAATAAACTGCACCCGGACCCAGAA |
| | | TACCAAGTGCAGATGTAAACCAAACTTTTTTTGTAACTCTACTGT |
| | | ATGTGAACACTGTGACCCTTGCACCAAATGTGAACATGGAATCAT |
| | | CAAGGAATGCACACTCACCAGCAACACCAAGTGCAAAGAGGAAGG |
| | | ATCCAGATCTAACTTGGGGTGGCTTTGTCTTCTTCTTTTGCCAAT |
| | | TCCACTAATTGTTTGGGTGAAGAGAAAGGAAGTACAGAAAACATG |
| | | CAGAAAGCACAGAAAGGAAAACCAAGGTTCTCATGAATCTCCAAC |
| | | TTTAAATCCTGAAACAGTGGCAATAAATTTATCTGATGTTGACTT |
| | | GAGTAAATATATCACCACTATTGCTGGAGTCATGACACTAAGTCA |
| | | AGTTAAAGGCTTTGTTCGAAAGAATGGTGTCAATGAAGCCAAAAT |
| | | AGATGAGATCAAGAATGACAATGTCCAAGACACAGCAGAACAGAA |
| | | AGTTCAACTGCTTCGTAATTGGCATCAACTTCATGGAAAGAAAGA |
| | | AGCGTATGACACATTGATTAAAGATCTCAAAAAAGCCAATCTTTG |
| | | TACTCTTGCAGAGAAAATTCAGACTATCATCCTCAAGGACATTAC |
| | | TAGTGACTCAGAAAATTCAAACTTCAGAAATGAAATCCAAAGCTT |
| | | GGTCTAGAGTGAAAAACAACAAATTCAGTTCTGAGTATATGCAAT |
| | | TAGTGTTTGAAAAGATTCTTAATAGCTGGCTGTAAATACTGCTTG |
| | | GTTTTTTACTGGGTACATTTTATCATTTATTAGCGCTGAAGAGCC |
| | | AACATATTTGTAGATTTTTAATATCTCATGATTCTGCCTCCAAGG |
| | | ATGTTTAAAATCTAGTTGGGAAAACAAACTTCATCAAGAGTAAAT |
| | | GCAGTGGCATGCTAAGTACCCAAATAGGAGTGTATGCAGAGGATG |
| | | AAAGATTAAGATTATGCTCTGGCATCTAACATATGATTCTGTAGT |
| | | ATGAATGTAATCAGTGTATGTTAGTACAAATGTCTATCCACAGGC |
| | | TAACCCCACTCTATGAATCAATAGAAGAAGCTATGACCTTTTGCT |
| | | GAAATATCAGTTACTGAACAGGCAGGCCACTTTGCCTCTAAATTA |
| | | CCTCTGATAATTCTAGAGATTTTACCATATTTCTAAACTTTGTTT |
| | | ATAACTCTGAGAAGATCATATTTATGTAAAGTATATGTATTTGAG |
| | | TGCAGAATTTAAATAAGGCTCTACCTCAAAGACCTTTGCACAGTT |
| | | TATTGGTGTCATATTATACAATATTTCAATTGTGAATTCACATAG |
| | | AAAACATTAAATTATAATGTTTGACTATTATATATGTGTATGCAT |
| | | TTTACTGGCTCAAAACTACCTACTTCTTTCTCAGGCATCAAAAGC |
| | | ATTTTGAGCAGGAGAGTATTACTAGAGCTTTGCCACCTCTCCATT |
| | | TTTGCCTTGGTGCTCATCTTAATGGCCTAATGCACCCCCAAACAT |
| | | GGAAATATCACCAAAAAATACTTAATAGTCCACCAAAAGGCAAGA |
| | | CTGCCCTTAGAAATTCTAGCCTGGTTTGGAGATACTAACTGCTCT |
| | | CAGAGAAAGTAGCTTTGTGACATGTCATGAACCCATGTTTGCAAT |
| | | CAAAGATGATAAAATAGATTCTTATTTTTCCCCCACCCCCGAAAA |
| | | TGTTCAATAATGTCCCATGTAAAACCTGCTACAAATGGCAGCTTA |
| | | TACATAGCAATGGTAAAATCATCATCTGGATTTAGGAATTGCTCT |
| | | TGTCATACCCCCAAGTTTCTAAGATTTAAGATTCTCCTTACTACT |
| | | ATCCTACGTTTAAATATCTTTGAAAGTTTGTATTAAATGTGAATT |
| | | TTAAGAAATAATATTTATATTTCTGTAAATGTAAACTGTGAAGAT |
| | | AGTTATAAACTGAAGCAGATACCTGGAACCACCTAAAGAACTTCC |
| | | ATTTATGGAGGATTTTTTTGCCCCTTGTGTTTGGAATTATAAAAT |
| | | ATAGGTAAAAGTACGTAATTAAATAATGTTTTTGGTATTTCTGGT |
| | | TTTCTCTTTTTTGGTAGGGGCTTGCTTTTTGGTTTTGTCTTCCTT |
| | | TTCTCTAACTGATGCTAAATATAACTTGTCTTTAATGCTTCTTGG |
| | | ATCCCTTAGAAGGTACTTCCTTTTTAACCTTAACCCTTTTAGTAG |
| | | TTAAATAATTATTTCCATAGGTTGCTATTGCCAAGAAGACCTCTT |
| | | CCAAACAGCACATGATTATTCGTCAAACAGTTTCGTATTCCAGAT |
| | | ACTGGAATGTGGATAAGAAAGTATACATTTCAAGGGGTAGGTTTT |
| | | ATTATTAAGAAAGCCAAATGAGGATTTTGAAATATTCTTTCCTGC |
| | | ATATTATCCATTCTAGCTACATGCTGGCCAGTGGGCCACCTTTCT |
| | | TTTCTGCAATTTAATGCTAGTAATATATTCTATTTAACCCATGAG |
| | | TCCCAAAGTATTAGCATTTCAACATGTAAGCATGTCGGTAAGATA |
| | | GTTGTGCTTTGCTTAGGGTTCCCTCCTGTGTTATGGTCTGGAAAG |
| | | TGTCTTTAGGCAGAAAGTCTGAGTGATCACAGGGTTCACTCATTA |
| | | ATTTCTCTTTTCTGAGCCATCATAGTCTGTGCTGTCTGCTCTCCA |
| | | GTTTTCTATTTCTAGACAGAAGTAGGGCAAGTTAGGTACTAGTTA |
| | | TTCTTCATGGCCAGAAGTGCAAGTTCTACTTTGCAAGACAAGATT |
| | | AAGTTAGAGAACACCCTATTCCACTTTGGTGAACTCAGAGCAAGA |
| | | ACTTTGAGTTCCTTTGGGAGGAAGACAGTGGAGAAGTCTTTGTAC |
| | | TTGGTGATGTGGTTTTTTTCCTCATGGCTTCACCTAGTGGCCCCA |
| | | AGCATGACTTCTCCCATGTCAATGAGCACAGCCACATTCCCGAGT |
| | | TGAGGTGACCCCACGGTCCAGAATCATCCTCATTCTGGTGAACCT |
| | | GGTTCTCTTTGTGGTGGGCATACTGGGTAGGAGAATCACCCAAAG |
| | | GTCACCCATGAGCTGCAGAAAAAAAGGCTATTTGCAGAAGGAGCT |
| | | CACAGATCACATTGAAAGCATTGCATATTCAAACATCTTGGTCTT |
| | | CTTTATTGGCATGCCCACAGGGTCTTCTGACCTCTGATTAGATCA |
| | | GACACTTTTTAGATATTGAATCATCAGTTTCTGTACAACTATCTG |
| | | AATAAGGTATATAATCAATGAAATTTAGAATTTTTTTCTATGCTT |
| | | ACTCCTGATTGGTAATTTGTTTGGGTTTAGAATTCTATACAAGGC |
| | | CATTTGTAATTTTCCTCAGCACTTTAAAAATATTAAACCATGTTT |
| | | TCTTAA |
| 4 | PTPN2 mRNA | GCATGCGCCGCAGCGCCAGCGCTCTCCCCGGATCGTGCGGGGCCT |
| | NCBI | GAGCCTCTCCGCCGGCGCAGGCTCTGCTCGCGCCAGCTCGCTCCC |
| | NM_002828.4 | GCAGCCATGCCCACCACCATCGAGCGGGAGTTCGAAGAGTTGGAT |
| | | ACTCAGCGTCGCTGGCAGCCGCTGTACTTGGAAATTCGAAATGAG |
| | | TCCCATGACTATCCTCATAGAGTGGCCAAGTTTCCAGAAAACAGA |
| | | AATCGAAACAGATACAGAGATGTAAGCCCATATGATCACAGTCGT |
| | | GTTAAACTGCAAAATGCTGAGAATGATTATATTAATGCCAGTTTA |
| | | GTTGACATAGAAGAGGCACAAAGGAGTTACATCTTAACACAGGGT |
| | | CCACTTCCTAACACATGCTGCCATTTCTGGCTTATGGTTTGGCAG |
| | | CAGAAGACCAAAGCAGTTGTCATGCTGAACCGCATTGTGGAGAAA |
| | | GAATCGGTTAAATGTGCACAGTACTGGCCAACAGATGACCAAGAG |
| | | ATGCTGTTTAAAGAAACAGGATTCAGTGTGAAGCTCTTGTCAGAA |
| | | GATGTGAAGTCGTATTATACAGTACATCTACTACAATTAGAAAAT |
| | | ATCAATAGTGGTGAAACCAGAACAATATCTCACTTTCATTATACT |
| | | ACCTGGCCAGATTTTGGAGTCCCTGAATCACCAGCTTCATTTCTC |
| | | AATTTCTTGTTTAAAGTGAGAGAATCTGGCTCCTTGAACCCTGAC |
| | | CATGGGCCTGCGGTGATCCACTGTAGTGCAGGCATTGGGCGCTCT |
| | | GGCACCTTCTCTCTGGTAGACACTTGTCTTGTTTTGATGGAAAAA |
| | | GGAGATGATATTAACATAAAACAAGTGTTACTGAACATGAGAAAA |
| | | TACCGAATGGGTCTTATTCAGACCCCAGATCAACTGAGATTCTCA |
| | | TACATGGCTATAATAGAAGGAGCAAAATGTATAAAGGGAGATTCT |
| | | AGTATACAGAAACGATGGAAAGAACTTTCTAAGGAAGACTTATCT |
| | | CCTGCCTTTGATCATTCACCAAACAAAATAATGACTGAAAAATAC |
| | | AATGGGAACAGAATAGGTCTAGAAGAAGAAAAACTGACAGGTGAC |
| | | CGATGTACAGGACTTTCCTCTAAAATGCAAGATACAATGGAGGAG |
| | | AACAGTGAGAGTGCTCTACGGAAACGTATTCGAGAGGACAGAAAG |
| | | GCCACCACAGCTCAGAAGGTGCAGCAGATGAAACAGAGGCTAAAT |
| | | GAGAATGAACGAAAAAGAAAAAGGTGGTTATATTGGCAACCTATT |
| | | CTCACTAAGATGGGGTTTATGTCAGTCATTTTGGTTGGCGCTTTT |
| | | GTTGGCTGGACACTGTTTTTTCAGCAAAATGCCCTATAAACAATT |
| | | AATTTTGCCCAGCAAGCTTCTGCACTAGTAACTGACAGTGCTACA |
| | | TTAATCATAGGGGTTTGTCTGCAGCAAACGCCTCATATCCCAAAA |
| | | ACGGTGCAGTAGAATAGACATCAACCAGATAAGTGATATTTACAG |
| | | TCACAAGCCCAACATCTCAGGACTCTTGACTGCAGGTTCCTCTGA |
| | | ACCCCAAACTGTAAATGGCTGTCTAAAATAAAGACATTCATGTTT |
| | | GTTAAAAACTGGTAAATTTTGCAACTGTATTCATACATGTCAAAC |
| | | ACAGTATTTCACCTGACCAACATTGAGATATCCTTTATCACAGGA |
| | | TTTGTTTTTGGAGGCTATCTGGATTTTAACCTGCACTTGATATAA |
| | | GCAATAAATATTGTGGTTTTATCTACGTTATTGGAAAGAAAATGA |
| | | CATTTAAATAATGTGTGTAATGTATAATGTACTATTGACATGGGC |
| | | ATCAACACTTTTATTCTTAAGCATTTCAGGGTAAATATATTTTAT |
| | | AAGTATCTATTTAATCTTTTGTAGTTAACTGTACTTTTTAAGAGC |
| | | TCAATTTGAAAAATCTGTTACTAAAAAAATAAATTGTATGTCGAT |
| | | TGAATTGTACTGGATACATTTTCCATTTTTCTAAAGAGAAGTTTG |
| | | ATATGAGCAGTTAGAAGTTGGAATAAGCAATTTCTACTATATATT |
| | | GCATTTCTTTTATGTTTTACAGTTTTCCCCATTTTAAAAAGAAAA |
| | | GCAAACAAAGAAACAAAAGTTTTTCCTAAAAATATCTTTGAAGGA |
| | | AAATTCTCCTTACTGGGATAGTCAGGTAAACAGTTGGTCAAGACT |
| | | TTGTAAAGAAATTGGTTTCTGTAAATCCCATTATTGATATGTTTA |
| | | TTTTTCATGAAAATTTCAATGTAGTTGGGGTAGATTATGATTTAG |
| | | GAAGCAAAAGTAAGAAGCAGCATTTTATGATTCATAATTTCAGTT |
| | | TACTAGACTGAAGTTTTGAAGTAAACACTTTTCAGTTTCTTTCTA |
| | | CTTCAATAAATAGTATGATTATATGCAAACCTTACATTGTCATTT |
| | | TAACTTAATGAATATTTTTTAAAGCAAACTGTTTAATGAATTTAA |
| | | CTGCTCATTTGAATGCTAGCTTTCCTCAGATTTCAACATTCCATT |
| | | CAGTGTTTAATTTGTCTTACTTAAACTTGAAATTGTTGTTACAAA |
| | | TTTAATTGCTAGGAGGCATGGATAGCATACATTATTATGGATAGC |
| | | ATACCTTATTTCAGTGGTTTTCAAACTATGCTCATTGGATGTCCA |
| | | GGTGGGTCAAGAGGTTACTTTCAACCACAGCATCTCTGCCTTGTC |
| | | TCTTTATATGCCACATAAGATTTCTGCATAAGGCTTAAGTATTTT |
| | | AAAGGGGGCAGTTATCATTTAAAAACAGTTTGGTCGGGCGCGGTG |
| | | GCTCATGCCTGTAATCCCAGCACTTTGGGAGGCTGAAGTGGGCAG |
| | | ATCACCTGAGGTCAGGAGTTCAAGACCAGCCTGGCCAACGTGGTG |
| | | AAACACCATCTCTACTAAAAATGCAAAAATTAGCTGGGCATGGTG |
| | | GAGGGCACCTGTAATCTCAGCTACTCAGGAGGCTGAGGTAGGAGA |
| | | ATTGCTTGAACCCAGGAGATGGAGGTTGCAGTGAGCTGAGATCAC |
| | | GTCACTGCACTCCAGCCAGGGCGACAGAGCGAGACTCCATCTCAA |
| | | AAGAAACAAACAAAAAAAACAGTTTGGGCCGGGTGTGGTGGCTCA |
| | | CGCTTGTAATCCCAGCACTTCGGAAGGCCAAGGCGGGCGGATCAC |
| | | GAGGTCAAGAGATGGAGACTGTCCTGGCCAACATGGTGAAATCCC |
| | | TTCTTTACTAAAAATACAAAAATTATCTGGGCGTGGTGGTGCATG |
| | | CCTGTAGTCCCAGCTCCTTGGGAGGCTAAGGCAGGAGAATCACTT |
| | | GAACCCGGGAGGCAGAGGTTGCAGTGAGCCGAGATTGCACCACTG |
| | | CACTCCAGCCTGGCAACAGAGCAAGACTTCGTCTCAAAAAAAAAA |
| | | AAAAAAAAAGTTTGAAAACCATTGGTATAGATAGATATTTTGAAT |
| | | TGATTTGCATAGTCTCCTTGAATGTGTTAAATTATGTTGAAAGTA |
| | | TGAAAGCAGGATGTAGGTGGTACTACATATTAAATAAGATTTATA |
| | | TAACA |
| 5 | TOX mRNA | CTCTTCTTCTTAAACAAACCACAAACGGATGTGAGGGAAGGAAGG |
| | NCBI | TGTTTCTTTTACTCCTGAGCCCAGACACCTCACTCTGTTCCGTCT |
| | NM_014729.3 | AAGCTTGTTTTGCTGAACACTTTTTTTTAAAAAAGGAAAAAGAAA |
| | | AGGAGTTGCTTGATGTGAGAGTGAAATGGACGTAAGATTTTATCC |
| | | ACCTCCAGCCCAGCCCGCCGCTGCGCCCGACGCTCCCTGTCTGGG |
| | | ACCTTCTCCCTGCCTGGACCCCTACTATTGCAACAAGTTTGACGG |
| | | TGAGAACATGTATATGAGCATGACAGAGCCGAGCCAGGACTATGT |
| | | GCCAGCCAGCCAGTCCTACCCTGGTCCAAGCCTGGAAAGTGAAGA |
| | | CTTCAACATTCCACCAATTACTCCTCCTTCCCTCCCAGACCACTC |
| | | GCTGGTGCACCTGAATGAAGTTGAGTCTGGTTACCATTCTCTGTG |
| | | TCACCCCATGAACCATAATGGCCTGCTACCATTTCATCCACAAAA |
| | | CATGGACCTCCCTGAAATCACAGTCTCCAATATGCTGGGCCAGGA |
| | | TGGAACACTGCTTTCTAATTCCATTTCTGTGATGCCAGATATACG |
| | | AAACCCAGAAGGAACTCAGTACAGTTCCCATCCTCAGATGGCAGC |
| | | CATGAGACCAAGGGGCCAGCCTGCAGACATCAGGCAGCAGCCAGG |
| | | AATGATGCCACATGGCCAGCTGACTACCATTAACCAGTCACAGCT |
| | | AAGTGCTCAACTTGGTTTGAATATGGGAGGAAGCAATGTTCCCCA |
| | | CAACTCACCATCTCCACCTGGAAGCAAGTCTGCAACTCCTTCACC |
| | | ATCCAGTTCAGTGCATGAAGATGAAGGCGATGATACCTCTAAGAT |
| | | CAATGGTGGAGAGAAGCGGCCTGCCTCTGATATGGGGAAAAAACC |
| | | AAAAACTCCCAAAAAGAAGAAGAAGAAGGATCCCAATGAGCCCCA |
| | | GAAGCCTGTGTCTGCCTATGCGTTATTCTTTCGTGATACTCAGGC |
| | | CGCCATCAAGGGCCAAAATCCAAACGCTACCTTTGGCGAAGTCTC |
| | | TAAAATTGTGGCTTCAATGTGGGACGGTTTAGGAGAAGAGCAAAA |
| | | ACAGGTCTATAAAAAGAAAACCGAGGCTGCGAAGAAGGAGTACCT |
| | | GAAGCAACTCGCAGCATACAGAGCCAGCCTTGTATCCAAGAGCTA |
| | | CAGTGAACCTGTTGACGTGAAGACATCTCAACCTCCTCAGCTGAT |
| | | CAATTCGAAGCCGTCGGTGTTCCATGGGCCCAGCCAGGCCCACTC |
| | | GGCCCTGTACCTAAGTTCCCACTATCACCAACAACCGGGAATGAA |
| | | TCCTCACCTAACTGCCATGCATCCTAGTCTCCCCAGGAACATAGC |
| | | CCCCAAGCCGAATAACCAAATGCCAGTGACTGTCTCTATAGCAAA |
| | | CATGGCTGTGTCCCCTCCTCCTCCCCTCCAGATCAGCCCGCCTCT |
| | | TCACCAGCATCTCAACATGCAGCAGCACCAGCCGCTCACCATGCA |
| | | GCAGCCCCTTGGGAACCAGCTCCCCATGCAGGTCCAGTCTGCCTT |
| | | ACACTCACCCACCATGCAGCAAGGATTTACTCTTCAACCCGACTA |
| | | TCAGACTATTATCAATCCTACATCTACAGCTGCACAAGTTGTCAC |
| | | CCAGGCAATGGAGTATGTGCGTTCGGGGTGCAGAAATCCTCCCCC |
| | | ACAACCGGTGGACTGGAATAACGACTACTGCAGTAGTGGGGGCAT |
| | | GCAGAGGGACAAAGCACTGTACCTTACTTGAGAATCTGAACACCT |
| | | CTTCTTTCCACTGAGGAATTCAGGGAAGTGTTTTCACCATGGATT |
| | | GCTTTGTACAGTCAAGGCAGTTCTCCATTTTATTAGAAAATACAA |
| | | GTTGCTAAGCACTTAGGACCATTTGAGCTTGTGGGTCACCCACTC |
| | | TGGAAGAAATAGTCATGCTTCTTTATTATTTTTTTAATCCTTTAT |
| | | GGACATTGTTTTTCTTCTTCCCTGAAGGAAATTTGGACCATTCAG |
| | | ATTTTATGTTGGTTTTTTGCTGTGAAGTGCTGCGCTCTAGTAACT |
| | | GCCTTAGCAACTGTAGATGTCTCGGATAAAAGTCCTGGATTTTCC |
| | | ATTGGTTTTCATAATGGGTGTTTATATGAAACTACTAAAGACTTT |
| | | TTAAATGGCTTGATGTAGCAGTCATAGCAAGTTTGTAAATAGCAT |
| | | CTATGTTACACTCTCCTAGAGTATAAAATGTGAATGTTTTTGTAG |
| | | CTAAATTGTAATTGAAACTGGCTCATTCCAGTTTATTGATTTCAC |
| | | AATAGGGGTTAAATTGGCAAACATTCATATTTTTACTTCATTTTT |
| | | AAAACAACTGACTGATAGTTCTATATTTTCAAAATATTTGAAAAT |
| | | AAAAAGTATTCCCAAGTGATTTTAATTTAAAAACAAATTGGCTTT |
| | | GTCTCATTGATCAGACAAAAAGAAACTAGTATTAAGGGAAGCGCA |
| | | AACACATTTATTTTGTACTGCAGAAAAATTGCTTTTTTGTATCAC |
| | | TTTTTGTGTAATGGTTAGTAAATGTCATTTAAGTCCTTTTATGTA |
| | | TAAAACTGCCAAATGCTTACCTGGTATTTTATTAGATGCAGAAAC |
| | | AGATTGGAAACAGCTAAATTACAACTTTTACATATGGCTCTGTCT |
| | | TATTGTTTCTTCATACTGTGTCTGTATTTAATCTTTTTTTATGGA |
| | | ACCTGTTGCGCCTATTTATGAAATAATAAATATAGGTGTTTGTAA |
| | | GTAAATTTGTTAGTATTTGAAAGAGGTTTCTTTGATGTTTTAACT |
| | | TTTGCTGGCAAAAAAAAATTCACGCTTGGTGTGAATACTTTATTA |
| | | TTTAGTTTTTACAGTAACATGAATAAAGCCAAACCTGCTTTTCAT |
| | | TTAGCAGCAAATTAAAGTAACCAGTCCTTATTTCTGCATTTCTTT |
| | | GGTTGATGCAAACAAAAAACTATTATATTTAAGAACTTTATTTCT |
| | | TCATACGACATAACAGAATTGCCCTCCAAGTCACACAAGCTCCAA |
| | | GACTAAACAAACAGACAGGTCCTCTGTCTTAAAAAGGTTACTTCT |
| | | TGGTTCTCAGCTGGTTCTAGTCAATTCTGAACCACCACCCCCCGC |
| | | CCCCCGCAAAAAAGTAAAAGTCAAACCAAACTTCCTCAAGCTGCA |
| | | TGCTTTTCACAAAATCCAGAAAGCATTTAAGAATTGAACTAGGGG |
| | | CTGGAAGAAGTGAAAGGGAAGCATCTAAAAATGAAAGGTGAGTAA |
| | | CCAGATAGCAAAAGAAAAGGGAAAGCCATCCAAATTTGAAAGCTG |
| | | TTGATAGAAATTGAGATTCTTGCTGTCTTTTGTGCCTCTACAAGC |
| | | TACTACTCATTCCAGAATTCCTGGGTCTTCCAAGAGGATTCTTAA |
| | | GGTACCAGAGATTTGCTAGGGAACCAAAAGTGCTTGAGAATCTGC |
| | | CTGAGGGCTTGCATAGCTTTCACATTAAAAAAAGAAAAAGCTAGC |
| | | AGATTTACTCCTTTTTAGGGGATCATATCAAGAAAGTTAGTCTGG |
| | | TTGGAAACCAAGAGAATGGCTGATGTCTCTTTCTTGGAATATGTG |
| | | AAATAAATTTAGCAGTTTAACTAAATACAAATATATGCATTGTGT |
| | | AATCCACTCAGAATTAAACAGACAAAAGGTATGCTTGCTTTGGAA |
| | | TGATTTTAGGCATTGTACAACCTTGAATCACTTGAGCATGTAATA |
| | | ACTAATAAATAATGCAGATCCATGTGATTATTAAAATGACTGTAG |
| | | CTGAGAGCTCTAATTTTCCTGTCTTGAAACTGTATAAGAACTCAT |
| | | GTGATTAAGTTCACAGTTTATTGTTTGTCTGTTTAGTATTTTAGA |
| | | AATATACCAGCACTACTAATTAACTAATGTCTTTTATTTATTATA |
| | | TTATGATAAAGTAAAAATTTCACTTGCATTAAGTCTAAACTGAGA |
| | | AGGTAATTACTGGGAGGAGAATGAGCAGCTTTGACTTTGACAGGC |
| | | GGTTTGTGCAGGAAAGCACAGTGCCGTGTTGTTTACAGCTTTTCT |
| | | AGAGCAGCTGTGCGACCAGGGTAGAGAGTGTTGAAATTCAATACC |
| | | AAATACAGTAAAAACAAATGTAAATAAAAGAAAACACATCATCAA |
| | | TAAAACTGTTATTATGCGTGACCGTA |
| 6 | TGFBR1_8 | TTAAATGTAATTCATGTCTTAA |
| 7 | TGFBR1_9 | TTCAATGAAATTATGGTAACTA |
| 8 | TGFBR1_5 | TATAATTGTAACTCAATCTTTG |
| 9 | TGFBR1_3 | TTTGATTTCAAATCTCTATGAG |
| 10 | TGFBR1_2 | TTTAAATGTAATTCATGTCTTA |
| 11 | TGFBR1_6 | TTAAGTTGTAATAAACAGGTTG |
| 12 | TGFBR1_7 | TTTAATTGACTTTATTACACTG |
| 13 | TGFBR1_4 | TTAGTATCTCAAAATATCTGAG |
| 14 | TGFBR1_1 | TTAATTGACTTTATTACACTGC |
| 15 | TGFBR1_15 | TTAAATGACAAAATTATTACTC |
| 16 | TGFBR1_13 | TTAGTAATAAGACATGTTTCAT |
| 17 | TGFBR1_27 | TATTCAAATTATTAACTACCTT |
| 18 | TGFBR1_12 | TAAGTAATCAAAAAGGGATCCA |
| 19 | TGFBR1_14 | TTGTGTATAACTTTGTCTGTGG |
| 20 | TGFBR1_22 | TTTTAAATGTAATTCATGTCTT |
| 21 | TGFBR1_29 | TTAACATAAGCATCACTCTTCT |
| 22 | TGFBR1_18 | TTAAAGTACTATAAGCATCTAG |
| 23 | TGFBR1_11 | TATCTATTCAAATTATTAACTA |
| 24 | TGFBR1_17 | TCTTGTAACACAATAGTTCTCG |
| 25 | TGFBR1_20 | TTAAACAAGTAATACAACACAA |
| 26 | TGFBR1_30 | TAATAAAACCTGAAATCTGGGA |
| 27 | TGFBR1_23 | TACATTTTGTAATAAAACCTGA |
| 28 | TGFBR1_10 | TAGAATTACATTTTGATGCCTT |
| 29 | TGFBR1_28 | TTTAAGATCAAATAGTCTTTCA |
| 30 | TGFBR1_21 | TACAATTTAAAAAAACTTATGG |
| 31 | TGFBR1_16 | TAAAATTGTCTTTTGTACAGAG |
| 32 | TGFBR1_25 | TAAGTTGTAATAAACAGGTTGA |
| 33 | TGFBR1_19 | TAATGTACTGACAATCATCTTA |
| 34 | TGFBR1_24 | TTTCATTTTCAATATCAGCCAA |
| 35 | TGFBR1_26 | TTTTAAGATCAAATAGTCTTTC |
| 36 | TGFBR2_5 | TTTCTTACAAGGGACCTTGGTT |
| 37 | TGFBR2_9 | TATAAGCAAAAGAAACTTGGGC |
| 38 | TGFBR2_8 | TTTCTGGTTGTCACAGGTGGAA |
| 39 | TGFBR2_3 | TTGTTGATTAACAAGTTTCCTA |
| 40 | TGFBR2_1 | TAGAGTGAGAATTTGCTCCTTT |
| 41 | TGFBR2_4 | TTCAGGAATCTTCTCCTCCGAG |
| 42 | TGFBR2_2 | TTTAATACATGAGTACAGCTGA |
| 43 | TGFBR2_7 | TTTCTAAACTAGGTTGAGGGAG |
| 44 | TGFBR2_6 | TTGTCAGTGACTATCATGTCGT |
| 45 | TGFBR2_32 | TATTTTAATACATGAGTACAGC |
| 46 | TGFBR2_25 | TAAGCAAAAGAAACTTGGGCTA |
| 47 | TGFBR2_14 | TTTTAATACATGAGTACAGCTG |
| 48 | TGFBR2_11 | TTAAAAAAGAGATTACTTGCAG |
| 49 | TGFBR2_34 | TATTCATATTTATATACAGGCA |
| 50 | TGFBR2_49 | TTTTAAAAAAGAGATTACTTGC |
| 51 | TGFBR2_37 | TTATGTAAAAGACAAACAATGC |
| 52 | TGFBR2_30 | TTAAATGAAAAGTCCAGGAGAA |
| 53 | TGFBR2_16 | TTGATTAACAAGTTTCCTATGG |
| 54 | TGFBR2_48 | TTTAATTCCTAGACTGGGGTCC |
| 55 | TGFBR2_22 | TCAAAATCATATTATATACCCA |
| 56 | TGFBR2_45 | TATATATAGAGCTATAGACATA |
| 57 | TGFBR2_40 | TGTAATGAAAAAGTAAACACGA |
| 58 | TGFBR2_46 | TTCTAAACTAGGTTGAGGGAGA |
| 59 | TGFBR2_13 | TATTCTAGAAACTCACCACTAG |
| 60 | TGFBR2_38 | TTGAAATCCTCTTCATCAGGCC |
| 61 | TGFBR2_17 | TGAGAATACTCTTGAATCTTGA |
| 62 | TGFBR2_36 | TTGAATATCTCATGAATGGACC |
| 63 | TGFBR2_27 | TTTAAAAAAGAGATTACTTGCA |
| 64 | TGFBR2_24 | TTCACTTTTTGAAACGCTGTGC |
| 65 | TGFBR2_39 | TAATCTTTTACTTCTCCCACTG |
| 66 | TGFBR2_20 | TATATATAAAACATAGCTATTC |
| 67 | TGFBR2_21 | TATATATATAAAACATAGCTAT |
| 68 | TGFBR2_10 | TTGTGGTTGATGTTGTTGGCAC |
| 69 | TGFBR2_26 | TAGAATAAAAATAAGTGCTTGA |
| 70 | TGFBR2_44 | TAATGTTTACAGTGAGAAGTGA |
| 71 | TGFBR2_33 | TAGGGAAAGATCTTGACTGCCA |
| 72 | TGFBR2_31 | TAGAGTTTCTAAACTAGGTTGA |
| 73 | TGFBR2_42 | TTTTAGAAAAGGATGAAGGCTG |
| 74 | TGFBR2_47 | TTTAGAAAAGGATGAAGGCTGG |
| 75 | TGFBR2_35 | ATAGAATAAAAATAAGTGCTTG |
| 76 | TGFBR2_19 | TCTTGAATCTTGAATATCTCAT |
| 77 | TGFBR2_12 | TTTCATTAGTAGATGGTGGGGC |
| 78 | TGFBR2_43 | TTAGAAAAGGATGAAGGCTGGG |
| 79 | TGFBR2_41 | ATATATATATAAAACATAGCTA |
| 80 | TGFBR2_29 | TTATATTCTTCTGAGAAGATGA |
| 81 | TGFBR2_23 | TAAAAATCAATCTCATTTCCTG |
| 82 | TGFBR2_18 | TAATAAACATTTTATTTATGTA |
| 83 | TGFBR2_15 | TGATTAACAAGTTTCCTATGGA |
| 84 | TGFBR2_28 | TTAAAAATCTCAATAAAATGAG |
| 85 | FAS_1 guide | TAGATTTTAAACATCCTTGGAG |
| 86 | FAS_1 guide | TAGATTTTAAACATCCTTGGAG |
| 87 | FAS_6 guide | TTACTCTTGATGAAGTTTGTTT |
| 88 | FAS_7 guide | TTGAACTTTCTGTTCTGCTGTG |
| 89 | FAS_8 guide | TTGTCTGTGTACTCCTTCCCTT |
| 90 | FAS_9 guide | TCTTTGATTGCAAACATGGGTT |
| 91 | FAS_10 guide | TTGATCTCATCTATTTTGGCTT |
| 92 | FAS_11 guide | TTAAGAATCTTTTCAAACACTA |
| 93 | FAS_12 guide | TTCTATTGATTCATAGAGTGGG |
| 94 | FAS_13 guide | TAATCTTAATCTTTCATCCTCT |
| 95 | FAS_14 guide | TTAACTTGACTTAGTGTCATGA |
| 96 | FAS_15 guide | TTACATAAATATGATCTTCTCA |
| 97 | FAS_16 guide | TACATAAATATGATCTTCTCAG |
| 98 | FAS_18 guide | TAAAAATCTACAAATATGTTGG |
| 99 | FAS_19 guide | TTTGGTTTACATCTGCACTTGG |
| 100 | PTPN2_1 guide | TATAATACGACTTCACATCTTC |
| 101 | PTPN2_2 guide | TAGAAAGTTCTTTCCATCGTTT |
| 102 | PTPN2_4 guide | TTCTATGTCAACTAAACTGGCA |
| 103 | PTPN2_5 guide | TTAAACAGCATCTCTTGGTCAT |
| 104 | PTPN2_7 guide | TCGAATTTCCAAGTACAGCGGC |
| 105 | PTPN2_8 guide | TTAGAAAGTTCTTTCCATCGTT |
| 106 | PTPN2_9 guide | TAGATGTACTGTATAATACGAC |
| 107 | PTPN2_10 | TCTGTATACTAGAATCTCCCTT |
| | guide | |
| 108 | PTPN2_11 | TTTTATGTTAATATCATCTCCT |
| | guide | |
| 109 | PTPN2_13 | TGAGAATCTCAGTTGATCTGGG |
| | guide | |
| 110 | PTPN2_14 | TCTGACAAGAGCTTCACACTGA |
| | guide | |
| 111 | PTPN2_15 | TTCTATTATAGCCATGTATGAG |
| | guide | |
| 112 | PTPN2_16 | TGATATTTTCTAATTGTAGTAG |
| | guide | |
| 113 | TOX_2 guide | TAAAGTATTCACACCAAGCGTG |
| 114 | TOX_4 guide | TATGACTGCTACATCAAGCCAT |
| 115 | TOX_5 guide | TTAAATGACATTTACTAACCAT |
| 116 | TOX_6 guide | TTAAATTAAAATCACTTGGGAA |
| 117 | TOX_7 guide | TTTGCTCTTCTCCTAAACCGTC |
| 118 | TOX_8 guide | TTAGTTAATTAGTAGTGCTGGT |
| 119 | TOX_9 guide | TAGGTGAGGATTCATTCCCGGT |
| 120 | TOX_10 guide | TTAGTCTTGGAGCTTGTGTGAC |
| 121 | TOX_11 guide | TTTAAATTAAAATCACTTGGGA |
| 122 | TOX_12 guide | TTTTAAATTAAAATCACTTGGG |
| 123 | TOX_13 guide | TTCAATTACAATTTAGCTACAA |
| 124 | TOX_15 guide | TTTATTATTTCATAAATAGGCG |
| 125 | TOX_16 guide | TTACAAACTTGCTATGACTGCT |
| 126 | TOX_17 guide | TATTATTTCATAAATAGGCGCA |
| 127 | FAS_11 miR3G | GTAAGTCGACTCGTTGGATCCCCACTACCCGGATCAACGCCCTAG |
| | -PTPN2_14 | GTTTATGTTTGGATGAACTGACATACGCGTATCCGTCTTAAGAAT |
| | miRE Module | CTTTTCAAACACTAGTAGTGAAATATATATTAAACTAGTGTTTGA |
| | | AAAGATTCTTATTACGGTAACGCGGAATTCGCAACTATTTTATCA |
| | | ATTTTTTGCGTCGACACTTCAAGGGGCTTGCGGCCGCAACCATCT |
| | | CCATGGCTGTTTGAATGAGGCTTCAGTACTTTACAGAATCGTTGC |
| | | CTGCACATCTTGGAAACACTTGCTGGGATTACTTCGACTTCTTAA |
| | | CCCAACAGAAGGCTCGAGAAGGTATATTGCTGTTGACAGTGAGCG |
| | | CCAGTGTGAAGCTCTTGTCAGATAGTGAAGCCACAGATGTATCTG |
| | | ACAAGAGCTTCACACTGATGCCTACTGCCTCGGACTTCAAGGGGC |
| | | TAGAATTCGAGCAATTATCTTGTTTACTAAAACTGAATACCTTGC |
| | | TATCTCTTTGATACATTTTTACAAAGCTGAATTAAAATGGTATAA |
| | | ATTAAATCACTTTTTCATCTGACCAGTAGTGGACTAGTGTGACGC |
| | | TGCTGACCCCTTTCTTTCCCTTCTACAG |
| 128 | TGFBR2_23- | CCGGATCAACGCCCTAGGTTTATGTTTGGATGAACTGACATACGC |
| | TGBR2_37 | GTATCCGTCTAAAAATCAATCTCATTTCCTGGTAGTGAAATATAT |
| | miR3G Module | ATTAAACCAGGAAATGAGATTGATTTTTTTACGGTAACGCGGAAT |
| | | TCGCAACTATTTTATCAATTTTTTGCGTCGACGACTGTGACAGCA |
| | | GAGTATGCCGGATCAACGCCCTAGGTTTATGTTTGGATGAACTGA |
| | | CATACGCGTATCCGTCTTATGTAAAAGACAAACAATGCGTAGTGA |
| | | AATATATATTAAACGCATTGTTTGTCTTTTACATATTACGGTAAC |
| | | GCGGAATTCGCAACTATTTTATCAATTTTTTGCGTCGAC |
| 129 | FAS-PTPN2- | CCACTACCCGGATCAACGCCCTAGGTTTATGTTTGGATGAACTGA |
| | TGFBR2_23- | CATACGCGTATCCGTCTTAAGAATCTTTTCAAACACTAGTAGTGA |
| | TGBR2_37 | AATATATATTAAACTAGTGTTTGAAAAGATTCTTATTACGGTAAC |
| | miR3G Module | GCGGAATTCGCAACTATTTTATCAATTTTTTGCGTCGACACTTCA |
| | | AGGGGCTTGCGGCCGCAACCATCTCCATGGCTGTTTGAATGAGGC |
| | | TTCAGTACTTTACAGAATCGTTGCCTGCACATCTTGGAAACACTT |
| | | GCTGGGATTACTTCGACTTCTTAACCCAACAGAAGGCTCGAGAAG |
| | | GTATATTGCTGTTGACAGTGAGCGCCAGTGTGAAGCTCTTGTCAG |
| | | ATAGTGAAGCCACAGATGTATCTGACAAGAGCTTCACACTGATGC |
| | | CTACTGCCTCGGACTTCAAGGGGCTAGAATTCGAGCAATTATCTT |
| | | GTTTACTAAAACTGAATACCTTGCTATCTCTTTGATACATTTTTA |
| | | CAAAGCTGAATTAAAATGGTATAAATTAAATCACTTTGACGGTGA |
| | | CTTAGGAGTATGCCGGATCAACGCCCTAGGTTTATGTTTGGATGA |
| | | ACTGACATACGCGTATCCGTCTAAAAATCAATCTCATTTCCTGGT |
| | | AGTGAAATATATATTAAACCAGGAAATGAGATTGATTTTTTTACG |
| | | GTAACGCGGAATTCGCAACTATTTTATCAATTTTTTGCGTCGACG |
| | | ACTGTGACAGCAGAGTATGCCGGATCAACGCCCTAGGTTTATGTT |
| | | TGGATGAACTGACATACGCGTATCCGTCTTATGTAAAAGACAAAC |
| | | AATGCGTAGTGAAATATATATTAAACGCATTGTTTGTCTTTTACA |
| | | TATTACGGTAACGCGGAATTCGCAACTATTTTATCAATTTTTTGC |
| | | GTCGAC |
| 130 | FAS/TGFBR2/ | gtaagtcgactcgttggatccccactacccggatcaacgccctag |
| | TGFBR2 | gtttatgtttggatgaactgacatacgcgtatccgtcttaagaat |
| | shRNA cassette | cttttcaaacactagtagtgaaatatatattaaactagtgtttga |
| | | aaagattcttattacggtaacgcggaattcgcaactattttatca |
| | | attttttgcgtcgacgacggtgacttaggagtatgccggatcaac |
| | | gccctaggtttatgtttggatgaactgacatacgcgtatccgtct |
| | | aaaaatcaatctcatttcctggtagtgaaatatatattaaaccag |
| | | gaaatgagattgatttttttacggtaacgcggaattcgcaactat |
| | | tttatcaattttttgcgtcgacgactgtgacagcagagtatgccg |
| | | gatcaacgccctaggtttatgtttggatgaactgacatacgcgta |
| | | tccgtcttatgtaaaagacaaacaatgcgtagtgaaatatatatt |
| | | aaacgcattgtttgtcttttacatattacggtaacgcggaattcg |
| | | caactattttatcaattttttgcgtcgaccggaactatcttgaag |
| | | agtagtagtggactagtgtgacgctgctgacccctttctttccct |
| | | tctacaga |
| 131 | FAS/TGFBR2/ | gtaaGTcgactcgttggatccCCACTACCCGGATCAACGCCCTAG |
| | TGFBR2/PTPN | GTTTATGTTTGGATGAACTGACATACGCGTATCCGTCTTAAGAAT |
| | 2 shRNA | CTTTTCAAACACTAGTAGTGAAATATATATTAAACTAGTGTTTGA |
| | cassette | AAAGATTCTTATTACGGTAACGCGGAATTCGCAACTATTTTATCA |
| | | ATTTTTTGCGTCGACACTTCAAGGGGCTTGCGGCCGCAACCATCT |
| | | CCATGGCTGTTTGAATGAGGCTTCAGTACTTTACAGAATCGTTGC |
| | | CTGCACATCTTGGAAACACTTGCTGGGATTACTTCGACTTCTTAA |
| | | CCCAACAGAAGGCTCGAGAAGGTATATTGCTGTTGACAGTGAGCG |
| | | CCAGTGTGAAGCTCTTGTCAGATAGTGAAGCCACAGATGTATCTG |
| | | ACAAGAGCTTCACACTGATGCCTACTGCCTCGGACTTCAAGGGGC |
| | | TAGAATTCGAGCAATTATCTTGTTTACTAAAACTGAATACCTTGC |
| | | TATCTCTTTGATACATTTTTACAAAGCTGAATTAAAATGGTATAA |
| | | ATTAAATCACTTTGACGGTGACTTAGGAGTATGCCGGATCAACGC |
| | | CCTAGGTTTATGTTTGGATGAACTGACATACGCGTATCCGTCTAA |
| | | AAATCAATCTCATTTCCTGGTAGTGAAATATATATTAAACCAGGA |
| | | AATGAGATTGATTTTTTTACGGTAACGCGGAATTCGCAACTATTT |
| | | TATCAATTTTTTGCGTCGACGACTGTGACAGCAGAGTATGCCGGA |
| | | TCAACGCCCTAGGTTTATGTTTGGATGAACTGACATACGCGTATC |
| | | CGTCTTATGTAAAAGACAAACAATGCGTAGTGAAATATATATTAA |
| | | ACGCATTGTTTGTCTTTTACATATTACGGTAACGCGGAATTCGCA |
| | | ACTATTTTATCAATTTTTTGCGTCGACCGGAACTATCTTGAAGAG |
| | | TAGTAGTGGactagtgtgacgctgctgacccctttctttcccttc |
| | | tACAG |
| 132 | EF1A promoter | GGATCTGCGATCGCTCCGGTGCCCGTCAGTGGGCAGAGCGCACAT |
| | | CGCCCACAGTCCCCGAGAAGTTGGGGGGAGGGGTCGGCAATTGAA |
| | | CGGGTGCCTAGAGAAGGTGGCGCGGGGTAAACTGGGAAAGTGATG |
| | | TCGTGTACTGGCTCCGCCTTTTTCCCGAGGGTGGGGGAGAACCGT |
| | | ATATAAGTGCAGTAGTCGCCGTGAACGTTCTTTTTCGCAACGGGT |
| | | TTGCCGCCAGAACACAGCTGAAGCTTCGAGGGGCTCGCATCTCTC |
| | | CTTCACGCGCCCGCCGCCCTACCTGAGGCCGCCATCCACGCCGGT |
| | | TGAGTCGCGTTCTGCCGCCTCCCGCCTGTGGTGCCTCCTGAACTG |
| | | CGTCCGCCGTCTAG |
| 133 | FAS-PTPN2 | GGATCTGCGATCGCTCCGGTGCCCGTCAGTGGGCAGAGCGCACAT |
| | dual shRNA | CGCCCACAGTCCCCGAGAAGTTGGGGGGAGGGGTCGGCAATTGAA |
| | cassette | CGGGTGCCTAGAGAAGGTGGCGCGGGGTAAACTGGGAAAGTGATG |
| | FAS-TGFBR2- | TCGTGTACTGGCTCCGCCTTTTTCCCGAGGGTGGGGGAGAACCGT |
| | TGFBR2 | ATATAAGTGCAGTAGTCGCCGTGAACGTTCTTTTTCGCAACGGGT |
| | architecture 1 | TTGCCGCCAGAACACAGCTGAAGCTTCGAGGGGCTCGCATCTCTC |
| | (3G-3G-3G) | CTTCACGCGCCCGCCGCCCTACCTGAGGCCGCCATCCACGCCGGT |
| | triple shRNA | TGAGTCGCGTTCTGCCGCCTCCCGCCTGTGGTGCCTCCTGAACTG |
| | cassette | CGTCCGCCGTCTAGGTAAGTCGACTCGTTGGATCCCCACTACCCG |
| | | GATCAACGCCCTAGGTTTATGTTTGGATGAACTGACATACGCGTA |
| | | TCCGTCTTAAGAATCTTTTCAAACACTAGTAGTGAAATATATATT |
| | | AAACTAGTGTTTGAAAAGATTCTTATTACGGTAACGCGGAATTCG |
| | | CAACTATTTTATCAATTTTTTGCGTCGACACTTCAAGGGGCTTGC |
| | | GGCCGCAACCATCTCCATGGCTGTTTGAATGAGGCTTCAGTACTT |
| | | TACAGAATCGTTGCCTGCACATCTTGGAAACACTTGCTGGGATTA |
| | | CTTCGACTTCTTAACCCAACAGAAGGCTCGAGAAGGTATATTGCT |
| | | GTTGACAGTGAGCGCCAGTGTGAAGCTCTTGTCAGATAGTGAAGC |
| | | CACAGATGTATCTGACAAGAGCTTCACACTGATGCCTACTGCCTC |
| | | GGACTTCAAGGGGCTAGAATTCGAGCAATTATCTTGTTTACTAAA |
| | | ACTGAATACCTTGCTATCTCTTTGATACATTTTTACAAAGCTGAA |
| | | TTAAAATGGTATAAATTAAATCACTTTTTCATCTGACCAGTAGTG |
| | | GACTAGTGTGACGCTGCTGACCCCTTTCTTTCCCTTCTACAGATC |
| | | CAAGCTGTGACCGGCGCCTACACCTGCAGCCCAAGCTTACCNNNN |
| | | NNNNNNNNNNNNNNNNNNNNNNNNNNTAAAGGACGGGTGGCATCC |
| | | CTGTGACCCCTCCCCAGTGCCTCTCCTGGCCCTGGAAGTTGCCAC |
| | | TCCAGTGCCCACCAGCCTTGTCCTAATAAAATTAAGTTGCATCAT |
| | | TTTGTCTGACTAGGTGTCCTTCTATAATATTATGGGGTGGAGGGG |
| | | GGTGGTATGGAGCAAGGGGCAAGTTGGGAAGACAACCTGTAGGGC |
| | | CTGCGGGGTCTATTGGGAACCAAGCTGGAGTGCAGTGGCACAATC |
| | | TTGGCTCACTGCAATCTCCGCCTCCTGGGTTCAAGCGATTCTCCT |
| | | GCCTCAGCCTCCCGAGTTGTTGGGATTCCAGGCATGCATGACCAG |
| | | GCTCAGCTAATTTTTGTTTTTTTGGTAGAAACGGGGTTTCACCAT |
| | | ATTGGCCAGGCTGGTCTCCAACTCCTAATCTCAGGTGATCTACCC |
| | | ACCTTGGCCTCCCAAATTGCTGGGATTACAGGCGTGAACCACTGC |
| | | TCCCTTCCCTGTCCTTC |
| | | (NNNNNNNNNNNNNNNNNNNNNNNNNNNNNN can be absent |
| | | or can encode a transgene of any length) |
| 134 | | GGATCTGCGATCGCTCCGGTGCCCGTCAGTGGGCAGAGCGCACAT |
| | | CGCCCACAGTCCCCGAGAAGTTGGGGGGAGGGGTCGGCAATTGAA |
| | | CGGGTGCCTAGAGAAGGTGGCGCGGGGTAAACTGGGAAAGTGATG |
| | | TCGTGTACTGGCTCCGCCTTTTTCCCGAGGGTGGGGGAGAACCGT |
| | | ATATAAGTGCAGTAGTCGCCGTGAACGTTCTTTTTCGCAACGGGT |
| | | TTGCCGCCAGAACACAGCTGAAGCTTCGAGGGGCTCGCATCTCTC |
| | | CTTCACGCGCCCGCCGCCCTACCTGAGGCCGCCATCCACGCCGGT |
| | | TGAGTCGCGTTCTGCCGCCTCCCGCCTGTGGTGCCTCCTGAACTG |
| | | CGTCCGCCGTCTAGGTAAGTCGACTCGTTGGATCCCCACTACCCG |
| | | GATCAACGCCCTAGGTTTATGTTTGGATGAACTGACATACGCGTA |
| | | TCCGTCTTAAGAATCTTTTCAAACACTAGTAGTGAAATATATATT |
| | | AAACTAGTGTTTGAAAAGATTCTTATTACGGTAACGCGGAATTCG |
| | | CAACTATTTTATCAATTTTTTGCGTCGACGACGGTGACTTAGGAG |
| | | TATGCCGGATCAACGCCCTAGGTTTATGTTTGGATGAACTGACAT |
| | | ACGCGTATCCGTCTAAAAATCAATCTCATTTCCTGGTAGTGAAAT |
| | | ATATATTAAACCAGGAAATGAGATTGATTTTTTTACGGTAACGCG |
| | | GAATTCGCAACTATTTTATCAATTTTTTGCGTCGACGACTGTGAC |
| | | AGCAGAGTATGCCGGATCAACGCCCTAGGTTTATGTTTGGATGAA |
| | | CTGACATACGCGTATCCGTCTTATGTAAAAGACAAACAATGCGTA |
| | | GTGAAATATATATTAAACGCATTGTTTGTCTTTTACATATTACGG |
| | | TAACGCGGAATTCGCAACTATTTTATCAATTTTTTGCGTCGACCG |
| | | GAACTATCTTGAAGAGTAGTAGTGGACTAGTGTGACGCTGCTGAC |
| | | CCCTTTCTTTCCCTTCTACAGATCCAAGCTGTGACCGGCGCCTAC |
| | | ACCTGCAGCCCAAGCTTACCNNNNNNNNNNNNNNNNNNNNNNNNN |
| | | NNNNNTAAAGGACGGGTGGCATCCCTGTGACCCCTCCCCAGTGCC |
| | | TCTCCTGGCCCTGGAAGTTGCCACTCCAGTGCCCACCAGCCTTGT |
| | | CCTAATAAAATTAAGTTGCATCATTTTGTCTGACTAGGTGTCCTT |
| | | CTATAATATTATGGGGTGGAGGGGGGTGGTATGGAGCAAGGGGCA |
| | | AGTTGGGAAGACAACCTGTAGGGCCTGCGGGGTCTATTGGGAACC |
| | | AAGCTGGAGTGCAGTGGCACAATCTTGGCTCACTGCAATCTCCGC |
| | | CTCCTGGGTTCAAGCGATTCTCCTGCCTCAGCCTCCCGAGTTGTT |
| | | GGGATTCCAGGCATGCATGACCAGGCTCAGCTAATTTTTGTTTTT |
| | | TTGGTAGAAACGGGGTTTCACCATATTGGCCAGGCTGGTCTCCAA |
| | | CTCCTAATCTCAGGTGATCTACCCACCTTGGCCTCCCAAATTGCT |
| | | GGGATTACAGGCGTGAACCACTGCTCCCTTCCCTGTCCTTC |
| | | (NNNNNNNNNNNNNNNNNNNNNNNNNNNNNN can be absent |
| | | or can encode a transgene of any length) |
| 135 | TGFBR2- | GGATCTGCGATCGCTCCGGTGCCCGTCAGTGGGCAGAGCGCACAT |
| | TGFBR2-FAS | CGCCCACAGTCCCCGAGAAGTTGGGGGGAGGGGTCGGCAATTGAA |
| | architecture 2 | CGGGTGCCTAGAGAAGGTGGCGCGGGGTAAACTGGGAAAGTGATG |
| | triple shRNA | TCGTGTACTGGCTCCGCCTTTTTCCCGAGGGTGGGGGAGAACCGT |
| | cassette | ATATAAGTGCAGTAGTCGCCGTGAACGTTCTTTTTCGCAACGGGT |
| | | TTGCCGCCAGAACACAGCTGAAGCTTCGAGGGGCTCGCATCTCTC |
| | | CTTCACGCGCCCGCCGCCCTACCTGAGGCCGCCATCCACGCCGGT |
| | | TGAGTCGCGTTCTGCCGCCTCCCGCCTGTGGTGCCTCCTGAACTG |
| | | CGTCCGCCGTCTAGGTAAGTCGACTCGTTGGATCCCCACTACCCG |
| | | GATCAACGCCCTAGGTTTATGTTTGGATGAACTGACATACGCGTA |
| | | TCCGTCTAAAAATCAATCTCATTTCCTGGTAGTGAAATATATATT |
| | | AAACCAGGAAATGAGATTGATTTTTTTACGGTAACGCGGAATTCG |
| | | CAACTATTTTATCAATTTTTTGCGTCGACACTTCAAGGGGCTTGC |
| | | GGCCGCAACCATCTCCATGGCGACGGTGACTTAGGAGTATGCCGG |
| | | ATCAACGCCCTAGGTTTATGTTTGGATGAACTGACATACGCGTAT |
| | | CCGTCTTATGTAAAAGACAAACAATGCGTAGTGAAATATATATTA |
| | | AACGCATTGTTTGTCTTTTACATATTACGGTAACGCGGAATTCGC |
| | | AACTATTTTATCAATTTTTTGCGTCGACGACTGTGACAGCAGAGT |
| | | ATGCCGGATCAACGCCCTAGGTTTATGTTTGGATGAACTGACATA |
| | | CGCGTATCCGTCTTAAGAATCTTTTCAAACACTAGTAGTGAAATA |
| | | TATATTAAACTAGTGTTTGAAAAGATTCTTATTACGGTAACGCGG |
| | | AATTCGCAACTATTTTATCAATTTTTTGCGTCGACCGGAACTATC |
| | | TTGAAGAGTAGTAGTGGACTAGTGTGACGCTGCTGACCCCTTTCT |
| | | TTCCCTTCTACAGATCCAAGCTGTGACCGGCGCCTACACCTGCAG |
| | | CCCAAGCTTACCNNNNNNNNNNNNNNNNNNNNNNNNNNNNNNTAA |
| | | AGGACGGGTGGCATCCCTGTGACCCCTCCCCAGTGCCTCTCCTGG |
| | | CCCTGGAAGTTGCCACTCCAGTGCCCACCAGCCTTGTCCTAATAA |
| | | AATTAAGTTGCATCATTTTGTCTGACTAGGTGTCCTTCTATAATA |
| | | TTATGGGGTGGAGGGGGGTGGTATGGAGCAAGGGGCAAGTTGGGA |
| | | AGACAACCTGTAGGGCCTGCGGGGTCTATTGGGAACCAAGCTGGA |
| | | GTGCAGTGGCACAATCTTGGCTCACTGCAATCTCCGCCTCCTGGG |
| | | TTCAAGCGATTCTCCTGCCTCAGCCTCCCGAGTTGTTGGGATTCC |
| | | AGGCATGCATGACCAGGCTCAGCTAATTTTTGTTTTTTTGGTAGA |
| | | AACGGGGTTTCACCATATTGGCCAGGCTGGTCTCCAACTCCTAAT |
| | | CTCAGGTGATCTACCCACCTTGGCCTCCCAAATTGCTGGGATTAC |
| | | AGGCGTGAACCACTGCTCCCTTCCCTGTCCTTC |
| | | (NNNNNNNNNNNNNNNNNNNNNNNNNNNNNN can be absent |
| | | or can encode a transgene of any length) |
| 136 | FAS-TGFBR2- | GGATCTGCGATCGCTCCGGTGCCCGTCAGTGGGCAGAGCGCACAT |
| | TGFBR2 | CGCCCACAGTCCCCGAGAAGTTGGGGGGAGGGGTCGGCAATTGAA |
| | architecture 3 | CGGGTGCCTAGAGAAGGTGGCGCGGGGTAAACTGGGAAAGTGATG |
| | (3G-E-3G) | TCGTGTACTGGCTCCGCCTTTTTCCCGAGGGTGGGGGAGAACCGT |
| | triple shRNA | ATATAAGTGCAGTAGTCGCCGTGAACGTTCTTTTTCGCAACGGGT |
| | cassette | TTGCCGCCAGAACACAGCTGAAGCTTCGAGGGGCTCGCATCTCTC |
| | | CTTCACGCGCCCGCCGCCCTACCTGAGGCCGCCATCCACGCCGGT |
| | | TGAGTCGCGTTCTGCCGCCTCCCGCCTGTGGTGCCTCCTGAACTG |
| | | CGTCCGCCGTCTAGGTAAGTCGACTCGTTGGATCCCCACTACCCG |
| | | GATCAACGCCCTAGGTTTATGTTTGGATGAACTGACATACGCGTA |
| | | TCCGTCTTAAGAATCTTTTCAAACACTAGTAGTGAAATATATATT |
| | | AAACTAGTGTTTGAAAAGATTCTTATTACGGTAACGCGGAATTCG |
| | | CAACTATTTTATCAATTTTTTGCGTCGACACTTCAAGGGGCTTGC |
| | | GGCCGCAACCATCTCCATGGCGACGGTGACTTAGGAGTATGTGTT |
| | | TGAATGAGGCTTCAGTACTTTACAGAATCGTTGCCTGCACATCTT |
| | | GGAAACACTTGCTGGGATTACTTCGACTTCTTAACCCAACAGAAG |
| | | GCTCGAGAAGGTATATTGCTGTTGACAGTGAGCGCAGGAAATGAG |
| | | ATTGATTTTTTTAGTGAAGCCACAGATGTATAAAAATCAATCTCA |
| | | TTTCCTGTGCCTACTGCCTCGGACTTCAAGGGGCTAGAATTCGAG |
| | | CAATTATCTTGTTTACTAAAACTGAATACCTTGCTATCTCTTTGA |
| | | TACATTTTTACAAAGCTGAATTAAAATGGTATAAATTAAATCACT |
| | | TTGACTGTGACAGCAGAGTATGCCGGATCAACGCCCTAGGTTTAT |
| | | GTTTGGATGAACTGACATACGCGTATCCGTCTTATGTAAAAGACA |
| | | AACAATGCGTAGTGAAATATATATTAAACGCATTGTTTGTCTTTT |
| | | ACATATTACGGTAACGCGGAATTCGCAACTATTTTATCAATTTTT |
| | | TGCGTCGACCGGAACTATCTTGAAGAGTAGTAGTGGACTAGTGTG |
| | | ACGCTGCTGACCCCTTTCTTTCCCTTCTACAGATCCAAGCTGTGA |
| | | CCGGCGCCTACACCTGCAGCCCAAGCTTACCNNNNNNNNNNNNNN |
| | | NNNNNNNNNNNNNNNNTAAAGGACGGGTGGCATCCCTGTGACCCC |
| | | TCCCCAGTGCCTCTCCTGGCCCTGGAAGTTGCCACTCCAGTGCCC |
| | | ACCAGCCTTGTCCTAATAAAATTAAGTTGCATCATTTTGTCTGAC |
| | | TAGGTGTCCTTCTATAATATTATGGGGTGGAGGGGGGTGGTATGG |
| | | AGCAAGGGGCAAGTTGGGAAGACAACCTGTAGGGCCTGCGGGGTC |
| | | TATTGGGAACCAAGCTGGAGTGCAGTGGCACAATCTTGGCTCACT |
| | | GCAATCTCCGCCTCCTGGGTTCAAGCGATTCTCCTGCCTCAGCCT |
| | | CCCGAGTTGTTGGGATTCCAGGCATGCATGACCAGGCTCAGCTAA |
| | | TTTTTGTTTTTTTGGTAGAAACGGGGTTTCACCATATTGGCCAGG |
| | | CTGGTCTCCAACTCCTAATCTCAGGTGATCTACCCACCTTGGCCT |
| | | CCCAAATTGCTGGGATTACAGGCGTGAACCACTGCTCCCTTCCCT |
| | | GTCCTTC |
| | | (NNNNNNNNNNNNNNNNNNNNNNNNNNNNNN can be absent |
| | | or can encode a transgene of any length) |
| 137 | FAS-PTPN2- | GGATCTGCGATCGCTCCGGTGCCCGTCAGTGGGCAGAGCGCACAT |
| | TGFBR2- | CGCCCACAGTCCCCGAGAAGTTGGGGGGAGGGGTCGGCAATTGAA |
| | TGFBR2 | CGGGTGCCTAGAGAAGGTGGCGCGGGGTAAACTGGGAAAGTGATG |
| | quadruple | TCGTGTACTGGCTCCGCCTTTTTCCCGAGGGTGGGGGAGAACCGT |
| | shRNA cassette | ATATAAGTGCAGTAGTCGCCGTGAACGTTCTTTTTCGCAACGGGT |
| | | TTGCCGCCAGAACACAGCTGAAGCTTCGAGGGGCTCGCATCTCTC |
| | | CTTCACGCGCCCGCCGCCCTACCTGAGGCCGCCATCCACGCCGGT |
| | | TGAGTCGCGTTCTGCCGCCTCCCGCCTGTGGTGCCTCCTGAACTG |
| | | CGTCCGCCGTCTAGGTAAGTCGACTCGTTGGATCCCCACTACCCG |
| | | GATCAACGCCCTAGGTTTATGTTTGGATGAACTGACATACGCGTA |
| | | TCCGTCTTAAGAATCTTTTCAAACACTAGTAGTGAAATATATATT |
| | | AAACTAGTGTTTGAAAAGATTCTTATTACGGTAACGCGGAATTCG |
| | | CAACTATTTTATCAATTTTTTGCGTCGACACTTCAAGGGGCTTGC |
| | | GGCCGCAACCATCTCCATGGCTGTTTGAATGAGGCTTCAGTACTT |
| | | TACAGAATCGTTGCCTGCACATCTTGGAAACACTTGCTGGGATTA |
| | | CTTCGACTTCTTAACCCAACAGAAGGCTCGAGAAGGTATATTGCT |
| | | GTTGACAGTGAGCGCCAGTGTGAAGCTCTTGTCAGATAGTGAAGC |
| | | CACAGATGTATCTGACAAGAGCTTCACACTGATGCCTACTGCCTC |
| | | GGACTTCAAGGGGCTAGAATTCGAGCAATTATCTTGTTTACTAAA |
| | | ACTGAATACCTTGCTATCTCTTTGATACATTTTTACAAAGCTGAA |
| | | TTAAAATGGTATAAATTAAATCACTTTGACGGTGACTTAGGAGTA |
| | | TGCCGGATCAACGCCCTAGGTTTATGTTTGGATGAACTGACATAC |
| | | GCGTATCCGTCTAAAAATCAATCTCATTTCCTGGTAGTGAAATAT |
| | | ATATTAAACCAGGAAATGAGATTGATTTTTTTACGGTAACGCGGA |
| | | ATTCGCAACTATTTTATCAATTTTTTGCGTCGACGACTGTGACAG |
| | | CAGAGTATGCCGGATCAACGCCCTAGGTTTATGTTTGGATGAACT |
| | | GACATACGCGTATCCGTCTTATGTAAAAGACAAACAATGCGTAGT |
| | | GAAATATATATTAAACGCATTGTTTGTCTTTTACATATTACGGTA |
| | | ACGCGGAATTCGCAACTATTTTATCAATTTTTTGCGTCGACCGGA |
| | | ACTATCTTGAAGAGTAGTAGTGGACTAGTGTGACGCTGCTGACCC |
| | | CTTTCTTTCCCTTCTACAGATCCAAGCTGTGACCGGCGCCTACAC |
| | | CTGCAGCCCAAGCTTACCNNNNNNNNNNNNNNNNNNNNNNNNNNN |
| | | NNNTAAAGGACGGGTGGCATCCCTGTGACCCCTCCCCAGTGCCTC |
| | | TCCTGGCCCTGGAAGTTGCCACTCCAGTGCCCACCAGCCTTGTCC |
| | | TAATAAAATTAAGTTGCATCATTTTGTCTGACTAGGTGTCCTTCT |
| | | ATAATATTATGGGGTGGAGGGGGGTGGTATGGAGCAAGGGGCAAG |
| | | TTGGGAAGACAACCTGTAGGGCCTGCGGGGTCTATTGGGAACCAA |
| | | GCTGGAGTGCAGTGGCACAATCTTGGCTCACTGCAATCTCCGCCT |
| | | CCTGGGTTCAAGCGATTCTCCTGCCTCAGCCTCCCGAGTTGTTGG |
| | | GATTCCAGGCATGCATGACCAGGCTCAGCTAATTTTTGTTTTTTT |
| | | GGTAGAAACGGGGTTTCACCATATTGGCCAGGCTGGTCTCCAACT |
| | | CCTAATCTCAGGTGATCTACCCACCTTGGCCTCCCAAATTGCTGG |
| | | GATTACAGGCGTGAACCACTGCTCCCTTCCCTGTCCTTC |
| | | (NNNNNNNNNNNNNNNNNNNNNNNNNNNNNN can be absent |
| | | or can encode a transgene of any length) |
| 138 | human growth | CGGGTGGCATCCCTGTGACCCCTCCCCAGTGCCTCTCCTGGCCCT |
| | hormone (GH1) | GGAAGTTGCCACTCCAGTGCCCACCAGCCTTGTCCTAATAAAATT |
| | polyadenylation | AAGTTGCATCATTTTGTCTGACTAGGTGTCCTTCTATAATATTAT |
| | signal | GGGGTGGAGGGGGGTGGTATGGAGCAAGGGGCAAGTTGGGAAGAC |
| | | AACCTGTAGGGCCTGCGGGGTCTATTGGGAACCAAGCTGGAGTGC |
| | | AGTGGCACAATCTTGGCTCACTGCAATCTCCGCCTCCTGGGTTCA |
| | | AGCGATTCTCCTGCCTCAGCCTCCCGAGTTGTTGGGATTCCAGGC |
| | | ATGCATGACCAGGCTCAGCTAATTTTTGTTTTTTTGGTAGAAACG |
| | | GGGTTTCACCATATTGGCCAGGCTGGTCTCCAACTCCTAATCTCA |
| | | GGTGATCTACCCACCTTGGCCTCCCAAATTGCTGGGATTACAGGC |
| | | GTGAACCACTGCTCCCTTCCCTGTCCTTC |
| 139 | FAS-TGFBR2- | CGGATCAACGCCCTAGGTTTATGTTTGGATGAACTGACATACGCG |
| | TGFBR2 (3G- | TATCCGTCTTAAGAATCTTTTCAAACACTAGTAGTGAAATATATA |
| | E-3G) triple | TTAAACTAGTGTTTGAAAAGATTCTTATTACGGTAACGCGGAATT |
| | shRNA module | CGCAACTATTTTATCAATTTTTTGCGTCGACACTTCAAGGGGCTT |
| | | GCGGCCGCAACCATCTCCATGGCGACGGTGACTTAGGAGTATGTG |
| | | TTTGAATGAGGCTTCAGTACTTTACAGAATCGTTGCCTGCACATC |
| | | TTGGAAACACTTGCTGGGATTACTTCGACTTCTTAACCCAACAGA |
| | | AGGCTCGAGAAGGTATATTGCTGTTGACAGTGAGCGCAGGAAATG |
| | | AGATTGATTTTTTTAGTGAAGCCACAGATGTATAAAAATCAATCT |
| | | CATTTCCTGTGCCTACTGCCTCGGACTTCAAGGGGCTAGAATTCG |
| | | AGCAATTATCTTGTTTACTAAAACTGAATACCTTGCTATCTCTTT |
| | | GATACATTTTTACAAAGCTGAATTAAAATGGTATAAATTAAATCA |
| | | CTTTGACTGTGACAGCAGAGTATGCCGGATCAACGCCCTAGGTTT |
| | | ATGTTTGGATGAACTGACATACGCGTATCCGTCTTATGTAAAAGA |
| | | CAAACAATGCGTAGTGAAATATATATTAAACGCATTGTTTGTCTT |
| | | TTACATATTACGGTAACGCGGAATTCGCAACTATTTTATCAATTT |
| | | TTTGCGTCGAC |
| 140 | TGFBR2_23- | TGTTTGAATGAGGCTTCAGTACTTTACAGAATCGTTGCCTGCACA |
| | TGBR2_37 | TCTTGGAAACACTTGCTGGGATTACTTCGACTTCTTAACCCAACA |
| | miR3G and | GAAGGCTCGAGAAGGTATATTGCTGTTGACAGTGAGCGCAGGAAA |
| | miR3E Module | TGAGATTGATTTTTTTAGTGAAGCCACAGATGTATAAAAATCAAT |
| | | CTCATTTCCTGTGCCTACTGCCTCGGACTTCAAGGGGCTAGAATT |
| | | CGAGCAATTATCTTGTTTACTAAAACTGAATACCTTGCTATCTCT |
| | | TTGATACATTTTTACAAAGCTGAATTAAAATGGTATAAATTAAAT |
| | | CACTTTGACTGTGACAGCAGAGTATGCCGGATCAACGCCCTAGGT |
| | | TTATGTTTGGATGAACTGACATACGCGTATCCGTCTTATGTAAAA |
| | | GACAAACAATGCGTAGTGAAATATATATTAAACGCATTGTTTGTC |
| | | TTTTACATATTACGGTAACGCGGAATTCGCAACTATTTTATCAAT |
| | | TTTTTGCGTCGAC |
| 141 | TGFBR2- | CCGGATCAACGCCCTAGGTTTATGTTTGGATGAACTGACATACGC |
| | TGFBR2-FAS | GTATCCGTCTAAAAATCAATCTCATTTCCTGGTAGTGAAATATAT |
| | miR3G triple | ATTAAACCAGGAAATGAGATTGATTTTTTTACGGTAACGCGGAAT |
| | shRNA module | TCGCAACTATTTTATCAATTTTTTGCGTCGACACTTCAAGGGGCT |
| | | TGCGGCCGCAACCATCTCCATGGCGACGGTGACTTAGGAGTATGC |
| | | CGGATCAACGCCCTAGGTTTATGTTTGGATGAACTGACATACGCG |
| | | TATCCGTCTTATGTAAAAGACAAACAATGCGTAGTGAAATATATA |
| | | TTAAACGCATTGTTTGTCTTTTACATATTACGGTAACGCGGAATT |
| | | CGCAACTATTTTATCAATTTTTTGCGTCGACGACTGTGACAGCAG |
| | | AGTATGCCGGATCAACGCCCTAGGTTTATGTTTGGATGAACTGAC |
| | | ATACGCGTATCCGTCTTAAGAATCTTTTCAAACACTAGTAGTGAA |
| | | ATATATATTAAACTAGTGTTTGAAAAGATTCTTATTACGGTAACG |
| | | CGGAATTCGCAACTATTTTATCAATTTTTTGCGTCGAC |

1. One or more recombinant nucleic acids comprising a nucleic acid sequence at least 15 nucleotides in length complementary to an mRNA encoding human TGF-β Receptor 2 (TGFBR2) comprising the sequence set forth in SEQ ID NO: 2.

2. One or more recombinant nucleic acids comprising a nucleic acid sequence at least 15 nucleotides in length complementary to an mRNA encoding human TGF-β Receptor 1 (TGFBR1) comprising the sequence set forth in SEQ ID NO: 1.

3. One or more recombinant nucleic acids comprising:
a. a first nucleic acid sequence at least 15 nucleotides in length complementary to an mRNA encoding human TGF-β Receptor 2 (TGFBR2) comprising the sequence set forth in SEQ ID NO: 2, and
b. a second nucleic acid sequence at least 15 nucleotides in length complementary an mRNA encoding human TGF-β Receptor 2 (TGFBR2) comprising the sequence set forth in SEQ ID NO: 2.

4. One or more recombinant nucleic acids comprising:
a. a first nucleic acid sequence at least 15 nucleotides in length complementary an mRNA encoding human TGF-β Receptor 2 (TGFBR2) comprising the sequence set forth in SEQ ID NO: 2, and
b. a second nucleic acid sequence at least 15 nucleotides in length complementary to an mRNA encoding human TGF-β Receptor 1 (TGFBR1) comprising the sequence set forth in SEQ ID NO: 1.

5. The one or more recombinant nucleic acids of any one of claims 1 to 4, wherein the nucleic acid sequence is at least 16, 17, 18, 19, 20, 21, or 22 nucleotides in length.

6. The one or more recombinant nucleic acids of any one of claims 1 to 5, wherein the nucleic acid is a short hairpin RNA (shRNA), a small interfering RNA (siRNA), a double stranded RNA (dsRNA), or an antisense oligonucleotide.

7. The one or more recombinant nucleic acids of claim 6, wherein the nucleic acid is an shRNA.

8. The one or more recombinant nucleic acids of any one of claim 1 or 3-7, wherein the nucleic acid or first nucleic acid comprises a sequence selected from the group consisting of the sequences set forth in SEQ ID NOs: 6-84.

9. The one or more recombinant nucleic acids of any one of claims 3-8, wherein the first and second nucleic acids each comprises a sequence selected from the group consisting of the sequences set forth in SEQ ID NOs: 36-84.

10. The one or more recombinant nucleic acids of claim 8 or 9, wherein the nucleic acid or first nucleic acid comprises the sequence set forth in SEQ ID NO: 81.

11. The one or more recombinant nucleic acids of claim 8, 9, or 10, wherein the nucleic acid or first nucleic acid comprises the sequence set forth in SEQ ID NO: 51 or 53.

12. The one or more recombinant nucleic acids of any one of claims 8-11, wherein the nucleic acid, first nucleic acid, or second nucleic acid comprises the sequence set forth in SEQ ID NOs: 81 and 51 or 53.

13. The one or more recombinant nucleic acids of any one of claims 8-11, wherein the nucleic acid comprises the sequence set forth in SEQ ID NO: 128, 129, 130, 139, 140, or 141.

14. The one or more recombinant nucleic acids of any one of claim 1 or 3-13, wherein the nucleic acid, first nucleic acid, or second nucleic acid reduces expression of TGFBR2 in a cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the respective nucleic acid.

15. The one or more recombinant nucleic acids of any one of claim 2, or 4-8, wherein the nucleic acid or second nucleic acid comprises a sequence selected from the group consisting of the sequences set forth in SEQ ID NOs: 6-35.

16. The one or more recombinant nucleic acids of claim 15, wherein the nucleic acid or second nucleic acid comprises the sequence set forth in SEQ ID NO: 16 or 28.

17. The one or more recombinant nucleic acids of any one of claim 2, 4-8, 15, or 16, wherein the nucleic acid or second nucleic acid reduces expression of TGFBR1 in a cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the respective nucleic acid.

18. The one or more recombinant nucleic acids of any one of claim 4-14 or 16-17, wherein the first nucleic acid comprises the sequence set forth in SEQ ID NO: 81 and the second nucleic acid comprises the sequence set forth in SEQ ID NO: 16 or 28.

19. The one or more recombinant nucleic acids of any one of claim 3, 5-8, or 15-18, wherein the first and second nucleic acid reduces expression of TGFBR1 and TGFBR2 in a cell by at least 10%, 15%, 20%, 25%, 30%, 35%, 40%, 45%, 50%, 55%, 60%, 65%, 75%, 80%, 85%, 90%, 95%, or 99%, as compared to a control cell that does not comprise the respective nucleic acid.

20. The one or more recombinant nucleic acids of any one of claims 4-7, wherein the first nucleic acid comprises a sequence selected from the group consisting of the sequences set forth in SEQ ID NOs: 36-84 and the second nucleic acid comprises a sequence selected from the group consisting of the sequences set forth in SEQ ID NOs: 6-84.

21. The one or more recombinant nucleic acids of claim 20, wherein the first nucleic acid comprises the sequence set forth in SEQ ID NO: 81 and the second nucleic acid comprises the sequence set forth in SEQ ID NO: 28, 16, 51, or 53.

22. The one or more recombinant nucleic acids of any one of claims 1-21, wherein the nucleic acid, first nucleic acid, an/or second nucleic acid reduce expression of TFGBR2 in a cell by at least 10%, 15%, 20%, 25%, 30%, 35%, 40%, 45%, 50%, 55%, 60%, 65%, 75%, 80%, 85%, 90%, 95%, or 99% and the second nucleic acid reduces expression of TGFBR1 and/or TGFBR2 in a cell by at least 10%, 15%, 20%, 25%, 30%, 35%, 40%, 45%, 50%, 55%, 60%, 65%, 75%, 80%, 85%, 90%, 95%, or 99%, each as compared to a control cell that does not comprise the respective nucleic acid.

23. The one or more recombinant nucleic acids of any one of claims 1 to 22, further comprising at least a third nucleic acid sequence at least 15 nucleotides in length, wherein the at least a third nucleic acid sequence comprises a nucleic acid sequence complementary to nucleotides 1126 to 1364 of an mRNA encoding human Fas Cell Surface Death Receptor (FAS) comprising the sequence set forth in SEQ ID NO: 3.

24. The one or more recombinant nucleic acids of claim 23, wherein the third nucleic acid comprises a sequence selected from the group consisting of the sequences set forth in SEQ ID NOs: 85-99.

25. The one or more recombinant nucleic acids of claim 23 or 24, wherein the nucleic acid comprises the sequence set forth in SEQ ID NO: 92.

26. The one or more recombinant nucleic acids of any one of claims 23-25, wherein the nucleic acid comprises the sequence set forth in SEQ ID NO: 130, 139, 129, or 141.

27. The one or more recombinant nucleic acids of any one of claims 1 to 22, further comprising at least a third nucleic acid sequence at least 15 nucleotides in length, wherein the third nucleic acid sequence comprises one or more of: (1) a nucleic acid sequence complementary to nucleotides 1126 to 1364 of an mRNA encoding human Fas Cell Surface Death Receptor (FAS) comprising the sequence set forth in SEQ ID NO: 3, (2) a nucleic acid sequence complementary to nucleotides 518 to 559 of an mRNA encoding human Protein Tyrosine Phosphatase Non-Receptor Type 2 (PTPN2) comprising the sequence set forth in SEQ ID NO: 4; or (3) a nucleic acid sequence complementary to nucleotides 1294 to 2141 of an mRNA encoding human Thymocyte Selection Associated High Mobility Group Box (TOX) comprising the sequence set forth in SEQ ID NO: 5.

28. The one or more recombinant nucleic acids of claim 27, wherein the third nucleic acid comprises a nucleic acid sequence complementary to nucleotides 1126 to 1364 of an mRNA encoding human FAS comprising the sequence set forth in SEQ ID NO: 3.

29. The one or more recombinant nucleic acids of claim 27 or 28, wherein the third nucleic acid comprises a sequence selected from the group consisting of the sequences set forth in SEQ ID NOs: 85-99.

30. The one or more recombinant nucleic acids of any one of claims 27-29, wherein the nucleic acid comprises the sequence set forth in SEQ ID NO: 92.

31. The one or more recombinant nucleic acids of claim 27, wherein the third nucleic acid comprises a nucleic acid sequence complementary to nucleotides 518 to 559 of an mRNA encoding human Protein Tyrosine Phosphatase Non-Receptor Type 2 (PTPN2) comprising the sequence set forth in SEQ ID NO: 4.

32. The one or more recombinant nucleic acids of claim 27 or 31, wherein the third nucleic acid comprises a sequence selected from the group consisting of the sequences set forth in SEQ ID NOs: 100-112.

33. The one or more recombinant nucleic acids of any one of claim 27, 31, or 32, wherein the nucleic acid comprises the sequence set forth in SEQ ID NO: 110.

34. The one or more recombinant nucleic acids of any one of claims 27-33, wherein the nucleic acid comprises the sequence set forth in SEQ ID NO: 129.

35. The one or more recombinant nucleic acids of claim 27, wherein the third nucleic acid comprises a nucleic acid sequence complementary to nucleotides 1294 to 2141 of an mRNA encoding human Thymocyte Selection Associated High Mobility Group Box (TOX) comprising the sequence set forth in SEQ ID NO: 5.

36. The one or more recombinant nucleic acids of claim 27 or 35, wherein the third nucleic acid comprises a sequence selected from the group consisting of the sequences set forth in SEQ ID NOs: 113-126.

37. The one or more recombinant nucleic acids of any one of claims 1 to 27, further comprising at least a third and a fourth nucleic acid sequence at least 15 nucleotides in length, wherein the third nucleic acid sequence comprises a nucleic acid sequence complementary to nucleotides 1126 to 1364 of an mRNA encoding human Fas Cell Surface Death Receptor (FAS) comprising the sequence set forth in SEQ ID NO: 3, and the fourth nucleic acid sequence complementary to nucleotides 518 to 559 of an mRNA encoding human Protein Tyrosine Phosphatase Non-Receptor Type 2 (PTPN2) comprising the sequence set forth in SEQ ID NO: 4.

38. The one or more recombinant nucleic acids of claim 27 or 28, wherein the third nucleic acid comprises a sequence selected from the group consisting of the sequences set forth in SEQ ID NOs: 85-99.

39. The one or more recombinant nucleic acids of any one of claims 27-29, wherein the nucleic acid comprises the sequence set forth in SEQ ID NO: 92.

40. The one or more recombinant nucleic acids of claim 27 or 31, wherein the third nucleic acid comprises a sequence selected from the group consisting of the sequences set forth in SEQ ID NOs: 100-112.

41. The one or more recombinant nucleic acids of any one of claim 27, 31, or 32, wherein the nucleic acid comprises the sequence set forth in SEQ ID NO: 110.

42. The one or more recombinant nucleic acids of any one of claims 27-41, wherein the third nucleic acid reduces expression of FAS in a cell by at least 10%, 15%, 20%, 25%, 30%, 35%, 40%, 45%, 50%, 55%, 60%, 65%, 75%, 80%, 85%, 90%, 95%, or 99% and/or reduces expression of PTPN2 in a cell by at least 10%, 15%, 20%, 25%, 30%, 35%, 40%, 45%, 50%, 55%, 60%, 65%, 75%, 80%, 85%, 90%, 95%, or 99%, as compared to a control cell that does not comprise the nucleic acid.

43. The one or more recombinant nucleic acids of any one of claims 1 to 42, wherein each of the one or more nucleic acids are encoded on a plurality of different nucleic acid molecules.

44. The one or more recombinant nucleic acids of any one of claims 1 to 42, wherein each of the one or more nucleic acids are encoded on the same nucleic acid molecule.

45. The one or more recombinant nucleic acids of any one of claims 1 to 44, wherein the one or more recombinant nucleic acids are incorporated into a one or more expression cassettes or expression vectors.

46. The one or more recombinant nucleic acids of claim 45, wherein the expression cassette or the expression vector further comprises a constitutive promoter upstream of the one or more recombinant nucleic acids.

47. The one or more recombinant nucleic acids of claim 45 or 46, wherein the expression cassette comprises the sequence set forth in any one of SEQ ID NOs: 133-137.

48. An expression vector comprising the recombinant nucleic acid(s) of any one of claims 1-47.

49. An immune cell comprising one or more recombinant nucleic acids at least 15 nucleotides in length complementary to an mRNA encoding human TGFBR1 comprising the sequence set forth in SEQ ID NO: 1.

50. An immune cell comprising one or more recombinant nucleic acids at least 15 nucleotides in length complementary to an mRNA encoding human TGFBR2 comprising the sequence set forth in SEQ ID NO: 2.

51. An immune cell comprising one or more recombinant nucleic acids comprising: a first nucleic acid sequence at least 15 nucleotides in length complementary to of an mRNA encoding human TGFBR2 comprising the sequence set forth in SEQ ID NO: 2, and a second nucleic acid sequence at least 15 nucleotides in length, wherein the second nucleic acid sequence is
a. complementary to an mRNA encoding human TGFBR2 comprising the sequence set forth in SEQ ID NO: 2; or
b. complementary to an mRNA encoding human TGFBR1 comprising the sequence set forth in SEQ ID NO: 1.

52. The immune cell of claim 51, wherein the second nucleic acid sequence is complementary to an mRNA encoding human TGFBR2 comprising the sequence set forth in SEQ ID NO: 2.

53. The immune cell of claim 51, wherein the second nucleic acid sequence is complementary to an mRNA encoding human TGFBR1 comprising the sequence set forth in SEQ ID NO: 1.

54. An immune cell comprising one or more recombinant nucleic acids comprising: a first nucleic acid sequence at least 15 nucleotides in length complementary to an mRNA encoding human TGFBR2 comprising the sequence set forth in SEQ ID NO: 2, and a second nucleic acid sequence at least 15 nucleotides in length complementary to an mRNA encoding human TGFBR 1 comprising the sequence set forth in SEQ ID NO: 1.

55. An immune cell comprising one or more recombinant nucleic acids comprising: a first nucleic acid sequence and a second nucleic acid sequence at least 15 nucleotides in length complementary to an mRNA encoding human TGFBR2 comprising the sequence set forth in SEQ ID NO: 2.

56. The immune cell of any one of claims 49 to 55, wherein the cell further comprises at least a third nucleic acid sequence at least 15 nucleotides in length, wherein the third nucleic acid sequence is complementary to nucleotides 1126 to 1364 of an mRNA encoding human FAS comprising the sequence set forth in SEQ ID NO: 3.

57. The immune cell of any one of claims 49 to 55, wherein the cell further comprises at least a third nucleic acid sequence at least 15 nucleotides in length, wherein the third nucleic acid sequence is (1) complementary to nucleotides 1126 to 1364 of an mRNA encoding human FAS comprising the sequence set forth in SEQ ID NO: 3; (2) complementary to nucleotides 518 to 559 of an mRNA encoding human PTPN2 comprising the sequence set forth in SEQ ID NO: 4; or (3) complementary to nucleotides 1294 to 2141 of an mRNA encoding human Thymocyte Selection Associated High Mobility Group Box (TOX) comprising the sequence set forth in SEQ ID NO: 5.

58. The immune cell of any one of claims 49 to 57, wherein the cell further comprises at least a fourth nucleic acid sequence at least 15 nucleotides in length, wherein the fourth nucleic acid sequence is (1) complementary to nucleotides 1126 to 1364 of an mRNA encoding human FAS comprising the sequence set forth in SEQ ID NO: 3; (2) complementary to nucleotides 518 to 559 of an mRNA encoding human PTPN2 comprising the sequence set forth in SEQ ID NO: 4; or (3) complementary to nucleotides 1294 to 2141 of an mRNA encoding human Thymocyte Selection Associated High Mobility Group Box (TOX) comprising the sequence set forth in SEQ ID NO: 5.

59. The immune cell of any one of claims 49-58, wherein the first, second, third, and fourth nucleic acids are an shRNA, an siRNA, a dsRNA, or an antisense oligonucleotide.

60. The immune cell of claim 59, wherein the first, second, third, and fourth nucleic acids are shRNA.

61. The immune cell of any one of claims 51-60, wherein the first nucleic acid comprises a sequence selected from the group consisting of the sequences set forth in SEQ ID NOs: 36-84.

62. The immune cell of claim 61, wherein the first nucleic acid comprises the sequence set forth in SEQ ID NO: 81.

63. The immune cell of any one of claims 51-62, wherein the second nucleic acid comprises a sequence selected from the group consisting of the sequences set forth in SEQ ID NOs: 6-35.

64. The immune cell of claim 63, wherein the second nucleic acid comprises the sequence set forth in SEQ ID NO: 16 or 28.

65. The immune cell of any one of claims 51-64, wherein the first nucleic acid comprises a sequence selected from the group consisting of the sequences set forth in SEQ ID NOs: 36-84; and the second nucleic acid sequence comprises a sequence selected from the group consisting of the sequences set forth in SEQ ID NOs: 6-35.

66. The immune cell of any one of claims 51-65, wherein the first nucleic acid comprises the sequence set forth in SEQ ID NO: 81; and the second nucleic acid comprises the sequence set forth in SEQ ID NO: 16 or 28.

67. The immune cell of any one of claims 51-62, wherein the second nucleic acid comprises a sequence selected from the group consisting of the sequences set forth in SEQ ID NOs: 36-84.

68. The immune cell of claim 67, wherein the second nucleic acid comprises the sequence set forth in SEQ ID NO: 51 or 53.

69. The immune cell of any one of claim 51-62 or 67-68, wherein the first nucleic acid and second nucleic acids comprises a sequence selected from the group consisting of the sequences set forth in SEQ ID NOs: 36-84.

70. The immune cell of any one of claim 51-62 or 67-69, wherein the first nucleic acid comprises the sequence set forth in SEQ ID NO: 81; and the second nucleic acid comprises the sequence set forth in SEQ ID NO: 51 or 53.

71. The immune cell of any one of claim 51-62 or 67-70, wherein the one or more recombinant nucleic acids comprises the sequence set forth in SEQ ID NO: 128, 129, 130, 139, 140, or 141.

72. The immune cell of any one of claims 51-71 wherein the first nucleic acid reduces expression of TGFBR2 in a cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the first nucleic acid.

73. The immune cell of claim 72, wherein expression of TGFBR2 in a cell is reduced by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the first nucleic acid.

74. The immune cell of any one of claims 51-66, wherein the second nucleic acid reduces expression of TGFBR1 in a cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the second nucleic acid.

75. The immune cell of claim 74, wherein expression of TGFBR1 in a cell is reduced by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the second nucleic acid.

76. The immune cell of any one of claims 51-75, wherein the first nucleic acid reduces expression of TFGBR2 in a cell by at least 10%, 15%, 20%, 25%, 30%, 35%, 40%, 45%, 50%, 55%, 60%, 65%, 75%, 80%, 85%, 90%, 95%, or 99% and the second nucleic acid reduces expression of TGFBR1 in a cell by at least 10%, 15%, 20%, 25%, 30%, 35%, 40%, 45%, 50%, 55%, 60%, 65%, 75%, 80%, 85%, 90%, 95%, or 99%, each as compared to a control cell that does not comprise the respective nucleic acid.

77. The immune cell of any one of claims 56-76, wherein the third nucleic acid sequence is complementary to nucleotides 1126 to 1364 of an mRNA encoding human FAS comprising the sequence set forth in SEQ ID NO: 3.

78. The immune cell of claim 77, wherein the third nucleic acid comprises a sequence selected from the group consisting of the sequences set forth in SEQ ID NOs: 85-99.

79. The immune cell of claim 78, wherein the third nucleic acid comprises the sequence set forth in SEQ ID NO: 92.

80. The immune cell of any one of claims 56-79, wherein the one or more recombinant nucleic acids comprises the sequence set forth in SEQ ID NO: 129, 130, 139, or 141.

81. The immune cell of any one of claims 56-80 wherein the third nucleic acid reduces expression of FAS in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the third nucleic acid.

82. The immune cell of any one of claims 58-78, wherein the fourth nucleic acid sequence is complementary to nucleotides 518 to 559 of an mRNA encoding human PTPN2 comprising the sequence set forth in SEQ ID NO: 4.

83. The immune cell of claim 77, wherein the fourth nucleic acid comprises a sequence selected from the group consisting of the sequences set forth in SEQ ID NOs: 100-112.

84. The immune cell of claim 83, wherein the fourth nucleic acid comprises the sequence set forth in SEQ ID NO: 110.

85. The immune cell of any one of claims 58-84, wherein the one or more recombinant nucleic acids comprises the sequence set forth in SEQ ID NO: 129.

86. The immune cell of any one of claims 57-85 wherein the fourth nucleic acid reduces expression of PTPN2 in the immune cell by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the first nucleic acid.

87. The immune cell of any one of claims 58-78, wherein the fourth nucleic acid sequence is complementary to nucleotides 1294 to 2141 of an mRNA encoding human Thymocyte Selection Associated High Mobility Group Box (TOX) comprising the sequence set forth in SEQ ID NO: 5.

88. The immune cell of claim 87, wherein the fourth nucleic acid comprises a sequence selected from the group consisting of the sequences set forth in SEQ ID NOs: 113-126.

89. The immune cell of any one of claims 49-88, wherein expression of TGFBR1, TGFBR2, FAS and/or PTPN2 and/or TOX is determined by a nucleic acid assay or a protein assay.

90. The immune cell of claim 89, wherein the nucleic acid assay comprises at least one of polymerase chain reaction (PCR), quantitative PCR (qPCR), RT-qPCR, microarray, gene array, or RNAseq.

91. The immune cell of claim 89, wherein the protein assay comprises at least one of immunoblotting, fluorescence activated cell sorting, flow-cytometry, magnetic-activated cell sorting, or affinity-based cell separation.

92. The immune cell of any one of claims 49 to 91, wherein the immune cell is a primary human immune cell.

93. The immune cell of any one of claims 49-92, wherein the primary immune cell is a natural killer (NK) cell, a natural killer T (NKT) cell, a T cell, a γδ T cell, a CD8+ T cell, a CD4+ T cell, a primary T cell, a T cell progenitor, or an induced pluripotent stem cell (iPSC).

94. The immune cell of any one of claims 49-93, wherein the primary immune cell is a primary T cell.

95. The immune cell of any one of claims 49-94, wherein the primary immune cell is a primary human T cell.

96. The immune cell of any one of claims 49-95, wherein the immune cell is an autologous immune cell.

97. The immune cell of any one of claims 49-95, wherein the immune cell is an allogeneic immune cell.

98. A population of cells comprising a plurality of immune cells of any one of claims 49-97.

99. A pharmaceutical composition comprising the immune cell of any one of claims 49-97 or the population of cells of claim 98, and a pharmaceutically acceptable excipient.

100. A pharmaceutical composition comprising the one or more recombinant nucleic acids of any one of claims 1 to 47 or the vector of claim 48, and a pharmaceutically acceptable excipient.

101. A method of treating a disease in a subject comprising administering the immune cell(s) of any one of claims 51-98 or the pharmaceutical composition of claim 99 or 100 to the subject.

102. The method of claim 101, wherein the disease is cancer.

103. The method of claim 102, wherein the cancer is a solid cancer or a liquid cancer.

104. The method of claim 102 or 103, wherein the cancer is ovarian cancer, fallopian cancer, primary peritoneal cancer, uterine cancer, mesothelioma, cervical cancer, pancreatic, kidney cancer, lung cancer, prostate cancer, bladder cancer, breast cancer, brain cancer, leukemia, or lymphoma.

105. The method of any one of claims 101-104, wherein the administration of the cell(s) enhances an immune response.

106. The method of claim 105, wherein the enhanced immune response is an adaptive immune response.

107. The method of claim 105, wherein the enhanced immune response is an innate immune response.

108. A method of enhancing an immune response in a subject comprising administering the immune cell(s) of any one of claims 51-98 or the pharmaceutical composition of claim 99 or 100 to the subject.

109. The method of claim 108, wherein the enhanced immune response is an adaptive immune response.

110. The method of claim 109, wherein the enhanced immune response is an innate immune response.

111. The method of any one of claims 101-112, wherein expression of TGFBR2 in the immune cell is reduced by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the respective nucleic acid.

112. The method of any one of claims 101-110, wherein expression of TGFBR1 in the immune cell is reduced by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the respective nucleic acid.

113. The method of any one of claims 101-112, wherein expression of FAS in the immune cell is reduced by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the respective nucleic acid.

114. The method of any one of claims 101-112, wherein expression of PTPN2 in the immune cell is reduced by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the respective nucleic acid.

115. The method of any one of claims 101-112, wherein expression of TOX in the immune cell is reduced by at least 10%, 15%, 20%, 25%, 30%, 35%, 40%, 45%, 50%, 55%, 60%, 65%, 75%, 80%, 85%, 90%, 95%, or 99% as compared to a control cell that does not comprise the respective nucleic acid.

116. The method of any one of claims 112-115, wherein expression of TGFBR1, TGFBR2, FAS, PTPN2, and/or TOX in the immune cell is determined by a nucleic acid assay or a protein assay.

117. The method of claim 116, wherein the nucleic acid assay comprises at least one of polymerase chain reaction (PCR), quantitative PCR (qPCR), RT-qPCR, microarray, gene array, or RNAseq.

118. The method of claim 116, wherein the protein assay comprises at least one of immunoblotting, fluorescence activated cell sorting, flow-cytometry, magnetic-activated cell sorting, or affinity-based cell separation.
